SEQUENCE LISTING (200 sequences)

SEQ ID NO: 1 - protein, 675 aa, Streptococcus agalactiae
MKKINKYFAVFSALLLTVTSLFSVAPVFAEEAKTTDTVTLHKIVMPRTAFDGFTAGTKGKDNTDYVGKQIEDLKTYFGSGEAKEIAGACFAFKNEAGTKYITENGEEVDTLDTTDAKGCAVLKGLTTDNGFKFNTSKLTGTYQIVELKEKSTYNNDGSILADSKAVPVKITLPLVNDNGVVKDAHVYPKNTETKPQVDKNFADKELDYANNKKDKGTVSASVGDVKKYHVGTKILKGSDYKKLIWTDSMTKGLTFNNDIAVTLDGATLDATNYKLVADDQGFRLVLTDKGLEAVAKAAKTKDVEIKITYSATLNGSAVVEVLETNDVKLDYGNNPTIENEPKEGIPVDKKITVNKTWAVDGNEVNKADETVDAVFTLQVKDGDKWVNVDSAKATAATSFKHTFENLDNAKTYRVIERVSGYAPEYVSFVNGVVTIKNNKDSNEPTPINPSEPKVVTYGRKFVKTNKDGKERLAGATFLVKKDGKYLARKSGVATDAEKAAVDSTKSALDAAVKAYNDLTKEKQEGQDGKSALATVSEKQKAYNDAFVKANYSYEWVEDKNAKNVVKLISNDKGQFEITGLTEGQYSLEETQAPTGYAKLSGDVSFNVNATSYSKGSAQDIEYTQGSKTKDAQQVINKKVTIPQTGGIGTIFFTIIGLSIMLGAVVIMKRRQSEEV

SEQ ID NO: 2 - protein, 116 aa, Streptococcus agalactiae
GNNPTIENEPKEGIPVDKKITVNKTWAVDGNEVNKADETVDAVFTLQVKDGDKWVNVDSAKATAATSFKHTFENLDNAKTYRVIERVSGYAPEYVSFVNGVVTIKNNKDSNEPTPI

SEQ ID NO: 3 - protein, 135 aa, Streptococcus agalactiae
MGSSHHHHHHENLYFQGASGNNPTIENEPKEGIPVDKKITVNKTWAVDGNEVNKADETVDAVFTLQVKDGDKWVNVDSAKATAATSFKHTFENLDNAKTYRVIERVSGYAPEYVSFVNGVVTIKNNKDSNEPTPI

SEQ ID NO: 4 - protein, 13 aa, Streptococcus agalactiae
DGNEVNKADETVD

SEQ ID NO: 5 - protein, 5 aa, Streptococcus agalactiae
KDGDK

SEQ ID NO: 6 - protein, 7 aa, Streptococcus agalactiae
ATAATSF

SEQ ID NO: 7 - protein, 8 aa, Streptococcus agalactiae
ENLDNAKT

SEQ ID NO: 8 - protein, 5 aa, Streptococcus agalactiae
VSGYA

SEQ ID NO: 9 - protein, 4 aa, Streptococcus agalactiae
VNGV

SEQ ID NO: 10 - protein, 28 aa, Streptococcus agalactiae
GNNPTIENEPKEGIPVDKKITVNKTWAV

SEQ ID NO: 11 - protein, 7 aa, Streptococcus agalactiae
AVFTLQV

SEQ ID NO: 12 - protein, 8 aa, Streptococcus agalactiae
WVNVDSAK

SEQ ID NO: 13 - protein, 6 aa, Streptococcus agalactiae
YRVIER

SEQ ID NO: 14 - protein, 4 aa, Streptococcus agalactiae
KHTF

SEQ ID NO: 15 - protein, 6 aa, Streptococcus agalactiae
PEYVSF

SEQ ID NO: 16 - protein, 15 aa, Streptococcus agalactiae
VTIKNNKDSNEPTPI

SEQ ID NO: 17 - protein, 13 aa, synthetic construct
SVEHTDGKVSKVE

SEQ ID NO: 18 - protein, 15 aa, synthetic construct
GASVAGTNTGWGNKQ

SEQ ID NO: 19 - protein, 34 aa, synthetic construct
SPLKNTGANVNAWESGKFTGNVLEISGMAQREHR

SEQ ID NO: 20 - protein, 11 aa, synthetic construct
PKDNSGSNGES

SEQ ID NO: 21 - protein, 24 aa, synthetic construct
QRYGEGTKKIEYDGQTYSIPSLFV

SEQ ID NO: 22 - protein, 17 aa, synthetic construct
AKLYGAMSGNSHNSQTE

SEQ ID NO: 23 - protein, 17 aa, synthetic construct
GFKGTVDSANHDNTYDQ

SEQ ID NO: 24 - protein, 14 aa, synthetic construct
QEGKGADKIVSTAS

SEQ ID NO: 25 - protein, 47 aa, synthetic construct
YARYRKAAERITHDYPEPTGAKKGKISTVSDYFRNIRTHSIHAVSSL

SEQ ID NO: 26 - protein, 49 aa, synthetic construct
YARYRKWNNSKYSVNTKKVNENKGEKINVTQYLKAENQENGTFHAVSSL

SEQ ID NO: 27 - protein, 16 aa, synthetic construct
YLQSGKPSPIVRGSTL

SEQ ID NO: 28 - protein, 50 aa, synthetic construct
YARYRKAAERITHDYPEPTAPGKNKISTVSDYFRNIRTHSIHPRHAVSSL

SEQ ID NO: 29 - protein, 12 aa, synthetic construct
AYPSDADAAVTV

SEQ ID NO: 30 - protein, 45 aa, synthetic construct
YARYRKWNDNKYSVDIKELENKNQNKRDLKTENQENGSFHAVSSL

SEQ ID NO: 31 - protein, 15 aa, synthetic construct
HNWGRLENTRFKTHE

SEQ ID NO: 32 - protein, 15 aa, synthetic construct
HYWGRLENTRFKTHE

SEQ ID NO: 33 - protein, 50 aa, synthetic construct
YARYRKAYAYEHITRDYPDAAGANQGKKISTVSDYFKNIRTHSIHAVSSL

SEQ ID NO: 34 - protein, 14 aa, synthetic construct
TTEFLTAAGQDGGA

SEQ ID NO: 35 - protein, 53 aa, synthetic construct
YARYRKWHNNKYSVNIKELERKNNKTFGGNQLNIKYQKTEHQENGTFHAVSSL

SEQ ID NO: 36 - protein, 49 aa, synthetic construct
YARYRKAYEHITRDYPDAAGANQGKKISTVSDYFKNIRTRSVHHAVSSL

SEQ ID NO: 37 - protein, 53 aa, synthetic construct
YARYRKWHNNKYSVNIKELGRNDNSASDSKHLNIKTQKTEHQENGTFHAVSSL

SEQ ID NO: 38 - protein, 15 aa, synthetic construct
YPSDGSAKTSVPSEM

SEQ ID NO: 39 - protein, 49 aa, synthetic construct
YARYRKAAERITHDYPEPTGAKKDKKISTVSDYFRNIRTHSVHHAVSSL

SEQ ID NO: 40 - protein, 49 aa, synthetic construct
YARYRKWNNSKYSVNIKRVKENNGSGKKLTQDLKTENQENGTFHAVSSL

SEQ ID NO: 41 - protein, 12 aa, synthetic construct
VITAPPTTSDGA

SEQ ID NO: 42 - protein, 48 aa, synthetic construct
YARYRKAYEHITRDYPDAAGANKGKISTVSDYFRNIRTHSIHHAVSSL

SEQ ID NO: 43 - protein, 19 aa, synthetic construct
HSAGTKPTYYDDIDSGKTK

SEQ ID NO: 44 - protein, 53 aa, synthetic construct
YARYRKWNDNKYSVNIKELGRKDGTSSSGRYLNIQTRKTENQENGTFHAVSSL

SEQ ID NO: 45 - protein, 44 aa, synthetic construct
RPGRNPKTGDVVPVSARRVVGPSLFSLHHRQPRLGRNPKTGDSV

SEQ ID NO: 46 - protein, 50 aa, synthetic construct
LFMKTRNGSMKAADNFLDPNKASSLLSSGFSPDFATVITMDRKASKQQTN

SEQ ID NO: 47 - protein, 23 aa, synthetic construct
NWGPYDRDSWNPVYGNQLFMKTR

SEQ ID NO: 48 - protein, 31 aa, synthetic construct
NWGPYDRDSWNPVYGNQLFMKTRGSTNTKDK

SEQ ID NO: 49 - protein, 20 aa, synthetic construct
GQAEPDRAHYNIVTFCCKCD

SEQ ID NO: 50 - protein, 35 aa, synthetic construct
TGSQAIASPGNKRGFQENPFDYFNDEFFNRFFGLP

SEQ ID NO: 51 - protein, 20 aa, synthetic construct
NKRGFQENPFDYFNDEFFNR

SEQ ID NO: 52 - protein, 28 aa, synthetic construct
KYIKFKHDYNILEFNDGTFEYGARPQFN

SEQ ID NO: 53 - protein, 27 aa, synthetic construct
ETNAYNVTTHANGQVSYGARPTYKKPS

SEQ ID NO: 54 - protein, 145 aa, synthetic construct
MGSSHHHHHHENLYFQGASGNNPTIENEPKEGIPVDKKITVNKTWAVDGNEVNKADETVDAVFTLQVKSVEHTDGKVSKVEKWVNVDSAKATAATSFKHTFENLDNAKTYRVIERVSGYAPEYVSFVNGVVTIKNNKDSNEPTPI

SEQ ID NO: 55 - protein, 147 aa, synthetic construct
MGSSHHHHHHENLYFQGASGNNPTIENEPKEGIPVDKKITVNKTWAVDGNEVNKADETVDAVFTLQVKGASVAGTNTGWGNKQKWVNVDSAKATAATSFKHTFENLDNAKTYRVIERVSGYAPEYVSFVNGVVTIKNNKDSNEPTPI

SEQ ID NO: 56 - protein, 166 aa, synthetic construct
MGSSHHHHHHENLYFQGASGNNPTIENEPKEGIPVDKKITVNKTWAVDGNEVNKADETVDAVFTLQVKSPLKNTGANVNAWESGKFTGNVLEISGMAQREHRKWVNVDSAKATAATSFKHTFENLDNAKTYRVIERVSGYAPEYVSFVNGVVTIKNNKDSNEPTPI

SEQ ID NO: 57 - protein, 143 aa, synthetic construct
MGSSHHHHHHENLYFQGASGNNPTIENEPKEGIPVDKKITVNKTWAVDGNEVNKADETVDAVFTLQVKPKDNSGSNGESKWVNVDSAKATAATSFKHTFENLDNAKTYRVIERVSGYAPEYVSFVNGVVTIKNNKDSNEPTPI

SEQ ID NO: 58 - protein, 156 aa, synthetic construct
MGSSHHHHHHENLYFQGASGNNPTIENEPKEGIPVDKKITVNKTWAVDGNEVNKADETVDAVFTLQVKQRYGEGTKKIEYDGQTYSIPSLFVKWVNVDSAKATAATSFKHTFENLDNAKTYRVIERVSGYAPEYVSFVNGVVTIKNNKDSNEPTPI

SEQ ID NO: 59 - protein, 150 aa, synthetic construct
MGSSHHHHHHENLYFQGASGNNPTIENEPKEGIPVDKKITVNKTWAVDGNEVNKADETVDAVFTLQVKDAKLYGAMSGNSHNSQTEKWVNVDSAKATAATSFKHTFENLDNAKTYRVIERVSGYAPEYVSFVNGVVTIKNNKDSNEPTPI

SEQ ID NO: 60 - protein, 149 aa, synthetic construct
MGSSHHHHHHENLYFQGASGNNPTIENEPKEGIPVDKKITVNKTWAVDGNEVNKADETVDAVFTLQVKGFKGTVDSANHDNTYDQKWVNVDSAKATAATSFKHTFENLDNAKTYRVIERVSGYAPEYVSFVNGVVTIKNNKDSNEPTPI

SEQ ID NO: 61 - protein, 146 aa, synthetic construct
MGSSHHHHHHENLYFQGASGNNPTIENEPKEGIPVDKKITVNKTWAVDGNEVNKADETVDAVFTLQVKQEGKGADKIVSTASKWVNVDSAKATAATSFKHTFENLDNAKTYRVIERVSGYAPEYVSFVNGVVTIKNNKDSNEPTPI

SEQ ID NO: 62 - protein, 158 aa, synthetic construct
MGSSHHHHHHENLYFQGASGNNPTIENEPKEGIPVDKKITVNKTWAVDSPLKNTGANVNAWESGKFTGNVLEISGMAQREHRDAVFTLQVKDGDKWVNVDSAKATAATSFKHTFENLDNAKTYRVIERVSGYAPEYVSFVNGVVTIKNNKDSNEPTPI

SEQ ID NO: 63 - protein, 162 aa, synthetic construct
MGSSHHHHHHENLYFQGASGNNPTIENEPKEGIPVDKKITVNKTWAVDGNEVNKADETVDAVFTLQVKDGDKWVNVDSAKSPLKNTGANVNAWESGKFTGNVLEISGMAQREHRKHTFENLDNAKTYRVIERVSGYAPEYVSFVNGVVTIKNNKDSNEPTPI

SEQ ID NO: 64 - protein, 160 aa, synthetic construct
MGSSHHHHHHENLYFQGASGNNPTIENEPKEGIPVDKKITVNKTWAVDGNEVNKADETVDAVFTLQVKDGDKWVNVDSAKATAATSFKHTFSPLKNTGANVNAWESGKFTGNVLEISGMAQREHRRVIERVSGYAPEYVSFVNGVVTIKNNKDSNEPTPI

SEQ ID NO: 65 - protein, 163 aa, synthetic construct
MGSSHHHHHHENLYFQGASGNNPTIENEPKEGIPVDKKITVNKTWAVDGNEVNKADETVDAVFTLQVKDGDKWVNVDSAKATAATSFKHTFENLDNAKTYRVIESPLKNTGANVNAWESGKFTGNVLEISGMAQREHRPEYVSFVNGVVTIKNNKDSNEPTPI

SEQ ID NO: 66 - protein, 167 aa, synthetic construct
MGSSHHHHHHENLYFQGASGNNPTIENEPKEGIPVDKKITVNKTWAVDGNEVNKADETVDAVFTLQVKDGDKWVNVDSAKATAATSFKHTFENLDNAKTYRVIERVSGYAPEYVSFVSPLKNTGANVNAWESGKFTGNVLEISGMAQREHRVVTIKNNKDSNEPTPI

SEQ ID NO: 67 - protein, 179 aa, synthetic construct
MGSSHHHHHHENLYFQGASGNNPTIENEPKEGIPVDKKITVNKTWAVDGNEVNKADETVDAVFTLQVKYARYRKAAERITHDYPEPTGAKKGKISTVSDYFRNIRTHSIHAVSSLKWVNVDSAKATAATSFKHTFENLDNAKTYRVIERVSGYAPEYVSFVNGVVTIKNNKDSNEPTPI

SEQ ID NO: 68 - protein, 180 aa, synthetic construct
MGSSHHHHHHENLYFQASGNNPTIENEPKEGIPVDKKITVNKTWAVDGNEVNKADETVDAVFTLQVKYARYRKWNNSKYSVNTKKVNENKGEKINVTQYLKAENQENGTFHAVSSLKWVNVDSAKATAATSFKHTFENLDNAKTYRVIERVSGYAPEYVSFVNGVVTIKNNKDSNEPTPI

SEQ ID NO: 69 - protein, 148 aa, synthetic construct
MGSSHHHHHHENLYFQGASGNNPTIENEPKEGIPVDKKITVNKTWAVDGNEVNKADETVDAVFTLQVKYLQSGKPSPIVRGSTLKWVNVDSAKATAATSFKHTFENLDNAKTYRVIERVSGYAPEYVSFVNGVVTIKNNKDSNEPTPI

SEQ ID NO: 70 - protein, 181 aa, synthetic construct
MGSSHHHHHHENLYFQASGNNPTIENEPKEGIPVDKKITVNKTWAVDGNEVNKADETVDAVFTLQVKYARYRKAAERITHDYPEPTAPGKNKISTVSDYFRNIRTHSIHPRHAVSSLKWVNVDSAKATAATSFKHTFENLDNAKTYRVIERVSGYAPEYVSFVNGVVTIKNNKDSNEPTPI

SEQ ID NO: 71 - protein, 154 aa, synthetic construct
MGSSHHHHHHENLYFQGLYFQGLYFQGASGNNPTIENEPKEGIPVDKKITVNKTWAVDGNEVNKADETVDAVFTLQVKAYPSDADAAVTVKWVNVDSAKATAATSFKHTFENLDNAKTYRVIERVSGYAPEYVSFVNGVVTIKNNKDSNEPTPI

SEQ ID NO: 72 - protein, 187 aa, synthetic construct
MGSSHHHHHHENLYFQGLYFQGLYFQGASGNNPTIENEPKEGIPVDKKITVNKTWAVDGNEVNKADETVDAVFTLQVKYARYRKWNDNKYSVDIKELENKNQNKRDLKTENQENGSFHAVSSLKWVNVDSAKATAATSFKHTFENLDNAKTYRVIERVSGYAPEYVSFVNGVVTIKNNKDSNEPTPI

SEQ ID NO: 73 - protein, 157 aa, synthetic construct
MGSSHHHHHHENLYFQGLYFQGLYFQGASGNNPTIENEPKEGIPVDKKITVNKTWAVDGNEVNKADETVDAVFTLQVKHNWGRLENTRFKTHEKWVNVDSAKATAATSFKHTFENLDNAKTYRVIERVSGYAPEYVSFVNGVVTIKNNKDSNEPTPI

SEQ ID NO: 74 - protein, 157 aa, synthetic construct
MGSSHHHHHHENLYFQGLYFQGLYFQGASGNNPTIENEPKEGIPVDKKITVNKTWAVDGNEVNKADETVDAVFTLQVKHYWGRLENTRFKTHEKWVNVDSAKATAATSFKHTFENLDNAKTYRVIERVSGYAPEYVSFVNGVVTIKNNKDSNEPTPI

SEQ ID NO: 75 - protein, 182 aa, synthetic construct
MGSSHHHHHHENLYFQGASGNNPTIENEPKEGIPVDKKITVNKTWAVDGNEVNKADETVDAVFTLQVKYARYRKAYAYEHITRDYPDAAGANQGKKISTVSDYFKNIRTHSIHAVSSLKWVNVDSAKATAATSFKHTFENLDNAKTYRVIERVSGYAPEYVSFVNGVVTIKNNKDSNEPTPI

SEQ ID NO: 76 - protein, 176 aa, synthetic construct
MGSSHHHHHHENLYFQASGNNPTIENEPKEGIPVDKKITVNKTWAVDGNEVNKADETVDAVFTLQVKYARYRKWNDNKYSVDIKELENKNQNKRDLKTENQENGSFHAVSSLKWVNVDSAKATAATSFKHTFENLDNAKTYRVIERVSGYAPEYVSFVNGVVTIKNNKDSNEPTPI

SEQ ID NO: 77 - protein, 146 aa, synthetic construct
MGSSHHHHHHENLYFQGASGNNPTIENEPKEGIPVDKKITVNKTWAVDGNEVNKADETVDAVFTLQVKTTEFLTAAGQDGGAKWVNVDSAKATAATSFKHTFENLDNAKTYRVIERVSGYAPEYVSFVNGVVTIKNNKDSNEPTPI

SEQ ID NO: 78 - protein, 184 aa, synthetic construct
MGSSHHHHHHENLYFQASGNNPTIENEPKEGIPVDKKITVNKTWAVDGNEVNKADETVDAVFTLQVKYARYRKWHNNKYSVNIKELERKNNKTFGGNQLNIKYQKTEHQENGTFHAVSSLKWVNVDSAKATAATSFKHTFENLDNAKTYRVIERVSGYAPEYVSFVNGVVTIKNNKDSNEPTPI

SEQ ID NO: 79 - protein, 181 aa, synthetic construct
MGSSHHHHHHENLYFQGASGNNPTIENEPKEGIPVDKKITVNKTWAVDGNEVNKADETVDAVFTLQVKYARYRKAYEHITRDYPDAAGANQGKKISTVSDYFKNIRTRSVHHAVSSLKWVNVDSAKATAATSFKHTFENLDNAKTYRVIERVSGYAPEYVSFVNGVVTIKNNKDSNEPTPI

SEQ ID NO: 80 - protein, 184 aa, synthetic construct
MGSSHHHHHHENLYFQASGNNPTIENEPKEGIPVDKKITVNKTWAVDGNEVNKADETVDAVFTLQVKYARYRKWHNNKYSVNIKELGRNDNSASDSKHLNIKTQKTEHQENGTFHAVSSLKWVNVDSAKATAATSFKHTFENLDNAKTYRVIERVSGYAPEYVSFVNGVVTIKNNKDSNEPTPI

SEQ ID NO: 81 - protein, 147 aa, synthetic construct
MGSSHHHHHHENLYFQGASGNNPTIENEPKEGIPVDKKITVNKTWAVDGNEVNKADETVDAVFTLQVKYPSDGSAKTSVPSEMKWVNVDSAKATAATSFKHTFENLDNAKTYRVIERVSGYAPEYVSFVNGVVTIKNNKDSNEPTPI

SEQ ID NO: 82 - protein, 181 aa, synthetic construct
MGSSHHHHHHENLYFQGASGNNPTIENEPKEGIPVDKKITVNKTWAVDGNEVNKADETVDAVFTLQVKYARYRKAAERITHDYPEPTGAKKDKKISTVSDYFRNIRTHSVHHAVSSLKWVNVDSAKATAATSFKHTFENLDNAKTYRVIERVSGYAPEYVSFVNGVVTIKNNKDSNEPTPI

SEQ ID NO: 83 - protein, 180 aa, synthetic construct
MGSSHHHHHHENLYFQASGNNPTIENEPKEGIPVDKKITVNKTWAVDGNEVNKADETVDAVFTLQVKYARYRKWNNSKYSVNIKRVKENNGSGKKLTQDLKTENQENGTFHAVSSLKWVNVDSAKATAATSFKHTFENLDNAKTYRVIERVSGYAPEYVSFVNGVVTIKNNKDSNEPTPI

SEQ ID NO: 84 - protein, 144 aa, synthetic construct
MGSSHHHHHHENLYFQGASGNNPTIENEPKEGIPVDKKITVNKTWAVDGNEVNKADETVDAVFTLQVKVITAPPTTSDGAKWVNVDSAKATAATSFKHTFENLDNAKTYRVIERVSGYAPEYVSFVNGVVTIKNNKDSNEPTPI

SEQ ID NO: 85 - protein, 180 aa, synthetic construct
MGSSHHHHHHENLYFQGASGNNPTIENEPKEGIPVDKKITVNKTWAVDGNEVNKADETVDAVFTLQVKYARYRKAYEHITRDYPDAAGANKGKISTVSDYFRNIRTHSIHHAVSSLKWVNVDSAKATAATSFKHTFENLDNAKTYRVIERVSGYAPEYVSFVNGVVTIKNNKDSNEPTPI

SEQ ID NO: 86 - protein, 184 aa, synthetic construct; identical to SEQ ID NO: 78

SEQ ID NO: 87 - protein, 151 aa, synthetic construct
MGSSHHHHHHENLYFQGASGNNPTIENEPKEGIPVDKKITVNKTWAVDGNEVNKADETVDAVFTLQVKHSAGTKPTYYDDIDSGKTKKWVNVDSAKATAATSFKHTFENLDNAKTYRVIERVSGYAPEYVSFVNGVVTIKNNKDSNEPTPI

SEQ ID NO: 88 - protein, 184 aa, synthetic construct
MGSSHHHHHHENLYFQASGNNPTIENEPKEGIPVDKKITVNKTWAVDGNEVNKADETVDAVFTLQVKYARYRKWNDNKYSVNIKELGRKDGTSSSGRYLNIQTRKTENQENGTFHAVSSLKWVNVDSAKATAATSFKHTFENLDNAKTYRVIERVSGYAPEYVSFVNGVVTIKNNKDSNEPTPI

SEQ ID NO: 89 - protein, 176 aa, synthetic construct
MGSSHHHHHHENLYFQGASGNNPTIENEPKEGIPVDKKITVNKTWAVDGNEVNKADETVDAVFTLQVKRPGRNPKTGDVVPVSARRVVGPSLFSLHHRQPRLGRNPKTGDSVKWVNVDSAKATAATSFKHTFENLDNAKTYRVIERVSGYAPEYVSFVNGVVTIKNNKDSNEPTPI

SEQ ID NO: 90 - protein, 182 aa, synthetic construct
MGSSHHHHHHENLYFQGASGNNPTIENEPKEGIPVDKKITVNKTWAVDGNEVNKADETVDAVFTLQVKLFMKTRNGSMKAADNFLDPNKASSLLSSGFSPDFATVITMDRKASKQQTNKWVNVDSAKATAATSFKHTFENLDNAKTYRVIERVSGYAPEYVSFVNGVVTIKNNKDSNEPTPI

SEQ ID NO: 91 - protein, 155 aa, synthetic construct
MGSSHHHHHHENLYFQGASGNNPTIENEPKEGIPVDKKITVNKTWAVDGNEVNKADETVDAVFTLQVKNWGPYDRDSWNPVYGNQLFMKTRKWVNVDSAKATAATSFKHTFENLDNAKTYRVIERVSGYAPEYVSFVNGVVTIKNNKDSNEPTPI

SEQ ID NO: 92 - protein, 163 aa, synthetic construct
MGSSHHHHHHENLYFQGASGNNPTIENEPKEGIPVDKKITVNKTWAVDGNEVNKADETVDAVFTLQVKNWGPYDRDSWNPVYGNQLFMKTRGSTNTKDKKWVNVDSAKATAATSFKHTFENLDNAKTYRVIERVSGYAPEYVSFVNGVVTIKNNKDSNEPTPI

SEQ ID NO: 93 - protein, 152 aa, synthetic construct
MGSSHHHHHHENLYFQGASGNNPTIENEPKEGIPVDKKITVNKTWAVDGNEVNKADETVDAVFTLQVKGQAEPDRAHYNIVTFCCKCDKWVNVDSAKATAATSFKHTFENLDNAKTYRVIERVSGYAPEYVSFVNGVVTIKNNKDSNEPTPI

SEQ ID NO: 94 - protein, 167 aa, synthetic construct
MGSSHHHHHHENLYFQGASGNNPTIENEPKEGIPVDKKITVNKTWAVDGNEVNKADETVDAVFTLQVKTGSQAIASPGNKRGFQENPFDYFNDEFFNRFFGLPKWVNVDSAKATAATSFKHTFENLDNAKTYRVIERVSGYAPEYVSFVNGVVTIKNNKDSNEPTPI

SEQ ID NO: 95 - protein, 152 aa, synthetic construct
MGSSHHHHHHENLYFQGASGNNPTIENEPKEGIPVDKKITVNKTWAVDGNEVNKADETVDAVFTLQVKNKRGFQENPFDYFNDEFFNRKWVNVDSAKATAATSFKHTFENLDNAKTYRVIERVSGYAPEYVSFVNGVVTIKNNKDSNEPTPI

SEQ ID NO: 96 - protein, 155 aa, synthetic construct
MGSHHHHHHGGSASGNNPTIENEPKEGIPVDKKITVNKTWAVDGNEVNKADETVDAVFTLQVKKYIKFKHDYNILEFNDGTFEYGARPQFNKWVNVDSAKATAATSFKHTFENLDNAKTYRVIERVSGYAPEYVSFVNGVVTIKNNKDSNEPTPI

SEQ ID NO: 97 - protein, 154 aa, synthetic construct
MGSHHHHHHGGSASGNNPTIENEPKEGIPVDKKITVNKTWAVDGNEVNKADETVDAVFTLQVKETNAYNVTTHANGQVSYGARPTYKKPSKWVNVDSAKATAATSFKHTFENLDNAKTYRVIERVSGYAPEYVSFVNGVVTIKNNKDSNEPTPI

SEQ ID NO: 98 - protein, 358 aa, synthetic construct
MPTIENEPKEGIPVDKKITVNKTWAVDGNEVNKADETVDAVFTLQVKQRYGEGTKKIEYDGQTYSIPSLFVKWVNVDSAKATAATSFKHTFENLDNAKTYRVIERVSGYAPEYVSFVNGVVTIKNNKDSNEPTPIGGSGGSGGSGGSMKMEELFKKHKIVAVLRANSVEEAKKKALAVFLGGVHLIEITFTVPDADTVIKELSFLKEMGAIIGAGTVTSVEQARKAVESGAEFIVSPHLDEEISQFAKEKGVFYMPGVMTPTELVKAMKLGHTILKLFPGEVVGPQFVKAMKGPFPNVKFVPTGGVNLDNVCEWFKAGVLAVGVGSALVKGTPVEVAEKAKAFVEKIRGCTEHHHHHH

SEQ ID NO: 99 - protein, 368 aa, synthetic construct
MPTIENEPKEGIPVDKKITVNKTWAVDGNEVNKADETVDAVFTLQVKSPLKNTGANVNAWESGKFTGNVLEISGMAQREHRKWVNVDSAKATAATSFKHTFENLDNAKTYRVIERVSGYAPEYVSFVNGVVTIKNNKDSNEPTPIGGSGGSGGSGGSMKMEELFKKHKIVAVLRANSVEEAKKKALAVFLGGVHLIEITFTVPDADTVIKELSFLKEMGAIIGAGTVTSVEQARKAVESGAEFIVSPHLDEEISQFAKEKGVFYMPGVMTPTELVKAMKLGHTILKLFPGEVVGPQFVKAMKGPFPNVKFVPTGGVNLDNVCEWFKAGVLAVGVGSALVKGTPVEVAEKAKAFVEKIRGCTEHHHHHH

SEQ ID NO: 100 - protein, 693 aa, Streptococcus agalactiae
MKRINKYFAMFSALLLILTSLLSVAPVFAAEMGNITKTVTLHKIVQTSDNLAKPNFPGINGLNGTKYMGQKLTDISGYFGQGSKEIAGAFFAVMNESQTKYITESGTEVESIDAAGVLKGLTTENGITFNTANLKGTYQIVELLDKSNYKNGDKVLADSKAVPVKITLPLYNEEGIVVDAEVYPKNTEEAPQIDKNFAKANKLLNDSDNSAIAGGADYDKYQAEKAKATAEIGQEIPYEVKTKIQKGSKYKNLAWVDTMSNGLTMGNTVNLEASSGSFVEGTDYNVERDDRGFTLKFTDTGLTKLQKEAETQAVEFTLTYSATVNGAAIDDKPESNDIKLQYGNKPGKKVKEIPVTPSNGEITVSKTWDKGSDLENANVVYTLKDGGTAVASVSLTKTTPNGEINLGNGIKFTVTGAFAGKFSGLTDSKTYMISERIAGYGNTITTGAGSAAITNTPDSDNPTPLNPTEPKVVTHGKKFVKTSSTETERLQGAQFVVKDSAGKYLALKSSATISAQTTAYTNAKTALDAKIAAYNKLSADDQKGTKGETAKAEIKTAQDAYNAAFIVARTAYEWVTNKEDANVVKVTSNADGQFEVSGLATGDYKLEETQAPAGYAKLAGDVDFKVGNSSKADDSGNIDYTASSNKKDAQRIENKKVTIPQTGGIGTILFTIIGLSIMLGAVIIMKRRQSEEA

SEQ ID NO: 101 - protein, 122 aa, Streptococcus agalactiae
NKPGKKVKEIPVTPSNGEITVSKTWDKGSDLENANVVYTLKDGGTAVASVSLTKTTPNGEINLGNGIKFTVTGAFAGKFSGLTDSKTYMISERIAGYGNTITTGAGSAAITNTPDSDNPTPL

SEQ ID NO: 102 - protein, 23 aa, Streptococcus agalactiae
DKGSDLENANVVYTLKDGGTAVA

SEQ ID NO: 103 - protein, 4 aa, Streptococcus agalactiae
TPNG

SEQ ID NO: 104 - protein, 7 aa, Streptococcus agalactiae
FTVTGAF

SEQ ID NO: 105 - protein, 9 aa, Streptococcus agalactiae
SGLTDSKTY

SEQ ID NO: 106 - protein, 6 aa, Streptococcus agalactiae
RIAGYG

SEQ ID NO: 107 - protein, 6 aa, Streptococcus agalactiae
TGAGSA

SEQ ID NO: 108 - protein, 25 aa, Streptococcus agalactiae
NKPGKKVKEIPVTPSNGEITVSKTW

SEQ ID NO: 109 - protein, 7 aa, Streptococcus agalactiae
SVSLTKT

SEQ ID NO: 110 - protein, 9 aa, Streptococcus agalactiae
EINLGNGIK

SEQ ID NO: 111 - protein, 4 aa, Streptococcus agalactiae
AGKF

SEQ ID NO: 112 - protein, 4 aa, Streptococcus agalactiae
MISE

SEQ ID NO: 113 - protein, 4 aa, Streptococcus agalactiae
NTIT

SEQ ID NO: 114 - protein, 14 aa, Streptococcus agalactiae
AITNTPDSDNPTPL

SEQ ID NO: 115 - protein, 674 aa, Streptococcus agalactiae
MKKINKCLTMFSTLLLILTSLFSVAPAFADDATTDTVTLHKIVMPQAAFDNFTEGTKGKNDSDYVGKQINDLKSYFGSTDAKEIKGAFFVFKNETGTKFITENGKEVDTLEAKDAEGGAVLSGLTKDNGFVFNTAKLKGIYQIVELKEKSNYDNNGSILADSKAVPVKITLPLVNNQGVVKDAHIYPKNTETKPQVDKNFADKDLDYTDNRKDKGVVSATVGDKKEYIVGTKILKGSDYKKLVWTDSMTKGLTFNNNVKVTLDGEDFPVLNYKLVTDDQGFRLALNATGLAAVAAAAKDKDVEIKITYSATVNGSTTVEIPETNDVKLDYGNNPTEESEPQEGTPANQEIKVIKDWAVDGTITDANVAVKAIFTLQEKQTDGTWVNVASHEATKPSRFEHTFTGLDNAKTYRVVERVSGYTPEYVSFKNGVVTIKNNKNSNDPTPINPSEPKVVTYGRKFVKTNQANTERLAGATFLVKKEGKYLARKAGAATAEAKAAVKTAKLALDEAVKAYNDLTKEKQEGQEGKTALATVDQKQKAYNDAFVKANYSYEWVADKKADNVVKLISNAGGQFEITGLDKGTYGLEETQAPAGYATLSGDVNFEVTATSYSKGATTDIAYDKGSVKKDAQQVQNKKVTIPQTGGIGTILFTIIGLSIMLGAVVIMKKRQSEEA

SEQ ID NO: 116 - protein, 115 aa, Streptococcus agalactiae
NNPTEESEPQEGTPANQEIKVIKDWAVDGTITDANVAVKAIFTLQEKQTDGTWVNVASHEATKPSRFEHTFTGLDNAKTYRVVERVSGYTPEYVSFKNGVVTIKNNKNSNDPTPI

SEQ ID NO: 117 - protein, 14 aa, Streptococcus agalactiae
AVDGTITDANVAVK

SEQ ID NO: 118 - protein, 4 aa, Streptococcus agalactiae
QTDG

SEQ ID NO: 119 - protein, 6 aa, Streptococcus agalactiae
ATKPSR

SEQ ID NO: 120 - protein, 9 aa, Streptococcus agalactiae
TGLDNAKTY

SEQ ID NO: 121 - protein, 6 aa, Streptococcus agalactiae
RVSGYT

SEQ ID NO: 122 - protein, 4 aa, Streptococcus agalactiae
KNGV

SEQ ID NO: 123 - protein, 25 aa, Streptococcus agalactiae
NNPTEESEPQEGTPANQEIKVIKDW

SEQ ID NO: 124 - protein, 8 aa, Streptococcus agalactiae
AIFTLQEK

SEQ ID NO: 125 - protein, 9 aa, Streptococcus agalactiae
TWVNVASHE

SEQ ID NO: 126 - protein, 5 aa, Streptococcus agalactiae
FEHTF

SEQ ID NO: 127 - protein, 4 aa, Streptococcus agalactiae
RVVE

SEQ ID NO: 128 - protein, 6 aa, Streptococcus agalactiae
PEYVSF

SEQ ID NO: 129 - protein, 15 aa, Streptococcus agalactiae
VTIKNNKNSNDPTPI

SEQ ID NO: 130 - protein, 705 aa, Streptococcus agalactiae
MKRINKYFAMFSALLLTLTSLLSVAPAFADEATTNTVTLHKILQTESNLNKSNFPGTTGLNGKDYKGGAISDLAGYFGEGSKEIEGAFFALALKEDKSGKVQYVKAKEGNKLTPALINKDGTPEITVNIDEAVSGLTPEGDTGLVFNTKGLKGEFKIVEVKSKSTYNNNGSLLAASKAVPVNITLPLVNEDGVVADAHVYPKNTEEKPEIDKNFAKTNDLTALTDVNRLLTAGANYGNYARDKATATAEIGKVVPYEVKTKIHKGSKYENLVWTDIMSNGLTMGSTVSLKASGTTETFAKDTDYELSIDARGFTLKFTADGLGKLEKAAKTADIEFTLTYSATVNGQAIIDNPESNDIKLSYGNKPGKDLTELPVTPSKGEVTVAKTWSDGIAPDGVNVVYTLKDKDKTVASVSLTKTSKGTIDLGNGIKFEVSGNFSGKFTGLENKSYMISERVSGYGSAINLENGKVTITNTKDSDNPTPLNPTEPKVETHGKKFVKTNEQGDRLAGAQFVVKNSAGKYLALKADQSEGQKTLAAKKIALDEAIAAYNKLSATDQKGEKGITAKELIKTKQADYDAAFIEARTAYEWITDKARAITYTSNDQGQFEVTGLADGTYNLEETLAPAGFAKLAGNIKFVVNQGSYITGGNIDYVANSNQKDATRVENKKVTIPQTGGIGTILFTIIGLSIMLGAVVIMKRRQSKEA

SEQ ID NO: 131 - protein, 120 aa, Streptococcus agalactiae
NKPGKDLTELPVTPSKGEVTVAKTWSDGIAPDGVNVVYTLKDKDKTVASVSLTKTSKGTIDLGNGIKFEVSGNFSGKFTGLENKSYMISERVSGYGSAINLENGKVTITNTKDSDNPTPL

SEQ ID NO: 132 - protein, 13 aa, Streptococcus agalactiae
TWSDGIAPDGVNV

SEQ ID NO: 133 - protein, 9 aa, Streptococcus agalactiae
KDKDKTVAS

SEQ ID NO: 134 - protein, 20 aa, Streptococcus agalactiae
KTSKGTIDLGNGIKFEVSGN

SEQ ID NO: 135 - protein, 6 aa, Streptococcus agalactiae
TGLENK

SEQ ID NO: 136 - protein, 6 aa, Streptococcus agalactiae
RVSGYG

SEQ ID NO: 137 - protein, 4 aa, Streptococcus agalactiae
ENGK

SEQ ID NO: 138 - protein, 23 aa, Streptococcus agalactiae
NKPGKDLTELPVTPSKGEVTVAK

SEQ ID NO: 139 - protein, 4 aa, Streptococcus agalactiae
VYTL

SEQ ID NO: 140 - protein, 4 aa, Streptococcus agalactiae
VSLT

SEQ ID NO: 141 - protein, 5 aa, Streptococcus agalactiae
FSGKF

SEQ ID NO: 142 - protein, 6 aa, Streptococcus agalactiae
SYMISE

SEQ ID NO: 143 - protein, 5 aa, Streptococcus agalactiae
SAINL

SEQ ID NO: 144 - protein, 15 aa, Streptococcus agalactiae
VTITNTKDSDNPTPL

SEQ ID NO: 145 - protein, 704 aa, Streptococcus agalactiae
MKKINKYFAVFSALLLTVTSLLSVAPAFADEATTNTVTLHKILQTESNLNKSNFPGTTGLNGDDYKGESISDLAEYFGSGSKEIDGAFFALALEEEKDGVVQYVKAKANDKLTPDLITKGTPATTTKVEEAVGGLTTGTGIVFNTAGLKGNFKIIELKDKSTYNNNGSLLAASKAVPVKITLPLVSKDGVVKDAHVYPKNTETKPEVDKNFAKTNDLTALKDATLLKAGADYKNYSATKATVTAEIGKVIPYEVKTKVLKGSKYEKLVWTDTMSNGLTMGDDVNLAVSGTTTTFIKDIDYTLSIDDRGFTLKFKATGLDKLEEAAKASDVEFTLTYKATVNGQAIIDNPEVNDIKLDYGNKPGTDLSEQPVTPEDGEVKVTKTWAAGANKADAKVVYTLKNATKQVVASVALTAADTKGTINLGKGMTFEITGAFSGTFKGLQNKAYTVSERVAGYTNAINVTGNAVAITNTPDSDNPTPLNPTQPKVETHGKKFVKVGDADARLAGAQFVVKNSAGKFLALKEDAAVSGAQTELATAKTDLDNAIKAYNGLTKAQQEGADGTSAKELINTKQSAYDAAFIKARTAYTWVDEKTKAITFTSNNQGQFEVTGLEVGSYKLEETLAPAGYAKLSGDIEFTVGHDSYTSGDIKYKTDDASNNAQKVFNKKVTIPQTGGIGTILFTIIGLSIMLGAVVIMKRRQSEEA

SEQ ID NO: 146 - protein, 122 aa, Streptococcus agalactiae
NKPGTDLSEQPVTPEDGEVKVTKTWAAGANKADAKVVYTLKNATKQVVASVALTAADTKGTINLGKGMTFEITGAFSGTFKGLQNKAYTVSERVAGYTNAINVTGNAVAITNTPDSDNPTPL

SEQ ID NO: 147 - protein, 23 aa, Streptococcus agalactiae
AAGANKADAKVVYTLKNATKQVV

SEQ ID NO: 148 - protein, 4 aa, Streptococcus agalactiae
DTKG

SEQ ID NO: 149 - protein, 7 aa, Streptococcus agalactiae
FEITGAF

SEQ ID NO: 150 - protein, 8 aa, Streptococcus agalactiae
KGLQNKAY

SEQ ID NO: 151 - protein, 6 aa, Streptococcus agalactiae
RVAGYT

SEQ ID NO: 152 - protein, 4 aa, Streptococcus agalactiae
TGNA

SEQ ID NO: 153 - protein, 25 aa, Streptococcus agalactiae
NKPGTDLSEQPVTPEDGEVKVTKTW

SEQ ID NO: 154 - protein, 8 aa, Streptococcus agalactiae
ASVALTAA

SEQ ID NO: 155 - protein, 9 aa, Streptococcus agalactiae
TINLGKGMT

SEQ ID NO: 156 - protein, 4 aa, Streptococcus agalactiae
SGTF

SEQ ID NO: 157 - protein, 4 aa, Streptococcus agalactiae
TVSE

SEQ ID NO: 158 - protein, 5 aa, Streptococcus agalactiae
NAINV

SEQ ID NO: 159 - protein, 15 aa, Streptococcus agalactiae
VAITNTPDSDNPTPL

SEQ ID NO: 160 - protein, 682 aa, Streptococcus agalactiae
MKKINKFFVAFSALLLILTSLLSVAPAFAEEERTTETVTLHKILQTETNLKNSAFPGTKGLDGTEYDGKAIDKLDSYFGNDSKDIGGAYFILANSKGEYIKANDKNKLKPEFSGNTPKTTLNISEAVGGLTEENAGIKFETTGLRGDFQIIELKDKSTYNNGGAILADSKAVPVKITLPLINKDGVVKDAHVYPKNTETKPQIDKNFADKNLDYINNQKDKGTISATVGDVKKYTVGTKILKGSDYKKLVWTDSMTKGLTFNNDVTVTLDGANFEQSNYTLVADDQGFRLVLNATGLSKVAEAAKTKDVEIKINYSATVNGSTVVEKSENNDVKLDYGNNPTTENEPQTGNPVNKEITVRKTWAVDGNEVNKGDEKVDAVFTLQVKDSDKWVNVDSATATAATDFKYTFKNLDNAKTYRVVERVSGYAPAYVSFVGGVVTIKNNKNSNDPTPINPSEPKVVTYGRKFVKTNQDGSERLAGATFLVKNSQSQYLARKSGVATNEAHKAVTDAKVQLDEAVKAYNKLTKEQQESQDGKAALNLIDEKQTAYNEAFAKANYSYEWVVDKNAANVVKLISNTAGKFEITGLNAGEYSLEETQAPTGYAKLSSDVSFKVNDTSYSEGASNDIAYDKDSGKTDAQKVVNKKVTIPQTGGIGTILFTIIGLSIMLGAVVIMKRRQSEEA

SEQ ID NO: 161 - protein, 115 aa, Streptococcus agalactiae
NNPTTENEPQTGNPVNKEITVRKTWAVDGNEVNKGDEKVDAVFTLQVKDSDKWVNVDSATATAATDFKYTFKNLDNAKTYRVVERVSGYAPAYVSFVGGVVTIKNNKNSNDPTPI

SEQ ID NO: 162 - protein, 15 aa, Streptococcus agalactiae
VDGNEVNKGDEKVDA

SEQ ID NO: 163 - protein, 4 aa, Streptococcus agalactiae
DSDK

SEQ ID NO: 164 - protein, 9 aa, Streptococcus agalactiae
ATATAATDF

SEQ ID NO: 165 - protein, 9 aa, Streptococcus agalactiae
KNLDNAKTY

SEQ ID NO: 166 - protein, 6 aa, Streptococcus agalactiae
RVSGYA

SEQ ID NO: 167 - protein, 4 aa, Streptococcus agalactiae
VGGV

SEQ ID NO: 168 - protein, 26 aa, Streptococcus agalactiae
NNPTTENEPQTGNPVNKEITVRKTWA

SEQ ID NO: 169 - protein, 7 aa, Streptococcus agalactiae
VFTLQVK

SEQ ID NO: 170 - protein, 6 aa, Streptococcus agalactiae
WVNVDS

SEQ ID NO: 171 - protein, 4 aa, Streptococcus agalactiae
KYTF

SEQ ID NO: 172 - protein, 4 aa, Streptococcus agalactiae
RVVE

SEQ ID NO: 173 - protein, 6 aa, Streptococcus agalactiae
PAYVSF

SEQ ID NO: 174 - protein, 15 aa, Streptococcus agalactiae
VTIKNNKNSNDPTPI

SEQ ID NO: 175 - protein, 6 aa, synthetic construct
HHHHHH

SEQ ID NO: 176 - protein, 7 aa, synthetic construct
ENLYFQG

SEQ ID NO: 177 - protein, 7 aa, synthetic construct
  VARIANT 5: note = X2 is F, Y or W
  VARIANT 7: note = X3 is S, G, A, M, C or H
EXLYXQX

SEQ ID NO: 178 - protein, 4 aa, synthetic construct
GSGS

SEQ ID NO: 179 - protein, 6 aa, synthetic construct
GSGGGG

SEQ ID NO: 180 - protein, 8 aa, synthetic construct
GSGSGGGG

SEQ ID NO: 181 - protein, 12 aa, synthetic construct
GGSGGSGGSGGS

SEQ ID NO: 182 - protein, 19 aa, synthetic construct
PPSDIKGKYVKEVEVKGSG

SEQ ID NO: 183 - protein, 17 aa, synthetic construct
DAKDGKEIDTKHLPSTC

SEQ ID NO: 184 - protein, 170 aa, synthetic construct
MGSSHHHHHHENLYFQGLYFQGLYFQGASGNNPTIENEPKEGIPVDKKITVNKTWAVDGNEVNKADETVDAVFTLQVKDGDKWVNVDSAKATAATSFKHTFENLDNAKTYRVIERVSGYAPEYVSFVNGVVTIKNNKDSNEPTPIGSGGGGPPSDIKGKYVKEVEVKGSG

SEQ ID NO: 185 - protein, 168 aa, synthetic construct
MGSSHHHHHHENLYFQGLYFQGLYFQGASGNNPTIENEPKEGIPVDKKITVNKTWAVDGNEVNKADETVDAVFTLQVKDGDKWVNVDSAKATAATSFKHTFENLDNAKTYRVIERVSGYAPEYVSFVNGVVTIKNNKDSNEPTPIGSGGGGDAKDGKEIDTKHLPSTC

SEQ ID NO: 186 - protein, 159 aa, synthetic construct
MGSSHHHHHHENLYFQGLYFQGLYFQGASGNNPTIENEPKEGIPVDKKITVNKTWAVDGNEVNKADETVDAVFTLQVKDAKDGKEIDTKHLPSTCKWVNVDSAKATAATSFKHTFENLDNAKTYRVIERVSGYAPEYVSFVNGVVTIKNNKDSNEPTPI

SEQ ID NO: 187 - protein, 248 aa, synthetic construct
MQRYGEGTKKIEYDGQTYSIPSLFVGGSGGSGGSGGSMKMEELFKKHKIVAVLRANSVEEAKKKALAVFLGGVHLIEITFTVPDADTVIKELSFLKEMGAIIGAGTVTSVEQARKAVESGAEFIVSPHLDEEISQFAKEKGVFYMPGVMTPTELVKAMKLGHTILKLFPGEVVGPQFVKAMKGPFPNVKFVPTGGVNLDNVCEWFKAGVLAVGVGSALVKGTPVEVAEKAKAFVEKIRGCTEHHHHHH

SEQ ID NO: 188 - protein, 167 aa, synthetic construct
MGSSHHHHHHENLYFQGASGNNPTIENEPKEGIPVDKKITVNKTWAVDPRAVNTTPDNQLGAVDSTTPAADAVFTLQVKQRYGEGTKKIEYDGQTYSIPSLFVKWVNVDSAKATAATSFKHTFENLDNAKTYRVIERVSGYAPEYVSFVNGVVTIKNNKDSNEPTPI

SEQ ID NO: 189 - protein, 159 aa, synthetic construct
MGSSHHHHHHENLYFQGASGNNPTIENEPKEGIPVDKKITVNKTWAVDAAETGATINLPFEADAVFTLQVKQRYGEGTKKIEYDGQTYSIPSLFVKWVNVDSAKATAATSFKHTFENLDNAKTYRVIERVSGYAPEYVSFVNGVVTIKNNKDSNEPTPI

SEQ ID NO: 190 - protein, 159 aa, synthetic construct
MGSSHHHHHHENLYFQGNKPGKKVKEIPVTPSNGEITVSKTWDKGSDLENANVVYTLKQRYGEGTKKIEYDGQTYSIPSLFVAVASVSLTKTTPNGEINLGNGIKFTVTGAFAGKFSGLTDSKTYMISERIAGYGNTITTGAGSAAITNTPDSDNPTPL

SEQ ID NO: 191 - protein, 152 aa, synthetic construct
MGSSHHHHHHENLYFQGNNPTEESEPQEGTPANQEIKVIKDWAVDGTITDANVAVKAIFTLQEKQRYGEGTKKIEYDGQTYSIPSLFVTWVNVASHEATKPSRFEHTFTGLDNAKTYRVVERVSGYTPEYVSFKNGVVTIKNNKNSNDPTPI

SEQ ID NO: 192 - protein, 157 aa, synthetic construct
MGSSHHHHHHENLYFQGNKPGTDLSEQPVTPEDGEVKVTKTWAAGANKADAKVVYTLKQRYGEGTKKIEYDGQTYSIPSLFVVASVALTAADTKGTINLGKGMTFEITGAFSGTFKGLQNKAYTVSERVAGYTNAINVTGNAVAITNTPDSDNPTPL

SEQ ID NO: 193 - protein, 159 aa, synthetic construct
MGSSHHHHHHENLYFQGNKPGKDLTELPVTPSKGEVTVAKTWSDGIAPDGVNVVYTLKQRYGEGTKKIEYDGQTYSIPSLFVKTVASVSLTKTSKGTIDLGNGIKFEVSGNFSGKFTGLENKSYMISERVSGYGSAINLENGKVTITNTKDSDNPTPLP

SEQ ID NO: 194 - protein, 153 aa, synthetic construct
MGSSHHHHHHENLYFQGNNPTTENEPQTGNPVNKEITVRKTWAVDGNEVNKGDEKVDAVFTLQVKQRYGEGTKKIEYDGQTYSIPSLFVKWVNVDSATATAATDFKYTFKNLDNAKTYRVVERVSGYAPAYVSFVGGVVTIKNNKNSNDPTPI

SEQ ID NO: 195 - protein, 239 aa, synthetic construct
VSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYKG

SEQ ID NO: 196 - protein, 371 aa, synthetic construct
MGSSHHHHHHENLYFQGASGNNPTIENEPKEGIPVDKKITVNKTWAVDGNEVNKADETVDAVFTLQVKVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYKGKWVNVDSAKATAATSFKHTFENLDNAKTYRVIERVSGYAPEYVSFVNGVVTIKNNKDSNEPTPI

SEQ ID NO: 197 - protein, 43 aa, Chlamydia trachomatis
DRVLKTDVNKEFQMGAKPTTDTGNSAAPSTLTARENPAYGRHM

SEQ ID NO: 198 - protein, 41 aa, Chlamydia trachomatis
NSASFNLVGLFGDNENQKTVKAESVPNMSFDQSVVELYTDT

SEQ ID NO: 199 - protein, 48 aa, Chlamydia trachomatis
KPKVEELNVLCNAAEFTINKPKGYVGKEFPLDLTAGTDAATGTKDASI

SEQ ID NO: 200 - protein, 59 aa, Chlamydia trachomatis
DADTIRIAQPKSATAIFDTTTLNPTIAGAGDVKTGAEGQLGDTMQIVSLQLNKMKSRKS

(19) United States
(12) Patent Application Publication
(10) Pub. No.: US 2026/0151472 A1
(43) Pub. Date: Jun. 4, 2026

(54) POLYPEPTIDE SCAFFOLD

(71) Applicant: GLAXOSMITHKLINE BIOLOGICALS SA, Rixensart (BE)

(72) Inventors: Luigia CAPPELLI, Siena (IT); Roberta COZZI, Siena (IT)

(73) Assignee: GLAXOSMITHKLINE BIOLOGICALS SA, Rixensart (BE)

(21) Appl. No.: 19/122,292

(22) PCT Filed: Oct. 19, 2023
(86) PCT No.: PCT/EP2023/079083

§ 371 Date: Apr. 18, 2025

(30) Foreign Application Priority Data
Oct. 21, 2022 (GB) 2215634.3

(51) Int. Cl. A61K 39/095; A61K 39/00; A61K 39/385; A61K 47/69; A61P 31/04; C07K 14/315

(52) U.S. Cl. CPC A61K 39/095; A61K 39/385; A61K 47/6901; A61K 47/6929; A61P 31/04; C07K 14/315; A61K 2039/55555; A61K 2039/6068; C07K 2319/40

(57) ABSTRACT

The present invention relates to chimeric polypeptides comprising a scaffold polypeptide and one or more exogenous polypeptides, wherein the scaffold polypeptide comprises a backbone protein 2a (BP-2a) Domain 3 (D3) polypeptide in which at least one endogenous loop has been partially or wholly replaced by an exogenous polypeptide. The present invention also relates to nanoparticles, outer membrane vesicles, pharmaceutical compositions, vaccines, arrays and kits comprising said chimeric polypeptides, methods of their production, and uses thereof in therapy and antibody screening.

### TECHNICAL FIELD

The present invention relates to polypeptide scaffolds for the display of exogenous polypeptides (e.g., antigenic sequences or epitopes), nanoparticles and outer membrane vesicles comprising said scaffolds, methods of their production, vaccines, and therapeutic uses thereof.

### BACKGROUND

Preparations enriched by a specific protein are rarely easily obtained from natural host cells. Hence, recombinant protein production is generally the most optimal procedure.

In theory, the recombinant protein production process is simple. A gene encoding the protein of interest is cloned into an expression vector, the expression vector is inserted into a host cell (e.g., via transformation, transfection, transduction or conjugation) or cell-free expression system, expression of the gene is induced, the protein is produced and it can then be purified and characterised.

In practice, however, the recombinant protein production process often presents multiple challenges.

In bacterial hosts such as Escherichia coli, for example, overexpression of recombinant proteins can rapidly exhaust the bacterial protein quality control system, leading to an increased number of partially folded and misfolded proteins. These proteins tend to aggregate and form inclusion bodies, which pose a significant hurdle for protein production and purification. While it is possible to purify functional recombinant proteins from inclusion bodies, this process is labor intensive, and the yields of recombinant protein are often low.

In another example, recombinant production is made challenging by certain proteins having transmembrane domains or GPI-anchor sequences. These features lead the proteins to become inserted in the plasma membrane during recombinant protein production, which thus requires additional purification steps that reduce protein yields. Meanwhile, the hydrophobicity of the transmembrane domains can also affect stability, often leading to misfolding and aggregation.

Naturally, these issues present a bottleneck for recombinant protein production, especially at scale.

Other common expression systems and types of proteins present their own set of challenges.

One particular application that would benefit from improved methods of recombinant protein production is the production of protein-based vaccines.

When considering protein-based vaccines, virulence factors involved in bacterial adhesion, permeation, and evasion of immune system of host cells represent attractive targets. However, many of these protein antigens are anchored into the lipid bilayer through an extended hydrophobic portion.

The recombinant production of these membrane proteins is therefore complicated by their transmembrane region(s), as described above. However, relative to the protein as a whole, often only a few possible epitopes are accessible to antibodies during infection. These epitopes are found on those parts of the protein that face the extracellular environment (rather than buried in the membrane or facing intracellularly). These extracellular-facing regions often comprise a number of immunogenic loops that can be used to generate immune responses.

However, producing such immunogenic loops of interest on their own, i.e., separately to the whole protein, also presents challenges. For example, depending on the sequence length, recombinantly produced loops can be susceptible to degradation. The chemical synthesis and purification of peptides can also be difficult and highly sequence dependent.

### SUMMARY OF THE INVENTION

In view of these problems in the art, it is an object of the invention to provide a scaffold for facilitating the recombinant production of polypeptides including for example, antigenic sequences or epitopes derived from proteins of interest. By facilitating the effective display of a desired polypeptide (e.g., an epitope), the present invention is particularly beneficial for the production of protein-based vaccines, especially their production at large scale.

The present inventors surprisingly found that domain 3 (D3) of pilus 2a backbone protein (BP-2a), also referred to herein as “BP-2a D3”, can be advantageously used as a scaffold protein to facilitate the recombinant production of polypeptide sequences of interest (e.g., epitopes). BP-2a D3 natively contains a number of loops (referred to herein as “endogenous loops”) which the inventors identified can be partially or wholly replaced by one or more exogenous polypeptides. The resultant chimeric polypeptides can be recombinantly produced as highly stable and soluble proteins (e.g., using a polyhistidine tag and purifying by Ni-NTA affinity chromatography). Further advantageously, the chimeric polypeptides correctly display the exogenous polypeptides comprised therein.

Therefore, in a first aspect, the present invention provides a chimeric polypeptide comprising:
(i) a scaffold polypeptide; and
(ii) one or more exogenous polypeptide(s),
wherein the scaffold polypeptide comprises a backbone protein 2a (BP-2a) Domain 3 (D3) polypeptide, wherein in the chimeric polypeptide at least one endogenous loop of the BP-2a D3 polypeptide is partially or wholly replaced by an exogenous polypeptide.

In certain embodiments, the at least one endogenous loop of the BP-2a D3 polypeptide is selected from the first, second, third, fourth, fifth and sixth endogenous loops of the BP-2a D3 polypeptide, wherein for a given BP-2a D3 polypeptide:
(i) the first endogenous loop of the BP-2a D3 polypeptide spans the amino acids at positions corresponding to positions 360-372 of SEQ ID NO:1;
(ii) the second endogenous loop of the BP-2a D3 polypeptide spans the amino acids at positions corresponding to positions 380-384 of SEQ ID NO:1;
(iii) the third endogenous loop of the BP-2a D3 polypeptide spans the amino acids at positions corresponding to positions 393-399 of SEQ ID NO:1;
(iv) the fourth endogenous loop of the BP-2a D3 polypeptide spans the amino acids at positions corresponding to positions 404-411 of SEQ ID NO:1;
(v) the fifth endogenous loop of the BP-2a D3 polypeptide spans the amino acids at positions corresponding to positions 418-422 of SEQ ID NO:1; and
(vi) the sixth endogenous loop of the BP-2a D3 polypeptide spans the amino acids at positions corresponding to positions 429-432 of SEQ ID NO:1, wherein the amino acid positions are numbered according to the sequence shown in SEQ ID NO:1, wherein amino acids 332-447 are the native BP-2a D3 polypeptide from strain 515.

Said endogenous loops may be at equivalent positions within the native BP-2a D3 polypeptide from other GBS strains.

In certain embodiments, the BP-2a D3 polypeptide comprises seven scaffold regions, wherein for a given BP-2a D3 polypeptide:
(i) the first scaffold region of the BP-2a D3 polypeptide spans the amino acids at positions corresponding to positions 332-359 of SEQ ID NO:1;
(ii) the second scaffold region of the BP-2a D3 polypeptide spans the amino acids at positions corresponding to positions 373-379 of SEQ ID NO:1;
(iii) the third scaffold region of the BP-2a D3 polypeptide spans the amino acids at positions corresponding to positions 385-392 of SEQ ID NO:1;
(iv) the fourth scaffold region of the BP-2a D3 polypeptide spans the amino acids at positions corresponding to positions 400-403 of SEQ ID NO:1;
(v) the fifth scaffold region of the BP-2a D3 polypeptide spans the amino acids at positions corresponding to positions 412-417 of SEQ ID NO:1;
(vi) the sixth scaffold region spans the amino acids at positions corresponding to positions 423-428 of SEQ ID NO:1; and
(vii) the seventh scaffold region of the BP-2a D3 polypeptide spans the amino acids at positions corresponding to positions 433-447 of SEQ ID NO:1, wherein the amino acids are numbered according to the sequence shown in SEQ ID NO:1, wherein amino acids 332-447 are the native BP-2a D3 polypeptide from strain 515. Said scaffold regions may be at equivalent positions within the native BP-2a D3 polypeptide from other GBS strains.

[515] In preferred embodiments, the BP-2a D3 polypeptide is at least 40%, at least 50%, at least 60%, at least 70%, at least 80%, at least 90%, at least 95%, at least 97%, at least 98%, or at least 99% identical to the amino acid sequence shown in SEQ ID NO:2. In this context, the percentage identity is calculated with reference to the BP-2a D3 polypeptide only and excluding the one or more exogenous polypeptide sequences.

As described herein, the structure of the BP-2a D3 polypeptide has been elucidated following analysis of the crystal structure of the BP-2a protein from strain 515 (see Nuccitelli, A., et al. Natl Acad Sci USA, 2011. 108(25): p. 10278-83). With this knowledge, the endogenous loops and scaffold regions of the BP-2a D3 polypeptide have been identified with regards to the D3 sequence from strain 515. However, the present invention is not limited to a scaffold protein comprising the BP-2a D3 polypeptide from GBS strain 515 specifically. Instead the inventors have shown that the BP-2a D3 polypeptide sequence from any GBS strain can be utilised (e.g. see Example 9). Whilst the crystal structure of the BP-2a polypeptide has not been elucidated for all GBS strains, the location of the endogenous loops and scaffold regions can be predicted based on the equivalent positions of the endogenous loops and scaffold regions in these strains (see below with regards to strain [H36B], [CJB111], [CJB110], [2603] and [DK21].

[H36B] In certain embodiments, the BP-2a D3 polypeptide is at least 40%, at least 50%, at least 60%, at least 70%, at least 80%, at least 90%, at least 95%, at least 97%, at least 98%, or at least 99% identical to the amino acid sequence shown in SEQ ID NO:101. In this context, the percentage identity is calculated with reference to the BP-2a D3 polypeptide only and excluding the one or more exogenous polypeptide sequences.

[CJB11] In certain embodiments, the BP-2a D3 polypeptide is at least 40%, at least 50%, at least 60%, at least 70%, at least 80%, at least 90%, at least 95%, at least 97%, at least 98%, or at least 99% identical to the amino acid sequence shown in SEQ ID NO:116. In this context, the percentage identity is calculated with reference to the BP-2a D3 polypeptide only and excluding the one or more exogenous polypeptide sequences.

In certain embodiments, the BP-2a D3 polypeptide is at least 40%, at least 50%, at least 60%, at least 70%, at least 80%, at least 90%, at least 95%, at least 97%, at least 98%, or at least 99% identical to the amino acid sequence shown in SEQ ID NO:131. In this context, the percentage identity is calculated with reference to the BP-2a D3 polypeptide only and excluding the one or more exogenous polypeptide sequences.

[CJB110] In certain embodiments, the BP-2a D3 polypeptide is at least 40%, at least 50%, at least 60%, at least 70%, at least 80%, at least 90%, at least 95%, at least 97%, at least 98%, or at least 99% identical to the amino acid sequence shown in SEQ ID NO:146. In this context, the percentage identity is calculated with reference to the BP-2a D3 polypeptide only and excluding the one or more exogenous polypeptide sequences.

[DK21] In certain embodiments, the BP-2a D3 polypeptide is at least 40%, at least 50%, at least 60%, at least 70%, at least 80%, at least 90%, at least 95%, at least 97%, at least 98%, or at least 99% identical to the amino acid sequence shown in SEQ ID NO:161. In this context, the percentage identity is calculated with reference to the BP-2a D3 polypeptide only and excluding the one or more exogenous polypeptide sequences.

In certain embodiments, the BP-2a D3 polypeptide comprises or consists of the amino acid sequence shown in SEQ ID NO:2; SEQ ID NO:101; SEQ ID NO:116; SEQ ID NO:131; SEQ ID NO:146; or SEQ ID NO:161, but for the one or more exogenous polypeptides replacing (wholly or partially) at least one of the endogenous loops. In preferred embodiments, the BP-2a D3 polypeptide comprises or consists of the amino acid sequence shown in SEQ ID NO:2 but for the one or more exogenous polypeptides replacing (wholly or partially) at least one of the endogenous loops.

[515] In certain preferred embodiments, the BP-2a D3 polypeptide is at least 40%, at least 50%, at least 60%, at least 70%, at least 80%, at least 90%, at least 95%, at least 97%, at least 98%, or at least 99% identical to the amino acid sequence shown in SEQ ID NO:2, and the at least one endogenous loop of the BP-2a D3 polypeptide is selected from the first, second, third, fourth, fifth and sixth endogenous loops of the BP-2a D3 polypeptide, wherein:
(i) the first endogenous loop of the BP-2a D3 polypeptide spans the amino acids at positions 360-372;
(ii) the second endogenous loop of the BP-2a D3 polypeptide spans the amino acids at positions 380-384;
(iii) the third endogenous loop of the BP-2a D3 polypeptide spans the amino acids at positions 393-399;
(iv) the fourth endogenous loop of the BP-2a D3 polypeptide spans the amino acids at positions 404-411;
(v) the fifth endogenous loop of the BP-2a D3 polypeptide spans the amino acids at positions 418-422; and
(vi) the sixth endogenous loop of the BP-2a D3 polypeptide spans the amino acids at positions 429-432,
wherein the amino acids are numbered according to the sequence shown in SEQ ID NO:1, wherein amino acids 332-447 are the native BP-2a D3 polypeptide, or wherein said endogenous loops are at equivalent positions within the native BP-2a D3 polypeptide from other GBS strains.

[515] In certain preferred embodiments, the BP-2a D3 polypeptide is at least 40%, at least 50%, at least 60%, at least 70%, at least 80%, at least 90%, at least 95%, at least 97%, at least 98%, or at least 99% identical to the amino acid sequence shown in SEQ ID NO:2, and the BP-2a D3 polypeptide comprises seven scaffold regions, wherein:
(i) the first scaffold region spans the amino acids at positions 332-359;
(ii) the second scaffold region spans the amino acids at positions 373-379;
(iii) the third scaffold region spans the amino acids at positions 385-392;
(iv) the fourth scaffold region spans the amino acids at positions 400-403;
(v) the fifth scaffold region spans the amino acids at positions 412-417;
(vi) the sixth scaffold region spans the amino acids at positions 423-428; and
(vii) the seventh scaffold region spans the amino acids at positions 433-447,
wherein the amino acids are numbered according to the sequence shown in SEQ ID NO:1, wherein the native BP-2a D3 polypeptide spans amino acids 332-447 or wherein said scaffold regions are at equivalent positions within the native BP-2a D3 polypeptide from other GBS strains.

[515] In certain embodiments, the BP-2a D3 polypeptide comprises seven scaffold regions, wherein:
(i) the first scaffold region is at least 40%, at least 50%, at least 60%, at least 70%, at least 80%, at least 90%, at least 95%, at least 97%, at least 98%, or at least 99% identical to the amino acid sequence shown in SEQ ID NO:10;
(ii) the second scaffold region is at least 40%, at least 50%, at least 60%, at least 70%, at least 80%, at least 90%, at least 95%, at least 97%, at least 98%, or at least 99% identical to the amino acid sequence shown in SEQ ID NO:11;
(iii) the third scaffold region is at least 40%, at least 50%, at least 60%, at least 70%, at least 80%, at least 90%, at least 95%, at least 97%, at least 98%, or at least 99% identical to the amino acid sequence shown in SEQ ID NO:12;
(iv) the fourth scaffold region is at least 40%, at least 50%, at least 60%, at least 70%, at least 80%, at least 90%, at least 95%, at least 97%, at least 98%, or at least 99% identical to the amino acid sequence shown in SEQ ID NO:13;
(v) the fifth scaffold region is at least 40%, at least 50%, at least 60%, at least 70%, at least 80%, at least 90%, at least 95%, at least 97%, at least 98%, or at least 99% identical to the amino acid sequence shown in SEQ ID NO:14;
(vi) the sixth scaffold region is at least 40%, at least 50%, at least 60%, at least 70%, at least 80%, at least 90%, at least 95%, at least 97%, at least 98%, or at least 99% identical to the amino acid sequence shown in SEQ ID NO:15; and
(vii) the seventh scaffold region is at least 40%, at least 50%, at least 60%, at least 70%, at least 80%, at least 90%, at least 95%, at least 97%, at least 98%, or at least 99% identical to the amino acid sequence shown in SEQ ID NO:16.

[515] In certain embodiments, wherein the BP-2a D3 polypeptide comprises seven scaffold regions, wherein:
(i) the first scaffold region comprises or consists of the amino acid sequence shown in SEQ ID NO:10;
(ii) the second scaffold region comprises or consists of the amino acid sequence shown in SEQ ID NO:11;
(iii) the third scaffold region comprises or consists of the amino acid sequence shown in SEQ ID NO:12;
(iv) the fourth scaffold region comprises or consists of the amino acid sequence shown in SEQ ID NO:13;
(v) the fifth scaffold region comprises or consists of the amino acid sequence shown in SEQ ID NO:14;
(vi) the sixth scaffold region comprises or consists of the amino acid sequence shown in SEQ ID NO:15; and
(vii) the seventh scaffold region comprises or consists of the amino acid sequence shown in SEQ ID NO:16.

As described above, based on the location of the endogenous loops and scaffold regions within the BP-2a D3 polypeptide from strain 515 (elucidated by analysis of its crystal structure), it is within the remit of the person skilled in the art to determine the equivalent positions of the endogenous loops and scaffold regions within the BP-2a D3 polypeptide of other GBS strain. For example, said equivalent positions with regards to strains [H36B], [CJB111], [CJB110], [2603] and [DK21] are predicted below.

[H36B] In certain preferred embodiments, the BP-2a D3 polypeptide is at least 40%, at least 50%, at least 60%, at least 70%, at least 80%, at least 90%, at least 95%, at least 97%, at least 98%, or at least 99% identical to the amino acid sequence shown in SEQ ID NO: 101, and the at least one endogenous loop of the BP-2a D3 polypeptide is selected from the first, second, third, fourth, fifth and sixth endogenous loops of the BP-2a D3 polypeptide, wherein:
(i) the first endogenous loop of the BP-2a D3 polypeptide spans the amino acids at positions 369-391;
(ii) the second endogenous loop of the BP-2a D3 polypeptide spans the amino acids at positions 399-402;
(iii) the third endogenous loop of the BP-2a D3 polypeptide spans the amino acids at positions 412-418;
(iv) the fourth endogenous loop of the BP-2a D3 polypeptide spans the amino acids at positions 423-431;
(v) the fifth endogenous loop of the BP-2a D3 polypeptide spans the amino acids at positions 436-441; and
(vi) the sixth endogenous loop of the BP-2a D3 polypeptide spans the amino acids at positions 446-451,
wherein the amino acids are numbered according to the sequence shown in SEQ ID NO:100, wherein amino acids 344-465 are the native BP-2a D3 polypeptide, or wherein said endogenous loops are at equivalent positions within the native BP-2a D3 polypeptide from other GBS strains.

[H36B] In certain preferred embodiments, the BP-2a D3 polypeptide is at least 40%, at least 50%, at least 60%, at least 70%, at least 80%, at least 90%, at least 95%, at least 97%, at least 98%, or at least 99% identical to the amino acid sequence shown in SEQ ID NO: 101, and the BP-2a D3 polypeptide comprises seven scaffold regions, wherein:
(i) the first scaffold region spans the amino acids at positions 344-368;
(ii) the second scaffold region spans the amino acids at positions 392-398;
(iii) the third scaffold region spans the amino acids at positions 403-411;
(iv) the fourth scaffold region spans the amino acids at positions 419-422;
(v) the fifth scaffold region spans the amino acids at positions 432-435;
(vi) the sixth scaffold region spans the amino acids at positions 442-445; and
(vii) the seventh scaffold region spans the amino acids at positions 452-465,
wherein the amino acids are numbered according to the sequence shown in SEQ ID NO:100, wherein amino acids 344-465 are the native BP-2a D3 polypeptide, or wherein said scaffold regions are at equivalent positions within the native BP-2a D3 polypeptide from other GBS strains.

[H36B] In certain embodiments, the BP-2a D3 polypeptide comprises seven scaffold regions, wherein:
(i) the first scaffold region is at least 40%, at least 50%, at least 60%, at least 70%, at least 80%, at least 90%, at least 95%, at least 97%, at least 98%, or at least 99% identical to the amino acid sequence shown in SEQ ID NO:108;
(ii) the second scaffold region is at least 40%, at least 50%, at least 60%, at least 70%, at least 80%, at least 90%, at least 95%, at least 97%, at least 98%, or at least 99% identical to the amino acid sequence shown in SEQ ID NO:109;
(iii) the third scaffold region is at least 40%, at least 50%, at least 60%, at least 70%, at least 80%, at least 90%, at least 95%, at least 97%, at least 98%, or at least 99% identical to the amino acid sequence shown in SEQ ID NO: 110;
(iv) the fourth scaffold region is at least 40%, at least 50%, at least 60%, at least 70%, at least 80%, at least 90%, at least 95%, at least 97%, at least 98%, or at least 99% identical to the amino acid sequence shown in SEQ ID NO: 111;
(v) the fifth scaffold region is at least 40%, at least 50%, at least 60%, at least 70%, at least 80%, at least 90%, at least 95%, at least 97%, at least 98%, or at least 99% identical to the amino acid sequence shown in SEQ ID NO: 112;
(vi) the sixth scaffold region is at least 40%, at least 50%, at least 60%, at least 70%, at least 80%, at least 90%, at least 95%, at least 97%, at least 98%, or at least 99% identical to the amino acid sequence shown in SEQ ID NO: 113; and
(vii) the seventh scaffold region is at least 40%, at least 50%, at least 60%, at least 70%, at least 80%, at least 90%, at least 95%, at least 97%, at least 98%, or at least 99% identical to the amino acid sequence shown in SEQ ID NO: 114.

[H36B] In certain embodiments, wherein the BP-2a D3 polypeptide comprises seven scaffold regions, wherein:
(i) the first scaffold region comprises or consists of the amino acid sequence shown in SEQ ID NO:108;
(ii) the second scaffold region comprises or consists of the amino acid sequence shown in SEQ ID NO:109;
(iii) the third scaffold region comprises or consists of the amino acid sequence shown in SEQ ID NO:110;
(iv) the fourth scaffold region comprises or consists of the amino acid sequence shown in SEQ ID NO:111;
(v) the fifth scaffold region comprises or consists of the amino acid sequence shown in SEQ ID NO:112;
(vi) the sixth scaffold region comprises or consists of the amino acid sequence shown in SEQ ID NO:113; and
(vii) the seventh scaffold region comprises or consists of the amino acid sequence shown in SEQ ID NO:114.

[CJB111] In certain preferred embodiments, the BP-2a D3 polypeptide is at least 40%, at least 50%, at least 60%, at least 70%, at least 80%, at least 90%, at least 95%, at least 97%, at least 98%, or at least 99% identical to the amino acid sequence shown in SEQ ID NO: 116, and the at least one endogenous loop of the BP-2a D3 polypeptide is selected from the first, second, third, fourth, fifth and sixth endogenous loops of the BP-2a D3 polypeptide, wherein:
(i) the first endogenous loop of the BP-2a D3 polypeptide spans the amino acids at positions 357-370;
(ii) the second endogenous loop of the BP-2a D3 polypeptide spans the amino acids at positions 379-382;
(iii) the third endogenous loop of the BP-2a D3 polypeptide spans the amino acids at positions 392-397;
(iv) the fourth endogenous loop of the BP-2a D3 polypeptide spans the amino acids at positions 403-411;
(v) the fifth endogenous loop of the BP-2a D3 polypeptide spans the amino acids at positions 416-421; and
(vi) the sixth endogenous loop of the BP-2a D3 polypeptide spans the amino acids at positions 428-431,
wherein the amino acids are numbered according to the sequence shown in SEQ ID NO:115, wherein amino acids 332-446 are the native BP-2a D3 polypeptide, or wherein said endogenous loops are at equivalent positions within the native BP-2a D3 polypeptide from other GBS strains.

[CJB111] In certain preferred embodiments, the BP-2a D3 polypeptide is at least 40%, at least 50%, at least 60%, at least 70%, at least 80%, at least 90%, at least 95%, at least 97%, at least 98%, or at least 99% identical to the amino acid sequence shown in SEQ ID NO: 116, and, the BP-2a D3 polypeptide comprises seven scaffold regions, wherein:
(i) the first scaffold region spans the amino acids at positions 332-356;
(ii) the second scaffold region spans the amino acids at positions 371-378;
(iii) the third scaffold region spans the amino acids at positions 383-391;
(iv) the fourth scaffold region spans the amino acids at positions 398-402;
(v) the fifth scaffold region spans the amino acids at positions 412-415;
(vi) the sixth scaffold region spans the amino acids at positions 422-427; and
(vii) the seventh scaffold region spans the amino acids at positions 432-446,
wherein the amino acids are numbered according to the sequence shown in SEQ ID NO:115, wherein amino acids 332-446 are the native BP-2a D3 polypeptide, or wherein said scaffold regions are at equivalent positions within the native BP-2a D3 polypeptide from other GBS strains.

[CJB111] In certain embodiments, the BP-2a D3 polypeptide comprises seven scaffold regions, wherein:
(i) the first scaffold region is at least 40%, at least 50%, at least 60%, at least 70%, at least 80%, at least 90%, at least 95%, at least 97%, at least 98%, or at least 99% identical to the amino acid sequence shown in SEQ ID NO:123;
(ii) the second scaffold region is at least 40%, at least 50%, at least 60%, at least 70%, at least 80%, at least 90%, at least 95%, at least 97%, at least 98%, or at least 99% identical to the amino acid sequence shown in SEQ ID NO:124;
(iii) the third scaffold region is at least 40%, at least 50%, at least 60%, at least 70%, at least 80%, at least 90%, at least 95%, at least 97%, at least 98%, or at least 99% identical to the amino acid sequence shown in SEQ ID NO:125;
(iv) the fourth scaffold region is at least 40%, at least 50%, at least 60%, at least 70%, at least 80%, at least 90%, at least 95%, at least 97%, at least 98%, or at least 99% identical to the amino acid sequence shown in SEQ ID NO:126;
(v) the fifth scaffold region is at least 40%, at least 50%, at least 60%, at least 70%, at least 80%, at least 90%, at least 95%, at least 97%, at least 98%, or at least 99% identical to the amino acid sequence shown in SEQ ID NO:127;
(vi) the sixth scaffold region is at least 40%, at least 50%, at least 60%, at least 70%, at least 80%, at least 90%, at least 95%, at least 97%, at least 98%, or at least 99% identical to the amino acid sequence shown in SEQ ID NO:128; and
(vii) the seventh scaffold region is at least 40%, at least 50%, at least 60%, at least 70%, at least 80%, at least 90%, at least 95%, at least 97%, at least 98%, or at least 99% identical to the amino acid sequence shown in SEQ ID NO:129.

[CJB111] In certain embodiments, wherein the BP-2a D3 polypeptide comprises seven scaffold regions, wherein:
(i) the first scaffold region comprises or consists of the amino acid sequence shown in SEQ ID NO:123;
(ii) the second scaffold region comprises or consists of the amino acid sequence shown in SEQ ID NO:124;
(iii) the third scaffold region comprises or consists of the amino acid sequence shown in SEQ ID NO:125;
(iv) the fourth scaffold region comprises or consists of the amino acid sequence shown in SEQ ID NO:126;
(v) the fifth scaffold region comprises or consists of the amino acid sequence shown in SEQ ID NO:127;
(vi) the sixth scaffold region comprises or consists of the amino acid sequence shown in SEQ ID NO:128; and
(vii) the seventh scaffold region comprises or consists of the amino acid sequence shown in SEQ ID NO:129.

In certain preferred embodiments, the BP-2a D3 polypeptide is at least 40%, at least 50%, at least 60%, at least 70%, at least 80%, at least 90%, at least 95%, at least 97%, at least 98%, or at least 99% identical to the amino acid sequence shown in SEQ ID NO: 131, and the at least one endogenous loop of the BP-2a D3 polypeptide is selected from the first, second, third, fourth, fifth and sixth endogenous loops of the BP-2a D3 polypeptide, wherein:
(i) the first endogenous loop of the BP-2a D3 polypeptide spans the amino acids at positions 387-399;
(ii) the second endogenous loop of the BP-2a D3 polypeptide spans the amino acids at positions 404-412;
(iii) the third endogenous loop of the BP-2a D3 polypeptide spans the amino acids at positions 417-436;
(iv) the fourth endogenous loop of the BP-2a D3 polypeptide spans the amino acids at positions 442-447;
(v) the fifth endogenous loop of the BP-2a D3 polypeptide spans the amino acids at positions 454-459; and
(vi) the sixth endogenous loop of the BP-2a D3 polypeptide spans the amino acids at positions 465-468,
wherein the amino acids are numbered according to the sequence shown in SEQ ID NO:130, wherein amino acids 364-483 are the native BP-2a D3 polypeptide, or wherein said endogenous loops are at equivalent positions within the native BP-2a D3 polypeptide from other GBS strains.

In certain preferred embodiments, the BP-2a D3 polypeptide is at least 40%, at least 50%, at least 60%, at least 70%, at least 80%, at least 90%, at least 95%, at least 97%, at least 98%, or at least 99% identical to the amino acid sequence shown in SEQ ID NO: 131, and the BP-2a D3 polypeptide comprises seven scaffold regions, wherein:
(i) the first scaffold region spans the amino acids at positions 364-386;
(ii) the second scaffold region spans the amino acids at positions 400-403;
(iii) the third scaffold region spans the amino acids at positions 413-416;
(iv) the fourth scaffold region spans the amino acids at positions 437-441;
(v) the fifth scaffold region spans the amino acids at positions 448-453;
(vi) the sixth scaffold region spans the amino acids at positions 460-464; and
(vii) the seventh scaffold region spans the amino acids at positions 469-483,
wherein the amino acids are numbered according to the sequence shown in SEQ ID NO:130, wherein amino acids 364-483 are the native BP-2a D3 polypeptide, or wherein said scaffold regions are at equivalent positions within the native BP-2a D3 polypeptide from other GBS strains.

In certain embodiments, the BP-2a D3 polypeptide comprises seven scaffold regions, wherein:
(i) the first scaffold region is at least 40%, at least 50%, at least 60%, at least 70%, at least 80%, at least 90%, at least 95%, at least 97%, at least 98%, or at least 99% identical to the amino acid sequence shown in SEQ ID NO:138;
(ii) the second scaffold region is at least 40%, at least 50%, at least 60%, at least 70%, at least 80%, at least 90%, at least 95%, at least 97%, at least 98%, or at least 99% identical to the amino acid sequence shown in SEQ ID NO:139;
(iii) the third scaffold region is at least 40%, at least 50%, at least 60%, at least 70%, at least 80%, at least 90%, at least 95%, at least 97%, at least 98%, or at least 99% identical to the amino acid sequence shown in SEQ ID NO:140;
(iv) the fourth scaffold region is at least 40%, at least 50%, at least 60%, at least 70%, at least 80%, at least 90%, at least 95%, at least 97%, at least 98%, or at least 99% identical to the amino acid sequence shown in SEQ ID NO:141;
(v) the fifth scaffold region is at least 40%, at least 50%, at least 60%, at least 70%, at least 80%, at least 90%, at least 95%, at least 97%, at least 98%, or at least 99% identical to the amino acid sequence shown in SEQ ID NO:142;
(vi) the sixth scaffold region is at least 40%, at least 50%, at least 60%, at least 70%, at least 80%, at least 90%, at least 95%, at least 97%, at least 98%, or at least 99% identical to the amino acid sequence shown in SEQ ID NO:143; and
(vii) the seventh scaffold region is at least 40%, at least 50%, at least 60%, at least 70%, at least 80%, at least 90%, at least 95%, at least 97%, at least 98%, or at least 99% identical to the amino acid sequence shown in SEQ ID NO:144.

In certain embodiments, wherein the BP-2a D3 polypeptide comprises seven scaffold regions, wherein:
(i) the first scaffold region comprises or consists of the amino acid sequence shown in SEQ ID NO:138;
(ii) the second scaffold region comprises or consists of the amino acid sequence shown in SEQ ID NO:139;
(iii) the third scaffold region comprises or consists of the amino acid sequence shown in SEQ ID NO:140;
(iv) the fourth scaffold region comprises or consists of the amino acid sequence shown in SEQ ID NO:141;
(v) the fifth scaffold region comprises or consists of the amino acid sequence shown in SEQ ID NO:142;
(vi) the sixth scaffold region comprises or consists of the amino acid sequence shown in SEQ ID NO:143; and
(vii) the seventh scaffold region comprises or consists of the amino acid sequence shown in SEQ ID NO:144.

[CJB110] In certain preferred embodiments, the BP-2a D3 polypeptide is at least 40%, at least 50%, at least 60%, at least 70%, at least 80%, at least 90%, at least 95%, at least 97%, at least 98%, or at least 99% identical to the amino acid sequence shown in SEQ ID NO: 146, and the at least one endogenous loop of the BP-2a D3 polypeptide is selected from the first, second, third, fourth, fifth and sixth endogenous loops of the BP-2a D3 polypeptide, wherein:
(i) the first endogenous loop of the BP-2a D3 polypeptide spans the amino acids at positions 385-407;
(ii) the second endogenous loop of the BP-2a D3 polypeptide spans the amino acids at positions 416-419;
(iii) the third endogenous loop of the BP-2a D3 polypeptide spans the amino acids at positions 429-435;
(iv) the fourth endogenous loop of the BP-2a D3 polypeptide spans the amino acids at positions 440-447;
(v) the fifth endogenous loop of the BP-2a D3 polypeptide spans the amino acids at positions 452-457; and
(vi) the sixth endogenous loop of the BP-2a D3 polypeptide spans the amino acids at positions 463-466,
wherein the amino acids are numbered according to the sequence shown in SEQ ID NO:145, wherein amino acids 360-481 are the native BP-2a D3 polypeptide, or wherein said endogenous loops are at equivalent positions within the native BP-2a D3 polypeptide from other GBS strains.

[CJB110] In certain preferred embodiments, the BP-2a D3 polypeptide is at least 40%, at least 50%, at least 60%, at least 70%, at least 80%, at least 90%, at least 95%, at least 97%, at least 98%, or at least 99% identical to the amino acid sequence shown in SEQ ID NO: 146, and the BP-2a D3 polypeptide comprises seven scaffold regions, wherein:
(i) the first scaffold region spans the amino acids at positions 360-384;
(ii) the second scaffold region spans the amino acids at positions 408-415;
(iii) the third scaffold region spans the amino acids at positions 420-428;
(iv) the fourth scaffold region spans the amino acids at positions 436-439;
(v) the fifth scaffold region spans the amino acids at positions 448-451;
(vi) the sixth scaffold region spans the amino acids at positions 458-462; and
(vii) the seventh scaffold region spans the amino acids at positions 467-481,
wherein the amino acids are numbered according to the sequence shown in SEQ ID NO:145, wherein amino acids 360-481 are the native BP-2a D3 polypeptide, or wherein said scaffold regions are at equivalent positions within the native BP-2a D3 polypeptide from other GBS strains.

[CJB110] In certain embodiments, the BP-2a D3 polypeptide comprises seven scaffold regions, wherein:
(i) the first scaffold region is at least 40%, at least 50%, at least 60%, at least 70%, at least 80%, at least 90%, at least 95%, at least 97%, at least 98%, or at least 99% identical to the amino acid sequence shown in SEQ ID NO:153;
(ii) the second scaffold region is at least 40%, at least 50%, at least 60%, at least 70%, at least 80%, at least 90%, at least 95%, at least 97%, at least 98%, or at least 99% identical to the amino acid sequence shown in SEQ ID NO:154;
(iii) the third scaffold region is at least 40%, at least 50%, at least 60%, at least 70%, at least 80%, at least 90%, at least 95%, at least 97%, at least 98%, or at least 99% identical to the amino acid sequence shown in SEQ ID NO:155;
(iv) the fourth scaffold region is at least 40%, at least 50%, at least 60%, at least 70%, at least 80%, at least 90%, at least 95%, at least 97%, at least 98%, or at least 99% identical to the amino acid sequence shown in SEQ ID NO:156;
(v) the fifth scaffold region is at least 40%, at least 50%, at least 60%, at least 70%, at least 80%, at least 90%, at least 95%, at least 97%, at least 98%, or at least 99% identical to the amino acid sequence shown in SEQ ID NO:157;
(vi) the sixth scaffold region is at least 40%, at least 50%, at least 60%, at least 70%, at least 80%, at least 90%, at least 95%, at least 97%, at least 98%, or at least 99% identical to the amino acid sequence shown in SEQ ID NO:158; and
(vii) the seventh scaffold region is at least 40%, at least 50%, at least 60%, at least 70%, at least 80%, at least 90%, at least 95%, at least 97%, at least 98%, or at least 99% identical to the amino acid sequence shown in SEQ ID NO:159.

[CJB110] In certain embodiments, wherein the BP-2a D3 polypeptide comprises seven scaffold regions, wherein:
(i) the first scaffold region comprises or consists of the amino acid sequence shown in SEQ ID NO:153;
(ii) the second scaffold region comprises or consists of the amino acid sequence shown in SEQ ID NO:154;
(iii) the third scaffold region comprises or consists of the amino acid sequence shown in SEQ ID NO:155;
(iv) the fourth scaffold region comprises or consists of the amino acid sequence shown in SEQ ID NO:156;
(v) the fifth scaffold region comprises or consists of the amino acid sequence shown in SEQ ID NO:157;
(vi) the sixth scaffold region comprises or consists of the amino acid sequence shown in SEQ ID NO:158; and
(vii) the seventh scaffold region comprises or consists of the amino acid sequence shown in SEQ ID NO:159.

[DK21] In certain preferred embodiments, the BP-2a D3 polypeptide is at least 40%, at least 50%, at least 60%, at least 70%, at least 80%, at least 90%, at least 95%, at least 97%, at least 98%, or at least 99% identical to the amino acid sequence shown in SEQ ID NO: 161, and the at least one endogenous loop of the BP-2a D3 polypeptide is selected from the first, second, third, fourth, fifth and sixth endogenous loops of the BP-2a D3 polypeptide, wherein:
(i) the first endogenous loop of the BP-2a D3 polypeptide spans the amino acids at positions 365-379;
(ii) the second endogenous loop of the BP-2a D3 polypeptide spans the amino acids at positions 387-390;
(iii) the third endogenous loop of the BP-2a D3 polypeptide spans the amino acids at positions 397-405;
(iv) the fourth endogenous loop of the BP-2a D3 polypeptide spans the amino acids at positions 410-418;
(v) the fifth endogenous loop of the BP-2a D3 polypeptide spans the amino acids at positions 423-428; and
(vi) the sixth endogenous loop of the BP-2a D3 polypeptide spans the amino acids at positions 435-438,
wherein the amino acids are numbered according to the sequence shown in SEQ ID NO:160, wherein amino acids 339-453 the native BP-2a D3 polypeptide, or wherein said endogenous loops are at equivalent positions within the native BP-2a D3 polypeptide from other GBS strains.

[DK21] In certain preferred embodiments, the BP-2a D3 polypeptide is at least 40%, at least 50%, at least 60%, at least 70%, at least 80%, at least 90%, at least 95%, at least 97%, at least 98%, or at least 99% identical to the amino acid sequence shown in SEQ ID NO: 161, and the BP-2a D3 polypeptide comprises seven scaffold regions, wherein:
(i) the first scaffold region spans the amino acids at positions 339-364;
(ii) the second scaffold region spans the amino acids at positions 380-386;
(iii) the third scaffold region spans the amino acids at positions 391-396;
(iv) the fourth scaffold region spans the amino acids at positions 406-409;
(v) the fifth scaffold region spans the amino acids at positions 419-422;
(vi) the sixth scaffold region spans the amino acids at positions 429-434; and
(vii) the seventh scaffold region spans the amino acids at positions 439-453,
wherein the amino acids are numbered according to the sequence shown in SEQ ID NO:160, wherein amino acids 339-453 are the native BP-2a D3 polypeptide, or wherein said endogenous loops are at equivalent positions within the native BP-2a D3 polypeptide from other GBS strains.

[DK21] In certain embodiments, the BP-2a D3 polypeptide comprises seven scaffold regions, wherein:
(i) the first scaffold region is at least 40%, at least 50%, at least 60%, at least 70%, at least 80%, at least 90%, at least 95%, at least 97%, at least 98%, or at least 99% identical to the amino acid sequence shown in SEQ ID NO:168;
(ii) the second scaffold region is at least 40%, at least 50%, at least 60%, at least 70%, at least 80%, at least 90%, at least 95%, at least 97%, at least 98%, or at least 99% identical to the amino acid sequence shown in SEQ ID NO:169;
(iii) the third scaffold region is at least 40%, at least 50%, at least 60%, at least 70%, at least 80%, at least 90%, at least 95%, at least 97%, at least 98%, or at least 99% identical to the amino acid sequence shown in SEQ ID NO:170;
(iv) the fourth scaffold region is at least 40%, at least 50%, at least 60%, at least 70%, at least 80%, at least 90%, at least 95%, at least 97%, at least 98%, or at least 99% identical to the amino acid sequence shown in SEQ ID NO:171;
(v) the fifth scaffold region is at least 40%, at least 50%, at least 60%, at least 70%, at least 80%, at least 90%, at least 95%, at least 97%, at least 98%, or at least 99% identical to the amino acid sequence shown in SEQ ID NO:172;
(vi) the sixth scaffold region is at least 40%, at least 50%, at least 60%, at least 70%, at least 80%, at least 90%, at least 95%, at least 97%, at least 98%, or at least 99% identical to the amino acid sequence shown in SEQ ID NO:173; and
(vii) the seventh scaffold region is at least 40%, at least 50%, at least 60%, at least 70%, at least 80%, at least 90%, at least 95%, at least 97%, at least 98%, or at least 99% identical to the amino acid sequence shown in SEQ ID NO:174.

[DK21] In certain embodiments, wherein the BP-2a D3 polypeptide comprises seven scaffold regions, wherein:
(i) the first scaffold region comprises or consists of the amino acid sequence shown in SEQ ID NO:168;
(ii) the second scaffold region comprises or consists of the amino acid sequence shown in SEQ ID NO:169;
(iii) the third scaffold region comprises or consists of the amino acid sequence shown in SEQ ID NO:170;
(iv) the fourth scaffold region comprises or consists of the amino acid sequence shown in SEQ ID NO:171;
(v) the fifth scaffold region comprises or consists of the amino acid sequence shown in SEQ ID NO:172;
(vi) the sixth scaffold region comprises or consists of the amino acid sequence shown in SEQ ID NO:173; and
(vii) the seventh scaffold region comprises or consists of the amino acid sequence shown in SEQ ID NO:174.

In certain embodiments, the at least one endogenous loop of the BP-2a D3 polypeptide replaced (wholly or partially) with an exogenous polypeptide is selected from the first, second, third, fifth and sixth endogenous loops.

In certain embodiments, two or more endogenous loops of the BP-2a D3 polypeptide are each independently partially or wholly replaced by an exogenous polypeptide.

In certain embodiments, the first endogenous loop of the BP-2a D3 polypeptide is partially or wholly replaced by an exogenous polypeptide.

In certain preferred embodiments, the second endogenous loop of the BP-2a D3 polypeptide is partially or wholly replaced by an exogenous polypeptide.

In certain embodiments, the third endogenous loop of the BP-2a D3 polypeptide is partially or wholly replaced by an exogenous polypeptide.

In certain embodiments, the fifth endogenous loop of the BP-2a D3 polypeptide is partially or wholly replaced by an exogenous polypeptide.

In certain embodiments, the sixth endogenous loop of the BP-2a D3 polypeptide is partially or wholly replaced by an exogenous polypeptide.

[515] In certain embodiments, when present:
(i) the first endogenous loop of the BP-2a D3 polypeptide is at least 40%, at least 50%, at least 60%, at least 70%, at least 80%, at least 90%, at least 95%, at least 97%, at least 98%, or at least 99% to the amino acid sequence shown in SEQ ID NO:4;
(ii) the second endogenous loop of the BP-2a D3 polypeptide is at least 40%, at least 50%, at least 60%, at least 70%, at least 80%, at least 90%, at least 95%, at least 97%, at least 98%, or at least 99% identical to the amino acid sequence shown in SEQ ID NO:5;
(iii) the third endogenous loop of the BP-2a D3 polypeptide is at least 40%, at least 50%, at least 60%, at least 70%, at least 80%, at least 90%, at least 95%, at least 97%, at least 98%, or at least 99% identical to the amino acid sequence shown in SEQ ID NO:6;
(iv) the fourth endogenous loop of the BP-2a D3 polypeptide is at least 40%, at least 50%, at least 60%, at least 70%, at least 80%, at least 90%, at least 95%, at least 97%, at least 98%, or at least 99% identical to the amino acid sequence shown in SEQ ID NO:7;
(v) the fifth endogenous loop of the BP-2a D3 polypeptide is at least 40%, at least 50%, at least 60%, at least 70%, at least 80%, at least 90%, at least 95%, at least 97%, at least 98%, or at least 99% identical to the amino acid sequence shown in SEQ ID NO:8; and
(vi) the sixth endogenous loop of the BP-2a D3 polypeptide is at least 40%, at least 50%, at least 60%, at least 70%, at least 80%, at least 90%, at least 95%, at least 97%, at least 98%, or at least 99% identical to the amino acid sequence shown in SEQ ID NO:9.

[515] In certain embodiments, when present:
(i) the first endogenous loop of the BP-2a D3 polypeptide comprises or consists of the amino acid sequence shown in SEQ ID NO:4;
(ii) the second endogenous loop of the BP-2a D3 polypeptide comprises or consists of the amino acid sequence shown in SEQ ID NO:5;
(iii) the third endogenous loop of the BP-2a D3 polypeptide comprises or consists of the amino acid sequence shown in SEQ ID NO:6;
(iv) the fourth endogenous loop of the BP-2a D3 polypeptide comprises or consists of the amino acid sequence shown in SEQ ID NO:7;
(v) the fifth endogenous loop of the BP-2a D3 polypeptide comprises or consists of the amino acid sequence shown in SEQ ID NO:8; and
(vi) the sixth endogenous loop of the BP-2a D3 polypeptide comprises or consists of the amino acid sequence shown in SEQ ID NO:9.

[515] In certain embodiments, the fourth endogenous loop of the BP-2a D3 polypeptide is present and is at least 40%, at least 50%, at least 60%, at least 70%, at least 80%, at least 90%, at least 95%, at least 97%, at least 98%, or at least 99% identical to the amino acid sequence shown in SEQ ID NO:7. In preferred such embodiments, the fourth endogenous loop of the BP-2a D3 polypeptide is present and comprises or consists of the amino acid sequence shown in SEQ ID NO:7.

Based on the sequences of the endogenous loops and scaffold regions within the BP-2a D3 polypeptide from strain 515 (elucidated following analysis of the crystal structure of BP-2a from stain 515 in Nuccitelli et al 2011, it is within the remit of the person skilled in the art to determine the sequences of the endogenous loops and scaffold regions for other GBS strains. For example, the sequences of the endogenous loops with regards to GBS strains [H36B], [CJB111], [CJB110], [2603] and [DK21] are predicted below.

[H36B] In certain embodiments, when present:
(i) the first endogenous loop of the BP-2a D3 polypeptide is at least 40%, at least 50%, at least 60%, at least 70%, at least 80%, at least 90%, at least 95%, at least 97%, at least 98%, or at least 99% to the amino acid sequence shown in SEQ ID NO:102;
(ii) the second endogenous loop of the BP-2a D3 polypeptide is at least 40%, at least 50%, at least 60%, at least 70%, at least 80%, at least 90%, at least 95%, at least 97%, at least 98%, or at least 99% identical to the amino acid sequence shown in SEQ ID NO:103;
(iii) the third endogenous loop of the BP-2a D3 polypeptide is at least 40%, at least 50%, at least 60%, at least 70%, at least 80%, at least 90%, at least 95%, at least 97%, at least 98%, or at least 99% identical to the amino acid sequence shown in SEQ ID NO:104;
(iv) the fourth endogenous loop of the BP-2a D3 polypeptide is at least 40%, at least 50%, at least 60%, at least 70%, at least 80%, at least 90%, at least 95%, at least 97%, at least 98%, or at least 99% identical to the amino acid sequence shown in SEQ ID NO:105;
(v) the fifth endogenous loop of the BP-2a D3 polypeptide is at least 40%, at least 50%, at least 60%, at least 70%, at least 80%, at least 90%, at least 95%, at least 97%, at least 98%, or at least 99% identical to the amino acid sequence shown in SEQ ID NO:106; and
(vi) the sixth endogenous loop of the BP-2a D3 polypeptide is at least 40%, at least 50%, at least 60%, at least 70%, at least 80%, at least 90%, at least 95%, at least 97%, at least 98%, or at least 99% identical to the amino acid sequence shown in SEQ ID NO:107.

[H36B] In certain embodiments, when present:
(i) the first endogenous loop of the BP-2a D3 polypeptide comprises or consists of the amino acid sequence shown in SEQ ID NO:102;
(ii) the second endogenous loop of the BP-2a D3 polypeptide comprises or consists of the amino acid sequence shown in SEQ ID NO:103;
(iii) the third endogenous loop of the BP-2a D3 polypeptide comprises or consists of the amino acid sequence shown in SEQ ID NO:104;
(iv) the fourth endogenous loop of the BP-2a D3 polypeptide comprises or consists of the amino acid sequence shown in SEQ ID NO:105;
(v) the fifth endogenous loop of the BP-2a D3 polypeptide comprises or consists of the amino acid sequence shown in SEQ ID NO:106; and
(vi) the sixth endogenous loop of the BP-2a D3 polypeptide comprises or consists of the amino acid sequence shown in SEQ ID NO:107.

[H36B] In certain embodiments, the fourth endogenous loop of the BP-2a D3 polypeptide is present and is at least 40%, at least 50%, at least 60%, at least 70%, at least 80%, at least 90%, at least 95%, at least 97%, at least 98%, or at least 99% identical to the amino acid sequence shown in SEQ ID NO:105. In preferred such embodiments, the fourth endogenous loop of the BP-2a D3 polypeptide is present and comprises or consists of the amino acid sequence shown in SEQ ID NO:105.

[CJB111] In certain embodiments, when present:
(i) the first endogenous loop of the BP-2a D3 polypeptide is at least 40%, at least 50%, at least 60%, at least 70%, at least 80%, at least 90%, at least 95%, at least 97%, at least 98%, or at least 99% to the amino acid sequence shown in SEQ ID NO:117;
(ii) the second endogenous loop of the BP-2a D3 polypeptide is at least 40%, at least 50%, at least 60%, at least 70%, at least 80%, at least 90%, at least 95%, at least 97%, at least 98%, or at least 99% identical to the amino acid sequence shown in SEQ ID NO:118;
(iii) the third endogenous loop of the BP-2a D3 polypeptide is at least 40%, at least 50%, at least 60%, at least 70%, at least 80%, at least 90%, at least 95%, at least 97%, at least 98%, or at least 99% identical to the amino acid sequence shown in SEQ ID NO:119;
(iv) the fourth endogenous loop of the BP-2a D3 polypeptide is at least 40%, at least 50%, at least 60%, at least 70%, at least 80%, at least 90%, at least 95%, at least 97%, at least 98%, or at least 99% identical to the amino acid sequence shown in SEQ ID NO:120;
(v) the fifth endogenous loop of the BP-2a D3 polypeptide is at least 40%, at least 50%, at least 60%, at least 70%, at least 80%, at least 90%, at least 95%, at least 97%, at least 98%, or at least 99% identical to the amino acid sequence shown in SEQ ID NO:121; and
(vi) the sixth endogenous loop of the BP-2a D3 polypeptide is at least 40%, at least 50%, at least 60%, at least 70%, at least 80%, at least 90%, at least 95%, at least 97%, at least 98%, or at least 99% identical to the amino acid sequence shown in SEQ ID NO:122.

[CJB111] In certain embodiments, when present:
(i) the first endogenous loop of the BP-2a D3 polypeptide comprises or consists of the amino acid sequence shown in SEQ ID NO:117;
(ii) the second endogenous loop of the BP-2a D3 polypeptide comprises or consists of the amino acid sequence shown in SEQ ID NO:118;
(iii) the third endogenous loop of the BP-2a D3 polypeptide comprises or consists of the amino acid sequence shown in SEQ ID NO:119;
(iv) the fourth endogenous loop of the BP-2a D3 polypeptide comprises or consists of the amino acid sequence shown in SEQ ID NO:120;
(v) the fifth endogenous loop of the BP-2a D3 polypeptide comprises or consists of the amino acid sequence shown in SEQ ID NO:121; and
(vi) the sixth endogenous loop of the BP-2a D3 polypeptide comprises or consists of the amino acid sequence shown in SEQ ID NO:122.

[CJB111] In certain embodiments, the fourth endogenous loop of the BP-2a D3 polypeptide is present and is at least 40%, at least 50%, at least 60%, at least 70%, at least 80%, at least 90%, at least 95%, at least 97%, at least 98%, or at least 99% identical to the amino acid sequence shown in SEQ ID NO:120. In preferred such embodiments, the fourth endogenous loop of the BP-2a D3 polypeptide is present and comprises or consists of the amino acid sequence shown in SEQ ID NO:120.

In certain embodiments, when present:
(i) the first endogenous loop of the BP-2a D3 polypeptide is at least 40%, at least 50%, at least 60%, at least 70%, at least 80%, at least 90%, at least 95%, at least 97%, at least 98%, or at least 99% to the amino acid sequence shown in SEQ ID NO:132;
(ii) the second endogenous loop of the BP-2a D3 polypeptide is at least 40%, at least 50%, at least 60%, at least 70%, at least 80%, at least 90%, at least 95%, at least 97%, at least 98%, or at least 99% identical to the amino acid sequence shown in SEQ ID NO:133;
(iii) the third endogenous loop of the BP-2a D3 polypeptide is at least 40%, at least 50%, at least 60%, at least 70%, at least 80%, at least 90%, at least 95%, at least 97%, at least 98%, or at least 99% identical to the amino acid sequence shown in SEQ ID NO:134;
(iv) the fourth endogenous loop of the BP-2a D3 polypeptide is at least 40%, at least 50%, at least 60%, at least 70%, at least 80%, at least 90%, at least 95%, at least 97%, at least 98%, or at least 99% identical to the amino acid sequence shown in SEQ ID NO:135;
(v) the fifth endogenous loop of the BP-2a D3 polypeptide is at least 40%, at least 50%, at least 60%, at least 70%, at least 80%, at least 90%, at least 95%, at least 97%, at least 98%, or at least 99% identical to the amino acid sequence shown in SEQ ID NO:136; and
(vi) the sixth endogenous loop of the BP-2a D3 polypeptide is at least 40%, at least 50%, at least 60%, at least 70%, at least 80%, at least 90%, at least 95%, at least 97%, at least 98%, or at least 99% identical to the amino acid sequence shown in SEQ ID NO:137.

In certain embodiments, when present:
(i) the first endogenous loop of the BP-2a D3 polypeptide comprises or consists of the amino acid sequence shown in SEQ ID NO:132;
(ii) the second endogenous loop of the BP-2a D3 polypeptide comprises or consists of the amino acid sequence shown in SEQ ID NO:133;
(iii) the third endogenous loop of the BP-2a D3 polypeptide comprises or consists of the amino acid sequence shown in SEQ ID NO:134;
(iv) the fourth endogenous loop of the BP-2a D3 polypeptide comprises or consists of the amino acid sequence shown in SEQ ID NO:135;
(v) the fifth endogenous loop of the BP-2a D3 polypeptide comprises or consists of the amino acid sequence shown in SEQ ID NO:136; and
(vi) the sixth endogenous loop of the BP-2a D3 polypeptide comprises or consists of the amino acid sequence shown in SEQ ID NO:137.

In certain embodiments, the fourth endogenous loop of the BP-2a D3 polypeptide is present and is at least 40%, at least 50%, at least 60%, at least 70%, at least 80%, at least 90%, at least 95%, at least 97%, at least 98%, or at least 99% identical to the amino acid sequence shown in SEQ ID NO:135. In preferred such embodiments, the fourth endogenous loop of the BP-2a D3 polypeptide is present and comprises or consists of the amino acid sequence shown in SEQ ID NO:135.

[CJB110] In certain embodiments, when present:
(i) the first endogenous loop of the BP-2a D3 polypeptide is at least 40%, at least 50%, at least 60%, at least 70%, at least 80%, at least 90%, at least 95%, at least 97%, at least 98%, or at least 99% to the amino acid sequence shown in SEQ ID NO:147;
(ii) the second endogenous loop of the BP-2a D3 polypeptide is at least 40%, at least 50%, at least 60%, at least 70%, at least 80%, at least 90%, at least 95%, at least 97%, at least 98%, or at least 99% identical to the amino acid sequence shown in SEQ ID NO:148;
(iii) the third endogenous loop of the BP-2a D3 polypeptide is at least 40%, at least 50%, at least 60%, at least 70%, at least 80%, at least 90%, at least 95%, at least 97%, at least 98%, or at least 99% identical to the amino acid sequence shown in SEQ ID NO:149;
(iv) the fourth endogenous loop of the BP-2a D3 polypeptide is at least 40%, at least 50%, at least 60%, at least 70%, at least 80%, at least 90%, at least 95%, at least 97%, at least 98%, or at least 99% identical to the amino acid sequence shown in SEQ ID NO:150;
(v) the fifth endogenous loop of the BP-2a D3 polypeptide is at least 40%, at least 50%, at least 60%, at least 70%, at least 80%, at least 90%, at least 95%, at least 97%, at least 98%, or at least 99% identical to the amino acid sequence shown in SEQ ID NO:151; and
(vi) the sixth endogenous loop of the BP-2a D3 polypeptide is at least 40%, at least 50%, at least 60%, at least 70%, at least 80%, at least 90%, at least 95%, at least 97%, at least 98%, or at least 99% identical to the amino acid sequence shown in SEQ ID NO:152.

[CJB110] In certain embodiments, when present:
(i) the first endogenous loop of the BP-2a D3 polypeptide comprises or consists of the amino acid sequence shown in SEQ ID NO:147;
(ii) the second endogenous loop of the BP-2a D3 polypeptide comprises or consists of the amino acid sequence shown in SEQ ID NO:148;
(iii) the third endogenous loop of the BP-2a D3 polypeptide comprises or consists of the amino acid sequence shown in SEQ ID NO:149;
(iv) the fourth endogenous loop of the BP-2a D3 polypeptide comprises or consists of the amino acid sequence shown in SEQ ID NO:150;
(v) the fifth endogenous loop of the BP-2a D3 polypeptide comprises or consists of the amino acid sequence shown in SEQ ID NO:151; and
(vi) the sixth endogenous loop of the BP-2a D3 polypeptide comprises or consists of the amino acid sequence shown in SEQ ID NO:152.

[CJB110] In certain embodiments, the fourth endogenous loop of the BP-2a D3 polypeptide is present and is at least 40%, at least 50%, at least 60%, at least 70%, at least 80%, at least 90%, at least 95%, at least 97%, at least 98%, or at least 99% identical to the amino acid sequence shown in SEQ ID NO:150. In preferred such embodiments, the fourth endogenous loop of the BP-2a D3 polypeptide is present and comprises or consists of the amino acid sequence shown in SEQ ID NO:150.

[DK21] In certain embodiments, when present:
(i) the first endogenous loop of the BP-2a D3 polypeptide is at least 40%, at least 50%, at least 60%, at least 70%, at least 80%, at least 90%, at least 95%, at least 97%, at least 98%, or at least 99% to the amino acid sequence shown in SEQ ID NO:162;
(ii) the second endogenous loop of the BP-2a D3 polypeptide is at least 40%, at least 50%, at least 60%, at least 70%, at least 80%, at least 90%, at least 95%, at least 97%, at least 98%, or at least 99% identical to the amino acid sequence shown in SEQ ID NO:163;
(iii) the third endogenous loop of the BP-2a D3 polypeptide is at least 40%, at least 50%, at least 60%, at least 70%, at least 80%, at least 90%, at least 95%, at least 97%, at least 98%, or at least 99% identical to the amino acid sequence shown in SEQ ID NO:164;
(iv) the fourth endogenous loop of the BP-2a D3 polypeptide is at least 40%, at least 50%, at least 60%, at least 70%, at least 80%, at least 90%, at least 95%, at least 97%, at least 98%, or at least 99% identical to the amino acid sequence shown in SEQ ID NO:165;
(v) the fifth endogenous loop of the BP-2a D3 polypeptide is at least 40%, at least 50%, at least 60%, at least 70%, at least 80%, at least 90%, at least 95%, at least 97%, at least 98%, or at least 99% identical to the amino acid sequence shown in SEQ ID NO:166; and
(vi) the sixth endogenous loop of the BP-2a D3 polypeptide is at least 40%, at least 50%, at least 60%, at least 70%, at least 80%, at least 90%, at least 95%, at least 97%, at least 98%, or at least 99% identical to the amino acid sequence shown in SEQ ID NO:167.

[DK21] In certain embodiments, when present:
(i) the first endogenous loop of the BP-2a D3 polypeptide comprises or consists of the amino acid sequence shown in SEQ ID NO:162;
(ii) the second endogenous loop of the BP-2a D3 polypeptide comprises or consists of the amino acid sequence shown in SEQ ID NO:163;
(iii) the third endogenous loop of the BP-2a D3 polypeptide comprises or consists of the amino acid sequence shown in SEQ ID NO:164;
(iv) the fourth endogenous loop of the BP-2a D3 polypeptide comprises or consists of the amino acid sequence shown in SEQ ID NO:165;
(v) the fifth endogenous loop of the BP-2a D3 polypeptide comprises or consists of the amino acid sequence shown in SEQ ID NO:166; and
(vi) the sixth endogenous loop of the BP-2a D3 polypeptide comprises or consists of the amino acid sequence shown in SEQ ID NO:167.

[DK21] In certain embodiments, the fourth endogenous loop of the BP-2a D3 polypeptide is present and is at least 40%, at least 50%, at least 60%, at least 70%, at least 80%, at least 90%, at least 95%, at least 97%, at least 98%, or at least 99% identical to the amino acid sequence shown in SEQ ID NO:165. In preferred such embodiments, the fourth endogenous loop of the BP-2a D3 polypeptide is present and comprises or consists of the amino acid sequence shown in SEQ ID NO:165.

In a preferred embodiment, the BP-2a D3 polypeptide is from Streptococcus agalactiae. It will be understood that the BP-2a D3 polypeptide can be from any strain of Streptococcus agalactiae and it is within the remit of the person skilled in the art to identify the endogenous loops and scaffold regions across any strain of Streptococcus agalactiae. However, in certain embodiments, the BP-2a D3 polypeptide is from Streptococcus agalactiae strain H36B. In certain embodiments, the BP-2a D3 polypeptide is from Streptococcus agalactiae strain CJB111. In certain embodiments, the BP-2a D3 polypeptide is from Streptococcus agalactiae strain 2603. In certain embodiments, the BP-2a D3 polypeptide is from Streptococcus agalactiae strain CJB110. In certain embodiments, the BP-2a D3 polypeptide is from Streptococcus agalactiae strain DK21.

In preferred embodiments, the BP-2a D3 polypeptide is from Streptococcus agalactiae strain 515. Particularly preferred as embodiments of the invention are combinations of embodiments identified with the same strain reference, e.g. 515, H36B, CJB111, 2603, CJB110, and DK21. It is therefore particularly preferred to combine embodiments relating to D3 polypeptides related to strain 515 with other embodiments relating to strain 515. Similarly it is particularly preferred to combine embodiments relating to D3 polypeptides related to strain CJB111 with other embodiments relating to strain CJB111. Embodiments relating to a given strain are evident from the sequences of Table 1 and/or the paragraph labelling (e.g. [515]). It is particular preferred to combine embodiments where they are each directed to D3 polypeptides derived from the same strain. Nevertheless all combinations of all disclosed embodiments are disclosed and encompassed as part of the invention herein.

In certain embodiments, the at least one endogenous loop of the BP-2a D3 polypeptide is partially replaced by an exogenous polypeptide.

In certain embodiments, the at least one endogenous loop of the BP-2a D3 polypeptide is wholly replaced by an exogenous polypeptide.

In certain embodiments, the one or more exogenous polypeptide(s) each comprise at least 5 amino acids.

In certain embodiments, the one or more exogenous polypeptide(s) each comprise at most 300 amino acids.

In certain embodiments, the one or more exogenous polypeptides each independently comprise 5-300 amino acids. In certain embodiments, the one or more exogenous polypeptides each independently comprise 5-300 amino acids, optionally 6-250 amino acids, optionally 7-200 amino acids, optionally 8-150 amino acids, optionally 9-100 amino acids, optionally 10-75 amino acids, optionally 11-57 amino acids. In certain embodiments, the one or more exogenous polypeptides each independently comprise 11-57 amino acids.

In certain embodiments, when multiple exogenous polypeptides are present, the exogenous polypeptides are the same.

In certain embodiments, when multiple exogenous polypeptides are present, the exogenous polypeptides are different.

In certain embodiments, the exogenous polypeptide comprises a fragment, preferably an antigenic fragment, of a target protein. In preferred embodiments, the target protein is natively a surface-exposed protein.

In certain embodiments, the exogenous polypeptide comprises a fragment of a target protein, wherein the target protein is insoluble when recombinantly produced.

In certain embodiments, the antigenic fragment of the target protein is inaccessible to antibodies when comprised in the natively folded target protein.

In certain embodiments, the exogenous polypeptide comprises a fragment of a target protein, wherein the target protein is insoluble (when not comprised in the chimeric polypeptide). In preferred such embodiments, the target protein is insoluble in the absence of a solubilization agent (e.g., an amphiphile, such as a phospholipid, detergent, peptide surfactant, amphipol or styrene-maleic acid copolymer). In other preferred such embodiments, the target protein is insoluble in the absence of a chaotropic agent (e.g., urea or guanidine hydrochloride).

In certain embodiments, the target protein is insoluble when recombinantly produced in a host cell or a cell-free expression system (when not comprised in the chimeric polypeptide). In certain embodiments, “insoluble when recombinantly produced” refers to when recombinantly produced in the absence of a solubilization agent or a chaotropic agent.

In certain embodiments, the host cell is a bacterial cell, a yeast cell, a plant cell, an insect cell, or a mammalian cell. In preferred such embodiments, the bacterial cell is an Escherichia coli cell. In certain such embodiments, the exogenous polypeptide is obtainable from the inclusion bodies in the Escherichia coli cell when recombinantly produced therein.

In certain embodiments, the one or more exogenous polypeptide(s) comprise a cryptotope.

In certain embodiments, the target protein is a membrane protein.

In certain embodiments, the exogenous polypeptide comprises a fragment (e.g., an antigenic fragment) of a target protein that is from a microorganism or virus, optionally a pathogenic microorganism or virus. In preferred embodiments, the pathogenic microorganism or virus is selected from Neisseria gonorrhoeae, Neisseria meningitidis, Haemophilus influenzae (optionally non-typeable Haemophilus influenzae), Staphylococcus aureus, human papillomavirus (HPV), Chlamydia trachomatis, Chlamydia muridarum, Streptococcus pneumonia, and Streptococcus agalactiae.

In certain embodiments, the chimeric polypeptide comprises one or more exogenous polypeptide(s) each independently comprising or consisting of an amino acid sequence selected from: SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:51, SEQ ID NO:52, SEQ ID NO:53, SEQ ID NO:182, SEQ ID NO:183, SEQ ID NO: 195, SEQ ID NO: 197, SEQ ID NO: 198, SEQ ID NO: 199 and SEQ ID NO: 200.

In certain embodiments, the chimeric polypeptide comprises or consists of an amino acid sequence selected from: SEQ ID NO:54, SEQ ID NO:55, SEQ ID NO:56, SEQ ID NO:57, SEQ ID NO:58, SEQ ID NO:59, SEQ ID NO:60, SEQ ID NO:61, SEQ ID NO:62, SEQ ID NO:63, SEQ ID NO:64, SEQ ID NO:65, SEQ ID NO:66, SEQ ID NO:67, SEQ ID NO:68, SEQ ID NO:69, SEQ ID NO:70, SEQ ID NO:71, SEQ ID NO:72, SEQ ID NO:73, SEQ ID NO:74, SEQ ID NO:75, SEQ ID NO:76, SEQ ID NO:77, SEQ ID NO:78, SEQ ID NO:79, SEQ ID NO:80, SEQ ID NO:81, SEQ ID NO:82, SEQ ID NO:83, SEQ ID NO:84, SEQ ID NO:85, SEQ ID NO:86, SEQ ID NO:87, SEQ ID NO:88, SEQ ID NO:89, SEQ ID NO:90, SEQ ID NO:91, SEQ ID NO:92, SEQ ID NO:93, SEQ ID NO:94, SEQ ID NO:95, SEQ ID NO:96, SEQ ID NO:97, SEQ ID NO:184, SEQ ID NO:185, SEQ ID NO:186, SEQ ID NO: 190, SEQ ID NO: 191, SEQ ID NO: 192, SEQ ID NO: 193, SEQ ID NO: 194 and SEQ ID NO: 196.

In certain embodiments, the chimeric polypeptide comprises an exogenous polypeptide from a tumour antigen.

In certain embodiments, the scaffold polypeptide is 12-60 kDa. In certain embodiments, the scaffold polypeptide is 12-60 kDa, optionally 12-50 kDa, optionally 12-40 kDa, optionally 12-30 kDa, optionally 12-20 kDa, optionally 12-18 kDa, optionally 13-17 kDa, optionally 14-16 kDa, optionally around 15 kDa. In certain embodiments, the scaffold polypeptide is around 15 kDa.

In certain embodiments, the exogenous polypeptide is 0.5-35 kDa. In certain embodiments, the exogenous polypeptide is 0.5-35 kDa, optionally 0.75-25 kDa, optionally 1-15 kDa, optionally 1-12.5 kDa. In certain embodiments, the exogenous polypeptide is 1-12.5 kDa.

In certain embodiments, the chimeric polypeptide is 12.5-95 kDa. In certain embodiments, the chimeric polypeptide is 12.5-95 kDa, optionally 13-70 kDa, optionally 14-50 kDa, optionally 15-30 kDa. In certain embodiments, the chimeric polypeptide is 15-30 kDa.

In a preferred embodiment, the BP-2a D3 polypeptide comprises an intramolecular isopeptide bond. In preferred such embodiments, the intramolecular isopeptide bond is between K355 and N437, wherein the amino acids are numbered according to the sequence shown in SEQ ID NO:1.

In certain embodiments, the scaffold polypeptide comprises a fusion tag such as an epitope tag, an affinity tag, a fluorescent tag, or a bioluminescent tag. The fusion tag may, for example, facilitate the purification and/or detection (e.g., visualization) of the chimeric polypeptide. In certain embodiments, the fusion tag is a polyhistidine tag (i.e., Hisₙ where n=3, 4, 5, 6, 7, 8, 9, 10 or more), preferably HHHHHH (SEQ ID NO:175).

In certain embodiments, the scaffold polypeptide comprises an enzyme-cleavable amino acid sequence. In certain such embodiments, the enzyme-cleavable amino acid sequence is between the BP-2a D3 polypeptide and a fusion tag e.g. a polyhistidine tag. In certain embodiments, the enzyme-cleavable amino acid sequence is a TEV protease-cleavable amino acid sequence such as ENLYFQG (SEQ ID NO:176). In an embodiment the TEV protease-cleavable amino acid sequence is used to remove the polyhisitidine tag.

In certain embodiments, scaffold polypeptide comprises an amino acid linker (e.g., 1, 2, 3, 4, 5, 6, 7, 8, 9, 10 amino acids in length) between the C-terminus of a functional element (e.g., a fusion tag, an enzyme-cleavable amino acid sequence) and the N-terminus of the BP-2a D3 polypeptide. In certain embodiments, scaffold polypeptide comprises an amino acid linker (e.g., 1, 2, 3, 4, 5, 6, 7, 8, 9, 10 amino acids in length) between the C-terminus of the BP-2a D3 polypeptide and the N-terminus of a functional element (e.g., a fusion tag, an enzyme-cleavable amino acid sequence).

In a second aspect, the present invention provides a nanoparticle comprising a chimeric polypeptide according to the first aspect. In preferred embodiments, the nanoparticle is a self-assembling nanoparticle. In certain embodiments, the nanoparticle is an mI3 nanoparticle.

In certain embodiments, the nanoparticle comprises or consists of an amino acid sequence as shown in SEQ ID NO:98 or SEQ ID NO:99.

In a third aspect, the present invention provides an outer membrane vesicle comprising a chimeric polypeptide according to the first aspect, wherein the chimeric polypeptide is expressed on the surface of the outer membrane vesicle.

In certain embodiments, the outer membrane vesicle is a native outer membrane vesicle. In preferred such embodiments, the native outer membrane vesicle is obtained or obtainable without use of a solubilization agent, most preferably obtained or obtainable without use of a detergent.

In certain embodiments, the outer membrane vesicle is obtained or obtainable from a genetically modified gram-negative bacterium. In certain such embodiments, the gram-negative bacterium is Neisseria meningitidis, Neisseria gonorrhoeae, Escherichia coli, Bordetella pertussis, non-typhoidal Salmonella, Shigella sonnei, Klebsiella pneumoniae, Mycobacterium tuberculosis, or Vibrio cholerae. In preferred such embodiments, the genetic modification results in the gram-negative bacterium being hyper-blebbing. In most preferred such embodiments, the genetic modification is a deletion or inactivation of the ompA gene.

In a fourth aspect, the present invention provides an isolated polynucleotide encoding a chimeric polypeptide according to the first aspect or a nanoparticle according to the second aspect.

In a fifth aspect, the present invention provides an expression vector comprising a polynucleotide of the fourth aspect operably linked to regulatory sequences which permit expression of the chimeric polypeptide or nanoparticle.

In a sixth aspect, the present invention provides a host cell or cell-free expression system containing an expression vector according to the fifth aspect.

In a seventh aspect, the present invention provides a method of producing a chimeric polypeptide or nanoparticle comprising culturing a host cell or cell-free expression system according to the sixth aspect under conditions which permit expression of chimeric polypeptide or nanoparticle and recovering the expressed chimeric polypeptide or nanoparticle.

In an eighth aspect, the present invention provides a pharmaceutical composition comprising a chimeric polypeptide according to the first aspect, a nanoparticle according to the second aspect, or an outer membrane vesicle according to the third aspect.

In a ninth aspect, the present invention provides a method of treatment or prevention comprising administering a chimeric polypeptide according to the first aspect, a nanoparticle according to the second aspect, an outer membrane vesicle according to the third aspect, or a pharmaceutical composition according to the eighth aspect.

In certain embodiments, the method is for treating or preventing cancer.

In certain embodiments, the method is for treating or preventing a pathogenic infection. In preferred such embodiments, the pathogenic infection is caused by a pathogen from which one or more of the exogenous polypeptide(s) is derived.

In a tenth aspect, the present invention provides a chimeric polypeptide according to the first aspect, a nanoparticle according to the second aspect, an outer membrane vesicle according to the third aspect, or a pharmaceutical composition according to the eighth aspect, for use in a method according to the ninth aspect.

In an eleventh aspect, the present invention provides the use of a chimeric polypeptide according to the first aspect, a nanoparticle according to the second aspect, an outer membrane vesicle according to the third aspect, or a pharmaceutical composition according to the eighth aspect in the manufacture of a medicament.

In certain embodiments, the medicament is for treating or preventing cancer.

In certain embodiments, the medicament is for treating or preventing a pathogenic infection. In preferred such embodiments, the pathogenic infection is caused by a pathogen from which one or more of the exogenous polypeptide(s) is derived.

In a twelfth aspect, the present invention provides a method for raising an immune response in a mammal, comprising administering a chimeric polypeptide according to the first aspect, a nanoparticle according to the second aspect, an outer membrane vesicle according to the third aspect, or a pharmaceutical composition according to the eighth aspect.

In a thirteenth aspect, the present invention provides a vaccine comprising a chimeric polypeptide of a chimeric polypeptide according to the first aspect, a nanoparticle according to the second aspect, an outer membrane vesicle according to the third aspect, or a pharmaceutical composition according to the eighth aspect.

In a fourteenth aspect, the present invention provides a method of screening for an antibody which binds a target protein, comprising:
(i) exposing a population of antibodies to a chimeric polypeptide according to the first aspect, a nanoparticle according to the second aspect, or an outer membrane vesicle according to the third aspect, wherein one or more of the exogenous polypeptide(s) of the chimeric polypeptide comprises an antigenic fragment of the target protein; and
(ii) identifying those antibodies which bind to the chimeric polypeptide as binding to the target protein.

In a fifteenth aspect, the present invention provides an array comprising a plurality of chimeric polypeptides according to the first aspect, a plurality of nanoparticles according to the second aspect, or a plurality of outer membrane vesicles according to the third aspect.

In a sixteenth aspect, the present invention provides a kit comprising any of the chimeric polypeptides, nanoparticles, outer membrane vesicles, pharmaceutical compositions, vaccines, or arrays described herein (e.g., in a container, pack, dispenser, microplate). The kits optionally include instructions for use.

### DETAILED DESCRIPTION

### Brief Description of the Drawings

FIG. 1: Shows the chimeras tested in PIMS 20200341 (FIG. 1A) and PIMS 20200600 (FIG. 1B) in vivo studies. 10 μg of each purified recombinant chimera mixed with alum adjuvant was used to intraperitoneally immunize 10 female CD1 mice at 7 weeks old. Three different immunizations were performed at days 0, 21 and 37 assuming that no animal welfare concerns were observed following the second dose. Sera was collected on days 21 (post-2) and 37 (post-3) in addition to a pre-immunization collection (at day −1).

The chimeric polypeptides used in the study “PIMS 20200341” were as follows. Group 1: SEQ ID NO:3; Group 2: SEQ ID NO:55; Group 3: SEQ ID NO:56; Group 4: SEQ ID NO:57; Group 5: SEQ ID NO:61; Group 6: SEQ ID NO:72; Group 7: SEQ ID NO:71; Group 8: SEQ ID NO:184; Group 9: SEQ ID NO:185; Group 10: SEQ ID NO:74; Group 11: SEQ ID NO:77; Group 12: SEQ ID NO:84; Group 13: SEQ ID NO:87; Group 14: SEQ ID NO:73; Group 15: SEQ ID NO:186; and Group 16: SEQ ID NO:60.

The chimeric polypeptides used in the study “PIMS 20200600” were as follows. Group 1: SEQ ID NO:54; Group 2: SEQ ID NO:58; Group 3: SEQ ID NO:59; Group 4: SEQ ID NO:69; and Group 5: SEQ ID NO:70.

FIG. 2: (A) 3D structure of GBS pilus backbone protein type 2a (GBS BP-2a; PDB code 2XTL). Domain 2 (D2) light grey, domain 3 (D3) black, domain 4 (D4) grey. (B) D3 magnification reporting the identified loops in grey. (C) Magnification of isopeptide bond occurring between Lys355 and Asn437. Images obtained with ChimeraX.

FIG. 3: Predicted 3D structures of PorB1b and rOpaB proteins from N. gonorrhoeae. (A) PorB1b is predicted to form a homotrimer in the outer membrane. (B) Identification of 8 extracellular loops of PorB1b reported in black. Magnification of Loop3 and Loop5 highlighting the secondary structures therein. (C) Side and top view of predicted model of OpaB. (D) Identification of 4 extracellular loops of OpaB reported in black. Magnification of Loop2 and Loop3 presenting a secondary structure. Images obtained with ChimeraX.

FIG. 4: (A) SDS-PAGE analysis of expression and solubility of chimeric polypeptides displaying PorB1b loop3 inserted into 6 different sites in the BP-2a D3 polypeptide (partially replacing endogenous loops 1-6, respectively). The chimeric polypeptides with PorB1b loop3 inserted at sites 1-6 correspond to SEQ ID Nos: 62, 56, 63, 64, 65, and 66, respectively. The D3 construct used as a control corresponds to SEQ ID NO:3. M: Novex sharp protein marker (Invitrogen LC5800); T: total fractions, S: soluble fractions of E. coli extract. (B) Unfolding profiles of each chimeric polypeptide evaluated with NanoDSF. (C) Inflection temperatures corresponding to the melting temperature (Tm) of each chimeric polypeptide calculated as inflection point of the corresponding curve. (D) Predicted 3D structure of BP-2a D3 polypeptide displaying PorB1b loop3 (in black) at each D3 site (where the endogenous loop of the same number has been partially replaced) and PorB1b. Black arrows indicate the two α-helices structures of loop3 of PorB1b detected in the native PorB1b protein and maintained after the insertion of PorB1b loop3 into D3 sites 2 and 6 (whereby the endogenous loop of the same number has been partially replaced). Images obtained with Pymol.

FIG. 5: Thermal stability analysis of chimeric polypeptides displaying OpaB loops (A) and PorB1b loops (B) with their respective inflection temperatures (Tm). (C) Western Blot analysis of chimeric polypeptides displaying PorB1b or OpaB loops. (C-i-ii) Chimeric polypeptides displaying PorB1b loops tested with α-rPorB1b and α-OMV-FA1090 sera. (C-iii) Chimeric polypeptides displaying OpaB loops tested with α-OMV-FA1090 sera. (C-iv-v) Total cell extracts and purified OMV-FA1090 tested with α-PorB loop5 and α-OpaB loop2 sera. (D) Luminex assay on chimeric polypeptides displaying PorB1b or OpaB loops testing α-OMV-FA1090 serum. (E) Partial amino acid sequence alignment obtained with clustalW between PorB1b (above) and OpaB (below) proteins produced by N. gonorrhoeae strains F62 and FA1090. Rectangles highlight loop5 of PorB1b and loop2 of OpaB.

FIG. 6: Crystal structure resolution of a chimeric polypeptide displaying PorB loop 5 (D3PorBloop5) (SEQ ID NO:58). (A) Two chains (A, dark grey and B, light grey) were detected in the crystal structure. Compared with the sequence of recombinant protein, in the crystal some residues at N- and C-terminus were absent (rectangles). The connection between one chain and another are highlighted in the magnification reported in the left part of the section. Residues involved in the inter-chain bonds are reported as black sticks. (B) Identification of an internal isopeptide bond occurring within residues K43 and N146 of each chain. (C) Structural comparison of computationally predicted D3PorBloop5 models and resolved crystal structure and evaluation of RMSDs. (D) Density map shown for the entire crystal (above) and a magnification of density map around PorB loop5 (below). Graphical representation and structural analysis performed with Pymol.

FIG. 7: (A) Design and Rosetta homology modelling derived 3D structure prediction of a chimeric mI3 nanoparticle displaying PorB1b loop5 alone (mI3-PorBLoop5, SEQ ID NO: 187) versus a chimeric mI3 nanoparticle displaying a chimeric polypeptide comprising a BP-2a D3 polypeptide in which PorB loop 5 was inserted (mI3-D3PorBLoop5; SEQ ID NO:98). Images obtained with Chimera. (B) SDS-PAGE analysis of expression and solubility of each chimera extracted with cell-lytic detergent, T: total fraction, S: soluble fraction. (C) SDS-PAGE analysis of purified proteins after SEC. The controls used were a naked mI3 nanoparticle, and a monomer of D3 PorB1b loop5 (SEQ ID NO:58). (D) Western blot of purified protein using an α-His antibody. (E) Negative stain electron microscopy of mI3-D3PorBloop5 (SEQ ID NO:98). (F) Electron microscopy in negative staining of mI3-PorBloop5 (SEQ ID NO:187).

FIG. 8: (A) Predicted 3D models of chimeric polypeptides simultaneously displaying Chlamydia VD1 and PorBloop5 (partially replacing endogenous loops 1 and 2 of D3, respectively, see SEQ ID NO: 188), or Chlamydia VD3 and PorBloop5 (partially replacing endogenous loops 1 and 2 of D3, respectively, see SEQ ID NO: 189). Above arrows: PorB loop5; Below arrows: VD1 (left) and VD3 (right). (B) SDS-PAGE analysis of purification fractions collected during IMAC. S: soluble, FT: flowthrough, E: elution, D3L1V D1L2Loop5: a chimeric polypeptide wherein VD1 partially replaced loop 1 of D3 and PorB loop5 partially replaced loop 2 of D3, D3L1VD3L2Loop5: a chimeric polypeptide wherein VD3 partially replaced loop 1 of D3 and PorB loop5 partially replaced loop 2 of D3. (C) Thermal stability evaluation with NanoDSF and respective inflection temperatures evaluated.

FIG. 9: SDS-PAGE analysis of expression and solubility of empty D3 and chimeric D3 from different GBS strains displaying PorB.1 b Loop5. Soluble fractions of E. coli extract were loaded.

FIG. 10: Cartoon representation of D3 structure from strain 515 aligned with the structure of D3 from the other 5 D3 variants. In black it is highlighted the loop engineered with foreign epitopes. Root mean square deviation (RMSD) scores are also provided.

FIG. 11: D3 correctly displays folded GFP: (A) Structural model of D3-GFP generated with AlphaFold2 and visualized with Pymol. D3 structure is coloured in dark grey and GFP in light grey. (B) SDS-PAGE of purified D3-GFP, GFP alone and D3 empty (C) Fluorescence of GFP constructs (D) Thermal stability (nano-DSF) of D3-GFP construct.

FIG. 12: SDS-PAGE demonstrating E. Coli OMVs expressing D3, His-D3 empty and His-D3-PorBloop5. OMVs were produced at 20° C. and 37° C.

FIG. 13: (A) Western blot analysis with anti-D3 serum shows expression of empty D3 or Chimeric D3-PorBloop5 in E. coli OMV. (B) Western blot analysis with anti-Gonococcal OMV (containing specific anti-PorB Abs) serum recognizes Chimeric D3-PorBloop5 in E. coli OMV and the control recombinant protein.

### DEFINITIONS

The terms as used herein are given their conventional definition in the art as understood by the skilled person, unless otherwise defined below. In the case of any inconsistency or doubt, the definition as provided herein should take precedence.

As used in this specification, the singular forms “a,” “an,” and “the” include plural referents unless expressly and unequivocally limited to one referent. The term “or” is used interchangeably with the term “and/or” unless the context clearly indicates otherwise.

Various publications are cited in this specification, each of which is incorporated by reference herein in its entirety.

“Conservative amino acid substitution”—As used herein, a “conservative amino acid substitution” is one in which the amino acid residue is replaced with an amino acid residue having a similar side chain. Families of amino acid residues having similar side chains have been defined in the art, including basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine).

“Chimeric”—As used herein, a “chimeric” polypeptide comprises a first amino acid sequence linked to a second amino acid sequence with which it is not naturally linked in nature. The amino acid sequences may normally exist in separate proteins that are brought together in the fusion polypeptide or they may normally exist in the same protein but are placed in a new arrangement in the fusion polypeptide. A chimeric polypeptide may be created, for example, by chemical synthesis, or by creating and translating a polynucleotide in which the peptide regions are encoded in the desired relationship.

“Endogenous loop”—As used herein, the term “endogenous loop” refers to the amino acid sequence that is present and forms a disordered loop in the native protein. For example, a loop in domain 3 (D3) of pilus 2a backbone protein (BP-2a).

“Exogenous polypeptide”—As used herein, the term “exogenous polypeptide” refers to an amino acid sequence that is not natively found in domain 3 (D3) of pilus 2a backbone protein (BP-2a). As used herein, the term “exogenous polypeptide” includes exogenous peptides, oligopeptides, and longer peptide chains, including full length exogenous protein sequences and fragments thereof. For example, an exogenous polypeptide may be 5-300 amino acids in length.

“Fragment”—As used herein, the term “fragment” refers to a part or portion of a protein or polypeptide comprising fewer amino acid residues than an intact or complete protein or polypeptide. For example, a fragment may be 5-300 amino acids in length.

“Native”—As used herein, the term “native” with respect to an amino acid sequence refers to the order of amino acids in the sequence as found in nature (i.e., without any engineered mutations). The native BP-2a sequence from GBS strain 515 is shown in SEQ ID NO:1. The native BP-2a D3 sequence from GBS strain 515 is shown in SEQ ID NO:2. The native BP-2a sequence from GBS strain H36B is shown in SEQ ID NO:100. The native BP-2a D3 sequence from GBS strain H36B is shown in SEQ ID NO:101. The native BP-2a sequence from GBS strain CJB111 is shown in SEQ ID NO:115. The native BP-2a D3 sequence from GBS strain CJB111 is shown in SEQ ID NO:116. The native BP-2a sequence from GBS strain 2603 is shown in SEQ ID NO:130. The native BP-2a D3 sequence from GBS strain 2603 is shown in SEQ ID NO:131. The native BP-2a sequence from GBS strain CJB110 is shown in SEQ ID NO:145. The native BP-2a D3 sequence from GBS strain CJB110 is shown in SEQ ID NO:146. The native BP-2a sequence from GBS strain DK21 is shown in SEQ ID NO:160. The native BP-2a D3 sequence from GBS strain DK21 is shown in SEQ ID NO:161.

With respect to outer membrane vesicles, “native” refers to having a membrane composition reflective of that found in nature as a result of the method by which the outer membrane vesicle is produced.

“(Percentage) sequence identity”—As used herein, the term “sequence identity” refers to the degree of sameness of two sequences, such as amino acid sequences. This may be determined by comparing the two sequences aligned in an optimum manner and in which the sequence to be compared can comprise additions or deletions with respect to the reference sequence for an optimum alignment between these two sequences. The percentage of identity is calculated by determining the number of identical positions for which the residue is identical between the two sequences, dividing this number of identical positions by the total number of positions in the longer of the two sequences and multiplying the result obtained by 100 in order to obtain the percentage sequence identity between these two sequences. For example, it is possible to use the BLAST program, available on the website, https://blast.ncbi.nlm.nih.gov/Blast.cgi, the parameters used being those given by default; the matrix chosen for an amino acid sequence alignment being, for example, the matrix “BLOSUM 62” proposed by the program), the percentage of identity between the two sequences to be compared being calculated directly by the program.

The following embodiments are embodiments according to each aspect of the present invention, and any embodiment may be combined with any other embodiment unless explicitly stated otherwise. Any preferred embodiment may be combined with any other preferred embodiment unless explicitly stated otherwise.

### Chimeric Polypeptides

It is an object of the invention to provide a scaffold for facilitating the recombinant production of polypeptides (e.g., antigenic sequences or epitopes) derived from proteins of interest. By facilitating the effective display of a desired polypeptide (such as a protein antigen or epitope), the present invention is particularly beneficial for the production of protein-based vaccines, especially their production at large scale.

The present inventors have surprisingly found that domain 3 (D3) of pilus 2a backbone protein (BP-2a), also referred to herein as “BP-2a D3”, can be advantageously used as a scaffold protein to facilitate the recombinant production of polypeptide sequences of interest (e.g., epitopes). BP-2a D3 natively contains a number of loops (referred to herein as “endogenous loops”). The inventors identified that one or more endogenous loops can be partially or wholly replaced an exogenous polypeptide while maintaining overall stability and solubility. Further advantageously, the resultant chimeric polypeptides are readily producible recombinantly (e.g., in E. coli) and correctly display exogenous polypeptides (e.g., epitopes) comprised therein.

In a first aspect, the present invention provides a chimeric polypeptide comprising:
(i) a scaffold polypeptide; and
(ii) one or more exogenous polypeptide(s),
wherein the scaffold polypeptide comprises a backbone protein 2a (BP-2a) Domain 3 (D3) polypeptide, wherein in the chimeric polypeptide at least one endogenous loop of the BP-2a D3 polypeptide is partially or wholly replaced by an exogenous polypeptide.

As used herein, an “exogenous polypeptide” refers to any amino acid sequence that is not natively found in domain 3 (D3) of pilus 2a backbone protein (BP-2a), including full length exogenous proteins, as well as fragments or epitopes thereof. An “exogenous polypeptide” includes, for example, exogenous peptides, oligopeptides, and longer peptide chains.

Any number of the amino acids natively found in an endogenous loop of D3 may be replaced by any number of amino acids not natively found in the endogenous loop at the same amino acid positions. Replacement of an amino acid does not necessarily require 1:1 replacement but does not exclude 1:1 replacement. As used herein, “partial replacement” of an endogenous loop refers to replacement of at least a single amino acid within the endogenous loop. Partial replacement includes replacement of more than one amino acid within the endogenous loop, up to replacement of all but one of the amino acids natively found in the endogenous loop. Preferably, when an endogenous loop is partially replaced, the amino acid or amino acids not replaced is/are found at the same position in the endogenous loop as natively found in D3 of BP-2a. “Whole replacement” of an endogenous loop refers to replacement of all the amino acids that are natively present in the endogenous loop with amino acids that are not natively present in the endogenous loop at the same positions.

As described herein, native BP-2a D3 polypeptides have the structure: scaffold1-loop1-scaffold2-loop2-scaffold3-loop3-scaffold4-loop4-scaffold5-loop5-scaffold6-loop6-scaffold7, where the scaffold regions are structured and the loops are disordered. Identifying the disordered loop regions of a given BP-2a D3 protein is within the ability of the skilled person in view of the information provided herein.

With reference to the numbering of amino acids in the amino acid sequence of a native BP-2a polypeptide shown in SEQ ID NO:1 (strain 515), the native BP-2a D3 polypeptide therein corresponds to amino acids 332-447. Analysis of the crystal structure of BP-2a D3 from strain 515 enabled the determination of the residue positions of the endogenous loop regions. The first endogenous loop of the BP-2a D3 polypeptide spans the amino acids at positions 360-372; the second endogenous loop of the BP-2a D3 polypeptide spans the amino acids at positions 380-384; the third endogenous loop of the BP-2a D3 polypeptide spans the amino acids at positions 393-399; the fourth endogenous loop of the BP-2a D3 polypeptide spans the amino acids at positions 404-411; the fifth endogenous loop of the BP-2a D3 polypeptide spans the amino acids at positions 418-422; and the sixth endogenous loop of the BP-2a D3 polypeptide spans the amino acids at positions 429-432. The endogenous loops include the amino acids at the recited end-point positions. The invention described herein is not however limited to a scaffold polypeptide comprising only this particular BP-2a D3 polypeptide sequence (strain 515). Other BP-2a D3 polypeptide sequences can be utilised and within these sequences the endogenous loop regions are at positions equivalent to the positions disclosed above with respect to SEQ ID NO: 1. Predicted endogenous loops and scaffold regions are disclosed herein for the BP-2a D3 sequence from five additional GBS strains (H36B, CJB111, CJB110, 2603, DK21).

For other BP-2a polypeptides, identification of amino acids at positions corresponding to those identified positions of SEQ ID NO:1 is within the ability of the skilled person in view of the information provided herein. For example, tools for modelling the structural homology of a BP-2a polypeptide to the D3 of strain 515 are known to the skilled person and include e.g. the RosettaCM program described below.

By way of example, amino acid positions of a given BP-2a D3 corresponding to the residues of the first endogenous loop of SEQ ID NO:1 are the positions that, when the overall D3 structure is modelled (e.g. by RosettaCM), are flanked by the first two ordered regions corresponding to scaffold1 and scaffold2, but themselves show no stable 3D structure—that is, the first disordered region. Likewise, amino acid positions of a given BP-2a D3 corresponding to the residues of the first scaffold region of SEQ ID NO: 1 are the positions that, when the overall D3 structure is modelled (e.g. by RosettaCM), show stable 3D structure and are followed by the first disordered region. The amino acids corresponding to the other scaffold regions and endogenous loops can be determined in the same manner.

An endogenous loop does not need to be of the same length between D3 proteins. For example, for the native BP-2a D3 protein from strain H36B, amino acid positions 369-391 correspond to amino acid positions 360-372 of SEQ ID NO:1.

Corresponding comparisons can be made by the skilled person to identify the amino acids of any given D3 polypeptide that form the other endogenous loops, and thus correspond to the identified positions of SEQ ID NO:1.

The sequences of different BP-2a and BP-2a D3 polypeptides, as well as their respective scaffold and loop sequences, are provided in Table 1. For clarity, the scaffold and loop sequences for strains H36B (SEQ ID NO: 102-114), CJB111 (SEQ ID NO: 117-129), CJB110 (SEQ ID NO: 147-159), 2603 (SEQ ID NO: 132-144), DK21 (SEQ ID NO: 162-174) have been predicted based on their equivalency to strain 515.

TABLE 1
BP-2a and D3

| SEQ ID | Construct Name | Amino Acid Sequence |
|---|---|---|
| NO | Native BP-2a (GBS strain | |
| 1 | 515) | MKKINKYFAVFSALLLTVTSLFSVAPVFAEEAKTTDTVTLHKI |
| | | VMPRTAFDGFTAGTKGKDNTDYVGKQIEDLKTYFGSGEAKEI |
| | | AGACFAFKNEAGTKYITENGEEVDTLDTTDAKGCAVLKGLTT |
| | | DNGFKFNTSKLTGTYQIVELKEKSTYNNDGSILADSKAVPVKI |
| | | TLPLVNDNGVVKDAHVYPKNTETKPQVDKNFADKELDYANN |
| | | KKDKGTVSASVGDVKKYHVGTKILKGSDYKKLIWTDSMTKG |
| | | LTFNNDIAVTLDGATLDATNYKLVADDQGFRLVLTDKGLEAV |
| | | AKAAKTKDVEIKITYSATLNGSAVVEVLETNDVKLDYGNNPTI |
| | | ENEPKEGIPVDKKITVNKTWAVDGNEVNKADETVDAVFTLQ |
| | | VKDGDKWVNVDSAKATAATSFKHTFENLDNAKTYRVIERV |
| | | SGYAPEYVSFVNGVVTIKNNKDSNEPTPINPSEPKVVTYGR |
| | | KFVKTNKDGKERLAGATFLVKKDGKYLARKSGVATDAEKAA |
| | | VDSTKSALDAAVKAYNDLTKEKQEGQDGKSALATVSEKQKA |
| | | YNDAFVKANYSYEWVEDKNAKNVVKLISNDKGQFEITGLTE |
| | | GQYSLEETQAPTGYAKLSGDVSFNVNATSYSKGSAQDIEYT |
| | | QGSKTKDAQQVINKKVTIPQTGGIGTIFFTIIGLSIMLGAV |
| | | VIMKRRQSEEV |
| 2 | Native D3 (GBS strain | GNNPTIENEPKEGIPVDKKITVNKTWAVDGNEVNKADETVDA |
| | 515) | VFTLQVKDGDKWVNVDSAKATAATSFKHTFENLDNAKTYRV |
| | | IERVSGYAPEYVSFVNGVVTIKNNKDSNEPTPI |
| 3 | Recombinant D3 (GBS | MGSSHHHHHHENLYFQGASGNNPTIENEPKEGIPVDKKITV |
| | strain 515) | NKTWAVDGNEVNKADETVDAVFTLQVKDGDKWVNVDSAK |
| | | ATAATSFKHTFENLDNAKTYRVIERVSGYAPEYVSFVNGVV |
| | | TIKNNKDSNEPTPI |
| 4 | First endogenous loop | DGNEVNKADETVD |
| | (GBS strain 515) | |
| 5 | Second endogenous loop | KDGDK |
| | (GBS strain 515) | |
| 6 | Third endogenous loop | ATAATSF |
| | (GBS strain 515) | |
| 7 | Fourth endogenous loop | ENLDNAKT |
| | (GBS strain 515) | |
| 8 | Fifth endogenous loop | VSGYA |
| | (GBS strain 515) | |
| 9 | Sixth endogenous loop | VNGV |
| | (GBS strain 515) | |
| 10 | First Scaffold Region | GNNPTIENEPKEGIPVDKKITVNKTWAV |
| | (GBS strain 515) | |
| 11 | Second Scaffold Region | AVFTLQV |
| | (GBS strain 515) | |
| 12 | Third Scaffold Region | WVNVDSAK |
| | (GBS strain 515) | |
| 13 | Fourth Scaffold Region | YRVIER |
| | (GBS strain 515) | |
| 14 | Fifth Scaffold Region | KHTF |
| | (GBS strain 515) | |
| 15 | Sixth Scaffold Region | PEYVSF |
| | (GBS strain 515) | |
| 16 | Seventh Scaffold Region | VTIKNNKDSNEPTPI |
| | (GBS strain 515) | |
| 100 | Native BP-2a (GBS strain | MKRINKYFAMFSALLLILTSLLSVAPVFAAEMGNITKTVTLHKI |
| | H36B) | VQTSDNLAKPNFPGINGLNGTKYMGQKLTDISGYFGQGSKE |
| | | IAGAFFAVMNESQTKYITESGTEVESIDAAGVLKGLTTENGIT |
| | | FNTANLKGTYQIVELLDKSNYKNGDKVLADSKAVPVKITLPLY |
| | | NEEGIVVDAEVYPKNTEEAPQIDKNFAKANKLLNDSDNSAIA |
| | | GGADYDKYQAEKAKATAEIGQEIPYEVKTKIQKGSKYKNLA |
| | | WVDTMSNGLTMGNTVNLEASSGSFVEGTDYNVERDDRGFT |
| | | LKFTDTGLTKLQKEAETQAVEFTLTYSATVNGAAIDDKPESN |
| | | DIKLQYGNKPGKKVKEIPVTPSNGEITVSKTWDKGSDLENA |
| | | NVVYTLKDGGTAVASVSLTKTTPNGEINLGNGIKFTVTGAF |
| | | AGKFSGLTDSKTYMISERIAGYGNTITTGAGSAAITNTPDSD |
| | | NPTPLNPTEPKVVTHGKKFVKTSSTETERLQGAQFVVKDSA |
| | | GKYLALKSSATISAQTTAYTNAKTALDAKIAAYNKLSADDQK |
| | | GTKGETAKAEIKTAQDAYNAAFIVARTAYEWVTNKEDANVV |
| | | KVTSNADGQFEVSGLATGDYKLEETQAPAGYAKLAGDVDFK |
| | | VGNSSKADDSGNIDYTASSNKKDAQRIENKKVTIPQTGGIGTI |
| | | LFTIIGLSIMLGAVIIMKRRQSEEA |
| 101 | Native D3 (GBS strain | NKPGKKVKEIPVTPSNGEITVSKTWDKGSDLENANVVYTLKD |
| | H36B) | GGTAVASVSLTKTTPNGEINLGNGIKFTVTGAFAGKFSGLTD |
| | | SKTYMISERIAGYGNTITTGAGSAAITNTPDSDNPTPL |
| 102 | First endogenous loop | DKGSDLENANVVYTLKDGGTAVA |
| | (GBS strain H36B) | |
| 103 | Second endogenous loop | TPNG |
| | (GBS strain H36B) | |
| 104 | Third endogenous loop | FTVTGAF |
| | (GBS strain H36B) | |
| 105 | Fourth endogenous loop | SGLTDSKTY |
| | (GBS strain H36B) | |
| 106 | Fifth endogenous loop | RIAGYG |
| | (GBS strain H36B) | |
| 107 | Sixth endogenous loop | TGAGSA |
| | (GBS strain H36B) | |
| 108 | First Scaffold Region | NKPGKKVKEIPVTPSNGEITVSKTW |
| | (GBS strain H36B) | |
| 109 | Second Scaffold Region | SVSLTKT |
| | (GBS strain H36B) | |
| 110 | Third Scaffold Region | EINLGNGIK |
| | (GBS strain H36B) | |
| 111 | Fourth Scaffold Region | AGKF |
| | (GBS strain H36B) | |
| 112 | Fifth Scaffold Region | MISE |
| | (GBS strain H36B) | |
| 113 | Sixth Scaffold Region | NTIT |
| | (GBS strain H36B) | |
| 114 | Seventh Scaffold Region | AITNTPDSDNPTPL |
| | (GBS strain H36B) | |
| 115 | Native BP-2a (GBS strain | MKKINKCLTMFSTLLLILTSLFSVAPAFADDATTDTVTLHKIVM |
| | CJB111) | PQAAFDNFTEGTKGKNDSDYVGKQINDLKSYFGSTDAKEIK |
| | | GAFFVFKNETGTKFITENGKEVDTLEAKDAEGGAVLSGLTKD |
| | | NGFVFNTAKLKGIYQIVELKEKSNYDNNGSILADSKAVPVKIT |
| | | LPLVNNQGVVKDAHIYPKNTETKPQVDKNFADKDLDYTDNR |
| | | KDKGVVSATVGDKKEYIVGTKILKGSDYKKLVWTDSMTKGL |
| | | TFNNNVKVTLDGEDFPVLNYKLVTDDQGFRLALNATGLAAV |
| | | AAAAKDKDVEIKITYSATVNGSTTVEIPETNDVKLDYGNNPTE |
| | | ESEPQEGTPANQEIKVIKDWAVDGTITDANVAVKAIFTLQEK |
| | | QTDGTWVNVASHEATKPSRFEHTFTGLDNAKTYRVVERVS |
| | | GYTPEYVSFKNGVVTIKNNKNSNDPTPINPSEPKVVTYGRK |
| | | FVKTNQANTERLAGATFLVKKEGKYLARKAGAATAEAKAAV |
| | | KTAKLALDEAVKAYNDLTKEKQEGQEGKTALATVDQKQKAY |
| | | NDAFVKANYSYEWVADKKADNVVKLISNAGGQFEITGLDKG |
| | | TYGLEETQAPAGYATLSGDVNFEVTATSYSKGATTDIAYDK |
| | | GSVKKDAQQVQNKKVTIPQTGGIGTILFTIIGLSIMLGAVV |
| | | IMKKRQSEEA |
| 116 | Native D3 (GBS strain | NNPTEESEPQEGTPANQEIKVIKDWAVDGTITDANVAVKAIF |
| | CJB111) | TLQEKQTDGTWVNVASHEATKPSRFEHTFTGLDNAKTYRVV |
| | | ERVSGYTPEYVSFKNGVVTIKNNKNSNDPTPI |
| 117 | First endogenous loop | AVDGTITDANVAVK |
| | (GBS strain CJB111) | |
| 118 | Second endogenous loop | QTDG |
| | (GBS strain CJB111) | |
| 119 | Third endogenous loop | ATKPSR |
| | (GBS strain CJB111) | |
| 120 | Fourth endogenous loop | TGLDNAKTY |
| | (GBS strain CJB111) | |
| 121 | Fifth endogenous loop | RVSGYT |
| | (GBS strain CJB111) | |
| 122 | Sixth endogenous loop | KNGV |
| | (GBS strain CJB111) | |
| 123 | First Scaffold Region | NNPTEESEPQEGTPANQEIKVIKDW |
| | (GBS strain CJB111) | |
| 124 | Second Scaffold Region | AIFTLQEK |
| | (GBS strain CJB111) | |
| 125 | Third Scaffold Region | TWVNVASHE |
| | (GBS strain CJB111) | |
| 126 | Fourth Scaffold Region | FEHTF |
| | (GBS strain CJB111) | |
| 127 | Fifth Scaffold Region | RVVE |
| | (GBS strain CJB111) | |
| 128 | Sixth Scaffold Region | PEYVSF |
| | (GBS strain CJB111) | |
| 129 | Seventh Scaffold Region | VTIKNNKNSNDPTPI |
| | (GBS strain CJB111) | |
| 130 | Native BP-2a (GBS strain | MKRINKYFAMFSALLLTLTSLLSVAPAFADEATTNTVTLHKIL |
| | 2603) | QTESNLNKSNFPGTTGLNGKDYKGGAISDLAGYFGEGSKEI |
| | | EGAFFALALKEDKSGKVQYVKAKEGNKLTPALINKDGTPEIT |
| | | VNIDEAVSGLTPEGDTGLVFNTKGLKGEFKIVEVKSKSTYNN |
| | | NGSLLAASKAVPVNITLPLVNEDGWVADAHVYPKNTEEKPEI |
| | | DKNFAKTNDLTALTDVNRLLTAGANYGNYARDKATATAEIGK |
| | | VVPYEVKTKIHKGSKYENLVWTDIMSNGLTMGSTVSLKASG |
| | | TTETFAKDTDYELSIDARGFTLKFTADGLGKLEKAAKTADIEF |
| | | TLTYSATVNGQAIIDNPESNDIKLSYGNKPGKDLTELPVTPS |
| | | KGEVTVAKTWSDGIAPDGVNVVYTLKDKDKTVASVSLTKT |
| | | SKGTIDLGNGIKFEVSGNFSGKFTGLENKSYMISERVSGYG |
| | | SAINLENGKVTITNTKDSDNPTPLNPTEPKVETHGKKFVKTN |
| | | EQGDRLAGAQFVVKNSAGKYLALKADQSEGQKTLAAKKIAL |
| | | DEAIAAYNKLSATDQKGEKGITAKELIKTKQADYDAAFIEART |
| | | AYEWITDKARAITYTSNDQGQFEVTGLADGTYNLEETLAPAG |
| | | FAKLAGNIKFVVNQGSYITGGNIDYVANSNQKDATRVENKKV |
| | | TIPQTGGIGTILFTIIGLSIMLGAVVIMKRRQSKEA |
| 131 | Native D3 (GBS strain | NKPGKDLTELPVTPSKGEVTVAKTWSDGIAPDGVNVVYTLK |
| | 2603) | DKDKTVASVSLTKTSKGTIDLGNGIKFEVSGNFSGKFTGLEN |
| | | KSYMISERVSGYGSAINLENGKVTITNTKDSDNPTPL |
| 132 | First endogenous loop | TWSDGIAPDGVNV |
| | (GBS strain 2603) | |
| 133 | Second endogenous loop | KDKDKTVAS |
| | (GBS strain 2603) | |
| 134 | Third endogenous loop | KTSKGTIDLGNGIKFEVSGN |
| | (GBS strain 2603) | |
| 135 | Fourth endogenous loop | TGLENK |
| | (GBS strain 2603) | |
| 136 | Fifth endogenous loop | RVSGYG |
| | (GBS strain 2603) | |
| 137 | Sixth endogenous loop | ENGK |
| | (GBS strain 2603) | |
| 138 | First Scaffold Region | NKPGKDLTELPVTPSKGEVTVAK |
| | (GBS strain 2603) | |
| 139 | Second Scaffold Region | VYTL |
| | (GBS strain 2603) | |
| 140 | Third Scaffold Region | VSLT |
| | (GBS strain 2603) | |
| 141 | Fourth Scaffold Region | FSGKF |
| | (GBS strain 2603) | |
| 142 | Fifth Scaffold Region | SYMISE |
| | (GBS strain 2603) | |
| 143 | Sixth Scaffold Region | SAINL |
| | (GBS strain 2603) | |
| 144 | Seventh Scaffold Region | VTITNTKDSDNPTPL |
| | (GBS strain 2603) | |
| 145 | Native BP-2a (GBS strain | MKKINKYFAVFSALLLTVTSLLSVAPAFADEATTNTVTLHKILQ |
| | CJB110) | TESNLNKSNFPGTTGLNGDDYKGESISDLAEYFGSGSKEID |
| | | GAFFALALEEEKDGVVQYVKAKANDKLTPDLITKGTPATTTK |
| | | VEEAVGGLTTGTGIVENTAGLKGNFKIIELKDKSTYNNNGSLL |
| | | AASKAVPVKITLPLVSKDGVVKDAHVYPKNTETKPEVDKNFA |
| | | KTNDLTALKDATLLKAGADYKNYSATKATVTAEIGKVIPYEVK |
| | | TKVLKGSKYEKLVWTDTMSNGLTMGDDVNLAVSGTTTTFIK |
| | | DIDYTLSIDDRGFTLKFKATGLDKLEEAAKASDVEFTLTYKAT |
| | | VNGQAIIDNPEVNDIKLDYGNKPGTDLSEQPVTPEDGEVKVT |
| | | KTWAAGANKADAKVVYTLKNATKQVVASVALTAADTKGTI |
| | | NLGKGMTFEITGAFSGTFKGLQNKAYTVSERVAGYTNAINV |
| | | TGNAVAITNTPDSDNPTPLNPTQPKVETHGKKFVKVGDADA |
| | | RLAGAQFVVKNSAGKFLALKEDAAVSGAQTELATAKTDLDN |
| | | AIKAYNGLTKAQQEGADGTSAKELINTKQSAYDAAFIKARTA |
| | | YTWVDEKTKAITFTSNNQGQFEVTGLEVGSYKLEETLAPAG |
| | | YAKLSGDIEFTVGHDSYTSGDIKYKTDDASNNAQKVFNKKVT |
| | | IPQTGGIGTILFTIIGLSIMLGAVVIMKRRQSEEA |
| 146 | Native D3 (GBS strain | NKPGTDLSEQPVTPEDGEVKVTKTWAAGANKADAKVVYTL |
| | CJB110) | KNATKQVVASVALTAADTKGTINLGKGMTFEITGAFSGTFKG |
| | | LQNKAYTVSERVAGYTNAINVTGNAVAITNTPDSDNPTPL |
| 147 | First endogenous loop | AAGANKADAKVVYTLKNATKQVV |
| | (GBS strain CJB110) | |
| 148 | Second endogenous loop | DTKG |
| | (GBS strain CJB110) | |
| 149 | Third endogenous loop | FEITGAF |
| | (GBS strain CJB110) | |
| 150 | Fourth endogenous loop | KGLQNKAY |
| | (GBS strain CJB110) | |
| 151 | Fifth endogenous loop | RVAGYT |
| | (GBS strain CJB110) | |
| 152 | Sixth endogenous loop | TGNA |
| | (GBS strain CJB110) | |
| 153 | First Scaffold Region | NKPGTDLSEQPVTPEDGEVKVTKTW |
| | (GBS strain CJB110) | |
| 154 | Second Scaffold Region | ASVALTAA |
| | (GBS strain CJB110) | |
| 155 | Third Scaffold Region | TINLGKGMT |
| | (GBS strain CJB110) | |
| 156 | Fourth Scaffold Region | SGTF |
| | (GBS strain CJB110) | |
| 157 | Fifth Scaffold Region | TVSE |
| | (GBS strain CJB110) | |
| 158 | Sixth Scaffold Region | NAINV |
| | (GBS strain CJB110) | |
| 159 | Seventh Scaffold Region | VAITNTPDSDNPTPL |
| | (GBS strain CJB110) | |
| 160 | Native BP-2a (GBS strain | MKKINKFFVAFSALLLILTSLLSVAPAFAEEERTTETVTLHKIL |
| | DK21) | QTETNLKNSAFPGTKGLDGTEYDGKAIDKLDSYFGNDSKDI |
| | | GGAYFILANSKGEYIKANDKNKLKPEFSGNTPKTTLNISEAVG |
| | | GLTEENAGIKFETTGLRGDFQIIELKDKSTYNNGGAILADSKA |
| | | VPVKITLPLINKDGVVKDAHVYPKNTETKPQIDKNFADKNLDY |
| | | INNQKDKGTISATVGDVKKYTVGTKILKGSDYKKLVWTDSMT |
| | | KGLTFNNDVTVTLDGANFEQSNYT |
| | | LVADDQGFRLVLNATGLSKVAEAAKTKDVEIKINYSATVNGS |
| | | TVVEKSENNDVKLDYGNNPTTENEPQTGNPVNKEITVRKTW |
| | | AVDGNEVNKGDEKVDAVFTLQVKDSDKWVNVDSATATAA |
| | | TDFKYTFKNLDNAKTYRVVERVSGYAPAYVSFVGGVVTIKN |
| | | NKNSNDPTPINPSEPKVVTYGRKFVKTNQDGSERLAGATFL |
| | | VKNSQSQYLARKSGVATNEAHKAVTDAKVQLDEAVKAYNKL |
| | | TKEQQESQDGKAALNLIDEKQTAYNEAFAKANYSYEWVVDK |
| | | NAANVVKLISNTAGKFEITGLNAGEYSLEETQAPTGYAKLSS |
| | | DVSFKVNDTSYSEGASNDIAYDKDSGKTDAQKVVNKKVTIP |
| | | QTGGIGTILFTIIGLSIMLGAVVIMKRRQSEEA |
| 161 | Native D3 (GBS strain | NNPTTENEPQTGNPVNKEITVRKTWAVDGNEVNKGDEKVD |
| | DK21) | AVFTLQVKDSDKWVNVDSATATAATDFKYTFKNLDNAKTYR |
| | | VVERVSGYAPAYVSFVGGVVTIKNNKNSNDPTPI |
| 162 | First endogenous loop | VDGNEVNKGDEKVDA |
| | (GBS strain DK21) | |
| 163 | Second endogenous loop | DSDK |
| | (GBS strain DK21) | |
| 164 | Third endogenous loop | ATATAATDF |
| | (GBS strain DK21) | |
| 165 | Fourth endogenous loop | KNLDNAKTY |
| | (GBS strain DK21) | |
| 166 | Fifth endogenous loop | RVSGYA |
| | (GBS strain DK21) | |
| 167 | Sixth endogenous loop | VGGV |
| | (GBS strain DK21) | |
| 168 | First Scaffold Region | NNPTTENEPQTGNPVNKEITVRKTWA |
| | (GBS strain DK21) | |
| 169 | Second Scaffold Region | VFTLQVK |
| | (GBS strain DK21) | |
| 170 | Third Scaffold Region | WVNVDS |
| | (GBS strain DK21) | |
| 171 | Fourth Scaffold Region | KYTF |
| | (GBS strain DK21) | |
| 172 | Fifth Scaffold Region | RVVE |
| | (GBS strain DK21) | |
| 173 | Sixth Scaffold Region | PAYVSF |
| | (GBS strain DK21) | |
| 174 | Seventh Scaffold Region | VTIKNNKNSNDPTPI |
| | (GBS strain DK21) | |

In view of the information provided herein, the skilled person is readily able to identify domain 3 (D3) of BP-2a from any GBS strain.

The 3D structure of BP-2a from Streptococcus agalactiae strain 515 has been solved to high resolution (1.75 Å) (PDB: 2XTL; Nuccitelli, A. et al., (2011) Structure-Based Approach to Rationally Design a Chimeric Protein for an Effective Vaccine Against Group B Streptococcus Infections. Proc Natl Acad Sci USA 108(25), 10278-10283, incorporated herein by reference). Using the experimentally solved structure of BP-2a from 515 as a template starting point, comparative modelling methods can be used, for example, to generate models of other BP-2a proteins, in particular other BP-2a D3 domains. Comparative modelling proceeds in two steps: first, the protein sequence being modelled is aligned to evolutionarily related sequences with known structures, and second, three dimensional models are built guided by information from these structures (Song, Y., et al., (2013) High-resolution comparative modelling with RosettaCM. Structure 21(10), 1735-1742, incorporated herein by reference). Methods for comparative modelling include MODELLER, I-TASSER and RosettaCM. Preferably, the comparative modelling method used to predict the structure of BP-2a proteins from other GBS strains is RosettaCM (Song, Y., et al., (2013)).

RosettaCM was used successfully by Nuccitelli et al., to demonstrate that the overall topological organisation of two BP-2a variants (from two different GBS strains) is remarkably conserved. In particular, BP-2a proteins from GBS strains H36B and CJB111 were modelled, which are the most evolutionarily distant and evolutionarily closest variants to BP-2a from GBS strain 515, respectively. BP-2a from H36B shares 42.2% sequence identity with BP-2a from 515. However, according to the model, the protein possesses a similar four-domain organisation, including domain D3. As modelled, H36B domain 3 adopts IgG-like folds and forms internal isopeptide bonds, as in the D3 from strain 515 (Nuccitelli et al., (2011), Fig. S3). As expected from a protein sharing 76% sequence identity with BP-2a from GBS strain 515, BP-2a from GBS strain CJB111 was essentially superimposable with BP-2a from strain 515, including D3 (Nuccitelli et al., (2011), Fig. S3). Thus, the data in Nuccitelli et al. supports the use of comparative homology modelling based on BP-2a from GBS strain 515 as a template to model and identify the domains of BP-2a proteins from other strains, e.g., using RosettaCM. Furthermore, it is apparent to the skilled person that the D3 from any GBS strain can be engineered for use as a scaffold, as described herein.

In a native BP-2a D3 polypeptide, the seven scaffold regions alternate with the six endogenous loops, sequentially, in a single, contiguous polypeptide sequence, i.e., scaffold1-loop1-scaffold2-loop2-scaffold3-loop3-scaffold4-loop4-scaffold5-loop5-scaffold6-loop6-scaffold7. The scaffold regions adopt secondary structures (e.g., 3-strands), which are linked to one another by the intervening endogenous loops. The endogenous loops are predominantly if not completely disordered in terms of secondary structure. It is known that due to the relatively flexible nature and dynamics of protein loops, it is possible for said loops to form transient secondary structure. Nonetheless, the endogenous loops of BP-2a D3 are predominantly “unstructured”, i.e. in a state where no stable 3D structure is present, in contrast to the scaffold regions that are “structured”, i.e. in a state where stable 3D structure is present (see, e.g. FIG. 2).

The unstructured (loop) and structured (scaffold) regions of any given BP-2a D3 polypeptide can be determined by routine means. For example, the relative positions and amino acids in a BP-2a D3 sequence that correspond to endogenous loops versus scaffold regions can be determined by reference to a 3D structural model of BP-2a D3 or to a protein structure prediction tool. The 3D structural model may be obtained experimentally (e.g. X-ray crystallography) or via in silico modelling, e.g. as described above. Other protein secondary structure prediction tools are also available and would be readily implemented by the skilled person to identify corresponding positions in the BP-2a D3 polypeptide sequence from other GBS strains e.g. PSI-blast based secondary structure PREDiction (PSIPRED).

Exemplary BP-2a sequences from different GBS strains are provided in Table 1 above including the sequences of D3 and the scaffold regions and endogenous loops therein. In view of the information provided herein, the skilled person is readily able to identify the scaffold regions and endogenous loops of domain 3 (D3) of BP-2a from any GBS strain.

In a preferred embodiment, the positions in a BP-2a D3 polypeptide sequence that are spanned by the amino acids of the scaffold regions are determined by PyMOL or Chimera. In a preferred embodiment, the positions in a BP-2a D3 polypeptide sequence that are spanned by the amino acids of the endogenous loops are determined by PyMOL or Chimera.

In an embodiment the BP-2a D3 polypeptide is at least 40%, at least 50%, at least 60%, at least 70%, at least 80%, at least 90%, at least 95%, at least 97%, at least 98%, at least 99% or 100% identical to the amino acid sequence shown in SEQ ID NO:2; SEQ ID NO:101; SEQ ID NO:116; SEQ ID NO:131; SEQ ID NO:146; or SEQ ID NO:161, but for the one or more exogenous polypeptides replacing (wholly or partially) at least one of the endogenous loops. In a preferred embodiment, the BP-2a D3 polypeptide is at 90% or at least 95% identical to the amino acid sequence shown in SEQ ID NO:2; SEQ ID NO:101; SEQ ID NO:116; SEQ ID NO:131; SEQ ID NO:146; or SEQ ID NO:161,

[515] In preferred embodiments, the BP-2a D3 polypeptide is at least 40%, at least 50%, at least 60%, at least 70%, at least 80%, at least 90%, at least 95%, at least 97%, at least 98%, or at least 99% identical to the amino acid sequence shown in SEQ ID NO:2. In this context, the percentage identity is calculated with reference to the BP-2a D3 polypeptide only and excluding the one or more exogenous polypeptide sequences.

[H36B] In certain embodiments, the BP-2a D3 polypeptide is at least 40%, at least 50%, at least 60%, at least 70%, at least 80%, at least 90%, at least 95%, at least 97%, at least 98%, or at least 99% identical to the amino acid sequence shown in SEQ ID NO:101. In this context, the percentage identity is calculated with reference to the BP-2a D3 polypeptide only and excluding the one or more exogenous polypeptide sequences.

[CJB111] In certain embodiments, the BP-2a D3 polypeptide is at least 40%, at least 50%, at least 60%, at least 70%, at least 80%, at least 90%, at least 95%, at least 97%, at least 98%, or at least 99% identical to the amino acid sequence shown in SEQ ID NO:116. In this context, the percentage identity is calculated with reference to the BP-2a D3 polypeptide only and excluding the one or more exogenous polypeptide sequences.

In certain embodiments, the BP-2a D3 polypeptide is at least 40%, at least 50%, at least 60%, at least 70%, at least 80%, at least 90%, at least 95%, at least 97%, at least 98%, or at least 99% identical to the amino acid sequence shown in SEQ ID NO:131. In this context, the percentage identity is calculated with reference to the BP-2a D3 polypeptide only and excluding the one or more exogenous polypeptide sequences.

[CJB110] In certain embodiments, the BP-2a D3 polypeptide is at least 40%, at least 50%, at least 60%, at least 70%, at least 80%, at least 90%, at least 95%, at least 97%, at least 98%, or at least 99% identical to the amino acid sequence shown in SEQ ID NO:146. In this context, the percentage identity is calculated with reference to the BP-2a D3 polypeptide only and excluding the one or more exogenous polypeptide sequences.

[DK21] In certain embodiments, the BP-2a D3 polypeptide is at least 40%, at least 50%, at least 60%, at least 70%, at least 80%, at least 90%, at least 95%, at least 97%, at least 98%, or at least 99% identical to the amino acid sequence shown in SEQ ID NO:161. In this context, the percentage identity is calculated with reference to the BP-2a D3 polypeptide only and excluding the one or more exogenous polypeptide sequences.

References to a percentage sequence identity between two amino acid sequences means that, when aligned, that percentage of amino acids is the same in comparing the two sequences, as defined hereinabove.

In all embodiments wherein the recited amino acid sequence is not 100% identical to the recited reference sequence, the amino acid sequence variation is preferably attributable to one or more conservative amino acid substitutions. In certain such embodiments, a non-essential amino acid in the polypeptide may be replaced with another amino acid from the same side chain family. In certain embodiments, a string of amino acids can be replaced with a structurally similar string that differs in order and/or composition of side chain family members. A non-essential amino acid is an amino acid the-53-utationn of which does not cause loss of structure or function.

In certain embodiments, the BP-2a D3 polypeptide comprises or consists of the amino acid sequence shown in SEQ ID NO:2; SEQ ID NO:101; SEQ ID NO:116; SEQ ID NO:131; SEQ ID NO:146; or SEQ ID NO:161, but for the one or more exogenous polypeptides replacing (wholly or partially) at least one of the endogenous loops. In preferred embodiments, the BP-2a D3 polypeptide comprises or consists of the amino acid sequence shown in SEQ ID NO:2 but for the one or more exogenous polypeptides replacing (wholly or partially) at least one of the endogenous loops.

In certain embodiments, the BP-2a D3 polypeptide lacks one or more amino acids (e.g., 1, 2, 3, 4, 5, 6, 7, 8, 9 or 10 amino acids) from the N-terminus of the amino acid sequence shown in SEQ ID NO:2. In certain embodiments, the BP-2a D3 polypeptide lacks one or more amino acids (e.g., 1, 2, 3, 4, 5, 6, 7, 8, 9 or 10 amino acids) from the N-terminus of the amino acid sequence shown in SEQ ID NO:101. In certain embodiments, the BP-2a D3 polypeptide lacks one or more amino acids (e.g., 1, 2, 3, 4, 5, 6, 7, 8, 9 or 10 amino acids) from the N-terminus of the amino acid sequence shown in SEQ ID NO:116. In certain embodiments, the BP-2a D3 polypeptide lacks one or more amino acids (e.g., 1, 2, 3, 4, 5, 6, 7, 8, 9 or 10 amino acids) from the N-terminus of the amino acid sequence shown in SEQ ID NO:131. In certain embodiments, the BP-2a D3 polypeptide lacks one or more amino acids (e.g., 1, 2, 3, 4, 5, 6, 7, 8, 9 or 10 amino acids) from the N-terminus of the amino acid sequence shown in SEQ ID NO:146. In certain embodiments, the BP-2a D3 polypeptide lacks one or more amino acids (e.g., 1, 2, 3, 4, 5, 6, 7, 8, 9 or 10 amino acids) from the N-terminus of the amino acid sequence shown in SEQ ID NO:161.

In certain embodiments, the BP-2a D3 polypeptide lacks one or more amino acids (e.g., 1, 2, 3, 4, 5, 6, 7, 8, 9 or 10 amino acids) from the C-terminus of the amino acid sequence shown in SEQ ID NO:2. In certain embodiments, the BP-2a D3 polypeptide lacks one or more amino acids (e.g., 1, 2, 3, 4, 5, 6, 7, 8, 9 or 10 amino acids) from the C-terminus of the amino acid sequence shown in SEQ ID NO:101. In certain embodiments, the BP-2a D3 polypeptide lacks one or more amino acids (e.g., 1, 2, 3, 4, 5, 6, 7, 8, 9 or 10 amino acids) from the C-terminus of the amino acid sequence shown in SEQ ID NO:116. In certain embodiments, the BP-2a D3 polypeptide lacks one or more amino acids (e.g., 1, 2, 3, 4, 5, 6, 7, 8, 9 or 10 amino acids) from the C-terminus of the amino acid sequence shown in SEQ ID NO:131. In certain embodiments, the BP-2a D3 polypeptide lacks one or more amino acids (e.g., 1, 2, 3, 4, 5, 6, 7, 8, 9 or 10 amino acids) from the C-terminus of the amino acid sequence shown in SEQ ID NO:146. In certain embodiments, the BP-2a D3 polypeptide lacks one or more amino acids (e.g., 1, 2, 3, 4, 5, 6, 7, 8, 9 or 10 amino acids) from the C-terminus of the amino acid sequence shown in SEQ ID NO:161.

### Endogenous Loops

According to the first aspect, at least one endogenous loop of the BP-2a D3 polypeptide is partially or wholly replaced by an exogenous polypeptide, where the at least one endogenous loop is selected from the first, second, third, fourth, fifth and sixth endogenous loops of the BP-2a D3 polypeptide.

As described herein, native BP-2a D3 polypeptides have the structure: scaffold1-loop1-scaffold2-loop2-scaffold3-loop3-scaffold4-loop4-scaffold5-loop5-scaffold6-loop6-scaffold7, where the scaffold regions are structured and the loops are disordered. Identifying the disordered loop regions of a given BP-2a D3 protein is within the ability of the skilled person in view of the information provided herein.

In certain embodiments, for a given BP-2a D3 polypeptide:
(i) the first endogenous loop of the BP-2a D3 polypeptide spans the amino acids at positions corresponding to positions 360-372 of SEQ ID NO:1;
(ii) the second endogenous loop of the BP-2a D3 polypeptide spans the amino acids at positions corresponding to positions 380-384 of SEQ ID NO:1;
(iii) the third endogenous loop of the BP-2a D3 polypeptide spans the amino acids at positions corresponding to positions 393-399 of SEQ ID NO:1;
(iv) the fourth endogenous loop of the BP-2a D3 polypeptide spans the amino acids at positions corresponding to positions 404-411 of SEQ ID NO:1;
(v) the fifth endogenous loop of the BP-2a D3 polypeptide spans the amino acids at positions corresponding to positions 418-422 of SEQ ID NO:1; and
(vi) the sixth endogenous loop of the BP-2a D3 polypeptide spans the amino acids at positions corresponding to positions 429-432 of SEQ ID NO:1,
wherein the amino acid positions are numbered according to the sequence shown in SEQ ID NO:1, wherein amino acids 332-447 are the native BP-2a D3 polypeptide.

Identification of amino acids at positions corresponding to those identified positions of SEQ ID NO:1 is within the ability of the skilled person in view of the information provided herein. For example, tools for modelling the structural homology of a BP-2a polypeptide to the D3 of strain 515 are known to the skilled person and include e.g. the RosettaCM program.

By way of example, amino acid positions of a given BP-2a D3 corresponding to the residues of the first endogenous loop of SEQ ID NO:1 are the positions that, when the overall D3 structure is modelled (e.g. by RosettaCM), are flanked by the first two ordered regions corresponding to scaffold1 and scaffold2, but themselves show no stable 3D structure—that is, the first disordered region.

An endogenous loop does not need to be of the same length between D3 proteins. For example, for the BP-2a D3 protein from strain H36B, amino acid positions 369-391 correspond to amino acid positions 360-372 of SEQ ID NO:1.

Corresponding comparisons can be made by the skilled person to identify the amino acids of any given D3 polypeptide that form the other endogenous loops, and thus correspond to the identified positions of SEQ ID NO:1.

In certain preferred embodiments, the BP-2a D3 polypeptide is at least 40%, at least 50%, at least 60%, at least 70%, at least 80%, at least 90%, at least 95%, at least 97%, at least 98%, or at least 99% identical to the amino acid sequence shown in SEQ ID NO: 2, and the at least one endogenous loop of the BP-2a D3 polypeptide that is partially or wholly replaced by an exogenous polypeptide is at least one endogenous loop selected from the first, second, third, fourth, fifth and sixth endogenous loops of the BP-2a D3 polypeptide, wherein:
(i) the first endogenous loop of the BP-2a D3 polypeptide spans the amino acids at positions 360-372;
(ii) the second endogenous loop of the BP-2a D3 polypeptide spans the amino acids at positions 380-384;
(iii) the third endogenous loop of the BP-2a D3 polypeptide spans the amino acids at positions 393-399;
(iv) the fourth endogenous loop of the BP-2a D3 polypeptide spans the amino acids at positions 404-411;
(v) the fifth endogenous loop of the BP-2a D3 polypeptide spans the amino acids at positions 418-422; and
(vi) the sixth endogenous loop of the BP-2a D3 polypeptide spans the amino acids at positions 429-432,
wherein the amino acids are numbered according to the sequence shown in SEQ ID NO:1, wherein amino acids 332-447 are the native BP-2a D3 polypeptide.

In this context, the percentage identity is calculated with reference to the BP-2a D3 polypeptide only and excluding the one or more exogenous polypeptide sequences.

Considering the locations of the endogenous loops that was discovered in respect of SEQ ID NO: 1 (i.e. BP-2a from GBS strain 515) the location of the endogenous loops and scaffold regions can be predicted based on the equivalent positions of the endogenous loops and scaffold regions in these strains. This has been performed below for [H36B], [CJB111], [CJB110], [2603] and [DK21].

[H36B] In certain preferred embodiments, the BP-2a D3 polypeptide is at least 40%, at least 50%, at least 60%, at least 70%, at least 80%, at least 90%, at least 95%, at least 97%, at least 98%, or at least 99% identical to the amino acid sequence shown in SEQ ID NO: 101, and the at least one endogenous loop of the BP-2a D3 polypeptide that is partially or wholly replaced by an exogenous polypeptide is at least one endogenous loop is selected from the first, second, third, fourth, fifth and sixth endogenous loops of the BP-2a D3 polypeptide, wherein:
(i) the first endogenous loop of the BP-2a D3 polypeptide spans the amino acids at positions 369-391;
(ii) the second endogenous loop of the BP-2a D3 polypeptide spans the amino acids at positions 399-402;
(iii) the third endogenous loop of the BP-2a D3 polypeptide spans the amino acids at positions 412-418;
(iv) the fourth endogenous loop of the BP-2a D3 polypeptide spans the amino acids at positions 423-431;
(v) the fifth endogenous loop of the BP-2a D3 polypeptide spans the amino acids at positions 436-441; and
(vi) the sixth endogenous loop of the BP-2a D3 polypeptide spans the amino acids at positions 446-451,
wherein the amino acids are numbered according to the sequence shown in SEQ ID NO:100, wherein amino acids 344-465 are the native BP-2a D3 polypeptide.

[CJB111] In certain preferred embodiments, the BP-2a D3 polypeptide is at least 40%, at least 50%, at least 60%, at least 70%, at least 80%, at least 90%, at least 95%, at least 97%, at least 98%, or at least 99% identical to the amino acid sequence shown in SEQ ID NO: 116, and the at least one endogenous loop of the BP-2a D3 polypeptide that is partially or wholly replaced by an exogenous polypeptide is at least one endogenous loop is selected from the first, second, third, fourth, fifth and sixth endogenous loops of the BP-2a D3 polypeptide, wherein:
(i) the first endogenous loop of the BP-2a D3 polypeptide spans the amino acids at positions 357-370;
(ii) the second endogenous loop of the BP-2a D3 polypeptide spans the amino acids at positions 379-382;
(iii) the third endogenous loop of the BP-2a D3 polypeptide spans the amino acids at positions 392-397;
(iv) the fourth endogenous loop of the BP-2a D3 polypeptide spans the amino acids at positions 403-411;
(v) the fifth endogenous loop of the BP-2a D3 polypeptide spans the amino acids at positions 416-421; and
(vi) the sixth endogenous loop of the BP-2a D3 polypeptide spans the amino acids at positions 428-431,
wherein the amino acids are numbered according to the sequence shown in SEQ ID NO:115, wherein amino acids 332-446 are the native BP-2a D3 polypeptide.

In certain preferred embodiments, the BP-2a D3 polypeptide is at least 40%, at least 50%, at least 60%, at least 70%, at least 80%, at least 90%, at least 95%, at least 97%, at least 98%, or at least 99% identical to the amino acid sequence shown in SEQ ID NO: 131, and the at least one endogenous loop of the BP-2a D3 polypeptide that is partially or wholly replaced by an exogenous polypeptide is at least one endogenous loop is selected from the first, second, third, fourth, fifth and sixth endogenous loops of the BP-2a D3 polypeptide, wherein:
(i) the first endogenous loop of the BP-2a D3 polypeptide spans the amino acids at positions 387-399;
(ii) the second endogenous loop of the BP-2a D3 polypeptide spans the amino acids at positions 404-412;
(iii) the third endogenous loop of the BP-2a D3 polypeptide spans the amino acids at positions 417-436;
(iv) the fourth endogenous loop of the BP-2a D3 polypeptide spans the amino acids at positions 442-447;
(v) the fifth endogenous loop of the BP-2a D3 polypeptide spans the amino acids at positions 454-459; and
(vi) the sixth endogenous loop of the BP-2a D3 polypeptide spans the amino acids at positions 465-468,
wherein the amino acids are numbered according to the sequence shown in SEQ ID NO:130, wherein amino acids 364-483 are the native BP-2a D3 polypeptide.

[CJB110] In certain preferred embodiments, the BP-2a D3 polypeptide is at least 40%, at least 50%, at least 60%, at least 70%, at least 80%, at least 90%, at least 95%, at least 97%, at least 98%, or at least 99% identical to the amino acid sequence shown in SEQ ID NO: 146, and the at least one endogenous loop of the BP-2a D3 polypeptide that is partially or wholly replaced by an exogenous polypeptide is at least one endogenous loop is selected from the first, second, third, fourth, fifth and sixth endogenous loops of the BP-2a D3 polypeptide, wherein:
(i) the first endogenous loop of the BP-2a D3 polypeptide spans the amino acids at positions 385-407;
(ii) the second endogenous loop of the BP-2a D3 polypeptide spans the amino acids at positions 416-419;
(iii) the third endogenous loop of the BP-2a D3 polypeptide spans the amino acids at positions 429-435;
(iv) the fourth endogenous loop of the BP-2a D3 polypeptide spans the amino acids at positions 440-447;
(v) the fifth endogenous loop of the BP-2a D3 polypeptide spans the amino acids at positions 452-457; and
(vi) the sixth endogenous loop of the BP-2a D3 polypeptide spans the amino acids at positions 463-466,
wherein the amino acids are numbered according to the sequence shown in SEQ ID NO:145, wherein amino acids 360-481 are the native BP-2a D3 polypeptide.

[DK21] In certain preferred embodiments, the BP-2a D3 polypeptide is at least 40%, at least 50%, at least 60%, at least 70%, at least 80%, at least 90%, at least 95%, at least 97%, at least 98%, or at least 99% identical to the amino acid sequence shown in SEQ ID NO: 161, and the at least one endogenous loop of the BP-2a D3 polypeptide that is partially or wholly replaced by an exogenous polypeptide is at least one endogenous loop is selected from the first, second, third, fourth, fifth and sixth endogenous loops of the BP-2a D3 polypeptide, wherein:
(i) the first endogenous loop of the BP-2a D3 polypeptide spans the amino acids at positions 365-379;
(ii) the second endogenous loop of the BP-2a D3 polypeptide spans the amino acids at positions 387-390;
(iii) the third endogenous loop of the BP-2a D3 polypeptide spans the amino acids at positions 397-405;
(iv) the fourth endogenous loop of the BP-2a D3 polypeptide spans the amino acids at positions 410-418;
(v) the fifth endogenous loop of the BP-2a D3 polypeptide spans the amino acids at positions 423-428; and
(vi) the sixth endogenous loop of the BP-2a D3 polypeptide spans the amino acids at positions 435-438,
wherein the amino acids are numbered according to the sequence shown in SEQ ID NO:160, wherein amino acids 339-453 the native BP-2a D3 polypeptide.

In certain embodiments, the at least one endogenous loop of the BP-2a D3 polypeptide that is partially or wholly replaced with an exogenous polypeptide is selected from the first, second, third, fifth and sixth endogenous loops. In certain preferred embodiments, the at least one endogenous loop of the BP-2a D3 polypeptide that is partially or wholly replaced with an exogenous polypeptide is the first endogenous loop. In more preferred embodiments, the at least one endogenous loop of the BP-2a D3 polypeptide that is partially or wholly replaced with an exogenous polypeptide is the second endogenous loop. In certain embodiments, the at least one endogenous loop of the BP-2a D3 polypeptide that is partially or wholly replaced with an exogenous polypeptide is the third endogenous loop. In certain embodiments, the at least one endogenous loop of the BP-2a D3 polypeptide that is partially or wholly replaced with an exogenous polypeptide is the fifth endogenous loop. In certain preferred embodiments, the at least one endogenous loop of the BP-2a D3 polypeptide that is partially or wholly replaced with an exogenous polypeptide is the sixth endogenous loop. In certain embodiments, the at least one endogenous loop of the BP-2a D3 polypeptide that is partially or wholly replaced with an exogenous polypeptide is the fourth endogenous loop. However, in most preferred embodiments, the fourth endogenous loop is not partially or wholly replaced.

In certain preferred embodiments, no more than one endogenous loop of the BP-2a D3 polypeptide is partially or wholly replaced by an exogenous polypeptide. In certain other embodiments, two or more endogenous loops of the BP-2a D3 polypeptide are each independently partially or wholly replaced by an exogenous polypeptide. In preferred such embodiments, two endogenous loops of the BP-2a D3 polypeptide are each independently partially or wholly replaced by an exogenous polypeptide.

In certain embodiments, any two of the first, second, third, fifth, and sixth endogenous loops of the BP-2a D3 polypeptide are each independently partially or wholly replaced by an exogenous polypeptide. In certain embodiments, the first and third endogenous loops of the BP-2a D3 polypeptide are each independently partially or wholly replaced by an exogenous polypeptide. In certain embodiments, the first and fifth endogenous loops of the BP-2a D3 polypeptide are each independently partially or wholly replaced by an exogenous polypeptide. In certain embodiments, the first and sixth endogenous loops of the BP-2a D3 polypeptide are each independently partially or wholly replaced by an exogenous polypeptide. In certain embodiments, the second and third endogenous loops of the BP-2a D3 polypeptide are each independently partially or wholly replaced by an exogenous polypeptide. In certain embodiments, the second and fifth endogenous loops of the BP-2a D3 polypeptide are each independently partially or wholly replaced by an exogenous polypeptide. In certain embodiments, the second and sixth endogenous loops of the BP-2a D3 polypeptide are each independently partially or wholly replaced by an exogenous polypeptide. In certain embodiments, the third and fifth endogenous loops of the BP-2a D3 polypeptide are each independently partially or wholly replaced by an exogenous polypeptide. In certain embodiments, the third and sixth endogenous loops of the BP-2a D3 polypeptide are each independently partially or wholly replaced by an exogenous polypeptide. In certain embodiments, the fifth and sixth endogenous loops of the BP-2a D3 polypeptide are each independently partially or wholly replaced by an exogenous polypeptide. In preferred embodiments, the first and second endogenous loops of the BP-2a D3 polypeptide are each independently partially or wholly replaced by an exogenous polypeptide.

In certain embodiments, any three of the first, second, third, fifth, and sixth endogenous loops of the BP-2a D3 polypeptide are each independently partially or wholly replaced by an exogenous polypeptide. In certain embodiments, any four of the first, second, third, fifth, and sixth endogenous loops of the BP-2a D3 polypeptide are each independently partially or wholly replaced by an exogenous polypeptide. In certain embodiments, the first, second, third, fifth, and sixth endogenous loops of the BP-2a D3 polypeptide are each independently partially or wholly replaced by an exogenous polypeptide.

In certain embodiments, when present:
(i) the first endogenous loop of the BP-2a D3 polypeptide is at least 40%, at least 50%, at least 60%, at least 70%, at least 80%, at least 90%, at least 95%, at least 97%, at least 98%, or at least 99% to the amino acid sequence shown in SEQ ID NO:4, 102, 117, 132, 147, or 162;
(ii) the second endogenous loop of the BP-2a D3 polypeptide is at least 40%, at least 50%, at least 60%, at least 70%, at least 80%, at least 90%, at least 95%, at least 97%, at least 98%, or at least 99% identical to the amino acid sequence shown in SEQ ID NO:5, 103, 118, 133, 148, or 163;
(iii) the third endogenous loop of the BP-2a D3 polypeptide is at least 40%, at least 50%, at least 60%, at least 70%, at least 80%, at least 90%, at least 95%, at least 97%, at least 98%, or at least 99% identical to the amino acid sequence shown in SEQ ID NO:6, 104, 119, 134, 149, or 164;
(iv) the fourth endogenous loop of the BP-2a D3 polypeptide is at least 40%, at least 50%, at least 60%, at least 70%, at least 80%, at least 90%, at least 95%, at least 97%, at least 98%, or at least 99% identical to the amino acid sequence shown in SEQ ID NO:7, 105, 120, 135, 150, or 165;
(v) the fifth endogenous loop of the BP-2a D3 polypeptide is at least 40%, at least 50%, at least 60%, at least 70%, at least 80%, at least 90%, at least 95%, at least 97%, at least 98%, or at least 99% identical to the amino acid sequence shown in SEQ ID NO:8, 106, 121, 136, 151, or 166; and
(vi) the sixth endogenous loop of the BP-2a D3 polypeptide is at least 40%, at least 50%, at least 60%, at least 70%, at least 80%, at least 90%, at least 95%, at least 97%, at least 98%, or at least 99% identical to the amino acid sequence shown in SEQ ID NO:9, 107, 122, 137, 152, or 167.

[515] In certain embodiments, when present:
(i) the first endogenous loop of the BP-2a D3 polypeptide is at least 40%, at least 50%, at least 60%, at least 70%, at least 80%, at least 90%, at least 95%, at least 97%, at least 98%, or at least 99% to the amino acid sequence shown in SEQ ID NO:4;
(ii) the second endogenous loop of the BP-2a D3 polypeptide is at least 40%, at least 50%, at least 60%, at least 70%, at least 80%, at least 90%, at least 95%, at least 97%, at least 98%, or at least 99% identical to the amino acid sequence shown in SEQ ID NO:5;
(iii) the third endogenous loop of the BP-2a D3 polypeptide is at least 40%, at least 50%, at least 60%, at least 70%, at least 80%, at least 90%, at least 95%, at least 97%, at least 98%, or at least 99% identical to the amino acid sequence shown in SEQ ID NO:6;
(iv) the fourth endogenous loop of the BP-2a D3 polypeptide is at least 40%, at least 50%, at least 60%, at least 70%, at least 80%, at least 90%, at least 95%, at least 97%, at least 98%, or at least 99% identical to the amino acid sequence shown in SEQ ID NO:7;
(v) the fifth endogenous loop of the BP-2a D3 polypeptide is at least 40%, at least 50%, at least 60%, at least 70%, at least 80%, at least 90%, at least 95%, at least 97%, at least 98%, or at least 99% identical to the amino acid sequence shown in SEQ ID NO:8; and
(vi) the sixth endogenous loop of the BP-2a D3 polypeptide is at least 40%, at least 50%, at least 60%, at least 70%, at least 80%, at least 90%, at least 95%, at least 97%, at least 98%, or at least 99% identical to the amino acid sequence shown in SEQ ID NO:9.

[515] In certain embodiments, when present:
(i) the first endogenous loop of the BP-2a D3 polypeptide comprises or consists of the amino acid sequence shown in SEQ ID NO:4;
(ii) the second endogenous loop of the BP-2a D3 polypeptide comprises or consists of the amino acid sequence shown in SEQ ID NO:5;
(iii) the third endogenous loop of the BP-2a D3 polypeptide comprises or consists of the amino acid sequence shown in SEQ ID NO:6;
(iv) the fourth endogenous loop of the BP-2a D3 polypeptide comprises or consists of the amino acid sequence shown in SEQ ID NO:7;
(v) the fifth endogenous loop of the BP-2a D3 polypeptide comprises or consists of the amino acid sequence shown in SEQ ID NO:8; and
(vi) the sixth endogenous loop of the BP-2a D3 polypeptide comprises or consists of the amino acid sequence shown in SEQ ID NO:9.

Based on the sequences of the endogenous loops and scaffold regions within the BP-2a D3 polypeptide from strain 515 (elucidated by X-Ray crystallography), it is within the remit of the person skilled in the art to determine the sequences of the endogenous loops and scaffold regions for other GBS strains. For example, the sequences of the endogenous loops with regards to GBS strains [H36B], [CJB111], [CJB110], [2603] and [DK21] are predicted below.

[H36B] In certain embodiments, when present:
(vii) the first endogenous loop of the BP-2a D3 polypeptide is at least 40%, at least 50%, at least 60%, at least 70%, at least 80%, at least 90%, at least 95%, at least 97%, at least 98%, or at least 99% to the amino acid sequence shown in SEQ ID NO: 102;
(viii) the second endogenous loop of the BP-2a D3 polypeptide is at least 40%, at least 50%, at least 60%, at least 70%, at least 80%, at least 90%, at least 95%, at least 97%, at least 98%, or at least 99% identical to the amino acid sequence shown in SEQ ID NO:103;
(ix) the third endogenous loop of the BP-2a D3 polypeptide is at least 40%, at least 50%, at least 60%, at least 70%, at least 80%, at least 90%, at least 95%, at least 97%, at least 98%, or at least 99% identical to the amino acid sequence shown in SEQ ID NO:104;
(x) the fourth endogenous loop of the BP-2a D3 polypeptide is at least 40%, at least 50%, at least 60%, at least 70%, at least 80%, at least 90%, at least 95%, at least 97%, at least 98%, or at least 99% identical to the amino acid sequence shown in SEQ ID NO:105;
(xi) the fifth endogenous loop of the BP-2a D3 polypeptide is at least 40%, at least 50%, at least 60%, at least 70%, at least 80%, at least 90%, at least 95%, at least 97%, at least 98%, or at least 99% identical to the amino acid sequence shown in SEQ ID NO:106; and
(xii) the sixth endogenous loop of the BP-2a D3 polypeptide is at least 40%, at least 50%, at least 60%, at least 70%, at least 80%, at least 90%, at least 95%, at least 97%, at least 98%, or at least 99% identical to the amino acid sequence shown in SEQ ID NO:107.

[H36B] In certain embodiments, when present:
(vii) the first endogenous loop of the BP-2a D3 polypeptide comprises or consists of the amino acid sequence shown in SEQ ID NO:102;
(viii) the second endogenous loop of the BP-2a D3 polypeptide comprises or consists of the amino acid sequence shown in SEQ ID NO:103;
(ix) the third endogenous loop of the BP-2a D3 polypeptide comprises or consists of the amino acid sequence shown in SEQ ID NO:104;
(x) the fourth endogenous loop of the BP-2a D3 polypeptide comprises or consists of the amino acid sequence shown in SEQ ID NO:105;
(xi) the fifth endogenous loop of the BP-2a D3 polypeptide comprises or consists of the amino acid sequence shown in SEQ ID NO:106; and
(xii) the sixth endogenous loop of the BP-2a D3 polypeptide comprises or consists of the amino acid sequence shown in SEQ ID NO:107.

[CJB111] In certain embodiments, when present:
(i) the first endogenous loop of the BP-2a D3 polypeptide is at least 40%, at least 50%, at least 60%, at least 70%, at least 80%, at least 90%, at least 95%, at least 97%, at least 98%, or at least 99% to the amino acid sequence shown in SEQ ID NO: 117;
(ii) the second endogenous loop of the BP-2a D3 polypeptide is at least 40%, at least 50%, at least 60%, at least 70%, at least 80%, at least 90%, at least 95%, at least 97%, at least 98%, or at least 99% identical to the amino acid sequence shown in SEQ ID NO:118;
(iii) the third endogenous loop of the BP-2a D3 polypeptide is at least 40%, at least 50%, at least 60%, at least 70%, at least 80%, at least 90%, at least 95%, at least 97%, at least 98%, or at least 99% identical to the amino acid sequence shown in SEQ ID NO:119;
(iv) the fourth endogenous loop of the BP-2a D3 polypeptide is at least 40%, at least 50%, at least 60%, at least 70%, at least 80%, at least 90%, at least 95%, at least 97%, at least 98%, or at least 99% identical to the amino acid sequence shown in SEQ ID NO:120;
(v) the fifth endogenous loop of the BP-2a D3 polypeptide is at least 40%, at least 50%, at least 60%, at least 70%, at least 80%, at least 90%, at least 95%, at least 97%, at least 98%, or at least 99% identical to the amino acid sequence shown in SEQ ID NO:121; and
(vi) the sixth endogenous loop of the BP-2a D3 polypeptide is at least 40%, at least 50%, at least 60%, at least 70%, at least 80%, at least 90%, at least 95%, at least 97%, at least 98%, or at least 99% identical to the amino acid sequence shown in SEQ ID NO:122.

[CJB111] In certain embodiments, when present:
(i) the first endogenous loop of the BP-2a D3 polypeptide comprises or consists of the amino acid sequence shown in SEQ ID NO:117;
(ii) the second endogenous loop of the BP-2a D3 polypeptide comprises or consists of the amino acid sequence shown in SEQ ID NO:118;
(iii) the third endogenous loop of the BP-2a D3 polypeptide comprises or consists of the amino acid sequence shown in SEQ ID NO:119;
(iv) the fourth endogenous loop of the BP-2a D3 polypeptide comprises or consists of the amino acid sequence shown in SEQ ID NO:120;
(v) the fifth endogenous loop of the BP-2a D3 polypeptide comprises or consists of the amino acid sequence shown in SEQ ID NO:121; and
(vi) the sixth endogenous loop of the BP-2a D3 polypeptide comprises or consists of the amino acid sequence shown in SEQ ID NO:122.

In certain embodiments, when present:
(i) the first endogenous loop of the BP-2a D3 polypeptide is at least 40%, at least 50%, at least 60%, at least 70%, at least 80%, at least 90%, at least 95%, at least 97%, at least 98%, or at least 99% to the amino acid sequence shown in SEQ ID NO: 132;
(ii) the second endogenous loop of the BP-2a D3 polypeptide is at least 40%, at least 50%, at least 60%, at least 70%, at least 80%, at least 90%, at least 95%, at least 97%, at least 98%, or at least 99% identical to the amino acid sequence shown in SEQ ID NO:133;
(iii) the third endogenous loop of the BP-2a D3 polypeptide is at least 40%, at least 50%, at least 60%, at least 70%, at least 80%, at least 90%, at least 95%, at least 97%, at least 98%, or at least 99% identical to the amino acid sequence shown in SEQ ID NO:134;
(iv) the fourth endogenous loop of the BP-2a D3 polypeptide is at least 40%, at least 50%, at least 60%, at least 70%, at least 80%, at least 90%, at least 95%, at least 97%, at least 98%, or at least 99% identical to the amino acid sequence shown in SEQ ID NO:135;
(v) the fifth endogenous loop of the BP-2a D3 polypeptide is at least 40%, at least 50%, at least 60%, at least 70%, at least 80%, at least 90%, at least 95%, at least 97%, at least 98%, or at least 99% identical to the amino acid sequence shown in SEQ ID NO:136; and
(vi) the sixth endogenous loop of the BP-2a D3 polypeptide is at least 40%, at least 50%, at least 60%, at least 70%, at least 80%, at least 90%, at least 95%, at least 97%, at least 98%, or at least 99% identical to the amino acid sequence shown in SEQ ID NO:137.

In certain embodiments, when present:
(i) the first endogenous loop of the BP-2a D3 polypeptide comprises or consists of the amino acid sequence shown in SEQ ID NO:132;
(ii) the second endogenous loop of the BP-2a D3 polypeptide comprises or consists of the amino acid sequence shown in SEQ ID NO:133;
(iii) the third endogenous loop of the BP-2a D3 polypeptide comprises or consists of the amino acid sequence shown in SEQ ID NO:134;
(iv) the fourth endogenous loop of the BP-2a D3 polypeptide comprises or consists of the amino acid sequence shown in SEQ ID NO:135;
(v) the fifth endogenous loop of the BP-2a D3 polypeptide comprises or consists of the amino acid sequence shown in SEQ ID NO:136; and
(vi) the sixth endogenous loop of the BP-2a D3 polypeptide comprises or consists of the amino acid sequence shown in SEQ ID NO:137.

[CJB110] In certain embodiments, when present:
(i) the first endogenous loop of the BP-2a D3 polypeptide is at least 40%, at least 50%, at least 60%, at least 70%, at least 80%, at least 90%, at least 95%, at least 97%, at least 98%, or at least 99% to the amino acid sequence shown in SEQ ID NO: 147;
(ii) the second endogenous loop of the BP-2a D3 polypeptide is at least 40%, at least 50%, at least 60%, at least 70%, at least 80%, at least 90%, at least 95%, at least 97%, at least 98%, or at least 99% identical to the amino acid sequence shown in SEQ ID NO:148;
(iii) the third endogenous loop of the BP-2a D3 polypeptide is at least 40%, at least 50%, at least 60%, at least 70%, at least 80%, at least 90%, at least 95%, at least 97%, at least 98%, or at least 99% identical to the amino acid sequence shown in SEQ ID NO:149;
(iv) the fourth endogenous loop of the BP-2a D3 polypeptide is at least 40%, at least 50%, at least 60%, at least 70%, at least 80%, at least 90%, at least 95%, at least 97%, at least 98%, or at least 99% identical to the amino acid sequence shown in SEQ ID NO:150;
(v) the fifth endogenous loop of the BP-2a D3 polypeptide is at least 40%, at least 50%, at least 60%, at least 70%, at least 80%, at least 90%, at least 95%, at least 97%, at least 98%, or at least 99% identical to the amino acid sequence shown in SEQ ID NO:151; and
(vi) the sixth endogenous loop of the BP-2a D3 polypeptide is at least 40%, at least 50%, at least 60%, at least 70%, at least 80%, at least 90%, at least 95%, at least 97%, at least 98%, or at least 99% identical to the amino acid sequence shown in SEQ ID NO:152.

[CJB110] In certain embodiments, when present:
(i) the first endogenous loop of the BP-2a D3 polypeptide comprises or consists of the amino acid sequence shown in SEQ ID NO:147;
(ii) the second endogenous loop of the BP-2a D3 polypeptide comprises or consists of the amino acid sequence shown in SEQ ID NO:148;
(iii) the third endogenous loop of the BP-2a D3 polypeptide comprises or consists of the amino acid sequence shown in SEQ ID NO:149;
(iv) the fourth endogenous loop of the BP-2a D3 polypeptide comprises or consists of the amino acid sequence shown in SEQ ID NO:150;
(v) the fifth endogenous loop of the BP-2a D3 polypeptide comprises or consists of the amino acid sequence shown in SEQ ID NO:151; and
(vi) the sixth endogenous loop of the BP-2a D3 polypeptide comprises or consists of the amino acid sequence shown in SEQ ID NO:152.

[DK21] In certain embodiments, when present:
(i) the first endogenous loop of the BP-2a D3 polypeptide is at least 40%, at least 50%, at least 60%, at least 70%, at least 80%, at least 90%, at least 95%, at least 97%, at least 98%, or at least 99% to the amino acid sequence shown in SEQ ID NO: 162;
(ii) the second endogenous loop of the BP-2a D3 polypeptide is at least 40%, at least 50%, at least 60%, at least 70%, at least 80%, at least 90%, at least 95%, at least 97%, at least 98%, or at least 99% identical to the amino acid sequence shown in SEQ ID NO:163;
(iii) the third endogenous loop of the BP-2a D3 polypeptide is at least 40%, at least 50%, at least 60%, at least 70%, at least 80%, at least 90%, at least 95%, at least 97%, at least 98%, or at least 99% identical to the amino acid sequence shown in SEQ ID NO:164;
(iv) the fourth endogenous loop of the BP-2a D3 polypeptide is at least 40%, at least 50%, at least 60%, at least 70%, at least 80%, at least 90%, at least 95%, at least 97%, at least 98%, or at least 99% identical to the amino acid sequence shown in SEQ ID NO:165;
(v) the fifth endogenous loop of the BP-2a D3 polypeptide is at least 40%, at least 50%, at least 60%, at least 70%, at least 80%, at least 90%, at least 95%, at least 97%, at least 98%, or at least 99% identical to the amino acid sequence shown in SEQ ID NO:166; and
(vi) the sixth endogenous loop of the BP-2a D3 polypeptide is at least 40%, at least 50%, at least 60%, at least 70%, at least 80%, at least 90%, at least 95%, at least 97%, at least 98%, or at least 99% identical to the amino acid sequence shown in SEQ ID NO:167.

[DK21] In certain embodiments, when present:
(i) the first endogenous loop of the BP-2a D3 polypeptide comprises or consists of the amino acid sequence shown in SEQ ID NO:162;
(ii) the second endogenous loop of the BP-2a D3 polypeptide comprises or consists of the amino acid sequence shown in SEQ ID NO:163;
(iii) the third endogenous loop of the BP-2a D3 polypeptide comprises or consists of the amino acid sequence shown in SEQ ID NO:164;
(iv) the fourth endogenous loop of the BP-2a D3 polypeptide comprises or consists of the amino acid sequence shown in SEQ ID NO:165;
(v) the fifth endogenous loop of the BP-2a D3 polypeptide comprises or consists of the amino acid sequence shown in SEQ ID NO:166; and
(vi) the sixth endogenous loop of the BP-2a D3 polypeptide comprises or consists of the amino acid sequence shown in SEQ ID NO:167.

In certain embodiments, the fourth endogenous loop of the BP-2a D3 polypeptide is present and is at least 40%, at least 50%, at least 60%, at least 70%, at least 80%, at least 90%, at least 95%, at least 97%, at least 98%, or at least 99% identical to the amino acid sequence shown in SEQ ID NO:7, 105, 120, 135, 150, or 165, preferably SEQ ID NO: 7. In certain embodiments, the fourth endogenous loop of the BP-2a D3 polypeptide is present and comprises or consists of the amino acid sequence shown in SEQ ID NO:7.

In certain embodiments, the at least one endogenous loop of the BP-2a D3 polypeptide is partially replaced by an exogenous polypeptide. In certain embodiments, the at least one endogenous loop of the BP-2a D3 polypeptide is wholly replaced by an exogenous polypeptide. In certain embodiments one endogenous loop is partially replaced while another endogenous loop is wholly replaced by the respective exogenous polypeptides. In other embodiments, multiple endogenous loops are partially replaced while multiple other endogenous loops are wholly replaced by the respective exogenous polypeptides.

### Scaffold Polypeptides and BP-2a D3 Polypeptides

As described hereinabove, the chimeric polypeptide according to the first aspect of the invention comprises a scaffold polypeptide. The scaffold polypeptide comprises or consists of a BP-2a D3 polypeptide.

As used herein, a “scaffold” refers broadly to a substrate to which one or more additional elements may be attached. A “scaffold polypeptide” as used herein refers to a first polypeptide to which one or more exogenous polypeptides, inter alia, may be attached. As described above, the attachment of the exogenous polypeptide(s) to the scaffold polypeptide is by partial or complete replacement of one or more endogenous loop(s) in the BP-2a D3 polypeptide. As described elsewhere herein, elements other than the exogenous polypeptide(s) may be attached to the scaffold polypeptide and/or comprised therein, e.g. a fusion tag.

The BP-2a D3 polypeptide may be characterised as comprising seven amino acid sequences that form the scaffold regions of the BP-2a D3 polypeptide. In native BP-2a D3 polypeptides, for example, the seven scaffold regions alternate with the six endogenous loops, sequentially, in a single, contiguous polypeptide sequence, i.e., scaffold1-loop1-scaffold2-loop2-scaffold3-loop3-scaffold4-loop4-scaffold5-loop5-scaffold6-loop6-scaffold7, where the scaffold regions are structured and the loops are disordered. Identifying the structured regions of a given BP-2a D3 protein is within the ability of the skilled person in view of the information provided herein.

In certain embodiments, for a given BP-2a D3 polypeptide:
(i) the first scaffold region of the BP-2a D3 polypeptide spans the amino acids at positions corresponding to positions 332-359 of SEQ ID NO:1;
(ii) the second scaffold region of the BP-2a D3 polypeptide spans the amino acids at positions corresponding to positions 373-379 of SEQ ID NO:1;
(iii) the third scaffold region of the BP-2a D3 polypeptide spans the amino acids at positions corresponding to positions 385-392 of SEQ ID NO:1;
(iv) the fourth scaffold region of the BP-2a D3 polypeptide spans the amino acids at positions corresponding to positions 400-403 of SEQ ID NO:1;
(v) the fifth scaffold region of the BP-2a D3 polypeptide spans the amino acids at positions corresponding to positions 412-417 of SEQ ID NO:1;
(vi) the sixth scaffold region spans the amino acids at positions corresponding to positions 423-428 of SEQ ID NO:1; and
(vii) the seventh scaffold region of the BP-2a D3 polypeptide spans the amino acids at positions corresponding to positions 433-447 of SEQ ID NO:1,
wherein the amino acids are numbered according to the sequence shown in SEQ ID NO:1, wherein amino acids 332-447 are the native BP-2a D3 polypeptide from strain 515. Said endogenous loops may be at equivalent positions within the native BP-2a D3 polypeptide from other GBS strains.

Identification of amino acids at positions corresponding to those identified positions of SEQ ID NO:1 is within the ability of the skilled person in view of the information provided herein. For example, tools for modelling the structural homology of a BP-2a polypeptide to the D3 of strain 515 are known to the skilled person and include e.g. the RosettaCM program.

By way of example, amino acid positions of a given BP-2a D3 corresponding to the amino acids of the first scaffold region of SEQ ID NO:1 are the positions that, when the overall D3 structure is modelled (e.g. by RosettaCM) form a first structured region comprising a stable 3D structure. The first position of the first scaffold region is the N-terminal residue of the BP-2a D3. The last position of the first scaffold region corresponds to the last amino acid in the first structured region of D3 immediately N-terminal to the first amino acid of the first disordered region of D3 (corresponding to endogenous loop 1).

A scaffold region does not need to be of the same length between D3 proteins. For example, for the BP-2a D3 protein from strain H36B, amino acid positions 344-368 correspond to amino acid positions 332-359 of SEQ ID NO:1.

Corresponding comparisons can be made by the skilled person to identify the amino acids of any given D3 polypeptide that form the other scaffold regions, and thus correspond to the identified positions of SEQ ID NO:1.

In certain preferred embodiments, the BP-2a D3 polypeptide is at least 40%, at least 50%, at least 60%, at least 70%, at least 80%, at least 90%, at least 95%, at least 97%, at least 98%, or at least 99% identical to the amino acid sequence shown in SEQ ID NO:2 and comprises seven scaffold regions, wherein:
(i) the first scaffold region spans the amino acids at positions 332-359;
(ii) the second scaffold region spans the amino acids at positions 373-379;
(iii) the third scaffold region spans the amino acids at positions 385-392;
(iv) the fourth scaffold region spans the amino acids at positions 400-403;
(v) the fifth scaffold region spans the amino acids at positions 412-417;
(vi) the sixth scaffold region spans the amino acids at positions 423-428; and
(vii) the seventh scaffold region spans the amino acids at positions 433-447,
wherein the amino acids are numbered according to the sequence shown in SEQ ID NO:1, wherein amino acids 332-447 are the native BP-2a D3 polypeptide.

In certain preferred embodiments, the BP-2a D3 polypeptide comprises seven scaffold regions, wherein:
(i) the first scaffold region spans the amino acids at positions 332-359;
(ii) the second scaffold region spans the amino acids at positions 373-379;
(iii) the third scaffold region spans the amino acids at positions 385-392;
(iv) the fourth scaffold region spans the amino acids at positions 400-403;
(v) the fifth scaffold region spans the amino acids at positions 412-417;
(vi) the sixth scaffold region spans the amino acids at positions 423-428; and
(vii) the seventh scaffold region spans the amino acids at positions 433-447, wherein the amino acids are numbered according to the sequence shown in SEQ ID NO:1, wherein amino acids 332-447 are the native BP-2a D3 polypeptide.

In certain alternative embodiments, the first scaffold region is from residue 333, 334, 335, 336, 337, or 338 and terminates at residue 359. The remaining scaffold regions remain as previously described.

[515] In certain embodiments, the BP-2a D3 polypeptide comprises seven scaffold regions, wherein:
(i) the first scaffold region is at least 40%, at least 50%, at least 60%, at least 70%, at least 80%, at least 90%, at least 95%, at least 97%, at least 98%, or at least 99% identical to the amino acid sequence shown in SEQ ID NO:10;
(ii) the second scaffold region is at least 40%, at least 50%, at least 60%, at least 70%, at least 80%, at least 90%, at least 95%, at least 97%, at least 98%, or at least 99% identical to the amino acid sequence shown in SEQ ID NO:11;
(iii) the third scaffold region is at least 40%, at least 50%, at least 60%, at least 70%, at least 80%, at least 90%, at least 95%, at least 97%, at least 98%, or at least 99% identical to the amino acid sequence shown in SEQ ID NO:12;
(iv) the fourth scaffold region is at least 40%, at least 50%, at least 60%, at least 70%, at least 80%, at least 90%, at least 95%, at least 97%, at least 98%, or at least 99% identical to the amino acid sequence shown in SEQ ID NO:13;
(v) the fifth scaffold region is at least 40%, at least 50%, at least 60%, at least 70%, at least 80%, at least 90%, at least 95%, at least 97%, at least 98%, or at least 99% identical to the amino acid sequence shown in SEQ ID NO:14;
(vi) the sixth scaffold region is at least 40%, at least 50%, at least 60%, at least 70%, at least 80%, at least 90%, at least 95%, at least 97%, at least 98%, or at least 99% identical to the amino acid sequence shown in SEQ ID NO:15; and
(vii) the seventh scaffold region is at least 40%, at least 50%, at least 60%, at least 70%, at least 80%, at least 90%, at least 95%, at least 97%, at least 98%, or at least 99% identical to the amino acid sequence shown in SEQ ID NO:16.

In certain embodiments, the BP-2a D3 polypeptide comprises seven scaffold regions, wherein:
(i) the first scaffold region comprises or consists of the amino acid sequence shown in SEQ ID NO:10;
(ii) the second scaffold region comprises or consists of the amino acid sequence shown in SEQ ID NO:11;
(iii) the third scaffold region comprises or consists of the amino acid sequence shown in SEQ ID NO:12;
(iv) the fourth scaffold region comprises or consists of the amino acid sequence shown in SEQ ID NO:13;
(v) the fifth scaffold region comprises or consists of the amino acid sequence shown in SEQ ID NO:14;
(vi) the sixth scaffold region comprises or consists of the amino acid sequence shown in SEQ ID NO:15; and
(vii) the seventh scaffold region comprises or consists of the amino acid sequence shown in SEQ ID NO:16.

Based on the location and sequences of the endogenous loops and scaffold regions within the BP-2a D3 sequence of SEQ ID NO: 1 (i.e. strain 515) it has been possible to predict the equivalent location and sequences of the endogenous loops and scaffold regions within the BP-2a D3 sequence from other GBS strains. These have been outlined below for strains [H36B], [CJB111], [CJB110], [2603] and [DK21].

[H36B] In certain preferred embodiments, the BP-2a D3 polypeptide is at least 40%, at least 50%, at least 60%, at least 70%, at least 80%, at least 90%, at least 95%, at least 97%, at least 98%, or at least 99% identical to the amino acid sequence shown in SEQ ID NO: 101, and the BP-2a D3 polypeptide comprises seven scaffold regions, wherein:
(i) the first scaffold region spans the amino acids at positions 344-368;
(ii) the second scaffold region spans the amino acids at positions 392-398;
(iii) the third scaffold region spans the amino acids at positions 403-411;
(iv) the fourth scaffold region spans the amino acids at positions 419-422;
(v) the fifth scaffold region spans the amino acids at positions 432-435;
(vi) the sixth scaffold region spans the amino acids at positions 442-445; and
(vii) the seventh scaffold region spans the amino acids at positions 452-465,
wherein the amino acids are numbered according to the sequence shown in SEQ ID NO:100, wherein amino acids 344-465 are the native BP-2a D3 polypeptide.

[H36B] In certain embodiments, the BP-2a D3 polypeptide comprises seven scaffold regions, wherein:
(i) the first scaffold region is at least 40%, at least 50%, at least 60%, at least 70%, at least 80%, at least 90%, at least 95%, at least 97%, at least 98%, or at least 99% identical to the amino acid sequence shown in SEQ ID NO:108;
(ii) the second scaffold region is at least 40%, at least 50%, at least 60%, at least 70%, at least 80%, at least 90%, at least 95%, at least 97%, at least 98%, or at least 99% identical to the amino acid sequence shown in SEQ ID NO:109;
(iii) the third scaffold region is at least 40%, at least 50%, at least 60%, at least 70%, at least 80%, at least 90%, at least 95%, at least 97%, at least 98%, or at least 99% identical to the amino acid sequence shown in SEQ ID NO: 110;
(iv) the fourth scaffold region is at least 40%, at least 50%, at least 60%, at least 70%, at least 80%, at least 90%, at least 95%, at least 97%, at least 98%, or at least 99% identical to the amino acid sequence shown in SEQ ID NO: 111;
(v) the fifth scaffold region is at least 40%, at least 50%, at least 60%, at least 70%, at least 80%, at least 90%, at least 95%, at least 97%, at least 98%, or at least 99% identical to the amino acid sequence shown in SEQ ID NO: 112;
(vi) the sixth scaffold region is at least 40%, at least 50%, at least 60%, at least 70%, at least 80%, at least 90%, at least 95%, at least 97%, at least 98%, or at least 99% identical to the amino acid sequence shown in SEQ ID NO:113; and
(vii) the seventh scaffold region is at least 40%, at least 50%, at least 60%, at least 70%, at least 80%, at least 90%, at least 95%, at least 97%, at least 98%, or at least 99% identical to the amino acid sequence shown in SEQ ID NO:114.

[H36B] In certain embodiments, wherein the BP-2a D3 polypeptide comprises seven scaffold regions, wherein:
(i) the first scaffold region comprises or consists of the amino acid sequence shown in SEQ ID NO:108;
(ii) the second scaffold region comprises or consists of the amino acid sequence shown in SEQ ID NO:109;
(iii) the third scaffold region comprises or consists of the amino acid sequence shown in SEQ ID NO:110;
(iv) the fourth scaffold region comprises or consists of the amino acid sequence shown in SEQ ID NO:111;
(v) the fifth scaffold region comprises or consists of the amino acid sequence shown in SEQ ID NO:112;
(vi) the sixth scaffold region comprises or consists of the amino acid sequence shown in SEQ ID NO:113; and
(vii) the seventh scaffold region comprises or consists of the amino acid sequence shown in SEQ ID NO:114.

[CJB111] In certain preferred embodiments, the BP-2a D3 polypeptide is at least 40%, at least 50%, at least 60%, at least 70%, at least 80%, at least 90%, at least 95%, at least 97%, at least 98%, or at least 99% identical to the amino acid sequence shown in SEQ ID NO: 116, and, the BP-2a D3 polypeptide comprises seven scaffold regions, wherein:
(i) the first scaffold region spans the amino acids at positions 332-356;
(ii) the second scaffold region spans the amino acids at positions 371-378;
(iii) the third scaffold region spans the amino acids at positions 383-391;
(iv) the fourth scaffold region spans the amino acids at positions 398-402;
(v) the fifth scaffold region spans the amino acids at positions 412-415;
(vi) the sixth scaffold region spans the amino acids at positions 422-427; and
(vii) the seventh scaffold region spans the amino acids at positions 432-446,
wherein the amino acids are numbered according to the sequence shown in SEQ ID NO:115, wherein amino acids 332-446 are the native BP-2a D3 polypeptide.

[CJB111] In certain embodiments, the BP-2a D3 polypeptide comprises seven scaffold regions, wherein:
(i) the first scaffold region is at least 40%, at least 50%, at least 60%, at least 70%, at least 80%, at least 90%, at least 95%, at least 97%, at least 98%, or at least 99% identical to the amino acid sequence shown in SEQ ID NO:123;
(ii) the second scaffold region is at least 40%, at least 50%, at least 60%, at least 70%, at least 80%, at least 90%, at least 95%, at least 97%, at least 98%, or at least 99% identical to the amino acid sequence shown in SEQ ID NO:124;
(iii) the third scaffold region is at least 40%, at least 50%, at least 60%, at least 70%, at least 80%, at least 90%, at least 95%, at least 97%, at least 98%, or at least 99% identical to the amino acid sequence shown in SEQ ID NO:125;
(iv) the fourth scaffold region is at least 40%, at least 50%, at least 60%, at least 70%, at least 80%, at least 90%, at least 95%, at least 97%, at least 98%, or at least 99% identical to the amino acid sequence shown in SEQ ID NO:126;
(v) the fifth scaffold region is at least 40%, at least 50%, at least 60%, at least 70%, at least 80%, at least 90%, at least 95%, at least 97%, at least 98%, or at least 99% identical to the amino acid sequence shown in SEQ ID NO:127;
(vi) the sixth scaffold region is at least 40%, at least 50%, at least 60%, at least 70%, at least 80%, at least 90%, at least 95%, at least 97%, at least 98%, or at least 99% identical to the amino acid sequence shown in SEQ ID NO:128; and
(vii) the seventh scaffold region is at least 40%, at least 50%, at least 60%, at least 70%, at least 80%, at least 90%, at least 95%, at least 97%, at least 98%, or at least 99% identical to the amino acid sequence shown in SEQ ID NO:129.

[CJB111] In certain embodiments, wherein the BP-2a D3 polypeptide comprises seven scaffold regions, wherein:
(i) the first scaffold region comprises or consists of the amino acid sequence shown in SEQ ID NO:123;
(ii) the second scaffold region comprises or consists of the amino acid sequence shown in SEQ ID NO:124;
(iii) the third scaffold region comprises or consists of the amino acid sequence shown in SEQ ID NO:125;
(iv) the fourth scaffold region comprises or consists of the amino acid sequence shown in SEQ ID NO:126;
(v) the fifth scaffold region comprises or consists of the amino acid sequence shown in SEQ ID NO:127;
(vi) the sixth scaffold region comprises or consists of the amino acid sequence shown in SEQ ID NO:128; and
(vii) the seventh scaffold region comprises or consists of the amino acid sequence shown in SEQ ID NO:129.

In certain preferred embodiments, the BP-2a D3 polypeptide is at least 40%, at least 50%, at least 60%, at least 70%, at least 80%, at least 90%, at least 95%, at least 97%, at least 98%, or at least 99% identical to the amino acid sequence shown in SEQ ID NO: 131, and the BP-2a D3 polypeptide comprises seven scaffold regions, wherein:
(i) the first scaffold region spans the amino acids at positions 364-386;
(ii) the second scaffold region spans the amino acids at positions 400-403;
(iii) the third scaffold region spans the amino acids at positions 413-416;
(iv) the fourth scaffold region spans the amino acids at positions 437-441;
(v) the fifth scaffold region spans the amino acids at positions 448-453;
(vi) the sixth scaffold region spans the amino acids at positions 460-464; and
(vii) the seventh scaffold region spans the amino acids at positions 469-483,
wherein the amino acids are numbered according to the sequence shown in SEQ ID NO:130, wherein amino acids 364-483 are the native BP-2a D3 polypeptide.

In certain embodiments, the BP-2a D3 polypeptide comprises seven scaffold regions, wherein:
(i) the first scaffold region is at least 40%, at least 50%, at least 60%, at least 70%, at least 80%, at least 90%, at least 95%, at least 97%, at least 98%, or at least 99% identical to the amino acid sequence shown in SEQ ID NO:138;
(ii) the second scaffold region is at least 40%, at least 50%, at least 60%, at least 70%, at least 80%, at least 90%, at least 95%, at least 97%, at least 98%, or at least 99% identical to the amino acid sequence shown in SEQ ID NO:139;
(iii) the third scaffold region is at least 40%, at least 50%, at least 60%, at least 70%, at least 80%, at least 90%, at least 95%, at least 97%, at least 98%, or at least 99% identical to the amino acid sequence shown in SEQ ID NO:140;
(iv) the fourth scaffold region is at least 40%, at least 50%, at least 60%, at least 70%, at least 80%, at least 90%, at least 95%, at least 97%, at least 98%, or at least 99% identical to the amino acid sequence shown in SEQ ID NO:141;
(v) the fifth scaffold region is at least 40%, at least 50%, at least 60%, at least 70%, at least 80%, at least 90%, at least 95%, at least 97%, at least 98%, or at least 99% identical to the amino acid sequence shown in SEQ ID NO:142;
(vi) the sixth scaffold region is at least 40%, at least 50%, at least 60%, at least 70%, at least 80%, at least 90%, at least 95%, at least 97%, at least 98%, or at least 99% identical to the amino acid sequence shown in SEQ ID NO:143; and
(vii) the seventh scaffold region is at least 40%, at least 50%, at least 60%, at least 70%, at least 80%, at least 90%, at least 95%, at least 97%, at least 98%, or at least 99% identical to the amino acid sequence shown in SEQ ID NO:144.

In certain embodiments, wherein the BP-2a D3 polypeptide comprises seven scaffold regions, wherein:
(i) the first scaffold region comprises or consists of the amino acid sequence shown in SEQ ID NO:138;
(ii) the second scaffold region comprises or consists of the amino acid sequence shown in SEQ ID NO:139;
(iii) the third scaffold region comprises or consists of the amino acid sequence shown in SEQ ID NO:140;
(iv) the fourth scaffold region comprises or consists of the amino acid sequence shown in SEQ ID NO:141;
(v) the fifth scaffold region comprises or consists of the amino acid sequence shown in SEQ ID NO:142;
(vi) the sixth scaffold region comprises or consists of the amino acid sequence shown in SEQ ID NO:143; and
(vii) the seventh scaffold region comprises or consists of the amino acid sequence shown in SEQ ID NO:144.

[CJB110] In certain preferred embodiments, the BP-2a D3 polypeptide is at least 40%, at least 50%, at least 60%, at least 70%, at least 80%, at least 90%, at least 95%, at least 97%, at least 98%, or at least 99% identical to the amino acid sequence shown in SEQ ID NO: 146, and the BP-2a D3 polypeptide comprises seven scaffold regions, wherein:
(i) the first scaffold region spans the amino acids at positions 360-384;
(ii) the second scaffold region spans the amino acids at positions 408-415;
(iii) the third scaffold region spans the amino acids at positions 420-428;
(iv) the fourth scaffold region spans the amino acids at positions 436-439;
(v) the fifth scaffold region spans the amino acids at positions 448-451;
(vi) the sixth scaffold region spans the amino acids at positions 458-462; and
(vii) the seventh scaffold region spans the amino acids at positions 467-481,
wherein the amino acids are numbered according to the sequence shown in SEQ ID NO:145, wherein amino acids 360-481 are the native BP-2a D3 polypeptide.

[CJB110] In certain embodiments, the BP-2a D3 polypeptide comprises seven scaffold regions, wherein:
(i) the first scaffold region is at least 40%, at least 50%, at least 60%, at least 70%, at least 80%, at least 90%, at least 95%, at least 97%, at least 98%, or at least 99% identical to the amino acid sequence shown in SEQ ID NO:153;
(ii) the second scaffold region is at least 40%, at least 50%, at least 60%, at least 70%, at least 80%, at least 90%, at least 95%, at least 97%, at least 98%, or at least 99% identical to the amino acid sequence shown in SEQ ID NO:154;
(iii) the third scaffold region is at least 40%, at least 50%, at least 60%, at least 70%, at least 80%, at least 90%, at least 95%, at least 97%, at least 98%, or at least 99% identical to the amino acid sequence shown in SEQ ID NO:155;
(iv) the fourth scaffold region is at least 40%, at least 50%, at least 60%, at least 70%, at least 80%, at least 90%, at least 95%, at least 97%, at least 98%, or at least 99% identical to the amino acid sequence shown in SEQ ID NO:156;
(v) the fifth scaffold region is at least 40%, at least 50%, at least 60%, at least 70%, at least 80%, at least 90%, at least 95%, at least 97%, at least 98%, or at least 99% identical to the amino acid sequence shown in SEQ ID NO:157;
(vi) the sixth scaffold region is at least 40%, at least 50%, at least 60%, at least 70%, at least 80%, at least 90%, at least 95%, at least 97%, at least 98%, or at least 99% identical to the amino acid sequence shown in SEQ ID NO:158; and
(vii) the seventh scaffold region is at least 40%, at least 50%, at least 60%, at least 70%, at least 80%, at least 90%, at least 95%, at least 97%, at least 98%, or at least 99% identical to the amino acid sequence shown in SEQ ID NO:159.

[CJB110] In certain embodiments, wherein the BP-2a D3 polypeptide comprises seven scaffold regions, wherein:
(i) the first scaffold region comprises or consists of the amino acid sequence shown in SEQ ID NO:153;
(ii) the second scaffold region comprises or consists of the amino acid sequence shown in SEQ ID NO:154;
(iii) the third scaffold region comprises or consists of the amino acid sequence shown in SEQ ID NO:155;
(iv) the fourth scaffold region comprises or consists of the amino acid sequence shown in SEQ ID NO:156;
(v) the fifth scaffold region comprises or consists of the amino acid sequence shown in SEQ ID NO:157;
(vi) the sixth scaffold region comprises or consists of the amino acid sequence shown in SEQ ID NO:158; and
(vii) the seventh scaffold region comprises or consists of the amino acid sequence shown in SEQ ID NO:159.

[DK21] In certain preferred embodiments, the BP-2a D3 polypeptide is at least 40%, at least 50%, at least 60%, at least 70%, at least 80%, at least 90%, at least 95%, at least 97%, at least 98%, or at least 99% identical to the amino acid sequence shown in SEQ ID NO: 161, and the BP-2a D3 polypeptide comprises seven scaffold regions, wherein:
(i) the first scaffold region spans the amino acids at positions 339-364;
(ii) the second scaffold region spans the amino acids at positions 380-386;
(iii) the third scaffold region spans the amino acids at positions 391-396;
(iv) the fourth scaffold region spans the amino acids at positions 406-409;
(v) the fifth scaffold region spans the amino acids at positions 419-422;
(vi) the sixth scaffold region spans the amino acids at positions 429-434; and
(vii) the seventh scaffold region spans the amino acids at positions 439-453,
wherein the amino acids are numbered according to the sequence shown in SEQ ID NO:160, wherein amino acids 339-453 are the native BP-2a D3 polypeptide.

[DK21] In certain embodiments, the BP-2a D3 polypeptide comprises seven scaffold regions, wherein:
(i) the first scaffold region is at least 40%, at least 50%, at least 60%, at least 70%, at least 80%, at least 90%, at least 95%, at least 97%, at least 98%, or at least 99% identical to the amino acid sequence shown in SEQ ID NO:168;
(ii) the second scaffold region is at least 40%, at least 50%, at least 60%, at least 70%, at least 80%, at least 90%, at least 95%, at least 97%, at least 98%, or at least 99% identical to the amino acid sequence shown in SEQ ID NO:169;
(iii) the third scaffold region is at least 40%, at least 50%, at least 60%, at least 70%, at least 80%, at least 90%, at least 95%, at least 97%, at least 98%, or at least 99% identical to the amino acid sequence shown in SEQ ID NO:170;
(iv) the fourth scaffold region is at least 40%, at least 50%, at least 60%, at least 70%, at least 80%, at least 90%, at least 95%, at least 97%, at least 98%, or at least 99% identical to the amino acid sequence shown in SEQ ID NO:171;
(v) the fifth scaffold region is at least 40%, at least 50%, at least 60%, at least 70%, at least 80%, at least 90%, at least 95%, at least 97%, at least 98%, or at least 99% identical to the amino acid sequence shown in SEQ ID NO:172;
(vi) the sixth scaffold region is at least 40%, at least 50%, at least 60%, at least 70%, at least 80%, at least 90%, at least 95%, at least 97%, at least 98%, or at least 99% identical to the amino acid sequence shown in SEQ ID NO:173; and
(vii) the seventh scaffold region is at least 40%, at least 50%, at least 60%, at least 70%, at least 80%, at least 90%, at least 95%, at least 97%, at least 98%, or at least 99% identical to the amino acid sequence shown in SEQ ID NO:174.

[DK21] In certain embodiments, wherein the BP-2a D3 polypeptide comprises seven scaffold regions, wherein:
(i) the first scaffold region comprises or consists of the amino acid sequence shown in SEQ ID NO:168;
(ii) the second scaffold region comprises or consists of the amino acid sequence shown in SEQ ID NO:169;
(iii) the third scaffold region comprises or consists of the amino acid sequence shown in SEQ ID NO:170;
(iv) the fourth scaffold region comprises or consists of the amino acid sequence shown in SEQ ID NO:171;
(v) the fifth scaffold region comprises or consists of the amino acid sequence shown in SEQ ID NO:172;
(vi) the sixth scaffold region comprises or consists of the amino acid sequence shown in SEQ ID NO:173; and
(vii) the seventh scaffold region comprises or consists of the amino acid sequence shown in SEQ ID NO:174.

In a preferred embodiment, the BP-2a D3 polypeptide is from Streptococcus agalactiae. Streptococcus agalactiae may also be referred to as Group B Streptococcus (GBS). “Streptococcus agalactiae”, “Group B Streptococcus” and “GBS” are used interchangeably herein throughout.

In certain embodiments, the BP-2a D3 polypeptide is from Streptococcus agalactiae strain H36B. In certain embodiments, the BP-2a D3 polypeptide is from Streptococcus agalactiae strain CJB111. In certain embodiments, the BP-2a D3 polypeptide is from Streptococcus agalactiae strain 2603. In certain embodiments, the BP-2a D3 polypeptide is from Streptococcus agalactiae strain CJB110. In certain embodiments, the BP-2a D3 polypeptide is from Streptococcus agalactiae strain DK21.

In preferred embodiments, the BP-2a D3 polypeptide is from Streptococcus agalactiae strain 515.

In preferred embodiments, each scaffold region comprises a β-strand. In preferred such embodiments, the scaffold regions of the BP-2a D3 polypeptide form a β-barrel (otherwise referred to as an IgG-like fold).

In a preferred embodiment, the BP-2a D3 polypeptide comprises an intramolecular isopeptide bond. In certain such embodiments, the isopeptide bond is between scaffold region 1 and scaffold region 7. In preferred such embodiments, the intramolecular isopeptide bond is between K355 and N437, wherein the amino acids are numbered according to the sequence shown in SEQ ID NO:1, wherein amino acids 332-447 are the native BP-2a D3 polypeptide. In an embodiment, the isopeptide bond is between K24 and N106, wherein the amino acids are numbered according to the sequence shown in SEQ ID NO:2. In an embodiment, the isopeptide bond is located at the corresponding residues within the disclosed chimeric polypeptides, for example the isopeptide bond is between K43 and N146 in the D3PorBloop5 chimera as disclosed in SEQ ID NO:58.

Without wishing to be bound by theory, it is thought that the intramolecular isopeptide bond contributes to the thermodynamic and proteolytic stability of the chimeric polypeptide but is not required for the correct folding of the chimeric polypeptide nor for its solubility. This was indirectly demonstrated in a study in which the isopeptide bonds in wild type BP-2a (including domain 3) were abolished by alanine-substitution of the lysine residues involved in isopeptide bond formation (Nuccitelli et al., (2011) Structure-based approach to rationally design a chimeric protein for an effective vaccine against Group B Streptococcus infections. Proc Natl Acad Sci USA 108(25):10278-83. Doi: 10.1073/pnas.1106590108). The alanine-mutant expressed well in E. coli as a soluble protein and could still elicit opsonophagocytic antibodies and protection in the animal model (Nuccitelli et al., (2011)).

In certain embodiments, the scaffold polypeptide comprises a fusion tag such as an epitope tag, an affinity tag, a fluorescent tag, or a bioluminescent tag. The fusion tag may, for example, facilitate the purification and/or detection (e.g., visualization) of the chimeric polypeptide. Examples of epitope tags include ALFA-tag, V5-tag, Myc-tag, HA-tag, Spot-tag, T7-tag NE-tag, FLAG-tag and VA-tag. Examples of affinity tags include GST-tag, SBP-tag, polyhistidine-tag and strep-tag. Examples of fluorescent tags include GFPs (e.g., EGFP, sfGFP), BFPs (e.g., EBFP, EBFP2, Azurite, mKalamal), CFPs (e.g., ECFP, Cerulean, CyPet, mTurquoise2), RFPs including DsRed and variants thereof (e.g., mCherry, mOrange, mRaspberry, mApple, mKO, TagRFP, mKate, mRuby, FusionRed, mScarlet and DsRed-Express), YFPs (e.g., Citrine, Venus, Ypet). Examples of bioluminescent tags include luciferases (e.g., RIuc). A single fusion tag may fall in more than one category, e.g., a polyhistidine-tag may be used as both an epitope tag and an affinity tag.

In certain embodiments, the scaffold polypeptide comprises a signal peptide to facilitate trafficking of the chimeric polypeptide. Protein sorting, translocation and secretion mechanisms are known to a person skilled in the art.

The chimeric polypeptide may alternatively or additionally be labelled by any other known means, as appropriate, e.g., radiolabelling, self-labelling protein tags, click chemistry. In certain embodiments, the scaffold polypeptide comprises a tag or ligand for non-covalent conjugation (e.g., His-tag/Ni-NTA, biotin-avidin). In certain embodiments, the scaffold polypeptide comprises a tag or ligand for covalent conjugation (e.g., Halo-tag, SNAP-tag, Sortase, Split-inteins, SpyTag/SpyCatcher). In embodiments wherein the scaffold polypeptide comprises a tag or ligand for non-covalent or covalent conjugation, said conjugation is preferably conjugation to a nanoparticle according to the second aspect of the invention. Therefore, in certain embodiments, the nanoparticle according to the second aspect of the invention comprises a tag or ligand as described above which is suitable for conjugation to a chimeric polypeptide according to the first aspect.

In certain embodiments, the scaffold polypeptide comprises an enzyme-cleavable amino acid sequence. In certain such embodiments, the enzyme-cleavable amino acid sequence is between the BP-2a D3 polypeptide and a fusion tag. In certain embodiments, the enzyme-cleavable amino acid sequence is a TEV protease-cleavable amino acid sequence, preferably EX₁LYX₂Q\X₃ (SEQ ID NO:177) where X₁ is any amino acid, X₂ is F, Y or W, and X₃ is S, G, A, M, C or H, and “\” indicates the cleavage site. In certain embodiments, the TEV-cleavable amino acid sequence is ENLYFQG (SEQ ID NO:176).

In certain embodiments, the scaffold polypeptide comprises an amino acid linker (e.g., 1, 2, 3, 4, 5, 6, 7, 8, 9, 10 amino acids in length) between the C-terminus of a functional element (e.g., a fusion tag, an enzyme-cleavable amino acid sequence) and the N-terminus of the BP-2a D3 polypeptide. In certain embodiments, the scaffold polypeptide comprises an amino acid linker (e.g., 1, 2, 3, 4, 5, 6, 7, 8, 9, 10 amino acids in length) between the C-terminus of the BP-2a D3 polypeptide and the N-terminus of a functional element (e.g., a fusion tag, an enzyme-cleavable amino acid sequence sequence).

In preferred embodiments, the amino acid linker is 1-6 amino acids in length. In more preferred embodiments, the amino acid linker is 1-3 amino acids in length.

In certain embodiments, the scaffold polypeptide is at least 40%, at least 50%, at least 60%, at least 70%, at least 80%, at least 90%, at least 95%, at least 97%, at least 98%, or at least 99% identical to the amino acid sequence shown in SEQ ID NO:3 but for the one or more exogenous polypeptides replacing (wholly or partially) at least one of the endogenous loops.

In certain embodiments, the scaffold polypeptide comprises or consists of the amino acid sequence shown in SEQ ID NO:3 but for the one or more exogenous polypeptides replacing (wholly or partially) at least one of the endogenous loops.

In certain embodiments, the scaffold polypeptide is 12-60 kDa. In certain embodiments, the scaffold polypeptide is 12-60 kDa, optionally 12-50 kDa, optionally 12-40 kDa, optionally 12-30 kDa, optionally 12-20 kDa, optionally 12-18 kDa, optionally 13-17 kDa, optionally 14-16 kDa, optionally around 15 kDa. In certain embodiments, the scaffold polypeptide is around 15 kDa.

### Exogenous Polypeptides

A sequence of amino acids not natively found in the BP-2a D3 polypeptide (an “exogenous polypeptide”) is inserted into the BP-2a D3 scaffold to form the chimeric polypeptide according to the first aspect. The chimeric polypeptide facilitates the recombinant production of exogenous polypeptides. The chimeric polypeptide can also be used to present exogenous polypeptides of interest, e.g., for use as a vaccine, for vaccine development or antibody screening purposes. The skilled person is aware of routine methods of genetic engineering for inserting exogenous polypeptides into the chimeric polypeptide. For example, a polynucleotide sequence encoding the chimeric polypeptide can be synthesised chemically de novo, assembled from synthetic DNA parts or assembled according to any other known cloning/synthesis method. Synthetic polynucleotide sequences are readily obtainable commercially, for example, from GeneArt services (ThermoFisher Scientific). By way of example, a complete expression vector encoding the chimeric polypeptide may be obtained commercially and used to produce the chimeric polypeptide recombinantly. Alternatively, a shorter synthetic polynucleotide sequence encoding the chimeric polypeptide may be obtained from commercial sources and cloned by the skilled person into any suitable expression vector for recombinant production. Suitable E. coli expression vectors may include, for example, plasmids pET15b-TEV (Merck), pET21b(+) (Merck) and pET24b(+) (Merck). Methods of cloning, transformation/transduction/transfection, inducing expression, and protein purification are well known to the skilled person.

Each exogenous polypeptide incorporated into the chimeric polypeptide may consist of a short amino acid sequence (e.g., no less than 5 amino acids in length) or may be a longer polypeptide (e.g., up to 500 amino acids in length, up to 400 amino acids in length or up to 300 amino acids in length). In certain embodiments, the one or more exogenous polypeptides each independently comprise 5-300 amino acids. In certain embodiments, the one or more exogenous polypeptides each independently comprise 5-300 amino acids, optionally 6-250 amino acids, optionally 7-200 amino acids, optionally 8-150 amino acids, optionally 9-100 amino acids, optionally 10-75 amino acids, optionally 11-57 amino acids. In certain embodiments, the one or more exogenous polypeptides each independently comprise 11-57 amino acids.

It is explicitly contemplated as part of the invention that a chimeric polypeptide according to the first aspect may comprise any two exogenous polypeptide(s) disclosed herein. In preferred such embodiments, a first exogenous polypeptide replaces (partially or wholly) the first endogenous loop and a second exogenous polypeptide replaces (partially or wholly) the second endogenous loop.

In all embodiments according to the first aspect wherein the chimeric polypeptide comprises more than one exogenous polypeptide, the exogenous polypeptides may be the same or may be different.

In certain embodiments, the exogenous polypeptide comprises a fragment, preferably an antigenic fragment, of a target protein. In certain embodiments, the fragment is an endogenous loop of the target protein. In certain embodiments, the target protein is a cytosolic protein. In certain embodiments, the target protein is an antibody. In certain embodiments, the target protein is an enzyme. In certain embodiments, the target protein is a signalling protein. In certain embodiments, the target protein is a peptide hormone. In certain embodiments, the target protein is a structural protein. In certain embodiments, the target protein is a motor protein. In certain embodiments, the target protein is a storage protein. In certain embodiments, the target protein is a membrane protein. In certain embodiments, the target protein is a receptor protein. In certain embodiments, the target protein is a transport protein. In certain embodiments the target protein is a virulence factor of a pathogen. In preferred embodiments, the target protein is surface-exposed in its native environment (e.g. in the outer membrane of a gram-negative bacterium, the viral envelope or capsid). In certain embodiments, the target protein is insoluble when recombinantly produced.

In certain embodiments, any one exogenous polypeptide may comprise multiple target proteins as described herein, or fragments or epitopes thereof, for example as a fusion polypeptide. The multiple target proteins (or fragments or epitopes thereof) may be from the same or a different organism, protein, or polypeptide.

In certain embodiments, the antigenic fragment of the target protein is inaccessible to antibodies when comprised in the natively folded target protein.

In certain embodiments, the exogenous polypeptide comprises a fragment (e.g., an antigenic fragment) of a target protein that is from a microorganism or virus (i.e., the exogenous polypeptide comprises a fragment of a target protein natively expressed or encoded by a microorganism or virus, e.g., a part or the whole of a native peptide or protein). In certain embodiments, the microorganism or virus is a pathogenic microorganism or virus. In a preferred embodiment the exogenous polypeptide comprises a fragment (e.g., an antigenic fragment) of a target protein that is from a bacterium.

In preferred embodiments, the pathogenic microorganism or virus is selected from Neisseria gonorrhoeae, Neisseria meningitidis, Haemophilus influenzae (e.g., Non-typeable Haemophilus influenzae), Staphylococcus aureus, human papillomavirus (HPV), Chlamydia trachomatis, Chlamydia muridarum, Streptococcus pneumonia, Escherichia coli (e.g., pathogenic E. coli), Vibrio cholerae and Streptococcus agalactiae. The exogenous polypeptide is not a BP-2a D3 polypeptide.

In certain embodiments, the exogenous polypeptide comprises a fragment (e.g., an antigenic fragment) of a target protein that is a porin, preferably a bacterial porin. In preferred embodiments, the porin is an outer membrane porin from a gram-negative bacterium.

In certain embodiments, the porin is from a pathogenic Chlamydia species (e.g., Chlamydia trachomatis). In certain such embodiments, the porin is major outer membrane protein (also known as “MOMP”, encoded by the ompA gene). In an embodiment, the porin is MOMP from any Chlamydia trachomatis serovar. The amino acid sequences of MOMP from all Chlamydia trachomatis serovars are predominantly conserved but have four variable domains (VDs). Identification of the VD within different MOMP sequences (e.g. from different serovars) is within the remit of the skilled person, for example using the teaching of Yuan et al, Infect Immun. 1989 April; 57(4):1040-9.

In certain preferred embodiments, the exogenous polypeptide comprises a fragment of MOMP, wherein said fragment comprises a variable domain. In an embodiment the exogenous polypeptide comprises a fragment of MOMP, wherein said fragment comprises variable domain 1 (VD1). In an embodiment said fragment of MOMP that comprises VD1 comprises a sequence having at least 75%, 80%, 85%, 90%, 91%, 92%, 93%, 94%, 95%, 96%, 97% 98%, 99% or 100% identical to SEQ ID NO: 197. In certain preferred embodiments, the exogenous polypeptide comprises a fragment of MOMP wherein said fragment comprises variable domain 2 (VD2). In an embodiment said fragment of MOMP that comprises VD2 comprises a sequence having at least 75%, 80%, 85%, 90%, 91%, 92%, 93%, 94%, 95%, 96%, 97% 98%, 99% or 100% identical to SEQ ID NO: 198. In certain preferred embodiments, the exogenous polypeptide comprises a fragment of MOMP wherein said fragment comprises variable domain 3 (VD3). In an embodiment said fragment of MOMP that comprises VD3 comprises a sequence having at least 75%, 80%, 85%, 90%, 91%, 92%, 93%, 94%, 95%, 96%, 97% 98%, 99% or 100% identical to SEQ ID NO: 199. In certain preferred embodiments, the exogenous polypeptide comprises a fragment of MOMP wherein said fragment comprises variable domain 4 (VD4). In an embodiment said fragment of MOMP that comprises VD4 comprises a sequence having at least 75%, 80%, 85%, 90%, 91%, 92%, 93%, 94%, 95%, 96%, 97% 98%, 99% or 100% identical to SEQ ID NO: 200. The fragments of MOMP comprising VD1, VD2, VD3 and VD4 as laid out in SEQ ID Nos 197-200 are based on MOMP-D (serovar) but the equivalent positions in the MOMP sequence from other serovars are equally envisioned.

In certain embodiments, the chimeric polypeptide comprises a first exogenous polypeptide and a second exogenous polypeptide, wherein the first and second exogenous polypeptides each independently comprise a fragment of MOMP wherein said fragment comprises VD1, VD2, VD3, or VD4. In certain such embodiments, the first and second exogenous polypeptides comprise fragments of MOMP wherein said fragments comprise the same MOMP variable domain (VD). In certain embodiments, the first and second exogenous polypeptides are the same. In certain other embodiments, the first exogenous and second polypeptides respectively comprise fragments of MOMP wherein said fragments comprise VD1 and VD2, VD1 and VD3, VD1 and VD4, VD2 and VD3, VD2 and VD4, or VD3 and VD4. In certain embodiments, the chimeric polypeptide comprises three, four, five or six exogenous polypeptides. In such embodiments, the exogenous polypeptides may comprise fragments of MOMP wherein said fragments comprise VD1, VD2, VD3 and VD4 in any combination.

In certain preferred embodiments, the exogenous polypeptide comprises a fragment of MOMP variable domain 1 (VD1). In certain embodiments, the exogenous polypeptide comprises a fragment of MOMP variable domain 2 (VD2). In certain preferred embodiments, the exogenous polypeptide comprises a fragment of MOMP variable domain 3 (VD3). In certain embodiments, the exogenous polypeptide comprises a fragment of MOMP variable domain 4 (VD4).

In certain embodiments, the chimeric polypeptide comprises a first exogenous polypeptide and a second exogenous polypeptide, wherein the first and second exogenous polypeptides each independently comprise a fragment of MOMP VD1, VD2, VD3, or VD4. In certain such embodiments, the first and second exogenous polypeptides comprise fragments of the same MOMP variable domain (VD). In certain embodiments, the first and second exogenous polypeptides are the same. In certain other embodiments, the first exogenous and second polypeptides respectively comprise fragments of MOMP VD1 and VD2, VD1 and VD3, VD1 and VD4, VD2 and VD3, VD2 and VD4, or VD3 and VD4.

In certain embodiments, the chimeric polypeptide comprises three, four, five or six exogenous polypeptides. In such embodiments, the exogenous polypeptides may comprise fragments of MOMP VD1, VD2, VD3 and D4 in any combination.

In certain embodiments, the porin is from a pathogenic Neisseria species (e.g., Neisseria gonorrhoeae, Neisseria meningitidis). In certain such embodiments, the porin is PorB. In certain embodiments, the allelic form of PorB is PorB1a from N. gonorrhoeae. In preferred embodiments, the allelic form of PorB is PorB1b from N. gonorrhoeae. In preferred embodiments, the strain of N. gonorrhoeae is FA1090 or F62. In certain embodiments, the exogenous polypeptide is an endogenous loop from PorB. In certain such embodiments, the loop is selected from loop 1, loop 2, loop 3, loop 4, loop 5, loop 6, loop 7, and loop 8, preferably loop 5.

In certain embodiments, the target protein is an opacity-associated protein (Opa). In certain such embodiments, the Opa is selected from: OpaA, OpaB, OpaC, OpaD, OpaE, OpaF, OpaI, and OpaK, preferably OpaB. In preferred embodiments, the Opa protein is from a pathogenic Neisseria species (e.g., Neisseria gonorrhoeae, Neisseria meningitidis). In preferred embodiments, the strain of N. gonorrhoeae is FA1090 or F62. In certain embodiments, the exogenous polypeptide is an endogenous loop from OpaB. In certain such embodiments, the loop is selected from loop 1, loop 2, loop 3, loop 4, preferably loop 2.

In certain embodiments, the exogenous polypeptide comprises a fragment (e.g., an antigenic fragment) of a target protein that is a toxin, preferably a pore-forming toxin. In certain embodiments, the pore-forming toxin is α-hemolysin, β-hemolysin or γ-hemolysin. In preferred embodiments, the pore-forming toxin is α-hemolysin (Hla) (also known as Staphylococcus aureus alpha toxin).

In certain embodiments, the exogenous polypeptide comprises a fragment (e.g., an antigenic fragment) of a target protein that is an oncoprotein, preferably a bacterial or viral oncoprotein. In certain such embodiments, the oncoprotein is from a DNA oncovirus. In certain other embodiments, the oncoprotein is from an RNA oncovirus. In preferred embodiments, the oncoprotein is from HPV. In certain such embodiments, the oncoprotein is E6 or E7, preferably E7.

In certain embodiments, the exogenous polypeptide comprises a fragment (e.g., an antigenic fragment) of a target protein that is a bacterial serine protease. In certain such embodiments, the bacterial serine protease is from a gram-negative bacterium. In preferred embodiments, the bacterial serine protease is an HtrA protease. In preferred embodiments, the HtrA protease is from a pathogenic Chlamydia species, preferably Chlamydia trachomatis.

In certain embodiments, the exogenous polypeptide comprises a fragment (e.g., an antigenic fragment) of a target protein, wherein the target protein binds a (blood) coagulation factor. In certain embodiments, the coagulation factor is a mammalian coagulation factor, preferably a human coagulation factor. In preferred embodiments the coagulation factor is fibrinogen (also known as Factor I).

In certain embodiments, the target protein is a fibrinogen-binding protein from a gram-negative bacterium. In preferred such embodiments, the fibrinogen-binding protein is from a Staphylococcus species. In more preferred such embodiments, the fibrinogen-binding protein is from Staphylococcus aureus. In certain embodiments, the fibrinogen-binding protein is extracellular fibrinogen-binding protein (Efb).

In certain embodiments, the target protein is a coagulase, preferably a coagulase from a gram-negative bacterium. In preferred such embodiments, the coagulase is from a Staphylococcus species. In more preferred such embodiments, the coagulase is from Staphylococcus aureus.

The chimeric polypeptides according to the first aspect of the invention may comprise any two of the above-mentioned exogenous polypeptides.

In certain embodiments, the chimeric polypeptide comprises a first exogenous polypeptide comprising a fragment (e.g., an antigenic fragment) of a target protein from a first pathogenic microorganism or virus and a second exogenous polypeptide comprising a fragment (e.g., an antigenic fragment) of a target protein from a second pathogenic microorganism or virus. In preferred such embodiments, the first and the second pathogenic microorganism or virus are each independently selected from Neisseria gonorrhoeae, Neisseria meningitidis, Haemophilus influenzae (e.g., Non-typeable Haemophilus influenzae), Staphylococcus aureus, human papillomavirus (HPV), Chlamydia trachomatis, Chlamydia muridarum, Streptococcus pneumonia, Escherichia coli (e.g., pathogenic E. coli), Vibrio cholerae and Streptococcus agalactiae. In certain embodiments, the first and second pathogenic microorganism or virus are the same. In certain other embodiments, the first pathogenic microorganism or virus are different.

In certain embodiments, the first and the second pathogenic microorganism or virus are Chlamydia trachomatis and Neisseria gonorrhoeae, respectively. In certain embodiments, the first and the second pathogenic microorganism or virus are Chlamydia trachomatis and Neisseria meningitidis, respectively. In certain embodiments, the first and the second pathogenic microorganism or virus are Chlamydia trachomatis and Staphylococcus aureus, respectively. In certain embodiments, the first and the second pathogenic microorganism or virus are Chlamydia trachomatis and HPV, respectively. In certain embodiments, the first and the second pathogenic microorganism or virus are Neisseria gonorrhoeae and Neisseria meningitidis, respectively. In certain embodiments, the first and the second pathogenic microorganism or virus are Neisseria gonorrhoeae and Staphylococcus aureus, respectively. In certain embodiments, the first and the second pathogenic microorganism or virus are Neisseria gonorrhoeae and HPV, respectively. In certain embodiments, the first and the second pathogenic microorganism or virus are Neisseria meningitidis and Staphylococcus aureus, respectively. In certain embodiments, the first and the second pathogenic microorganism or virus are Neisseria meningitidis and HPV, respectively. In certain embodiments, the first and the second pathogenic microorganism or virus are Staphylococcus aureus and HPV, respectively.

In certain embodiments, the target protein from Neisseria gonorrhoeae or Neisseria meningitidis is PorB. In certain embodiments, the target protein from a pathogenic Neisseria gonorrhoeae or Neisseria meningitidis is OpaB. In certain embodiments, the target protein from Chlamydia trachomatis is MOMP. In certain embodiments, the target protein from Chlamydia trachomatis is HtrA. In certain embodiments, the target protein from HPV is E7. In certain embodiments, the target protein from Staphylococcus aureus is α-hemolysin. In certain embodiments, the target protein from Staphylococcus aureus is coagulase. In certain embodiments, the target protein from Staphylococcus aureus is extracellular fibrinogen-binding protein (Efb).

In certain embodiments, the exogenous polypeptide comprises a fragment (e.g., an antigenic fragment) of a target protein that is a tumour antigen.

In certain embodiments, the exogenous polypeptide comprises a fragment of a target protein wherein the target protein is insoluble when not comprised in the chimeric polypeptide. It is to be understood that in certain embodiments the target protein is insoluble (when not comprised in the chimeric polypeptide) but may be rendered soluble using a solubilization agent (e.g., an amphiphile, such as a detergent, peptide surfactant, amphipol or styrene-maleic acid copolymer) or a chaotropic agent (e.g., urea or guanidine hydrochloride). However, it is an advantage of the chimeric polypeptide according to the invention that a soluble sample of the exogenous polypeptide from the target protein can be recombinantly obtained without the use of a solubilization agent and/or a chaotropic agent.

In certain embodiments, the target protein is insoluble when recombinantly produced in a host cell or a cell-free expression system (when not comprised in the chimeric polypeptide). In certain embodiments, the target protein is insoluble when recombinantly produced in a host cell or a cell-free expression system (when not comprised in the chimeric polypeptide) and in the absence of a solubilization agent or a chaotropic agent. In certain embodiments, the host cell is a bacterial cell, a yeast cell, a plant cell, an insect cell, or a mammalian cell. In preferred such embodiments, the bacterial cell is an E. coli cell. In certain embodiments, the target protein is obtainable from the inclusion bodies in an E. coli cell when recombinantly produced therein (and not comprised in the chimeric polypeptide).

By recombinantly producing the exogenous polypeptide as part of the chimeric polypeptide, the proportion of exogenous polypeptide that is present in the soluble fraction (versus the insoluble fraction) during recombinant production (e.g., in E. coli) can be increased without use of a solubilization agent and/or a chaotropic agent. The (relative) amount of soluble exogenous polypeptide present in the soluble fraction can be determined by routine means (e.g., immunodetection, mass spectrometry, UV-Vis spectrophotometry, fluorescence spectroscopy).

In certain embodiments, the exogenous polypeptide is N- and C-terminally flanked by enzyme-cleavable amino acid sequences (e.g., for excision of the exogenous polypeptide from the chimeric polypeptide following recombinant production). In certain embodiments, the enzyme-cleavable amino acid sequence is a TEV protease-cleavable amino acid sequence, optionally ENLYFQG (SEQ ID NO:176).

In certain embodiments, when recombinantly produced when not comprised in the chimeric polypeptide, the exogenous polypeptide, or the target protein from which a sequence of the exogenous polypeptide is derived, does not adopt its native state or native three-dimensional structure. In certain embodiments, the exogenous polypeptide or target protein is misfolded (i.e., adopts a non-native three-dimensional structure) when recombinantly produced when not comprised in the chimeric polypeptide. In certain other embodiments, the exogenous polypeptide or target protein is non-natively unfolded, misfolded or denatured when recombinantly produced when not comprised in the chimeric polypeptide. In certain such embodiments, the exogenous polypeptide or target protein may aggregate with itself and/or other proteins. It is to be understood that the exogenous polypeptide or target protein may adopt the correct fold when recombinantly produced when not comprised in the chimeric polypeptide through use of a solubilization agent (e.g., an amphiphile, such as a detergent, peptide surfactant, amphipol or styrene-maleic acid copolymer) or a chaotropic agent (e.g., urea or guanidine hydrochloride). However, it is an advantage of the chimeric polypeptide according to the invention that the exogenous polypeptide or target protein can be recombinantly produced in its native state without the use of a solubilization agent and/or a chaotropic agent. The term “native three-dimensional structure” is to include normal flexibility of movement of polypeptides and their normal transitions in conformational states. An exogenous polypeptide or target protein that does not adopt its native state or native three-dimensional structure when recombinantly produced when not comprised in the chimeric polypeptide can be determined using routine biochemical or biophysical techniques. Suitable assays may include for example, immunoassays (e.g., ELISA), SPR, size-exclusion chromatography, gel electrophoresis, dynamic light scattering, circular dichroism spectroscopy, FTIR spectroscopy, thermal shift assays (e.g., FSEC-TS, DSF). The native state or native three-dimensional structure can be determined experimentally (e.g., by x-ray crystallography, NMR spectroscopy, electron microscopy) or by known protein structure prediction tools (e.g., AlphaFold, Phyre, RosettaCM, I-TASSER, Jpred).

In certain embodiments, the exogenous polypeptide comprises or consists of an epitope (also referred to as an “antigenic determinant”). In such embodiments, correct display of a known epitope can be determined, for example, using an antibody binding assay. In certain embodiments, the exogenous polypeptide comprises an amino acid sequence corresponding to a linear epitope. In certain embodiments, the exogenous polypeptide comprises an amino acid sequence that, when expressed in the chimeric polypeptide, displays a conformational epitope.

In certain embodiments, the exogenous polypeptide comprises a cryptotope (also known as a “cryptic epitope”). Cryptotopes are antigenic sites or epitopes masked within three-dimensional structures (e.g., in a protein or surface subunits of a virion) when expressed in their native proteins. Some infectious pathogens are known to escape immunological targeting by B-cells by masking antigen-binding sites as cryptotopes. Cryptotopes may become immune targets occasionally upon protein unfolding. Protein denaturation, proteolytic processing or presentation through antigen presenting cells (APCs) may expose hidden regions of a three-dimensional antigen to B-cell recognition. Viral replication can involve structural plasticity of virions and temporal exposure of ostensibly cryptotopes.

In certain embodiments, the exogenous polypeptide is susceptible to degradation (e.g., proteolytic degradation) when recombinantly produced when not comprised in the chimeric polypeptide. By recombinantly producing the exogenous polypeptide as part of the chimeric polypeptide, the amount of proteolytically degraded exogenous polypeptide can be decreased. The (relative) amount of proteolytically degraded exogenous polypeptide present in the soluble fraction can be determined by routine means (e.g., electrophoretic methods, mass spectrometry, size-exclusion chromatography).

In certain embodiments, the exogenous polypeptide is 0.5-35 kDa. In certain embodiments, the exogenous polypeptide is 0.5-35 kDa, optionally 0.75-25 kDa, optionally 1-15 kDa, optionally 1-12.5 kDa. In certain embodiments, the exogenous polypeptide is 1-12.5 kDa.

In certain embodiments, the chimeric polypeptide is 12.5-95 kDa. In certain embodiments, the chimeric polypeptide is 12.5-95 kDa, optionally 13-70 kDa, optionally 14-50 kDa, optionally 15-30 kDa. In certain embodiments, the chimeric polypeptide is 15-30 kDa.

In certain embodiments, the chimeric polypeptide comprises one or more exogenous polypeptide(s) each independently at least 40%, at least 50%, at least 60%, at least 70%, at least 80%, at least 90%, at least 95%, at least 97%, at least 98%, or at least 99% identical to an amino acid sequence selected from: SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47. SEQ ID NO:48, SEQ ID NO:49, SEQ ID NO:50, SEQ ID NO:51, SEQ ID NO:52, SEQ ID NO:53, SEQ ID NO:182, SEQ ID NO:183, SEQ ID NO: 195, SEQ ID NO: 197, SEQ ID NO: 198, SEQ ID NO: 199 and SEQ ID NO: 200.

In certain embodiments, the chimeric polypeptide comprises one or more exogenous polypeptide(s) each independently comprising or consisting of an amino acid sequence selected from: SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:51, SEQ ID NO:52, SEQ ID NO:53, SEQ ID NO:182, SEQ ID NO:183, SEQ ID NO: 195, SEQ ID NO: 197, SEQ ID NO: 198, SEQ ID NO: 199 and SEQ ID NO: 200.

In certain embodiments, the chimeric polypeptide comprises or consists of an amino acid sequence at least 40%, at least 50%, at least 60%, at least 70%, at least 80%, at least 90%, at least 95%, at least 97%, at least 98%, or at least 99% identical to an amino acid sequence selected from: SEQ ID NO:54, SEQ ID NO:55, SEQ ID NO:56, SEQ ID NO:57, SEQ ID NO:58, SEQ ID NO:59, SEQ ID NO:60, SEQ ID NO:61, SEQ ID NO:62, SEQ ID NO:63, SEQ ID NO:64, SEQ ID NO:65, SEQ ID NO:66, SEQ ID NO:67, SEQ ID NO:68, SEQ ID NO:69, SEQ ID NO:70, SEQ ID NO:71, SEQ ID NO:72, SEQ ID NO:73, SEQ ID NO:74, SEQ ID NO:75, SEQ ID NO:76, SEQ ID NO:77, SEQ ID NO:78, SEQ ID NO:79, SEQ ID NO:80, SEQ ID NO:81, SEQ ID NO:82, SEQ ID NO:83, SEQ ID NO:84, SEQ ID NO:85, SEQ ID NO:86, SEQ ID NO:87, SEQ ID NO:88, SEQ ID NO:89, SEQ ID NO:90, SEQ ID NO:91, SEQ ID NO:92, SEQ ID NO:93, SEQ ID NO:94, SEQ ID NO:95, SEQ ID NO:96, SEQ ID NO:97, SEQ ID NO:184, SEQ ID NO:185, and SEQ ID NO:186.

In certain embodiments, the chimeric polypeptide comprises or consists of an amino acid sequence selected from: SEQ ID NO:54, SEQ ID NO:55, SEQ ID NO:56, SEQ ID NO:57, SEQ ID NO:58, SEQ ID NO:59, SEQ ID NO:60, SEQ ID NO:61, SEQ ID NO:62, SEQ ID NO:63, SEQ ID NO:64, SEQ ID NO:65, SEQ ID NO:66, SEQ ID NO:67, SEQ ID NO:68, SEQ ID NO:69, SEQ ID NO:70, SEQ ID NO:71, SEQ ID NO:72, SEQ ID NO:73, SEQ ID NO:74, SEQ ID NO:75, SEQ ID NO:76, SEQ ID NO:77, SEQ ID NO:78, SEQ ID NO:79, SEQ ID NO:80, SEQ ID NO:81, SEQ ID NO:82, SEQ ID NO:83, SEQ ID NO:84, SEQ ID NO:85, SEQ ID NO:86, SEQ ID NO:87, SEQ ID NO:88, SEQ ID NO:89, SEQ ID NO:90, SEQ ID NO:91, SEQ ID NO:92, SEQ ID NO:93, SEQ ID NO:94, SEQ ID NO:95, SEQ ID NO:96, SEQ ID NO:97, SEQ ID NO:184, SEQ ID NO:185, and SEQ ID NO:186, SEQ ID NO: 188, SEQ ID NO: 189, SEQ ID NO: 190, SEQ ID NO: 191, SEQ ID NO: 192, SEQ ID NO: 193, SEQ ID NO: 194 or SEQ ID NO: 196.

TABLE 2
Exemplary Exogenous Polypeptides

| SEQ ID | Construct | Amino acid sequence: |
|---|---|---|
| NO: | (Target Protein) | |
| 17 | PorBLoop1 | SVEHTDGKVSKVE |
| | (PorB) | |
| 18 | PorBLoop2 | GASVAGTNTGWGNKQ |
| | (PorB) | |
| 19 | PorBloop3 | SPLKNTGANVNAWESGKFTGNVLEISGMAQREHR |
| | (PorB) | |
| 20 | PorBloop4: | PKDNSGSNGES |
| | (PorB) | |
| 21 | PorBloop5 | QRYGEGTKKIEYDGQTYSIPSLFV |
| | (PorB) | |
| 22 | PorBloop6 | AKLYGAMSGNSHNSQTE |
| | (PorB) | |
| 23 | PorBloop7 | GFKGTVDSANHDNTYDQ |
| | (PorB) | |
| 24 | PorBloop8 | QEGKGADKIVSTAS |
| | (PorB) | |
| 25 | Loop1OpaA/K | YARYRKAAERITHDYPEPTGAKKGKISTVSDYFRNIRTHSIHAVSS |
| | (OpaA/K) | L |
| 26 | Loop2OpaA | YARYRKWNNSKYSVNTKKVNENKGEKINVTQYLKAENQENGTFH |
| | (OpaA) | AVSSL |
| 27 | Loop3OpaA | YLQSGKPSPIVRGSTL |
| | (OpaA) | |
| 28 | Loop1OpaB/D | YARYRKAAERITHDYPEPTAPGKNKISTVSDYFRNIRTHSIHPRHA |
| | (OpaB/D) | VSSL |
| 29 | Loop3OpaB/D | AYPSDADAAVTV |
| | (OpaB/D) | |
| 30 | Loop2OpaB | YARYRKWNDNKYSVDIKELENKNQNKRDLKTENQENGSFHAVSS |
| | (OpaB) | L |
| 31 | Loop4OpaB | HNWGRLENTRFKTHE |
| | (OpaB) | |
| 32 | Loop4Univ. | HYWGRLENTRFKTHE |
| | (Opa) | |
| 33 | Loop1OpaC | YARYRKAYAYEHITRDYPDAAGANQGKKISTVSDYFKNIRTHSIHA |
| | (OpaC) | VSSL |
| 34 | Loop3OpaC | TTEFLTAAGQDGGA |
| | (OpaC) | |
| 35 | loop2OpaD | YARYRKWHNNKYSVNIKELERKNNKTFGGNQLNIKYQKTEHQEN |
| | (OpaD) | GTFHAVSSL |
| 36 | Loop1OpaE | YARYRKAYEHITRDYPDAAGANQGKKISTVSDYFKNIRTRSVHHA |
| | (OpaE) | VSSL |
| 37 | Loop2OpaE | YARYRKWHNNKYSVNIKELGRNDNSASDSKHLNIKTQKTEHQEN |
| | (OpaE) | GTFHAVSSL |
| 38 | Loop3OpaE/K | YPSDGSAKTSVPSEM |
| | (OpaE/K) | |
| 39 | Loop1OpaF | YARYRKAAERITHDYPEPTGAKKDKKISTVSDYFRNIRTHSVHHAV |
| | (OpaF) | SSL |
| 40 | Loop2OpaF | YARYRKWNNSKYSVNIKRVKENNGSGKKLTQDLKTENQENGTFH |
| | (OpaF) | AVSSL |
| 41 | Loop3OpaF | VITAPPTTSDGA |
| | (OpaF) | |
| 42 | Loop1Opal | YARYRKAYEHITRDYPDAAGANKGKISTVSDYFRNIRTHSIHHAVS |
| | (Opal) | SL |
| 43 | Loop3Opal | HSAGTKPTYYDDIDSGKTK |
| | (Opal) | |
| 44 | Loop2Opak | YARYRKWNDNKYSVNIKELGRKDGTSSSGRYLNIQTRKTENQEN |
| | (OpaK) | GTFHAVSSL |
| 45 | DNABP-pep | RPGRNPKTGDVVPVSARRVVGPSLFSLHHRQPRLGRNPKTGDS |
| | (Integration Host | V |
| | Factor) | |
| 46 | HLA220-270 | LFMKTRNGSMKAADNFLDPNKASSLLSSGFSPDFATVITMDRKAS |
| | (a-hemolysin) | KQQTN |
| 47 | HLA(177-200) | NWGPYDRDSWNPVYGNQLFMKTR |
| | (a-hemolysin) | |
| 48 | HLA177-200+ | NWGPYDRDSWNPVYGNQLFMKTRGSTNTKDK |
| | (a-hemolysin) | |
| 49 | E7 | GQAEPDRAHYNIVTFCCKCD |
| 50 | HtrA | TGSQAIASPGNKRGFQENPFDYFNDEFFNRFFGLP |
| 51 | HtrA+ | NKRGFQENPFDYFNDEFFNR |
| 52 | Fibrinogen-binding | KYIKFKHDYNILEFNDGTFEYGARPQFN |
| | protein | |
| 53 | CoA | ETNAYNVTTHANGQVSYGARPTYKKPS |
| | (coagulase) | |
| 182 | 8-D3Pil3 (69-84) | PPSDIKGKYVKEVEVKGSG |
| | (Pilin) | |
| +0 183 | 9-D3Pil2(135-151 | DAKDGKEIDTKHLPSTC |
| | C-term) | |
| | (Pilin) | |
| 195 | GFP | VSKGEELFTGVVPILVELDGDVNGHKFSVSGEGEGDATYGKLTLK |
| | | FICTTGKLPVPWPTLVTTLTYGVQCFSRYPDHMKQHDFFKSAMP |
| | | EGYVQERTIFFKDDGNYKTRAEVKFEGDTLVNRIELKGIDFKEDG |
| | | NILGHKLEYNYNSHNVYIMADKQKNGIKVNFKIRHNIEDGSVQLAD |
| | | HYQQNTPIGDGPVLLPDNHYLSTQSALSKDPNEKRDHMVLLEFVT |
| | | AAGITLGMDELYKG |
| 197 | MOMP loop 1 | DRVLKTDVNKEFQMGAKPTTDTGNSAAPSTLTARENPAYGRHM |
| | comprising VD1 | |
| 198 | MOMP loop 2 | NSASFNLVGLFGDNENQKTVKAESVPNMSFDQSVVELYTDT |
| | comprising VD2 | |
| 199 | MOMP loop 3 | KPKVEELNVLCNAAEFTINKPKGYVGKEFPLDLTAGTDAATGTKD |
| | comprising VD3 | ASI |
| 200 | MOMP loop 4 | DADTIRIAQPKSATAIFDTTTLNPTIAGAGDVKTGAEGQLGDTMQI |
| | comprising VD4 | VSLQLNKMKSRKS |

TABLE 3
Exemplary Chimeric Polypeptides
With regard to the sequences shown in table 3 below, optionally the His tag can be cleaved.
The His tag can be cleaved by methods known to the person skilled in the art for example by
cleavage at the TEV cleavage site as denoted by SEQ ID NO: 176. As such, in an embodiment
there is provided SEQ ID NO: 54, SEQ ID NO: 55, SEQ ID NO: 56, SEQ ID NO: 57, SEQ ID NO: 58,
SEQ ID NO: 59, SEQ ID NO: 60, SEQ ID NO: 61, SEQ ID NO: 62, SEQ ID NO: 63, SEQ ID NO: 64,
SEQ ID NO: 65, SEQ ID NO: 66, SEQ ID NO: 67, SEQ ID NO: 68, SEQ ID NO: 69, SEQ ID NO: 70,
SEQ ID NO: 71, SEQ ID NO: 72, SEQ ID NO: 73, SEQ ID NO: 74, SEQ ID NO: 75, SEQ ID NO: 76,
SEQ ID NO: 77, SEQ ID NO: 78, SEQ ID NO: 79, SEQ ID NO: 80, SEQ ID NO: 81, SEQ ID NO: 82,
SEQ ID NO: 83, SEQ ID NO: 84, SEQ ID NO: 85, SEQ ID NO: 86, SEQ ID NO: 87, SEQ ID NO: 88,
SEQ ID NO: 89, SEQ ID NO: 90, SEQ ID NO: 91, SEQ ID NO: 92, SEQ ID NO: 93, SEQ ID NO: 94,
SEQ ID NO: 95, SEQ ID NO: 96, SEQ ID NO: 97, SEQ ID NO: 184, SEQ ID NO: 185, SEQ ID NO: 186,
SEQ ID NO: 188, SEQ ID NO: 189, SEQ ID NO: 190, SEQ ID NO: 191, SEQ ID NO: 192, SEQ ID NO: 193,
SEQ ID NO: 194 or SEQ ID NO: 196 wherein the His tag has been cleaved, optionally wherein the
His tag has been cleaved at the TEV cleavage site denoted by SEQ ID NO: 176. As a result of
said cleavage the construct encoding the D3 chimera commences from the amino acid residue
immediately following the TEV cleavage site.

| SEQ ID | | |
|---|---|---|
| NO: | Construct | Amino acid sequence: |
| 54 | D3_PorBLoop1 | MGSSHHHHHHENLYFQGASGNNPTIENEPKEGIPVDKKITVNKTWAVD |
| | | GNEVNKADETVDAVFTLQVKSVEHTDGKVSKVEKWVNVDSAKATAATS |
| | | FKHTFENLDNAKTYRVIERVSGYAPEYVSFVNGVVTIKNNKDSNEPTP |
| | | I |
| 55 | D3_PorBLoop2 | MGSSHHHHHHENLYFQGASGNNPTIENEPKEGIPVDKKITVNKTWAVD |
| | | GNEVNKADETVDAVFTLQVKGASVAGTNTGWGNKQKWVNVDSAKAT |
| | | AATSFKHTFENLDNAKTYRVIERVSGYAPEYVSFVNGVVTIKNNKDSNE |
| | | PTPI |
| 56 | D3_PorBloop3 | MGSSHHHHHHENLYFQGASGNNPTIENEPKEGIPVDKKITVNKTWAVD |
| | | GNEVNKADETVDAVFTLQVKSPLKNTGANVNAWESGKFTGNVLEISG |
| | | MAQREHRKWVNVDSAKATAATSFKHTFENLDNAKTYRVIERVSGYAP |
| | | EYVSFVNGVVTIKNNKDSNEPTPI |
| 57 | D3_PorBloop4 | MGSSHHHHHHENLYFQGASGNNPTIENEPKEGIPVDKKITVNKTWAVD |
| | | GNEVNKADETVDAVFTLQVKPKDNSGSNGESKWVNVDSAKATAATSF |
| | | KHTFENLDNAKTYRVIERVSGYAPEYVSFVNGVVTIKNNKDSNEPTPI |
| 58 | D3_PorBloop5 | MGSSHHHHHHENLYFQGASGNNPTIENEPKEGIPVDKKITVNKTWAVD |
| | | GNEVNKADETVDAVFTLQVKQRYGEGTKKIEYDGQTYSIPSLFVKWVN |
| | | VDSAKATAATSFKHTFENLDNAKTYRVIERVSGYAPEYVSFVNGVVTIK |
| | | NNKDSNEPTPI |
| 59 | D3_PorBloop6 | MGSSHHHHHHENLYFQGASGNNPTIENEPKEGIPVDKKITVNKTWAVD |
| | | GNEVNKADETVDAVFTLQVKDAKLYGAMSGNSHNSQTEKWVNVDSA |
| | | KATAATSFKHTFENLDNAKTYRVIERVSGYAPEYVSFVNGVVTIKNNKD |
| | | SNEPTPI |
| 60 | D3_PorBloop7 | MGSSHHHHHHENLYFQGASGNNPTIENEPKEGIPVDKKITVNKTWAVD |
| | | GNEVNKADETVDAVFTLQVKGFKGTVDSANHDNTYDQKWVNVDSAK |
| | | ATAATSFKHTFENLDNAKTYRVIERVSGYAPEYVSFVNGVVTIKNNKDS |
| | | NEPTPI |
| 61 | D3_PorBloop8 | MGSSHHHHHHENLYFQGASGNNPTIENEPKEGIPVDKKITVNKTWAVD |
| | | GNEVNKADETVDAVFTLQVKQEGKGADKIVSTASKWVNVDSAKATAA |
| | | TSFKHTFENLDNAKTYRVIERVSGYAPEYVSFVNGVVTIKNNKDSNEPT |
| | | PI |
| 62 | D3-Loop1 | MGSSHHHHHHENLYFQGASGNNPTIENEPKEGIPVDKKITVNKTWAVD |
| | PorB Loop3 | SPLKNTGANVNAWESGKFTGNVLEISGMAQREHRDAVFTLQVKDGDK |
| | | WVNVDSAKATAATSFKHTFENLDNAKTYRVIERVSGYAPEYVSFVNGV |
| | | VTIKNNKDSNEPTPI |
| | D3-Loop2 | See SEQ ID NO: 56 |
| | PorB Loop3 | |
| 63 | D3-Loop3 | MGSSHHHHHHENLYFQGASGNNPTIENEPKEGIPVDKKITVNKTWAVD |
| | PorB Loop3 | GNEVNKADETVDAVFTLQVKDGDKWVNVDSAKSPLKNTGANVNAWE |
| | | SGKFTGNVLEISGMAQREHRKHTFENLDNAKTYRVIERVSGYAPEYVS |
| | | FVNGVVTIKNNKDSNEPTPI |
| 64 | D3-Loop4 | MGSSHHHHHHENLYFQGASGNNPTIENEPKEGIPVDKKITVNKTWAVD |
| | PorB Loop3 | GNEVNKADETVDAVFTLQVKDGDKWVNVDSAKATAATSFKHTFSPLK |
| | | NTGANVNAWESGKFTGNVLEISGMAQREHRRVIERVSGYAPEYVSFV |
| | | NGVVTIKNNKDSNEPTPI |
| 65 | D3-Loop5 | MGSSHHHHHHENLYFQGASGNNPTIENEPKEGIPVDKKITVNKTWAVD |
| | PorB Loop3 | GNEVNKADETVDAVFTLQVKDGDKWVNVDSAKATAATSFKHTFENLD |
| | | NAKTYRVIESPLKNTGANVNAWESGKFTGNVLEISGMAQREHRPEYVS |
| | | FVNGVVTIKNNKDSNEPTPI |
| 66 | D3-Loop6 | MGSSHHHHHHENLYFQGASGNNPTIENEPKEGIPVDKKITVNKTWAVD |
| | PorB Loop 3 | GNEVNKADETVDAVFTLQVKDGDKWVNVDSAKATAATSFKHTFENLD |
| | | NAKTYRVIERVSGYAPEYVSFVSPLKNTGANVNAWESGKFTGNVLEIS |
| | | GMAQREHRVVTIKNNKDSNEPTPI |
| 67 | D3_Loop1OpaA | MGSSHHHHHHENLYFQGASGNNPTIENEPKEGIPVDKKITVNKTWAVD |
| | /K | GNEVNKADETVDAVFTLQVKYARYRKAAERITHDYPEPTGAKKGKIST |
| | | VSDYFRNIRTHSIHAVSSLKWVNVDSAKATAATSFKHTFENLDNAKTYR |
| | | VIERVSGYAPEYVSFVNGVVTIKNNKDSNEPTPI |
| 68 | D3_Loop2OpaA | MGSSHHHHHHENLYFQASGNNPTIENEPKEGIPVDKKITVNKTWAVDG |
| | | NEVNKADETVDAVFTLQVKYARYRKWNNSKYSVNTKKVNENKGEKIN |
| | | VTQYLKAENQENGTFHAVSSLKWVNVDSAKATAATSFKHTFENLDNAK |
| | | TYRVIERVSGYAPEYVSFVNGVVTIKNNKDSNEPTPI |
| 69 | D3Loop3OpaA | MGSSHHHHHHENLYFQGASGNNPTIENEPKEGIPVDKKITVNKTWAVD |
| | | GNEVNKADETVDAVFTLQVKYLQSGKPSPIVRGSTLKWVNVDSAKATA |
| | | ATSFKHTFENLDNAKTYRVIERVSGYAPEYVSFVNGVVTIKNNKDSNEP |
| | | TPI |
| 70 | D3Loop1OpaB/ | MGSSHHHHHHENLYFQASGNNPTIENEPKEGIPVDKKITVNKTWAVDG |
| | D | NEVNKADETVDAVFTLQVKYARYRKAAERITHDYPEPTAPGKNKISTVS |
| | | DYFRNIRTHSIHPRHAVSSLKWVNVDSAKATAATSFKHTFENLDNAKTY |
| | | RVIERVSGYAPEYVSFVNGVVTIKNNKDSNEPTPI |
| 71 | D3Loop3OpaB/ | MGSSHHHHHHENLYFQGLYFQGLYFQGASGNNPTIENEPKEGIPVDK |
| | D | KITVNKTWAVDGNEVNKADETVDAVFTLQVKAYPSDADAAVTVKWVN |
| | | VDSAKATAATSFKHTFENLDNAKTYRVIERVSGYAPEYVSFVNGVVTIK |
| | | NNKDSNEPTPI |
| 72 | D3Loop2OpaB | MGSSHHHHHHENLYFQGLYFQGLYFQGASGNNPTIENEPKEGIPVDK |
| | | KITVNKTWAVDGNEVNKADETVDAVFTLQVKYARYRKWNDNKYSVDIK |
| | | ELENKNQNKRDLKTENQENGSFHAVSSLKWVNVDSAKATAATSFKHT |
| | | FENLDNAKTYRVIERVSGYAPEYVSFVNGVVTIKNNKDSNEPTPI |
| 73 | D3Loop4OpaB | MGSSHHHHHHENLYFQGLYFQGLYFQGASGNNPTIENEPKEGIPVDK |
| | | KITVNKTWAVDGNEVNKADETVDAVFTLQVKHNWGRLENTRFKTHEK |
| | | WVNVDSAKATAATSFKHTFENLDNAKTYRVIERVSGYAPEYVSFVNGV |
| | | VTIKNNKDSNEPTPI |
| 74 | D3Loop4Univ. | MGSSHHHHHHENLYFQGLYFQGLYFQGASGNNPTIENEPKEGIPVDK |
| | | KITVNKTWAVDGNEVNKADETVDAVFTLQVKHYWGRLENTRFKTHEK |
| | | WVNVDSAKATAATSFKHTFENLDNAKTYRVIERVSGYAPEYVSFVNGV |
| | | VTIKNNKDSNEPTPI |
| 75 | D3Loop1OpaC | MGSSHHHHHHENLYFQGASGNNPTIENEPKEGIPVDKKITVNKTWAVD |
| | | GNEVNKADETVDAVFTLQVKYARYRKAYAYEHITRDYPDAAGANQGK |
| | | KISTVSDYFKNIRTHSIHAVSSLKWVNVDSAKATAATSFKHTFENLDNA |
| | | KTYRVIERVSGYAPEYVSFVNGVVTIKNNKDSNEPTPI |
| 76 | D3Loop2OpaC | MGSSHHHHHHENLYFQASGNNPTIENEPKEGIPVDKKITVNKTWAVDG |
| | | NEVNKADETVDAVFTLQVKYARYRKWNDNKYSVDIKELENKNQNKRD |
| | | LKTENQENGSFHAVSSLKWVNVDSAKATAATSFKHTFENLDNAKTYRV |
| | | IERVSGYAPEYVSFVNGVVTIKNNKDSNEPTPI |
| 77 | D3Loop3OpaC | MGSSHHHHHHENLYFQGASGNNPTIENEPKEGIPVDKKITVNKTWAVD |
| | | GNEVNKADETVDAVFTLQVKTTEFLTAAGQDGGAKWVNVDSAKATAA |
| | | TSFKHTFENLDNAKTYRVIERVSGYAPEYVSFVNGVVTIKNNKDSNEPT |
| | | PI |
| 78 | D3loop2OpaD | MGSSHHHHHHENLYFQASGNNPTIENEPKEGIPVDKKITVNKTWAVDG |
| | | NEVNKADETVDAVFTLQVKYARYRKWHNNKYSVNIKELERKNNKTFG |
| | | GNQLNIKYQKTEHQENGTFHAVSSLKWVNVDSAKATAATSFKHTFENL |
| | | DNAKTYRVIERVSGYAPEYVSFVNGVVTIKNNKDSNEPTPI |
| 79 | D3Loop1OpaE | MGSSHHHHHHENLYFQGASGNNPTIENEPKEGIPVDKKITVNKTWAVD |
| | | GNEVNKADETVDAVFTLQVKYARYRKAYEHITRDYPDAAGANQGKKIS |
| | | TVSDYFKNIRTRSVHHAVSSLKWVNVDSAKATAATSFKHTFENLDNAK |
| | | TYRVIERVSGYAPEYVSFVNGVVTIKNNKDSNEPTPI |
| 80 | D3Loop2OpaE | MGSSHHHHHHENLYFQASGNNPTIENEPKEGIPVDKKITVNKTWAVDG |
| | | NEVNKADETVDAVFTLQVKYARYRKWHNNKYSVNIKELGRNDNSASD |
| | | SKHLNIKTQKTEHQENGTFHAVSSLKWVNVDSAKATAATSFKHTFENL |
| | | DNAKTYRVIERVSGYAPEYVSFVNGVVTIKNNKDSNEPTPI |
| 81 | D3Loop3OpaE/ | MGSSHHHHHHENLYFQGASGNNPTIENEPKEGIPVDKKITVNKTWAVD |
| | K | GNEVNKADETVDAVFTLQVKYPSDGSAKTSVPSEMKWVNVDSAKATA |
| | | ATSFKHTFENLDNAKTYRVIERVSGYAPEYVSFVNGVVTIKNNKDSNEP |
| | | TPI |
| 82 | D3Loop1OpaF | MGSSHHHHHHENLYFQGASGNNPTIENEPKEGIPVDKKITVNKTWAVD |
| | | GNEVNKADETVDAVFTLQVKYARYRKAAERITHDYPEPTGAKKDKKIST |
| | | VSDYFRNIRTHSVHHAVSSLKWVNVDSAKATAATSFKHTFENLDNAKT |
| | | YRVIERVSGYAPEYVSFVNGVVTIKNNKDSNEPTPI |
| 83 | D3Loop2OpaF | MGSSHHHHHHENLYFQASGNNPTIENEPKEGIPVDKKITVNKTWAVDG |
| | | NEVNKADETVDAVFTLQVKYARYRKWNNSKYSVNIKRVKENNGSGKK |
| | | LTQDLKTENQENGTFHAVSSLKWVNVDSAKATAATSFKHTFENLDNAK |
| | | TYRVIERVSGYAPEYVSFVNGVVTIKNNKDSNEPTPI |
| 84 | D3Loop3OpaF | MGSSHHHHHHENLYFQGASGNNPTIENEPKEGIPVDKKITVNKTWAVD |
| | | GNEVNKADETVDAVFTLQVKVITAPPTTSDGAKWVNVDSAKATAATSF |
| | | KHTFENLDNAKTYRVIERVSGYAPEYVSFVNGVVTIKNNKDSNEPTPI |
| 85 | D3Loop1Opal | MGSSHHHHHHENLYFQGASGNNPTIENEPKEGIPVDKKITVNKTWAVD |
| | | GNEVNKADETVDAVFTLQVKYARYRKAYEHITRDYPDAAGANKGKIST |
| | | VSDYFRNIRTHSIHHAVSSLKWVNVDSAKATAATSFKHTFENLDNAKTY |
| | | RVIERVSGYAPEYVSFVNGVVTIKNNKDSNEPTPI |
| 86 | D3Loop2Opal | MGSSHHHHHHENLYFQASGNNPTIENEPKEGIPVDKKITVNKTWAVDG |
| | | NEVNKADETVDAVFTLQVKYARYRKWHNNKYSVNIKELERKNNKTFG |
| | | GNQLNIKYQKTEHQENGTFHAVSSLKWVNVDSAKATAATSFKHTFENL |
| | | DNAKTYRVIERVSGYAPEYVSFVNGVVTIKNNKDSNEPTPI |
| 87 | D3Loop3Opal | MGSSHHHHHHENLYFQGASGNNPTIENEPKEGIPVDKKITVNKTWAVD |
| | | GNEVNKADETVDAVFTLQVKHSAGTKPTYYDDIDSGKTKKWVNVDSA |
| | | KATAATSFKHTFENLDNAKTYRVIERVSGYAPEYVSFVNGVVTIKNNKD |
| | | SNEPTPI |
| 88 | D3Loop2OpaK | MGSSHHHHHHENLYFQASGNNPTIENEPKEGIPVDKKITVNKTWAVDG |
| | | NEVNKADETVDAVFTLQVKYARYRKWNDNKYSVNIKELGRKDGTSSS |
| | | GRYLNIQTRKTENQENGTFHAVSSLKWVNVDSAKATAATSFKHTFENL |
| | | DNAKTYRVIERVSGYAPEYVSFVNGVVTIKNNKDSNEPTPI |
| 89 | D3DNABP-pep | MGSSHHHHHHENLYFQGASGNNPTIENEPKEGIPVDKKITVNKTWAVD |
| | | GNEVNKADETVDAVFTLQVKRPGRNPKTGDVVPVSARRVVGPSLFSL |
| | | HHRQPRLGRNPKTGDSVKWVNVDSAKATAATSFKHTFENLDNAKTYR |
| | | VIERVSGYAPEYVSFVNGVVTIKNNKDSNEPTPI-- |
| 90 | D3HLA220-270 | MGSSHHHHHHENLYFQGASGNNPTIENEPKEGIPVDKKITVNKTWAVD |
| | | GNEVNKADETVDAVFTLQVKLFMKTRNGSMKAADNFLDPNKASSLLSS |
| | | GFSPDFATVITMDRKASKQQTNKWVNVDSAKATAATSFKHTFENLDNA |
| | | KTYRVIERVSGYAPEYVSFVNGVVTIKNNKDSNEPTPI |
| 91 | D3HLA(177- | MGSSHHHHHHENLYFQGASGNNPTIENEPKEGIPVDKKITVNKTWAVD |
| | 200) | GNEVNKADETVDAVFTLQVKNWGPYDRDSWNPVYGNQLFMKTRKWV |
| | | NVDSAKATAATSFKHTFENLDNAKTYRVIERVSGYAPEYVSFVNGVVTI |
| | | KNNKDSNEPTPI-- |
| 92 | D3HLA177- | MGSSHHHHHHENLYFQGASGNNPTIENEPKEGIPVDKKITVNKTWAVD |
| | 200+ | GNEVNKADETVDAVFTLQVKNWGPYDRDSWNPVYGNQLFMKTRGST |
| | | NTKDKKWVNVDSAKATAATSFKHTFENLDNAKTYRVIERVSGYAPEYV |
| | | SFVNGVVTIKNNKDSNEPTPI-- |
| 93 | D3E7 | MGSSHHHHHHENLYFQGASGNNPTIENEPKEGIPVDKKITVNKTWAVD |
| | | GNEVNKADETVDAVFTLQVKGQAEPDRAHYNIVTFCCKCDKWVNVDS |
| | | AKATAATSFKHTFENLDNAKTYRVIERVSGYAPEYVSFVNGVVTIKNNK |
| | | DSNEPTPI-- |
| 94 | D3HtrA | MGSSHHHHHHENLYFQGASGNNPTIENEPKEGIPVDKKITVNKTWAVD |
| | | GNEVNKADETVDAVFTLQVKTGSQAIASPGNKRGFQENPFDYFNDEFF |
| | | NRFFGLPKWVNVDSAKATAATSFKHTFENLDNAKTYRVIERVSGYAPE |
| | | YVSFVNGVVTIKNNKDSNEPTPI-- |
| 95 | D3HtrA+ | MGSSHHHHHHENLYFQGASGNNPTIENEPKEGIPVDKKITVNKTWAVD |
| | | GNEVNKADETVDAVFTLQVKNKRGFQENPFDYFNDEFFNRKWVNVDS |
| | | AKATAATSFKHTFENLDNAKTYRVIERVSGYAPEYVSFVNGVVTIKNNK |
| | | DSNEPTPI-- |
| 96 | D3-Fibrinogen- | MGSHHHHHHGGSASGNNPTIENEPKEGIPVDKKITVNKTWAVDGNEV |
| | binding | NKADETVDAVFTLQVKKYIKFKHDYNILEFNDGTFEYGARPQFNKWVN |
| | protein | VDSAKATAATSFKHTFENLDNAKTYRVIERVSGYAPEYVSFVNGVVTIK |
| | | NNKDSNEPTPI-- |
| 97 | D3-CoA | MGSHHHHHHGGSASGNNPTIENEPKEGIPVDKKITVNKTWAVDGNEV |
| | | NKADETVDAVFTLQVKETNAYNVTTHANGQVSYGARPTYKKPSKWVN |
| | | VDSAKATAATSFKHTFENLDNAKTYRVIERVSGYAPEYVSFVNGVVTIK |
| | | NNKDSNEPTPI-- |
| 184 | 8-D3Pil3 | MGSSHHHHHHENLYFQGLYFQGLYFQGASGNNPTIENEPKEGIPVDK |
| | (69-84) | KITVNKTWAVDGNEVNKADETVDAVFTLQVKDGDKWVNVDSAKATAA |
| | | TSFKHTFENLDNAKTYRVIERVSGYAPEYVSFVNGVVTIKNNKDSNEPT |
| | | PIGSGGGGPPSDIKGKYVKEVEVKGSG |
| 185 | 9-D3Pil2 | MGSSHHHHHHENLYFQGLYFQGLYFQGASGNNPTIENEPKEGIPVDK |
| | (135- | KITVNKTWAVDGNEVNKADETVDAVFTLQVKDGDKWVNVDSAKATAA |
| | 151 C-term) | TSFKHTFENLDNAKTYRVIERVSGYAPEYVSFVNGVVTIKNNKDSNEPT |
| | | PIGSGGGGDAKDGKEIDTKHLPSTC |
| 186 | 15-D3Pil1 | MGSSHHHHHHENLYFQGLYFQGLYFQGASGNNPTIENEPKEGIPVDK |
| | (135-151) | KITVNKTWAVDGNEVNKADETVDAVFTLQVKDAKDGKEIDTKHLPSTC |
| | | KWVNVDSAKATAATSFKHTFENLDNAKTYRVIERVSGYAPEYVSFVNG |
| | | VVTIKNNKDSNEPTPI |
| 187 | ml3- | MQRYGEGTKKIEYDGQTYSIPSLFVGGSGGSGGSGGSMKMEELFKKHK |
| | PorBloop5: | IVAVLRANSVEEAKKKALAVFLGGVHLIEITFTVPDADTVIKELSFLKEM |
| | | GAIIGAGTVTSVEQARKAVESGAEFIVSPHLDEEISQFAKEKGVFYMPG |
| | | VMTPTELVKAMKLGHTILKLFPGEVVGPQFVKAMKGPFPNVKFVPTGG |
| | | VNLDNVCEWFKAGVLAVGVGSALVKGTPVEVAEKAKAFVEKIRGCTEH |
| | | HHHHH |
| 188 | D3L1VD1 | MGSSHHHHHHENLYFQGASGNNPTIENEPKEGIPVDKKITVNKTWAVD |
| | D3L2Loop5: | PRAVNTTPDNQLGAVDSTTPAADAVFTLQVKQRYGEGTKKIEYDGQTY |
| | | SIPSLFVKWVNVDSAKATAATSFKHTFENLDNAKTYRVIERVSGYAPEY |
| | | VSFVNGVVTIKNNKDSNEPTPI |
| 189 | D3L1VD3 | MGSSHHHHHHENLYFQGASGNNPTIENEPKEGIPVDKKITVNKTWAVD |
| | D3L2Loop5 | AAETGATINLPFEADAVFTLQVKQRYGEGTKKIEYDGQTYSIPSLFVKW |
| | | VNVDSAKATAATSFKHTFENLDNAKTYRVIERVSGYAPEYVSFVNGVV |
| | | TIKNNKDSNEPTPI |
| 190 | D3_H36B_PorB | MGSSHHHHHHENLYFQGNKPGKKVKEIPVTPSNGEITVSKTWDKGSD |
| | L5 | LENANVVYTLKQRYGEGTKKIEYDGQTYSIPSLFVAVASVSLTKTTPNG |
| | | EINLGNGIKFTVTGAFAGKFSGLTDSKTYMISERIAGYGNTITTGAGSAAI |
| | | TNTPDSDNPTPL |
| 191 | D3_CJB111_Po | MGSSHHHHHHENLYFQGNNPTEESEPQEGTPANQEIKVIKDWAVDGT |
| | rBL5 | ITDANVAVKAIFTLQEKQRYGEGTKKIEYDGQTYSIPSLFVTWVNVASH |
| | | EATKPSRFEHTFTGLDNAKTYRVVERVSGYTPEYVSFKNGVVTIKNNK |
| | | NSNDPTPI |
| 192 | D3_CJB110_Po | MGSSHHHHHHENLYFQGNKPGTDLSEQPVTPEDGEVKVTKTWAAGA |
| | rBL5 | NKADAKVVYTLKQRYGEGTKKIEYDGQTYSIPSLFVVASVALTAADTKG |
| | | TINLGKGMTFEITGAFSGTFKGLQNKAYTVSERVAGYTNAINVTGNAVA |
| | | ITNTPDSDNPTPL |
| 193 | D3_2603_PorB | MGSSHHHHHHENLYFQGNKPGKDLTELPVTPSKGEVTVAKTWSDGIA |
| | L5 | PDGVNVVYTLKQRYGEGTKKIEYDGQTYSIPSLFVKTVASVSLTKTSKG |
| | | TIDLGNGIKFEVSGNFSGKFTGLENKSYMISERVSGYGSAINLENGKVTI |
| | | TNTKDSDNPTPLP |
| 194 | D3_DK21_PorB | MGSSHHHHHHENLYFQGNNPTTENEPQTGNPVNKEITVRKTWAVDG |
| | L5 | NEVNKGDEKVDAVFTLQVKQRYGEGTKKIEYDGQTYSIPSLFVKWVNV |
| | | DSATATAATDFKYTFKNLDNAKTYRVVERVSGYAPAYVSFVGGVVTIK |
| | | NNKNSNDPTPI |
| 196 | D3Loop2_GFP | MGSSHHHHHHENLYFQGASGNNPTIENEPKEGIPVDKKITVNKTWAVD |
| | | GNEVNKADETVDAVFTLQVKVSKGEELFTGVVPILVELDGDVNGHKFS |
| | | VSGEGEGDATYGKLTLKFICTTGKLPVPWPTLVTTLTYGVQCFSRYPD |
| | | HMKQHDFFKSAMPEGYVQERTIFFKDDGNYKTRAEVKFEGDTLVNRIE |
| | | LKGIDFKEDGNILGHKLEYNYNSHNVYIMADKQKNGIKVNFKIRHNIEDG |
| | | SVQLADHYQQNTPIGDGPVLLPDNHYLSTQSALSKDPNEKRDHMVLLE |
| | | FVTAAGITLGMDELYKGKWVNVDSAKATAATSFKHTFENLDNAKTYRVI |
| | | ERVSGYAPEYVSFVNGVVTIKNNKDSNEPTPI |

### Nanoparticles

A nanoparticle (NP) can be defined as an ordered set of atoms or molecules with a diameter ranging from 1 to 100 nm. They can be easily produced with both top-down and bottom-up approaches using for example condensation/evaporation, pyrolytic methods, atomic layer deposition techniques. NPs include biopolymer-based NPs such as those formed by proteins (pNPs) like albumin, gliadin and ferritin. This class of cages present several advantages including biodegradability, stability, possibility to modify their surface, biocompatibility, ability to control of particle size and low toxicity and immunogenicity. The non-antigenic properties of pNPs makes them particularly useful for therapeutic applications including vaccines. The assembly of pNPs can be achieved using several different approaches including desolvation, coacervation, emulsification, nanoprecipitation and nano spray drying. In some cases, a sub-class of pNPs, known as self-assembling protein nanoparticles, are composed of proteins that spontaneously assemble into nanoparticles.

Self-assembling protein nanoparticles comprise a class of proteins able to spontaneously assemble into nanocages with precise shape and geometry. In vaccinology, they can be used both as carrier of adjuvant and as platform for antigen display. In fact, the highly symmetric and ordered structure allows the multicopy display of target antigen enabling an efficient activation of both B- and T-cell immune response. The nanoparticle surfaces can be decorated with functional protein antigens/epitopes. Methods of engineering NPs include chemical conjugation, protein ligation and genetic fusion. It is demonstrated herein that pNPs, particularly self-assembling pNPs, decorated with the chimeric polypeptides can be produced. Such NPs are particularly advantageous, for example, because of the increase in avidity and therefore potency of the immune response to the exogenous polypeptides displayed.

Therefore, in a second aspect, the present invention provides a nanoparticle comprising a chimeric polypeptide according to the first aspect.

### Chemical Conjugation for Chimeric Polypeptide Display

In certain embodiments, the chimeric polypeptide is conjugated to the nanoparticle chemically.

Chemical conjugation of the chimeric polypeptide and nanoparticle may involve specific chemical groups on the surface of the nanoparticle and the chimeric polypeptide (e.g., carboxyl, amine, hydroxyl or thiol group). For example, one amine-based conjugation method involves reacting a lysine present on one conjugation partner with an N-hydroxysuccinimide ester on the other conjugation partner, thereby forming an intermolecular amide bond linking the conjugation partners. The skilled person is aware of routine methods for performing such chemical conjugations.

Since amine group modification can be associated with possible protein structure disruption, a more site-directed conjugation can be performed by modifying thiols. The skilled person can readily assess whether a conjugation method employing amine group modification (e.g., as described above) disrupts protein structure and is therefore unsuitable for conjugating the chimeric polypeptide and nanoparticle. Thiols are not typically exposed on the nanoparticle surface and are often involved in the formation of disulphide bonds. If not natively present or available for reaction, thiol groups can be artificially created using a thiolation reagent that inserts new thiol groups into the protein or by disrupting native disulphide bonds with a reducing agent.

Alternatively or additionally, the carboxyl groups located at the C-terminus of each protein and in the side chains of aspartic and glutamic acids can be modified. Carboxyl groups can form amide bonds by reacting with an amine, but the reaction requires the addition of activation reagents.

In certain embodiments, NP surfaces are modified by attaching a chemical linker that makes available reactive chemical groups such as azides and maleimides for reaction with the chimeric polypeptide. In such embodiments, the chimeric polypeptide is suitably modified to react with the reactive chemical group on the NP to form an intermolecular bond.

The optimal conjugation approach depends on the nature of the NP and the chimeric polypeptide. The use of chemical conjugation for the display both protein/peptides on NP surfaces is well established in the art including, e.g., vaccines against asthma, hypertension nicotine, and immunotherapies for neurodegenerative diseases.

### Protein Ligation Systems for Chimeric Polypeptide Display

In certain embodiments, the chimeric polypeptide is conjugated to the nanoparticle via a protein ligation system.

In certain embodiments, the chimeric polypeptide and NP are separately produced and then mixed in vitro to facilitate the attachment of the chimeric polypeptide to the NP. In certain embodiments, the intermolecular bond between the chimeric polypeptide and the NP is non-covalent (e.g., via His-tag/Ni-NTA, biotin-avidin affinities). In certain embodiments, intermolecular bond between the chimeric polypeptide and the NP is covalent (e.g., via Halo-tag, SNAP-tag, Sortase, Split-inteins, SpyTag-SpyCatcher). In certain embodiments, multiple different chimeric polypeptides are covalently and/or non-covalently attached to the same nanoparticle.

### Genetic Fusion of Protein Antigens

In preferred embodiments, the chimeric polypeptide is genetically fused to the nanoparticle. In such embodiments, the nanoparticle is a protein nanoparticle. In preferred such embodiments, the nanoparticle is a self-assembling protein nanoparticle.

In certain embodiments, the chimeric polypeptide and the nanoparticle are produced in the same host cell or cell-free expression system. In certain embodiments, both the chimeric polypeptide and NP are encoded by the same plasmid. In preferred embodiments, the chimeric polypeptide and NP are produced in the same host cell or cell-free expression system. This approach simplifies the entire purification and characterization process; the number of chimeric polypeptides on a single NP is constant for all the NPs in the same sample and correctly assembled NPs can be easily purified by size (e.g., by size exclusion chromatography). In certain embodiments, a nucleic acid sequence encoding the chimeric polypeptide is genetically fused to a nucleic acid sequence encoding the protein nanoparticle. In preferred such embodiments, the chimeric polypeptide is genetically fused to the N- or C-terminus of the nanoparticle, preferably the N-terminus of the nanoparticle. In preferred embodiments, the nucleic acid encoding the chimeric polypeptide and nanoparticle also encodes an amino acid linker between the nanoparticle and the chimeric polypeptide. In certain such embodiments, the amino acid linker consists of glycine and/or serine residues. In certain such embodiments, the linker comprises poly-glycine (Glyn, where n=2, 3, 4, 5, 6, 7, 8, 9, 10 or more), GSGS (SEQ ID NO:178), GSGGGG (SEQ ID NO:179), or GSGSGGGG (SEQ ID NO:180). In preferred embodiments, the amino acid linker consists of 1, 2, 3, 4, 5, 6, 7, 8, 9 or 10 GGS repeats, optionally wherein the amino acid linker is GGSGGSGGSGGS (SEQ ID NO:181). Other suitable linker amino acid sequences will be apparent to those skilled in the art.

In certain embodiments, the nanoparticle comprises a fusion tag such as an epitope tag, an affinity tag, a fluorescent tag, or a bioluminescent tag. In certain such embodiments, the nanoparticle comprises a N-terminal or C-terminal polyhistidine tag (i.e., Hisₙ where n=3, 4, 5, 6, 7, 8, 9, 10 or more), preferably a C-terminal polyhistidine tag, preferably His6 i.e. HHHHHH (SEQ ID NO:175).

In certain embodiments, the nanoparticle comprises a signal peptide to facilitate trafficking. Protein sorting, translocation and secretion mechanisms are known to a person skilled in the art.

In preferred embodiments, the nanoparticle is a biopolymer-based NP. In preferred such embodiments, the biopolymer-based NP is formed by proteins. In more preferred embodiments, the nanoparticle is a self-assembling nanoparticle. In most preferred such embodiments, the nanoparticle is a self-assembling protein nanoparticle. In certain such embodiments, the nanoparticle is an mI3 nanoparticle.

mI3 (also known as “mutated i301”) is a NP based on an i301 scaffold wherein Cys76 and Cys100 are substituted with alanines thereby avoiding the formation of undesirable disulphide bond. SpyTag-SpyCatcher technology has been widely used for the display of protein antigens on the surface of mI3. Such chimeric forms of mI3 can be easily obtained in E. coli and its intrinsic stability make it stable at room temperature, resistant to freeze-thaw cycles and it can be also lyophilized without losing immunogenicity or activity.

In certain embodiments, the nanoparticle is at least 40%, at least 50%, at least 60%, at least 70%, at least 80%, at least 90%, at least 95%, at least 97%, at least 98%, or at least 99% identical to the amino acid sequence shown in SEQ ID NO:98 or SEQ ID NO:99.

In certain embodiments, the nanoparticle comprises or consists of an amino acid sequence as shown in SEQ ID NO:98 or SEQ ID NO:99.

TABLE 4
Exemplary Nanoparticles

| SEQ ID | | |
|---|---|---|
| NO: | Construct | Amino acid sequence: |
| 98 | MI3- | MPTIENEPKEGIPVDKKITVNKTWA |
| | D3PorBLoop5 | VDGNEVNKADETVDAVFTLQVKQRY |
| | | GEGTKKIEYDGQTYSIPSLFVKWVN |
| | | VDSAKATAATSFKHTFENLDNAKTY |
| | | RVIERVSGYAPEYVSFVNGVVTIKN |
| | | NKDSNEPTPIGGSGGSGGSGGSMKM |
| | | EELFKKHKIVAVLRANSVEEAKKKA |
| | | LAVFLGGVHLIEITFTVPDADTVIK |
| | | ELSFLKEMGAIIGAGTVTSVEQARK |
| | | AVESGAEFIVSPHLDEEISQFAKEK |
| | | GVFYMPGVMTPTELVKAMKLGHTIL |
| | | KLFPGEVVGPQFVKAMKGPFPNVKF |
| | | VPTGGVNLDNVCEWFKAGVLAVGVG |
| | | SALVKGTPVEVAEKAKAFVEKIRGC |
| | | TEHHHHHH |
| 99 | MI3- | MPTIENEPKEGIPVDKKITVNKTWA |
| | D3PorBLoop3 | VDGNEVNKADETVDAVFTLQVKSPL |
| | | KNTGANVNAWESGKFTGNVLEISGM |
| | | AQREHRKWVNVDSAKATAATSFKHT |
| | | FENLDNAKTYRVIERVSGYAPEYVS |
| | | FVNGVVTIKNNKDSNEPTPIGGSGG |
| | | SGGSGGSMKMEELFKKHKIVAVLRA |
| | | NSVEEAKKKALAVFLGGVHLIEITF |
| | | TVPDADTVIKELSFLKEMGAIIGAG |
| | | TVTSVEQARKAVESGAEFIVSPHLD |
| | | EEISQFAKEKGVFYMPGVMTPTELV |
| | | KAMKLGHTILKLFPGEVVGPQFVKA |
| | | MKGPFPNVKFVPTGGVNLDNVCEWF |
| | | KAGVLAVGVGSALVKGTPVEVAEKA |
| | | KAFVEKIRGCTEHHHHHH |

### Outer Membrane Vesicles

Pathogenic and nonpathogenic gram-negative bacteria are able to spontaneously release 25-250 nm vesicles during growth in a process of blebbing, especially during the end of log phase (Mancini F. et al., GMMA-Based Vaccines: The Known and The Unknown. Front Immunol. 2021 Aug. 3; 12:715393. doi: 10.3389/fimmu.2021.715393). Since they originate from the bacterial outer membrane, these vesicles are known as “outer membrane vesicles” (OMVs). OMVs reflect the bacterial membrane composition and contain bacterial antigens such as lipopolysaccharides (LPS) and proteins in their membrane environment, as well as other immunostimulatory molecules (e.g., lipoproteins, peptidoglycans). The OMVs shed from outer membranes are distinct from OMVs derived by detergent extraction of whole bacteria, e.g., due to the depletion of lipoproteins and lipooligosaccharides by the detergent. Gram-negative bacteria naturally release OMV but in relatively low amounts. However, using genetic modification, it is possible to induce high level shedding of OMVs. Gram-negative bacteria engineered to have such an over-vesiculating phenotype are referred to in the art as “Generalized Modules for Membrane Antigens” (GMMA) (Mancini F. et al., GMMA-Based Vaccines: The Known and The Unknown. Front Immunol. 2021 Aug. 3; 12:715393. doi: 10.3389/fimmu.2021.715393). GMMA faithfully resemble the outer membrane of the bacterial pathogen they shed from but lack the ability to cause the associated disease. However, they present to the immune system antigens in their natural environment and conformation, facilitating uptake by immune cells and inducing strong immune response. Thus, OMVs such as GMMA represent a promising vaccine platform.

Therefore, in a third aspect, the present invention provides an outer membrane vesicle comprising a chimeric polypeptide according to the first aspect, wherein the chimeric polypeptide is expressed on the surface of the outer membrane vesicle.

In certain embodiments, the outer membrane vesicle is a native outer membrane vesicle. In preferred such embodiments, the native outer membrane vesicle is obtained or obtainable without use of a solubilization agent, most preferably obtained or obtainable without use of a detergent.

In certain embodiments, the outer membrane vesicle is obtained or obtainable from gram-negative bacterium. In certain embodiments, the gram-negative bacterium is Neisseria meningitidis, Neisseria gonorrhoeae, Francisella novicida, Escherichia coli, Bordetella pertussis, Non-typhoidal salmonella, Haemophilus influenza, Shigella sonnei, Klebsiella pneumoniae, Mycobacterium tuberculosis, or Vibrio cholerae. In certain embodiments, the outer membrane vesicle is from Neisseria gonorrhoeae strain FA1090.

In preferred embodiments, the gram-negative bacterium is a genetically modified gram-negative bacterium. For example, in certain embodiments, the genetic modification is a deletion or inactivation of the ompA gene, preferably deletion of the ompA gene.

In certain such embodiments, the genetic modification results in the gram-negative bacterium being hyper-blebbing.

In certain embodiments, the genetic modification reduces the systemic reactogenicity of the OMV. In certain embodiments, the genetic modification reduces TLR4 activation by LPS, preferably reduced activation by lipid A. In certain embodiments, the genetic modification is deletion or inactivation of an acyltransferase gene (e.g. the Lpxl1 gene). In certain embodiment, the genetic modification results in an OMV that expresses a penta-acylated lipid A rather than a hexa-acylated lipid.

### Production of Chimeric Polypeptides

In a fourth aspect, the present invention provides an isolated polynucleotide encoding a chimeric polypeptide according to the first aspect or a nanoparticle according to the second aspect.

In a fifth aspect, the present invention provides an expression vector comprising the polynucleotide of the fourth aspect operably linked to regulatory sequences which permit expression of the chimeric polypeptide or nanoparticle.

In a sixth aspect, the present invention provides a host cell or cell-free expression system containing an expression vector according to the fifth aspect.

Polynucleotide molecules encoding chimeric polypeptides of the invention include, for example, recombinant DNA molecules. The terms “nucleic acid”, “polynucleotide” or a “polynucleotide molecule” as used herein interchangeably and refer to any DNA or RNA molecule, either single- or double-stranded and, if single-stranded, the molecule of its complementary sequence. In discussing nucleic acid molecules, a sequence or structure of a particular nucleic acid molecule may be described herein according to the normal convention of providing the sequence in the 5′ to 3′ direction. In some embodiments of the invention, nucleic acids or polynucleotides are “isolated.” This term, when applied to a nucleic acid molecule, refers to a nucleic acid molecule that is separated from sequences with which it is immediately contiguous in the naturally occurring genome of the organism in which it originated. For example, an “isolated nucleic acid” may comprise a DNA molecule inserted into a vector, such as a plasmid or virus vector, or integrated into the genomic DNA of a prokaryotic or eukaryotic cell or non-human host organism. When applied to RNA, the term “isolated polynucleotide” refers primarily to an RNA molecule encoded by an isolated DNA molecule as defined above. Alternatively, the term may refer to an RNA molecule that has been purified/separated from other nucleic acids with which it would be associated in its natural state (i.e., in cells or tissues). An isolated polynucleotide (either DNA or RNA) may further represent a molecule produced directly by biological or synthetic means and separated from other components present during its production.

For recombinant production of a chimeric polypeptide according to the invention, a recombinant polynucleotide encoding it may be prepared (using standard molecular biology techniques) and inserted into a replicable vector for expression in a chosen host cell, or a cell-free expression system. Suitable host cells may, for example, be bacteria, yeast, insect, plant or mammalian cells. In preferred embodiments, the host cell is a bacterial cell, preferably an Escherichia coli cell. It should be noted that the term “host cell” generally refers to in vitro cultured cells.

In a seventh aspect, the present invention provides a method of producing a chimeric polypeptide or nanoparticle comprising culturing the host cell or cell-free expression system according to the sixth aspect under conditions which permit expression of chimeric polypeptide or nanoparticle and recovering the expressed chimeric polypeptide or nanoparticle.

In a seventh aspect, the present invention provides a method of producing a chimeric polypeptide or nanoparticle comprising culturing the host cell or cell-free expression system according to the sixth aspect under conditions which permit expression of chimeric polypeptide or nanoparticle and recovering the expressed chimeric polypeptide or nanoparticle. In certain embodiments, the recovered chimeric polypeptide is comprised in an OMV according to the third aspect. This recombinant expression process can be used for large scale production of chimeric polypeptides according to the invention. Suitable vectors, cell lines and production processes for large scale manufacture of recombinant chimeric polypeptides, nanoparticles and OMVs suitable for in vivo therapeutic or prophylactic use are generally available in the art and will be well known to the skilled person.

### Pharmaceutical Compositions

In an eighth aspect, the present invention provides a pharmaceutical composition comprising a chimeric polypeptide according to the first aspect, a nanoparticle according to the second aspect, or an outer membrane vesicle according to the third aspect, formulated with one or more pharmaceutically acceptable carriers, adjuvants and/or excipients. Such compositions may include one or a combination of (e.g., two or more different) chimeric polypeptides, nanoparticles or outer membrane vesicles.

As used herein, “pharmaceutically acceptable” refers to a material that is not biologically or otherwise undesirable, e.g., the material may be administered to a subject along with the one or more of the chimeric polypeptides, nanoparticles or outer membrane vesicles without causing any undesirable biological effects or interacting in a deleterious manner with any of the other components of the pharmaceutical composition in which it is contained. Principles and considerations involved in preparing such compositions, as well as guidance in the choice of components are provided, for example, in Remington: The Science And Practice Of Pharmacy 19th ed. (Alfonso R. Gennaro, et ah, editors) Mack Pub. Co., Easton, Pa., 1995; Drug Absorption Enhancement: Concepts, Possibilities, Limitations, And Trends, Harwood Academic Publishers, Langhorne, Pa., 1994; and Peptide And Protein Drug Delivery (Advances In Parenteral Sciences, Vol. 4), 1991, M. Dekker, New York. “Excipients” as used herein are natural or synthetic substances included in a pharmaceutical formulation alongside an active ingredient (e.g., the one or more chimeric polypeptides, nanoparticles or outer membrane vesicles). The intended purpose of excipients is typically to act as a carrier (vehicle) for the active ingredient and, in so doing, to contribute to product attributes such as stability, biopharmaceutical profile, appearance and patient acceptability.

Excipients can also be useful in the manufacturing process, to aid in the handling of the active substance concerned, in addition to aiding in vitro stability, such as prevention of denaturation and/or aggregation of the antibody over the expected shelf life. Pharmaceutically acceptable excipients are well known in the art. A suitable excipient is therefore easily identifiable by one of ordinary skill in the art. By way of example, suitable pharmaceutically acceptable excipients include water, physiological buffers, stabilisers, tonicity agents, surfactants, and the like.

In further embodiments, the pharmaceutical compositions of the invention, may comprise carriers. As defined herein “carriers” are non-toxic to recipients at the dosages and concentrations employed and are compatible with other ingredients of the formulation. The term “carrier” denotes an organic or inorganic ingredient, natural or synthetic, with which the active ingredient is combined to facilitate the application. Pharmaceutically acceptable carriers are well known in the art. A suitable carrier is therefore easily identifiable by one of ordinary skill in the art.

The pharmaceutical compositions of the invention may comprise an adjuvant. As used herein “adjuvants” are pharmacological and/or immunological agents that modify the effect of other agents in a formulation. Pharmaceutically acceptable adjuvants are well known in the art.

Suitable adjuvants include an aluminum salt such as aluminum hydroxide gel or aluminum phosphate or alum, but may also be a salt of calcium, magnesium, iron or zinc, or may be an insoluble suspension of acylated tyrosine, or acylated sugars, cationically or anionically derivatized saccharides, or polyphosphazenes. In one embodiment, the chimeric polypeptide according to the first aspect, the nanoparticle according to the second aspect, or the outer membrane vesicle according to the third aspect may be adsorbed onto aluminium phosphate. In another embodiment, the chimeric polypeptide according to the first aspect, the nanoparticle according to the second aspect, or the outer membrane vesicle according to the third aspect may be adsorbed onto aluminium hydroxide.

Suitable adjuvant systems which promote a predominantly Th1 response include: non-toxic derivatives of lipid A, Monophosphoryl lipid A (MPL) or a derivative thereof, particularly 3-de-O-acylated monophosphoryl lipid A (3D-MPL) (for its preparation see GB 2220211 Å); and a combination of monophosphoryl lipid A, preferably 3-de-O-acylated monophosphoryl lipid A, together with either an aluminum salt (for instance aluminum phosphate or aluminum hydroxide) or an oil-in-water emulsion. In such combinations, antigen and 3D-MPL are contained in the same particulate structures, allowing for more efficient delivery of antigenic and immunostimulatory signals. Studies have shown that 3D-MPL is able to further enhance the immunogenicity of an alum-adsorbed antigen (Thoelen et al. Vaccine (1998) 16:708-14; EP 689454-B1).

AS01 is an Adjuvant System containing MPL (3-O-desacyl-4′-monophosphoryl lipid A), QS21 ((Quillaja saponaria Molina, fraction 21) Antigenics, New York, NY, USA) and liposomes. AS01 B is an Adjuvant System containing MPL, QS21 and liposomes (50 μg MPL and 50 μg QS21). AS01E is an Adjuvant System containing MPL, QS21 and liposomes (25 μg MPL and 25 μg QS21). In one embodiment, the immunogenic composition or vaccine comprises AS01. In another embodiment, the immunogenic composition or vaccine comprises AS01B or AS01E.

AS02 is an Adjuvant System containing MPL and QS21 in an oil/water emulsion. AS02V is an Adjuvant System containing MPL and QS21 in an oil/water emulsion (50 □g MPL and 50 □g QS21). AS03 is an Adjuvant System containing α-Tocopherol and squalene in an oil/water (o/w) emulsion. AS03_{A} is an Adjuvant System containing α-Tocopherol and squalene in an o/w emulsion (11.86 mg tocopherol). AS03_{B} is an Adjuvant System containing α-Tocopherol and squalene in an o/w emulsion (5.93 mg tocopherol). AS03c is an Adjuvant System containing α-Tocopherol and squalene in an o/w emulsion (2.97 mg tocopherol). In one embodiment, the immunogenic composition or vaccine comprises AS03.

AS04 is an Adjuvant System containing MPL (50 μg MPL) adsorbed on an aluminum salt (500 μg Al³⁺). In one embodiment, the immunogenic composition or vaccine comprises AS04.

In certain embodiments, the pharmaceutical compositions are formulated for administration to a subject via any suitable route of administration including but not limited to intramuscular, intravenous, intradermal, intraperitoneal injection, subcutaneous, epidural, nasal, aural, ocular, oral, rectal, vaginal, topical, inhalational, buccal (e.g., sublingual), and transdermal administration. In certain embodiments, the compositions are formulated as aqueous solutions, tablets, capsules, powders or any other suitable dosage form.

The pharmaceutical composition is preferably sterile. It is preferably pyrogen-free.

The pharmaceutical compositions may also be lyophilised (e.g., for long term storage), and reconstituted in a suitable diluent prior to use.

In an aspect, the invention provides a delivery device containing a pharmaceutical composition of the third aspect. The device may be, for example, a syringe or an inhaler.

### Therapeutic Uses

### Methods of Treatment or Prevention of a Disease or Condition

In a ninth aspect, the present invention provides a method of treatment or prevention comprising administering a chimeric polypeptide according to the first aspect, the nanoparticle according to the second aspect, the outer membrane vesicle according to the third aspect, or the pharmaceutical composition according to the eighth aspect.

In certain embodiments, the method is for treating or preventing a pathogenic infection. In preferred such embodiments, the pathogenic infection is caused by a pathogen from which one or more of the exogenous polypeptide(s) is derived.

In certain embodiments, the method is for treating or preventing cancer.

As used herein, “treatment” of a disease or condition means curing a disease or condition and/or, in a subject diagnosed with the disease or condition, alleviating or eradicating one or more symptoms associated with the disease or condition such that the subject's suffering is reduced.

As used herein, a method of “prevention” of a disease or condition means preventing the onset of the disease, preventing the worsening of symptoms, preventing the progression of the disease or condition or reducing the risk of a subject developing the disease or condition. That is, in some embodiments, the invention provides prophylactic methods to prevent pathogenic infection in a subject (e.g., a subject at risk of (or susceptible to) pathogenic infection). Administration of a prophylactic agent can occur prior to infection or prior to the development of symptoms characteristic of the pathogenic infection, such that a pathogen-related disease or pathogen-related disorder is prevented or, alternatively, delayed in its progression. Subjects at risk of a particular pathogenic infection, are subjects who have been or are likely to be exposed to the particular pathogen.

In a tenth aspect, the present invention provides a chimeric polypeptide according to the first aspect, a nanoparticle according to the second aspect, an outer membrane vesicle according to the third aspect, or a pharmaceutical composition according to the eighth aspect, for use in a method according to the ninth aspect.

In an eleventh aspect, the present invention provides the use of a chimeric polypeptide according to the first aspect, a nanoparticle according to the second aspect, an outer membrane vesicle according to the third aspect, or a pharmaceutical composition according to the eighth aspect in the manufacture of a medicament.

In certain embodiments, the medicament is for treating or preventing a pathogenic infection. In preferred such embodiments, the pathogenic infection is caused by a pathogen from which one or more of the exogenous polypeptide(s) is derived.

In certain embodiments, the medicament is for treating or preventing cancer.

### Immunogenic Compositions

As described elsewhere herein, the chimeric polypeptides, nanoparticles, OMVs and pharmaceutical compositions according to the invention can find particular use as immunogenic compositions.

In a twelfth aspect, the present invention provides a method for raising an immune response in a mammal comprising administering to the mammal a chimeric polypeptide according to the first aspect, a nanoparticle according to the second aspect, an outer membrane vesicle according to the third aspect, or a pharmaceutical composition according to the eighth aspect. The invention also provides a chimeric polypeptide according to the first aspect, a nanoparticle according to the second aspect, an outer membrane vesicle according to the third aspect, and a pharmaceutical composition according to the eighth aspect for use in a method according to the twelfth aspect. The mammal is preferably a human.

In preferred embodiments, the immune response is raised against the exogenous polypeptide. In preferred such embodiments, an immune response is not raised against the scaffold polypeptide.

In a thirteenth aspect, the present invention provides a vaccine or immunogenic composition comprising a chimeric polypeptide according to the first aspect, a nanoparticle according to the second aspect, an outer membrane vesicle according to the third aspect, or a pharmaceutical composition according to the eighth aspect.

Vaccines according to the invention are particularly useful for the treatment of or protecting against infections caused by the organism(s) or virus(es) from which the exogenous polypeptide(s) is/are derived.

In the methods of treatment, prevention or vaccination according to the invention, the subject to be treated is administered an amount effective to achieve the recited treatment, prevention or vaccination.

In the methods of treatment, prevention or vaccination according to the invention, the subject to be treated is preferably a mammal, more preferably a human.

### Methods of Antibody Screening

In a fourteenth aspect, the present invention provides a method of screening for an antibody which binds a target protein, comprising:
(i) exposing a population of antibodies to a chimeric polypeptide according to the first aspect, a nanoparticle according to the second aspect, or an outer membrane vesicle according to the third aspect,
   wherein one or more of the exogenous polypeptide(s) of the chimeric polypeptide comprises an antigenic fragment of the target protein; and
(ii) identifying those antibodies which bind to the chimeric polypeptide as binding to the target protein.

Chimeric polypeptides according to the first aspect can be used to screen for antibodies binding to the exogenous polypeptide(s) therein. Such screens may facilitate the localization and/or quantitation of the antibodies (e.g., for use in measuring levels of antibodies binding the exogenous polypeptide(s) within samples, for use in diagnostic methods, for use in imaging the antibodies, and the like). In other words, the chimeric polypeptides of the invention can be used for the detection and/or isolation of antibodies (or antigen-binding fragments thereof) in a sample. Detection can be facilitated by coupling (e.g., physically linking) the chimeric polypeptide(s), or the nanoparticle comprising the chimeric polypeptide, to a detectable substance (e.g., a fluorescence tag as described herein). Other examples of detectable substances include various enzymes, prosthetic groups, luminescent materials, bioluminescent materials, and radioactive materials that will be known to a person of ordinary skill in the art. In certain embodiments, the chimeric polypeptide or nanoparticle contains a detectable label. In this context, the term “labelled”, means direct labelling of the chimeric polypeptide or nanoparticle by coupling (e.g., physically linking) a detectable substance to the chimeric polypeptide or nanoparticle, as well as indirect labelling of the chimeric polypeptide or nanoparticle by reactivity with another reagent that is directly labelled (e.g., a fluorescently-labelled secondary antibody that binds to an epitope on the chimeric polypeptide or nanoparticle that is not present in the exogenous polypeptide).

In certain embodiments, the method comprises separating the antibodies bound to the chimeric polypeptide from a larger population of antibodies. To that end, in certain embodiments, the chimeric polypeptides and nanoparticles according to the invention may be immobilized, e.g., on magnetic beads or an agarose gel matrix for use with standard separation techniques.

In certain embodiments, the methods of antibody screening according to the invention comprise contacting a sample obtained from a subject with one or more of the chimeric polypeptides of the invention. In preferred embodiments, the step of contacting occurs under conditions permitting binding between reactive antibodies in the sample and the chimeric polypeptide(s).

As used herein, a “sample” includes any tissue or fluid sample obtainable from a subject, which contains detectable quantities of antibodies, under normal or pathological conditions. In certain embodiments the sample is a biological sample. The term “biological sample” as used herein includes tissues, cells and biological fluids isolated from a subject, as well as tissues, cells and biological fluids present within a subject. In preferred embodiments, the methods are for screening, detecting and/or isolating antibodies in vitro. “Biological fluid” as used herein includes for example saliva, mucus, urine, blood, lymphatic fluid and the like. “Biological sample” as used herein therefore includes blood and a fraction or component of blood such as blood serum, blood plasma, or lymph. In preferred embodiments, the biological sample is blood serum.

### Arrays

In a fifteenth aspect, the present invention provides an array comprising a plurality of chimeric polypeptides according to the first aspect, a plurality of nanoparticles according to the second aspect, or a plurality of outer membrane vesicles according to the third aspect.

Such arrays can be useful in the screening of antibodies as described hereinabove.

### Kits

Any of the chimeric polypeptides, nanoparticles, outer membrane vesicles, pharmaceutical compositions, vaccines, or arrays described herein can be packaged as a kit (e.g., in a container, pack, dispenser, microplate). The kits optionally include instructions for use.

The invention can also be understood by reference to the following clauses:
1. A chimeric polypeptide comprising:
   (i) a scaffold polypeptide; and
   (ii) one or more exogenous polypeptide(s),
   wherein the scaffold polypeptide comprises a backbone protein 2a (BP-2a) Domain 3 (D3) polypeptide, wherein in the chimeric polypeptide at least one endogenous loop of the BP-2a D3 polypeptide is partially or wholly replaced by an exogenous polypeptide.
2. The chimeric polypeptide of clause 1, wherein the at least one endogenous loop of the BP-2a D3 polypeptide is selected from the first, second, third, fourth, fifth and sixth endogenous loops of the BP-2a D3 polypeptide.
3. The chimeric polypeptide of clause 1 or clause 2, wherein the at least one endogenous loop of the BP-2a D3 polypeptide is selected from the first, second, third, fourth, fifth and sixth endogenous loops of the BP-2a D3 polypeptide, wherein:
   (i) the first endogenous loop of the BP-2a D3 polypeptide spans the amino acids at positions corresponding to positions 360-372 of SEQ ID NO:1;
   (ii) the second endogenous loop of the BP-2a D3 polypeptide spans the amino acids at positions corresponding to positions 380-384 of SEQ ID NO:1;
   (iii) the third endogenous loop of the BP-2a D3 polypeptide spans the amino acids at positions corresponding to positions 393-399 of SEQ ID NO:1;
   (iv) the fourth endogenous loop of the BP-2a D3 polypeptide spans the amino acids at positions corresponding to positions 404-411 of SEQ ID NO:1;
   (v) the fifth endogenous loop of the BP-2a D3 polypeptide spans the amino acids at positions corresponding to positions 418-422 of SEQ ID NO:1; and
   (vi) the sixth endogenous loop of the BP-2a D3 polypeptide spans the amino acids at positions corresponding to positions 429-432 of SEQ ID NO:1,
wherein the amino acid positions are numbered according to the sequence shown in SEQ ID NO:1, wherein amino acids 332-447 are the native BP-2a D3 polypeptide.
4. The chimeric polypeptide of any preceding clause, wherein the BP-2a D3 polypeptide comprises seven scaffold regions, wherein:
   (i) the first scaffold region of the BP-2a D3 polypeptide spans the amino acids at positions corresponding to positions 332-359 of SEQ ID NO:1;
   (ii) the second scaffold region of the BP-2a D3 polypeptide spans the amino acids at positions corresponding to positions 373-379 of SEQ ID NO:1;
   (iii) the third scaffold region of the BP-2a D3 polypeptide spans the amino acids at positions corresponding to positions 385-392 of SEQ ID NO:1;
   (iv) the fourth scaffold region of the BP-2a D3 polypeptide spans the amino acids at positions corresponding to positions 400-403 of SEQ ID NO:1;
   (v) the fifth scaffold region of the BP-2a D3 polypeptide spans the amino acids at positions corresponding to positions 412-417 of SEQ ID NO:1;
   (vi) the sixth scaffold region spans the amino acids at positions corresponding to positions 423-428 of SEQ ID NO:1; and
   (vii) the seventh scaffold region of the BP-2a D3 polypeptide spans the amino acids at positions corresponding to positions 433-447 of SEQ ID NO:1,
wherein the amino acids are numbered according to the sequence shown in SEQ ID NO:1, wherein amino acids 332-447 are the native BP-2a D3 polypeptide.
5. The chimeric polypeptide of any preceding clause, wherein the BP-2a D3 polypeptide is at least 40%, at least 50%, at least 60%, at least 70%, at least 80%, at least 90%, at least 95%, at least 97%, at least 98%, at least 99% or 100% identical to the amino acid sequence shown in SEQ ID NO:2; SEQ ID NO:101; SEQ ID NO:116; SEQ ID NO:131; SEQ ID NO:146; or SEQ ID NO:161, but for the one or more exogenous polypeptides replacing (wholly or partially) at least one of the endogenous loops.
6. The chimeric polypeptide of any preceding clause, wherein the at least one endogenous loop of the BP-2a D3 polypeptide is selected from the first, second, third, fourth, fifth and sixth endogenous loops of the BP-2a D3 polypeptide, wherein:
   (i) the first endogenous loop of the BP-2a D3 polypeptide spans the amino acids at positions 360-372;
   (ii) the second endogenous loop of the BP-2a D3 polypeptide spans the amino acids at positions 380-384;
   (iii) the third endogenous loop of the BP-2a D3 polypeptide spans the amino acids at positions 393-399;
   (iv) the fourth endogenous loop of the BP-2a D3 polypeptide spans the amino acids at positions 404-411;
   (v) the fifth endogenous loop of the BP-2a D3 polypeptide spans the amino acids at positions 418-422; and
   (vi) the sixth endogenous loop of the BP-2a D3 polypeptide spans the amino acids at positions 429-432,
      wherein the amino acids are numbered according to the sequence shown in SEQ ID NO:1, wherein amino acids 332-447 are the native BP-2a D3 polypeptide,
or wherein:
   (vii) the first endogenous loop of the BP-2a D3 polypeptide spans the amino acids at positions 369-391;
   (viii) the second endogenous loop of the BP-2a D3 polypeptide spans the amino acids at positions 399-402;
   (ix) the third endogenous loop of the BP-2a D3 polypeptide spans the amino acids at positions 412-418;
   (x) the fourth endogenous loop of the BP-2a D3 polypeptide spans the amino acids at positions 423-431;
   (xi) the fifth endogenous loop of the BP-2a D3 polypeptide spans the amino acids at positions 436-441; and
   (xii) the sixth endogenous loop of the BP-2a D3 polypeptide spans the amino acids at positions 446-451,
wherein the amino acids are numbered according to the sequence shown in SEQ ID NO:100, wherein amino acids 344-465 are the native BP-2a D3 polypeptide,
or wherein:
   (xiii) the first endogenous loop of the BP-2a D3 polypeptide spans the amino acids at positions 357-370;
   (xiv) the second endogenous loop of the BP-2a D3 polypeptide spans the amino acids at positions 379-382;
   (xv) the third endogenous loop of the BP-2a D3 polypeptide spans the amino acids at positions 392-397;
   (xvi) the fourth endogenous loop of the BP-2a D3 polypeptide spans the amino acids at positions 403-411;
   (xvii) the fifth endogenous loop of the BP-2a D3 polypeptide spans the amino acids at positions 416-421; and
   (xviii) the sixth endogenous loop of the BP-2a D3 polypeptide spans the amino acids at positions 428-431,
wherein the amino acids are numbered according to the sequence shown in SEQ ID NO:115, wherein amino acids 332-446 are the native BP-2a D3 polypeptide,
or wherein:
   (xix) the first endogenous loop of the BP-2a D3 polypeptide spans the amino acids at positions 387-399;
   (xx) the second endogenous loop of the BP-2a D3 polypeptide spans the amino acids at positions 404-412;
   (xxi) the third endogenous loop of the BP-2a D3 polypeptide spans the amino acids at positions 417-436;
   (xxii) the fourth endogenous loop of the BP-2a D3 polypeptide spans the amino acids at positions 442-447;
   (xxiii) the fifth endogenous loop of the BP-2a D3 polypeptide spans the amino acids at positions 454-459; and
   (xxiv) the sixth endogenous loop of the BP-2a D3 polypeptide spans the amino acids at positions 465-468,
wherein the amino acids are numbered according to the sequence shown in SEQ ID NO:130, wherein amino acids 364-483 are the native BP-2a D3 polypeptide,
or wherein:
   (xxv) the first endogenous loop of the BP-2a D3 polypeptide spans the amino acids at positions 385-407;
   (xxvi) the second endogenous loop of the BP-2a D3 polypeptide spans the amino acids at positions 416-419;
   (xxvii) the third endogenous loop of the BP-2a D3 polypeptide spans the amino acids at positions 429-435;
   (xxviii) the fourth endogenous loop of the BP-2a D3 polypeptide spans the amino acids at positions 440-447;
   (xxix) the fifth endogenous loop of the BP-2a D3 polypeptide spans the amino acids at positions 452-457; and
   (xxx) the sixth endogenous loop of the BP-2a D3 polypeptide spans the amino acids at positions 463-466,
wherein the amino acids are numbered according to the sequence shown in SEQ ID NO:145, wherein amino acids 360-481 are the native BP-2a D3 polypeptide,
or wherein:
   (xxxi) the first endogenous loop of the BP-2a D3 polypeptide spans the amino acids at positions 365-379;
   (xxxii) the second endogenous loop of the BP-2a D3 polypeptide spans the amino acids at positions 387-390;
   (xxxiii) the third endogenous loop of the BP-2a D3 polypeptide spans the amino acids at positions 397-405;
   (xxxiv) the fourth endogenous loop of the BP-2a D3 polypeptide spans the amino acids at positions 410-418;
   (xxxv) the fifth endogenous loop of the BP-2a D3 polypeptide spans the amino acids at positions 423-428; and
   (xxxvi) the sixth endogenous loop of the BP-2a D3 polypeptide spans the amino acids at positions 435-438,
wherein the amino acids are numbered according to the sequence shown in SEQ ID NO:160, wherein amino acids 339-453 are the native BP-2a D3 polypeptide.
7. The chimeric polypeptide of any preceding clause, wherein the BP-2a D3 polypeptide comprises seven scaffold regions, wherein:
   (i) the first scaffold region spans the amino acids at positions 332-359;
   (ii) the second scaffold region spans the amino acids at positions 373-379;
   (iii) the third scaffold region spans the amino acids at positions 385-392;
   (iv) the fourth scaffold region spans the amino acids at positions 400-403;
   (v) the fifth scaffold region spans the amino acids at positions 412-417;
   (vi) the sixth scaffold region spans the amino acids at positions 423-428; and
   (vii) the seventh scaffold region spans the amino acids at positions 433-447,
wherein the amino acids are numbered according to the sequence shown in SEQ ID NO:1, wherein amino acids 332-447 are the native BP-2a D3 polypeptide,
or wherein:
   (viii) the first scaffold region spans the amino acids at positions 344-368;
   (ix) the second scaffold region spans the amino acids at positions 392-398;
   (x) the third scaffold region spans the amino acids at positions 403-411;
   (xi) the fourth scaffold region spans the amino acids at positions 419-422;
   (xii) the fifth scaffold region spans the amino acids at positions 432-435;
   (xiii) the sixth scaffold region spans the amino acids at positions 442-445; and
   (xiv) the seventh scaffold region spans the amino acids at positions 452-465,
wherein the amino acids are numbered according to the sequence shown in SEQ ID NO:100, wherein amino acids 344-465 are the native BP-2a D3 polypeptide,
or wherein:
   (xv) the first scaffold region spans the amino acids at positions 332-356;
   (xvi) the second scaffold region spans the amino acids at positions 371-378;
   (xvii) the third scaffold region spans the amino acids at positions 383-391;
   (xviii) the fourth scaffold region spans the amino acids at positions 398-402;
   (xix) the fifth scaffold region spans the amino acids at positions 412-415;
   (xx) the sixth scaffold region spans the amino acids at positions 422-427; and
   (xxi) the seventh scaffold region spans the amino acids at positions 432-446,
wherein the amino acids are numbered according to the sequence shown in SEQ ID NO:115, wherein amino acids 332-446 are the native BP-2a D3 polypeptide,
or wherein:
   (xxii) the first scaffold region spans the amino acids at positions 364-386;
   (xxiii) the second scaffold region spans the amino acids at positions 400-403;
   (xxiv) the third scaffold region spans the amino acids at positions 413-416;
   (xxv) the fourth scaffold region spans the amino acids at positions 437-441;
   (xxvi) the fifth scaffold region spans the amino acids at positions 448-453;
   (xxvii) the sixth scaffold region spans the amino acids at positions 460-464; and
   (xxviii) the seventh scaffold region spans the amino acids at positions 469-483,
wherein the amino acids are numbered according to the sequence shown in SEQ ID NO:130, wherein amino acids 364-483 are the native BP-2a D3 polypeptide,
or wherein:
   (xxix) the first scaffold region spans the amino acids at positions 360-384;
   (xxx) the second scaffold region spans the amino acids at positions 408-415;
   (xxxi) the third scaffold region spans the amino acids at positions 420-428;
   (xxxii) the fourth scaffold region spans the amino acids at positions 436-439;
   (xxxiii) the fifth scaffold region spans the amino acids at positions 448-451;
   (xxxiv) the sixth scaffold region spans the amino acids at positions 458-462; and
   (xxxv) the seventh scaffold region spans the amino acids at positions 467-481,
wherein the amino acids are numbered according to the sequence shown in SEQ ID NO:145, wherein amino acids 360-481 are the native BP-2a D3 polypeptide,
or wherein:
   (xxxvi) the first scaffold region spans the amino acids at positions 339-364;
   (xxxvii) the second scaffold region spans the amino acids at positions 380-386;
   (xxxviii) the third scaffold region spans the amino acids at positions 391-396;
   (xxxix) the fourth scaffold region spans the amino acids at positions 406-409;
   (xl) the fifth scaffold region spans the amino acids at positions 419-422;
   (xli) the sixth scaffold region spans the amino acids at positions 429-434; and
   (xlii) the seventh scaffold region spans the amino acids at positions 439-453,
wherein the amino acids are numbered according to the sequence shown in SEQ ID NO:160, wherein amino acids 339-453 are the native BP-2a D3 polypeptide.
8. The chimeric polypeptide of any preceding clause, wherein the BP-2a D3 polypeptide comprises seven scaffold regions, wherein:
   (i) the first scaffold region is at least 40%, at least 50%, at least 60%, at least 70%, at least 80%, at least 90%, at least 95%, at least 97%, at least 98%, or at least 99% identical to the amino acid sequence shown in SEQ ID NO:10; SEQ ID NO:108; SEQ ID NO:123; SEQ ID NO:138; SEQ ID NO:153; or SEQ ID NO:168,
   (ii) the second scaffold region is at least 40%, at least 50%, at least 60%, at least 70%, at least 80%, at least 90%, at least 95%, at least 97%, at least 98%, or at least 99% identical to the amino acid sequence shown in SEQ ID NO:11; SEQ ID NO:109; SEQ ID NO:124; SEQ ID NO:139; SEQ ID NO:154; or SEQ ID NO:169,
   (iii) the third scaffold region is at least 40%, at least 50%, at least 60%, at least 70%, at least 80%, at least 90%, at least 95%, at least 97%, at least 98%, or at least 99% identical to the amino acid sequence shown in SEQ ID NO:12; SEQ ID NO:110; SEQ ID NO:125; SEQ ID NO:140; SEQ ID NO:155; or SEQ ID NO:170,
   (iv) the fourth scaffold region is at least 40%, at least 50%, at least 60%, at least 70%, at least 80%, at least 90%, at least 95%, at least 97%, at least 98%, or at least 99% identical to the amino acid sequence shown in SEQ ID NO:13; SEQ ID NO:111; SEQ ID NO:126; SEQ ID NO:141; SEQ ID NO:156; or SEQ ID NO:171,
   (v) the fifth scaffold region is at least 40%, at least 50%, at least 60%, at least 70%, at least 80%, at least 90%, at least 95%, at least 97%, at least 98%, or at least 99% identical to the amino acid sequence shown in SEQ ID NO:14; SEQ ID NO:112; SEQ ID NO:127; SEQ ID NO:142; SEQ ID NO:157; or SEQ ID NO:172,
   (vi) the sixth scaffold region is at least 40%, at least 50%, at least 60%, at least 70%, at least 80%, at least 90%, at least 95%, at least 97%, at least 98%, or at least 99% identical to the amino acid sequence shown in SEQ ID NO:15; SEQ ID NO:113; SEQ ID NO:128; SEQ ID NO:143; SEQ ID NO:158; or SEQ ID NO:173, and
   (vii) the seventh scaffold region is at least 40%, at least 50%, at least 60%, at least 70%, at least 80%, at least 90%, at least 95%, at least 97%, at least 98%, or at least 99% identical to the amino acid sequence shown in SEQ ID NO:16; SEQ ID NO:114; SEQ ID NO:129; SEQ ID NO:144; SEQ ID NO:159; or SEQ ID NO:174.
9. The chimeric polypeptide of any preceding clause, wherein the BP-2a D3 polypeptide comprises seven scaffold regions, wherein:
   (i) the first scaffold region comprises or consists of the amino acid sequence shown in SEQ ID NO:10; SEQ ID NO:108; SEQ ID NO:123; SEQ ID NO:138; SEQ ID NO:153; or SEQ ID NO:168,
   (ii) the second scaffold region comprises or consists of the amino acid sequence shown in SEQ ID NO:11; SEQ ID NO:109; SEQ ID NO:124; SEQ ID NO:139; SEQ ID NO:154; or SEQ ID NO:169,
   (iii) the third scaffold region comprises or consists of the amino acid sequence shown in SEQ ID NO:12; SEQ ID NO:110; SEQ ID NO:125; SEQ ID NO:140; SEQ ID NO:155; or SEQ ID NO:170,
   (iv) the fourth scaffold region comprises or consists of the amino acid sequence shown in SEQ ID NO:13; SEQ ID NO:111; SEQ ID NO:126; SEQ ID NO:141; SEQ ID NO:156; or SEQ ID NO:171,
   (v) the fifth scaffold region comprises or consists of the amino acid sequence shown in SEQ ID NO:14; SEQ ID NO:112; SEQ ID NO:127; SEQ ID NO:142; SEQ ID NO:157; or SEQ ID NO:172,
   (vi) the sixth scaffold region comprises or consists of the amino acid sequence shown in SEQ ID NO:15; SEQ ID NO:113; SEQ ID NO:128; SEQ ID NO:143; SEQ ID NO:158; or SEQ ID NO:173, and
   (vii) the seventh scaffold region comprises or consists of the amino acid sequence shown in SEQ ID NO:16; SEQ ID NO:114; SEQ ID NO:129; SEQ ID NO:144; SEQ ID NO:159; or SEQ ID NO:174.
10. The chimeric polypeptide of any one of clauses 2-9, wherein the at least one endogenous loop of the BP-2a D3 polypeptide partially or wholly replaced with an exogenous polypeptide is selected from the first, second, third, fifth and sixth endogenous loops.
11. The chimeric polypeptide of any preceding clause, wherein two or more endogenous loops of the BP-2a D3 polypeptide are each independently partially or wholly replaced by an exogenous polypeptide.
12. The chimeric polypeptide of any one of clauses 2-11, wherein the first endogenous loop of the BP-2a D3 polypeptide is partially or wholly replaced by an exogenous polypeptide.
13. The chimeric polypeptide of any one of clauses 2-12, wherein the second endogenous loop of the BP-2a D3 polypeptide is partially or wholly replaced by an exogenous polypeptide.
14. The chimeric polypeptide of any one of clauses 2-13, wherein the third, fifth, and/or sixth endogenous loops of the BP-2a D3 polypeptide are each independently partially or wholly replaced by an exogenous polypeptide.
15. The chimeric polypeptide of any one of clauses 2-14, wherein, when present:
   (i) the first endogenous loop of the BP-2a D3 polypeptide is at least 40%, at least 50%, at least 60%, at least 70%, at least 80%, at least 90%, at least 95%, at least 97%, at least 98%, or at least 99% to the amino acid sequence shown in SEQ ID NO:4, SEQ ID NO:102, SEQ ID NO:117, SEQ ID NO:132, SEQ ID NO:147, or SEQ ID NO:162,
   (ii) the second endogenous loop of the BP-2a D3 polypeptide is at least 40%, at least 50%, at least 60%, at least 70%, at least 80%, at least 90%, at least 95%, at least 97%, at least 98%, or at least 99% identical to the amino acid sequence shown in SEQ ID NO:5, SEQ ID NO:103, SEQ ID NO:118, SEQ ID NO:133, SEQ ID NO:148, or SEQ ID NO:163,
   (iii) the third endogenous loop of the BP-2a D3 polypeptide is at least 40%, at least 50%, at least 60%, at least 70%, at least 80%, at least 90%, at least 95%, at least 97%, at least 98%, or at least 99% identical to the amino acid sequence shown in SEQ ID NO:6, SEQ ID NO:104, SEQ ID NO:119, SEQ ID NO:134, SEQ ID NO:149, or SEQ ID NO:164,
   (iv) the fourth endogenous loop of the BP-2a D3 polypeptide is at least 40%, at least 50%, at least 60%, at least 70%, at least 80%, at least 90%, at least 95%, at least 97%, at least 98%, or at least 99% identical to the amino acid sequence shown in SEQ ID NO:7, SEQ ID NO:105, SEQ ID NO:120, SEQ ID NO:135, SEQ ID NO:150, or SEQ ID NO:165,
   (v) the fifth endogenous loop of the BP-2a D3 polypeptide is at least 40%, at least 50%, at least 60%, at least 70%, at least 80%, at least 90%, at least 95%, at least 97%, at least 98%, or at least 99% identical to the amino acid sequence shown in SEQ ID NO:8, SEQ ID NO:106, SEQ ID NO:121, SEQ ID NO:136, SEQ ID NO:151, or SEQ ID NO:166 and
   (vi) the sixth endogenous loop of the BP-2a D3 polypeptide is at least 40%, at least 50%, at least 60%, at least 70%, at least 80%, at least 90%, at least 95%, at least 97%, at least 98%, or at least 99% identical to the amino acid sequence shown in SEQ ID NO:9, SEQ ID NO:107, SEQ ID NO:122, SEQ ID NO:137, SEQ ID NO:152, or SEQ ID NO:167,
      optionally wherein, when present:
   (vii) the first endogenous loop of the BP-2a D3 polypeptide comprises the amino acid sequence shown in SEQ ID NO:4; SEQ ID NO:102, SEQ ID NO:117, SEQ ID NO:132, SEQ ID NO:147, or SEQ ID NO:162,
   (viii) the second endogenous loop of the BP-2a D3 polypeptide comprises the amino acid sequence shown in SEQ ID NO:5; SEQ ID NO:103, SEQ ID NO:118, SEQ ID NO:133, SEQ ID NO:148, or SEQ ID NO:163,
   (ix) the third endogenous loop of the BP-2a D3 polypeptide comprises the amino acid sequence shown in SEQ ID NO:6; SEQ ID NO:104, SEQ ID NO:119, SEQ ID NO:134, SEQ ID NO:149, or SEQ ID NO:164,
   (x) the fourth endogenous loop of the BP-2a D3 polypeptide comprises the amino acid sequence shown in SEQ ID NO:7; SEQ ID NO:105, SEQ ID NO:120, SEQ ID NO:135, SEQ ID NO:150, or SEQ ID NO:165,
   (xi) the fifth endogenous loop of the BP-2a D3 polypeptide comprises the amino acid sequence shown in SEQ ID NO:8; SEQ ID NO:106, SEQ ID NO:121, SEQ ID NO:136, SEQ ID NO:151, or SEQ ID NO:166 and
   (xii) the sixth endogenous loop of the BP-2a D3 polypeptide comprises the amino acid sequence shown in SEQ ID NO:9, SEQ ID NO:107, SEQ ID NO:122, SEQ ID NO:137, SEQ ID NO:152, or SEQ ID NO:167.
16. The chimeric polypeptide of any one of clauses 2-15, wherein, when present:
   (i) the first endogenous loop of the BP-2a D3 polypeptide comprises or consists of the amino acid sequence shown in SEQ ID NO:4, SEQ ID NO:102, SEQ ID NO:117, SEQ ID NO:132, SEQ ID NO:147, or SEQ ID NO:162;
   (ii) the second endogenous loop of the BP-2a D3 polypeptide comprises or consists of the amino acid sequence shown in SEQ ID NO:5; SEQ ID NO:103, SEQ ID NO:118, SEQ ID NO:133, SEQ ID NO:148, or SEQ ID NO:163;
   (iii) the third endogenous loop of the BP-2a D3 polypeptide comprises or consists of the amino acid sequence shown in SEQ ID NO:6; SEQ ID NO:104, SEQ ID NO:119, SEQ ID NO:134, SEQ ID NO:149, or SEQ ID NO:164;
   (iv) the fourth endogenous loop of the BP-2a D3 polypeptide comprises or consists of the amino acid sequence shown in SEQ ID NO:7; SEQ ID NO:105, SEQ ID NO:120, SEQ ID NO:135, SEQ ID NO:150, or SEQ ID NO:165;
   (v) the fifth endogenous loop of the BP-2a D3 polypeptide comprises or consists of the amino acid sequence shown in SEQ ID NO:8; SEQ ID NO:106, SEQ ID NO:121, SEQ ID NO:136, SEQ ID NO:151, or SEQ ID NO:166; and
   (vi) the sixth endogenous loop of the BP-2a D3 polypeptide comprises or consists of the amino acid sequence shown in SEQ ID NO:9, SEQ ID NO:107, SEQ ID NO:122, SEQ ID NO:137, SEQ ID NO:152, or SEQ ID NO:167.
17. The chimeric polypeptide of any one of clauses 2-15 wherein the fourth endogenous loop of the BP-2a D3 polypeptide is present and is at least 40%, at least 50%, at least 60%, at least 70%, at least 80%, at least 90%, at least 95%, at least 97%, at least 98%, or at least 99% identical to the amino acid sequence shown in SEQ ID NO:7, SEQ ID NO:105, SEQ ID NO:120, SEQ ID NO:135, SEQ ID NO:150, or SEQ ID NO:165, optionally wherein the fourth endogenous loop of the BP-2a D3 polypeptide is present and comprises or consists of the amino acid sequence shown in SEQ ID NO:7, SEQ ID NO:105, SEQ ID NO:120, SEQ ID NO:135, SEQ ID NO:150, or SEQ ID NO:165.
18. The chimeric polypeptide of any preceding clause, wherein the at least one endogenous loop of the BP-2a D3 polypeptide is partially replaced by an exogenous polypeptide.
19. The chimeric polypeptide of any preceding clause, wherein the at least one endogenous loop of the BP-2a D3 polypeptide is wholly replaced by an exogenous polypeptide.
20. The chimeric polypeptide of any preceding clause wherein the one or more exogenous polypeptide(s) each comprise at least 5 amino acids.
21. The chimeric polypeptide of any preceding clause wherein the one or more exogenous polypeptide(s) each comprise at most 300 amino acids.
22. The chimeric polypeptide of any preceding clause wherein the one or more exogenous polypeptides each independently comprise 5-300 amino acids, optionally 6-250 amino acids, optionally 7-200 amino acids, optionally 8-150 amino acids, optionally 9-100 amino acids, optionally 10-75 amino acids, optionally 11-57 amino acids.
23. The chimeric polypeptide of any preceding clause, wherein when multiple exogenous polypeptides are present, the exogenous polypeptides are the same.
24. The chimeric polypeptide of any preceding clause, wherein when multiple exogenous polypeptides are present, the exogenous polypeptides are different.
25. The chimeric polypeptide of any preceding clause, wherein the exogenous polypeptide comprises a fragment, preferably an antigenic fragment, of a target protein, optionally wherein the target protein is a membrane protein.
26. The chimeric polypeptide of clause 25, wherein the fragment (optionally the antigenic fragment) of the target protein is inaccessible to antibodies when comprised in the natively folded target protein.
27. The chimeric polypeptide of any preceding clause, wherein the exogenous polypeptide comprises a fragment of a target protein, wherein the target protein is insoluble when not comprised in the chimeric polypeptide.
28. The chimeric polypeptide of any preceding clause, wherein the one or more exogenous polypeptide(s) comprise a cryptotope.
29. The chimeric polypeptide of any preceding clause, wherein the exogenous polypeptide comprises a fragment of a target protein that is from microorganism or virus, optionally a pathogenic microorganism or virus.
30. The chimeric polypeptide of clause 29, wherein the pathogenic microorganism or virus is selected from Neisseria gonorrhoeae, Neisseria meningitidis, Non-typeable Haemophilus influenzae, Staphylococcus aureus, human papillomavirus (HPV), Chlamydia trachomatis, Chlamydia muridarum, Streptococcus pneumonia, and Streptococcus agalactiae.
31. The chimeric polypeptide of any preceding clause, comprising one or more exogenous polypeptide(s) each independently comprising an amino acid sequence selected from: SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:51, SEQ ID NO:52, SEQ ID NO:53, SEQ ID NO:182, SEQ ID NO:183, SEQ ID NO: 195, SEQ ID NO: 197, SEQ ID NO: 198, SEQ ID NO: 199 and SEQ ID NO: 200.
32. The chimeric polypeptide of clause 1 comprising an amino acid sequence selected from: SEQ ID NO:54, SEQ ID NO:55, SEQ ID NO:56, SEQ ID NO:57, SEQ ID NO:58, SEQ ID NO:59, SEQ ID NO:60, SEQ ID NO:61, SEQ ID NO:62, SEQ ID NO:63, SEQ ID NO:64, SEQ ID NO:65, SEQ ID NO:66, SEQ ID NO:67, SEQ ID NO:68, SEQ ID NO:69, SEQ ID NO:70, SEQ ID NO:71, SEQ ID NO:72, SEQ ID NO:73, SEQ ID NO:74, SEQ ID NO:75, SEQ ID NO:76, SEQ ID NO:77, SEQ ID NO:78, SEQ ID NO:79, SEQ ID NO:80, SEQ ID NO:81, SEQ ID NO:82, SEQ ID NO:83, SEQ ID NO:84, SEQ ID NO:85, SEQ ID NO:86, SEQ ID NO:87, SEQ ID NO:88, SEQ ID NO:89, SEQ ID NO:90, SEQ ID NO:91, SEQ ID NO:92, SEQ ID NO:93, SEQ ID NO:94, SEQ ID NO:95, SEQ ID NO:96, SEQ ID NO:97, SEQ ID NO:184, SEQ ID NO:185, SEQ ID NO:186, SEQ ID NO: 190, SEQ ID NO: 191, SEQ ID NO: 192, SEQ ID NO: 193, SEQ ID NO: 194 and SEQ ID NO: 196.
33. The chimeric polypeptide of any one of clauses 1-28 wherein the exogenous polypeptide comprises a fragment of a target protein that is a tumour antigen.
34. The chimeric polypeptide of any preceding clause, wherein the scaffold polypeptide is 12-60 kDa, optionally 12-50 kDa, optionally 12-40 kDa, optionally 12-30 kDa, optionally 12-20 kDa, optionally 12-18 kDa, optionally 13-17 kDa, optionally 14-16 kDa, optionally 15 kDa.
35. The chimeric polypeptide of any preceding clause, wherein the exogenous polypeptide is 0.5-35 kDa, optionally 0.75-25 kDa, optionally 1-15 kDa, optionally 1-12.5 kDa.
36. The chimeric polypeptide of any preceding clause, being 12.5-95 kDa, optionally 13-70 kDa, optionally 14-50 kDa, optionally 15-30 kDa.
37. The chimeric polypeptide of any preceding clause, wherein the BP-2a D3 polypeptide is from Streptococcus agalactiae, optionally Streptococcus agalactiae strain 515, Streptococcus agalactiae strain H36_{B}, Streptococcus agalactiae strain CJB111, Streptococcus agalactiae strain 2603, Streptococcus agalactiae strain CJB110, or Streptococcus agalactiae strain DK21, preferably Streptococcus agalactiae strain 515.
38. The chimeric polypeptide of any preceding clause wherein the BP-2a D3 polypeptide comprises an intramolecular isopeptide bond.
39. The chimeric polypeptide of clause 38 wherein the intramolecular isopeptide bond is between K355 and N437, wherein the amino acids are numbered according to the sequence shown in SEQ ID NO:1, wherein the native BP-2a D3 polypeptide spans amino acids 332-447.
40. The chimeric polypeptide of any preceding clause wherein the scaffold polypeptide comprises a fusion tag, optionally a peptide tag, optionally a polyhistidine-tag.
41. A nanoparticle comprising a chimeric polypeptide according to any preceding clause.
42. The nanoparticle of clause 41 being a self-assembling nanoparticle.
43. The nanoparticle of clause 42 being an mI3 nanoparticle.
44. The nanoparticle of any one of clauses 41-43 comprising or consisting of an amino acid sequence as shown in SEQ ID NO:98 or SEQ ID NO:99.
45. An outer membrane vesicle comprising a chimeric polypeptide according to any one of clauses 1-37, wherein the chimeric polypeptide is expressed on the surface of the outer membrane vesicle.
46. The outer membrane vesicle of clause 45 wherein the outer membrane vesicle is a native outer membrane vesicle, preferably obtained or obtainable without use of a solubilization agent, most preferably obtained or obtainable without use of a detergent.
47. The outer membrane vesicle of clause 45 or clause 46 wherein the outer membrane vesicle is obtained or obtainable from a genetically modified gram-negative bacterium.
48. The outer membrane vesicle of clause 47 wherein the gram-negative bacterium is Neisseria meningitidis, Neisseria gonorrhoeae, Escherichia coli, Bordetella pertussis, Non-typhoidal salmonella, Shigella sonnei, Klebsiella pneumoniae, Mycobacterium tuberculosis, or Vibrio cholerae.
49. The outer membrane vesicle of clause 48 wherein the genetic modification results in the gram-negative bacterium being hyper-blebbing, optionally wherein the genetic modification is a deletion or inactivation of the ompA gene, preferably deletion of the ompA gene.
50. An isolated polynucleotide encoding the chimeric polypeptide of any one of clauses 1-40 or the nanoparticle of any one of clauses 41-44.
51. An expression vector comprising the polynucleotide of clause 50 operably linked to regulatory sequences which permit expression of the chimeric polypeptide or nanoparticle.
52. A host cell or cell-free expression system containing the expression vector of clause 51.
53. A method of producing a chimeric polypeptide or nanoparticle comprising culturing the host cell or cell-free expression system of clause 52 under conditions which permit expression of chimeric polypeptide or nanoparticle and recovering the expressed chimeric polypeptide or nanoparticle.
54. A pharmaceutical composition comprising the chimeric polypeptide of any one of clauses 1-40, the nanoparticle of any one of clauses 41-44, or the outer membrane vesicle of any one of clauses 45-49, and at least one pharmaceutically acceptable carrier, adjuvant or excipient.
55. A chimeric polypeptide according to any one of clauses 1-40, a nanoparticle according to any one of clauses 41-44, an outer membrane vesicle according to any one of clauses 45-49, or a pharmaceutical composition according to clause 54 for use in therapy.
56. A chimeric polypeptide according to any one of clauses 1-40, a nanoparticle according to any one of clauses 41-44, an outer membrane vesicle according to any one of clauses 45-49, or a pharmaceutical composition according to clause 54 for use in treating or preventing cancer.
57. A chimeric polypeptide according to any one of clauses 1-40, a nanoparticle according to any one of clauses 41-44, an outer membrane vesicle according to any one of clauses 45-49, or a pharmaceutical composition according to clause 54 for r use in treating or preventing a pathogenic infection.
58. A chimeric polypeptide, nanoparticle, outer membrane vesicle, or pharmaceutical composition for use according to clause 57, wherein the pathogenic infection is caused by a pathogen from which one or more of the exogenous polypeptide(s) is derived.
59. A method of treatment or prevention comprising administering a chimeric polypeptide according to any one of clauses 1-40, a nanoparticle according to any one of clauses 41-44, an outer membrane vesicle according to any one of clauses 45-49, or a pharmaceutical composition according to clause 54.
60. A method of treating or preventing cancer comprising administering the chimeric polypeptide according to any one of clauses 1-40, a nanoparticle according to any one of clauses 41-44, an outer membrane vesicle according to any one of clauses 45-49, or a pharmaceutical composition according to clause 54.
61. A method of treating or preventing a pathogenic infection comprising administering a the chimeric polypeptide according to any one of clauses 1-40, a nanoparticle according to any one of clauses 41-44, an outer membrane vesicle according to any one of clauses 45-49, or a pharmaceutical composition according to clause 54.
62. A method of treatment or prevention according to clause 61, wherein the pathogenic infection is caused by a pathogen from which one or more of the exogenous polypeptide(s) is derived.
63. Use of a chimeric polypeptide according to any one of clauses 1-40, a nanoparticle according to any one of clauses 41-44, an outer membrane vesicle according to any one of clauses 45-49 in the manufacture of a medicament.
64. Use of a chimeric polypeptide according to any one of clauses 1-40, a nanoparticle according to any one of clauses 41-44, an outer membrane vesicle according to any one of clauses 45-49 in the manufacture of a medicament for treating or preventing cancer.
65. Use of a chimeric polypeptide according to any one of clauses 1-40, a nanoparticle according to any one of clauses 41-44, an outer membrane vesicle according to any one of clauses 45-49 in the manufacture of a medicament for treating or preventing a pathogenic infection.
66. The use according to clause 65, wherein the pathogenic infection is caused by a pathogen from which one or more of the exogenous polypeptide(s) is derived.
67. A method for raising an immune response in a mammal, comprising administering chimeric polypeptide according to any one of clauses 1-40, a nanoparticle according to any one of clauses 41-44, an outer membrane vesicle according to any one of clauses 45-49, or a pharmaceutical composition according to clause 54.
68. A vaccine or immunogenic composition comprising a chimeric polypeptide according to any one of clauses 1-40, a nanoparticle according to any one of clauses 41-44, an outer membrane vesicle according to any one of clauses 45-49, or a pharmaceutical composition according to clause 54.
69. A method of screening for an antibody which binds a target protein, comprising:
   (i) exposing a population of antibodies to a chimeric polypeptide according to any one of clauses 1-40, a nanoparticle according to any one of clauses 41-44, an outer membrane vesicle according to any one of clauses 45-49,
      wherein one or more of the exogenous polypeptide(s) of the chimeric polypeptide comprises an antigenic fragment of the target protein; and
   (ii) identifying those antibodies which bind to the chimeric polypeptide as binding to the target protein.
70. An array comprising a plurality of chimeric polypeptides according to any one of clauses 1-40, a plurality of nanoparticles according to any one of clauses 41-44, or a plurality of outer membrane vesicles according to any one of clauses 45-49.
71. A kit comprising one or more chimeric polypeptides according to any one of clauses 1-40, nanoparticles according to any one of clauses 41-44, or outer membrane vesicles according to any one of clauses 45-49, and optionally instructions for their use.

### EXAMPLES

The invention will be further understood with reference to the following non-limiting experimental examples.

### Example 1: Materials and Methods

### Epitope Identification and 3D Structure Prediction of Target Antigens

The amino acid sequences encoding for opaB and PorB proteins were extrapolated from the genome of N. gonorrhoeae strains FA1090 and F62 collected in public databases for molecular typing and microbial genome diversity (PubMLST). The leader sequences were removed from both protein sequences and the coding genes were used for the recombinant production of whole antigens. Instead, the sequences of OpaB and PorB target loops as well as the flexible regions of D3 scaffold were identified starting from their three-dimensional structures. As regarding the structures of PorB and OpaB, they were computationally predicted with Swiss model, AlphaFold or Rosetta comparative modelling, while the structure of D3 (in BP-2a) is available in the PDB (PDB code: 2XTL).

### Design and Production of Recombinant Soluble Proteins

Chimeric polypeptides displaying OpaB and PorB loops were designed so as to insert the OpaB and PorB loops (the target epitopes) into flexible sites (i.e. endogenous loops) of the BP-2a D3 polypeptide comprised in the chimeric polypeptide. The genes encoding for the designed chimeric molecules were codon optimised for E. coli expression and produced as DNA strings by GeneArt (Thermo Fisher Scientific). The DNA strings were cloned into pET15b+ (Merck-Sigma) using an enzyme-free cloning strategy available commercially as an In-fusion cloning kit (Takara). Modified plasmids also comprised an IPTG-inducible T7 promoter, a nucleotide sequence encoding a His6 tag, and an Ampicillin resistance gene.

Recombinant plasmids were propagated in E. coli Stellar cells grown on selective Luria-Bertani (LB) agar plates (Tryptone 10 g/L, yeast extract 5 g/L, NaCl 10 g/L 1.5% agar) containing ampicillin [100 μg/mL]. Grown colonies were screened through PCR. The recombinant plasmids from positive colonies were extracted with E.Z.N.A Plasmid DNA Mini Kit (Omega Biotech) following manufacturer instructions and the correct sequences were further confirmed by next generation sequencing (NGS).

Expression of the chimeric polypeptides was achieved in the cytoplasm of E. coli BL21(DE3)t1r obtained from NEB. Cells were grown in HTMC expression medium (Glycerol 15 g/L; Yeast Extract 30 g/L, MgSO₄×7H₂O 0.5 g/L; KH₂PO₄ 5 g/L; K₂HPO₄ 20 g/L; KOH 1M to pH final 7.35±0.1) for 18 h at 20° C., then induction was performed by adding IPTG 1 mM to the cell culture and incubating for 24 h. The soluble proteins were chemically extracted by using the CelLytic reagent (Sigma-Aldrich) solubilized in distilled water followed by centrifugation to remove cell debris. The recombinant soluble proteins were purified from the supernatant using IMAC chromatography eluting the protein of interest with 250 mM imidazole in PBS. Buffer was exchanged in PBS by using 10 kDa cut-off spin concentrator (Millipore Amicon Ultra).

For the recombinant production of OpaB and PorB in E. coli without use of the chimeric polypeptide scaffold, insoluble proteins were recovered from inclusion bodies after lysis in presence of 50 mM Tris-HCl and 8M urea at pH8. They were purified with IMAC chromatography in denaturing conditions maintaining 8M urea and eluting the protein of interest with 250 mM imidazole in PBS. The samples were then dialysed for about 24 h against PBS containing 0.05% n-Dodecyl-B-D-Maltoside (DDM) for OpaB and 1% N,N-Dimethyl-n-dodecylamine-N-oxide (LDAO) for PorB.

SDS-PAGE was performed to determine protein purity and the concentration was determined with Nanodrop absorbance at 280 nm.

### Immunization Protocols—In Vivo Studies

Animal treatments were performed in compliance with the Italian laws and approved by the institutional review board (Animal Ethical Committee) of GSK Vaccines Siena, Italy.

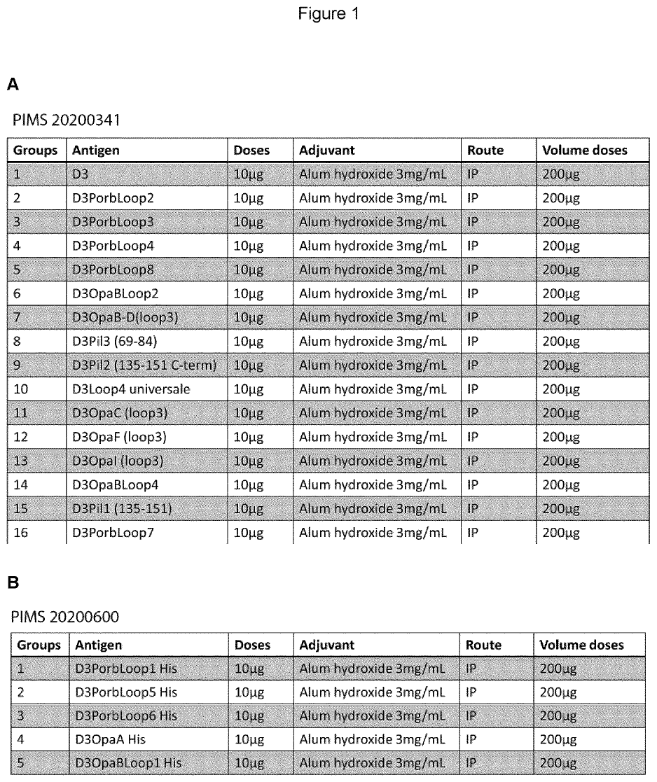

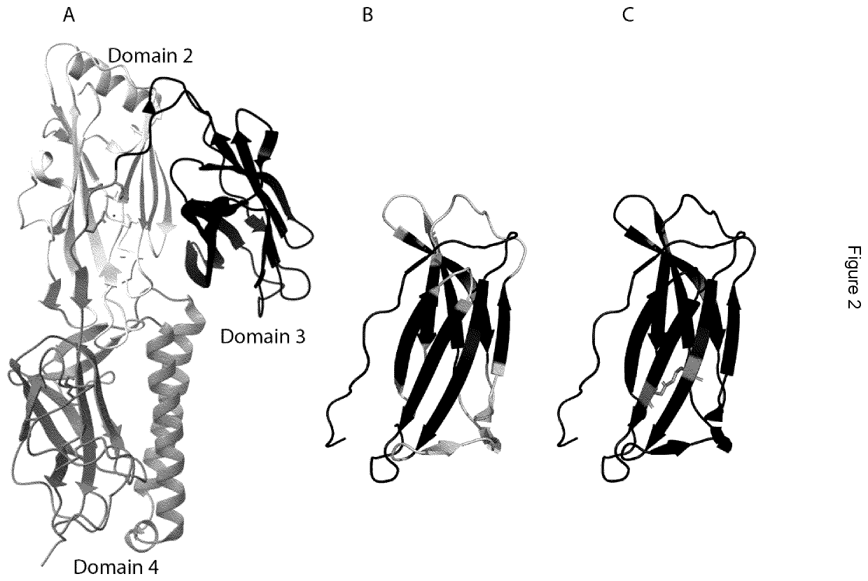

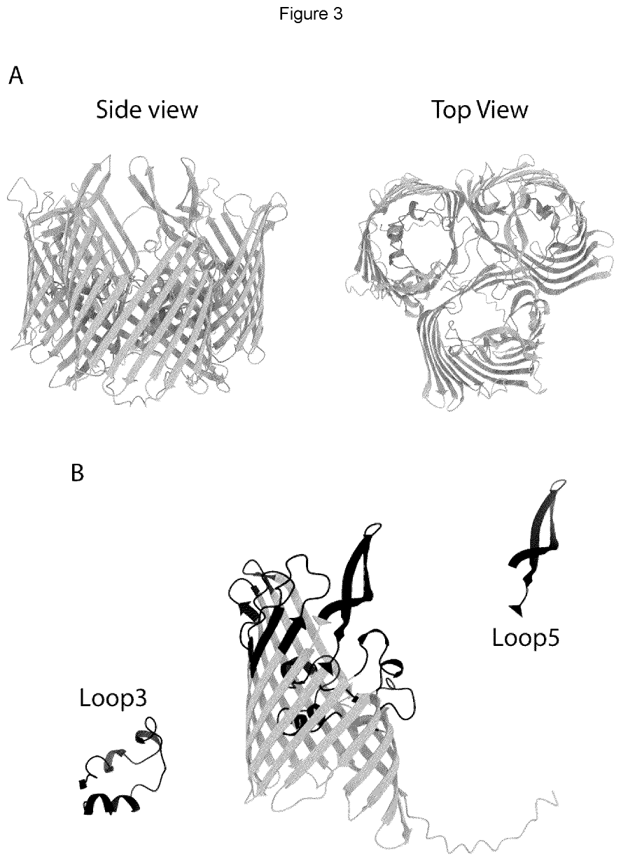

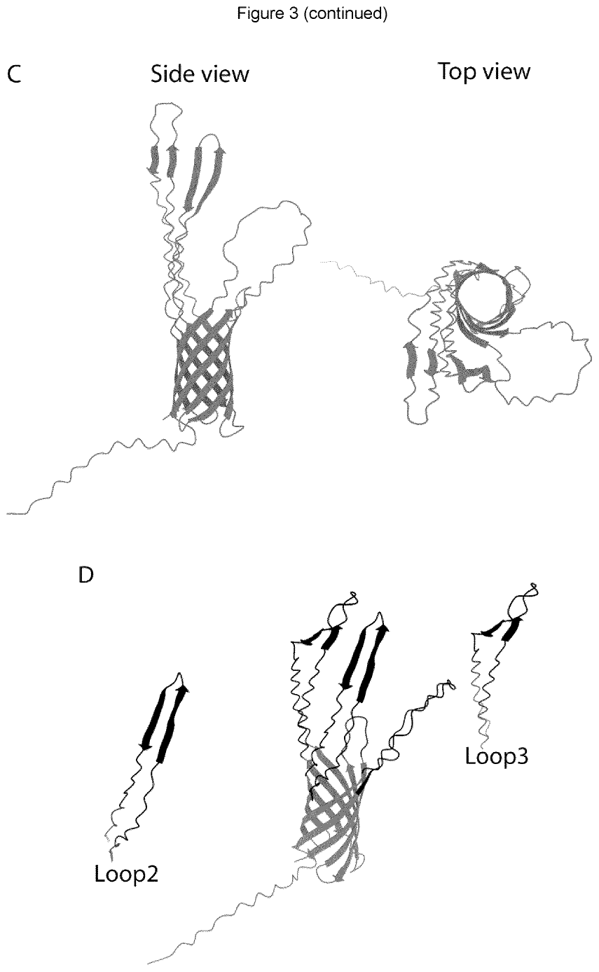

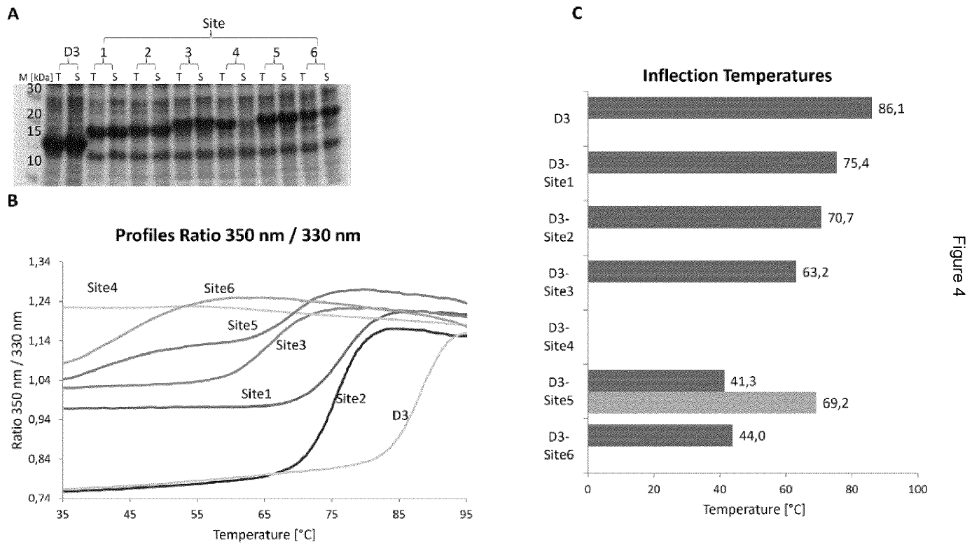

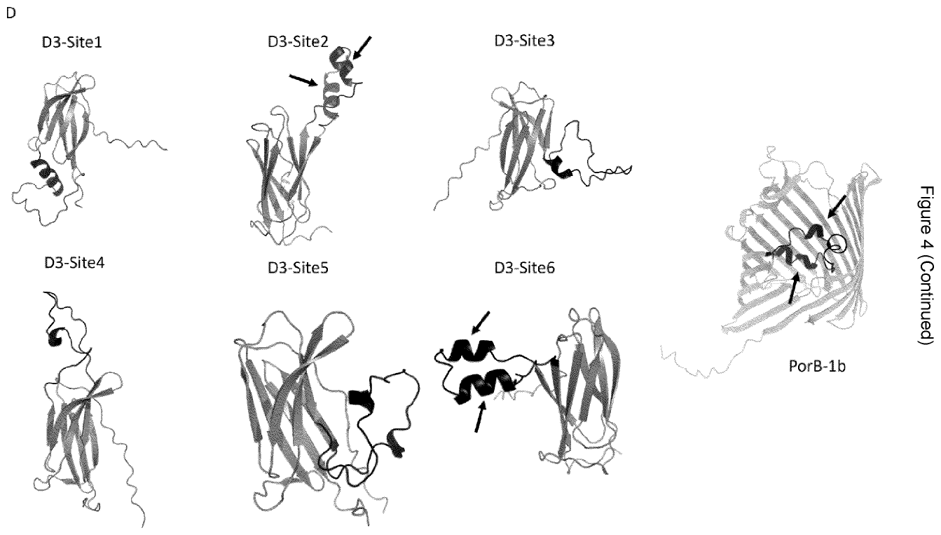

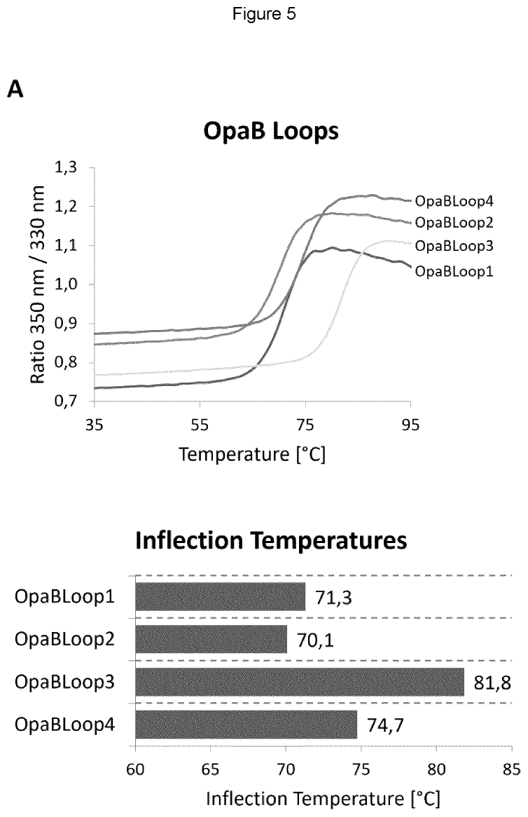

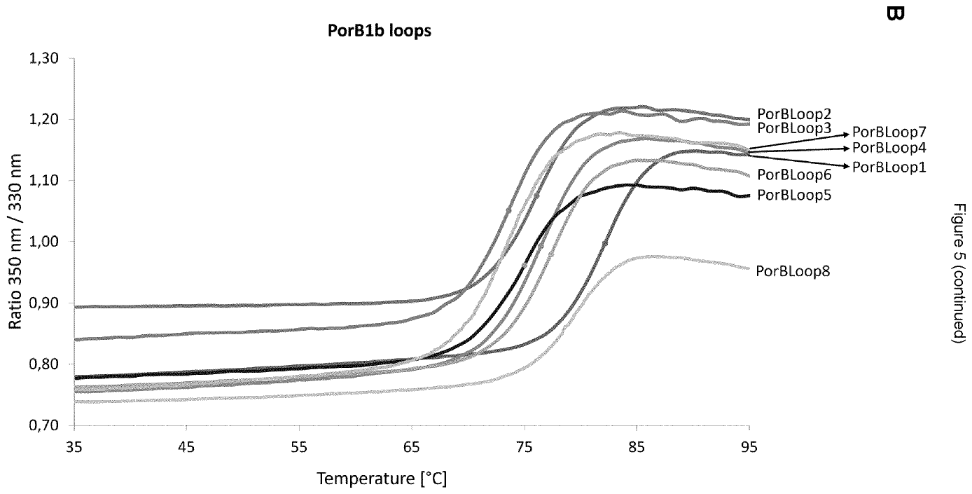

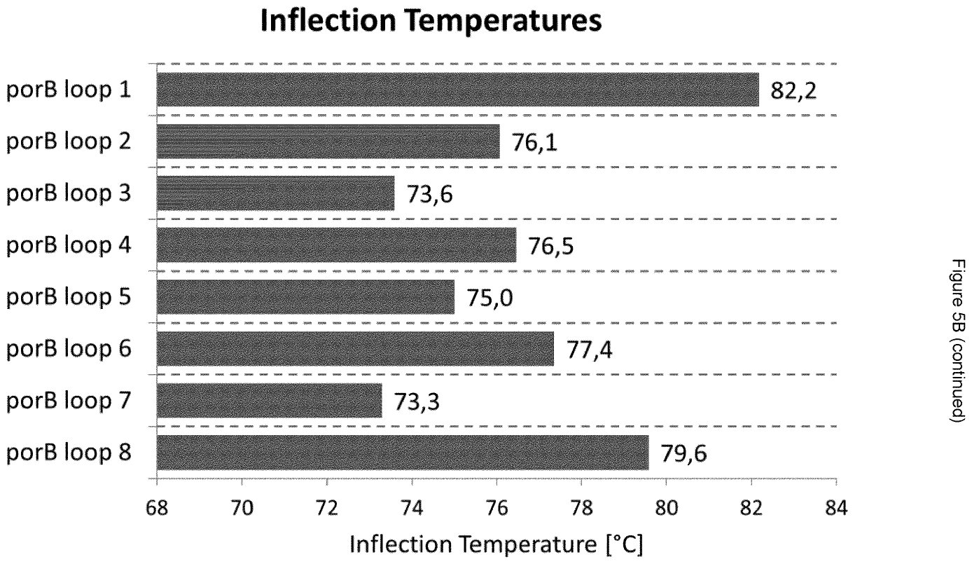

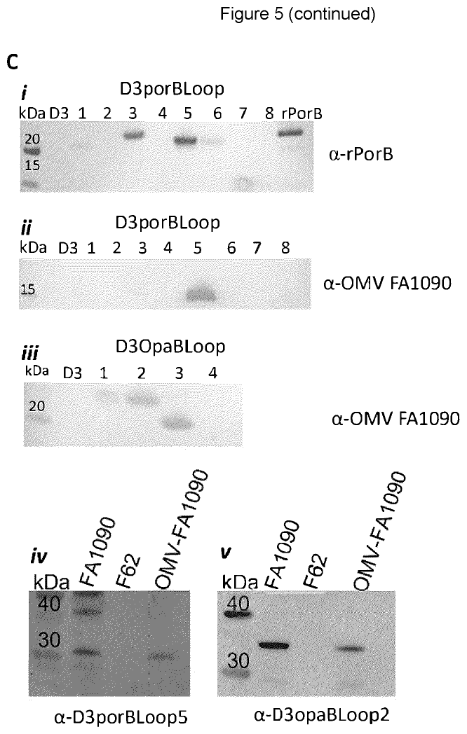

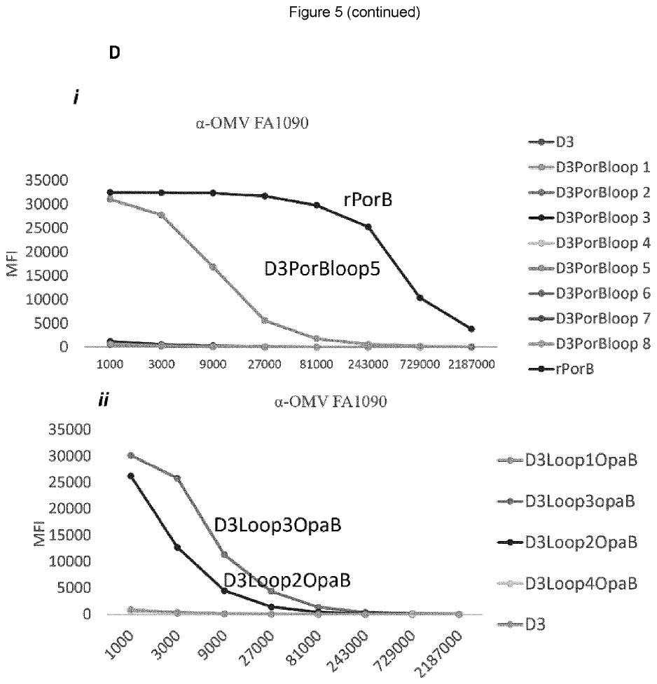

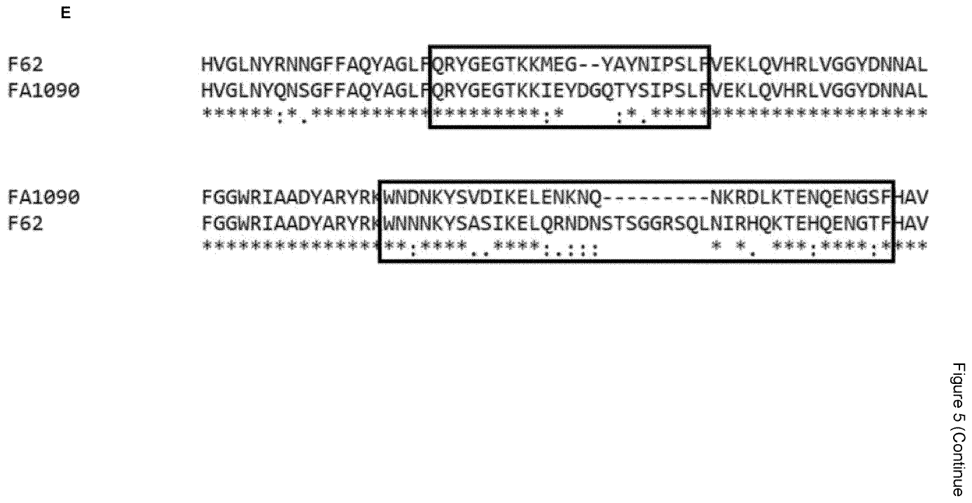

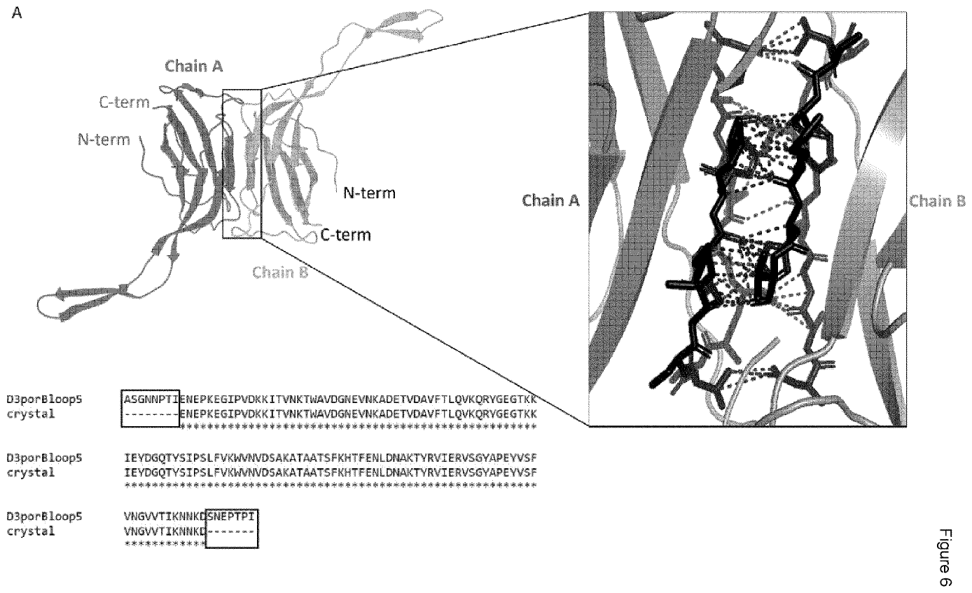

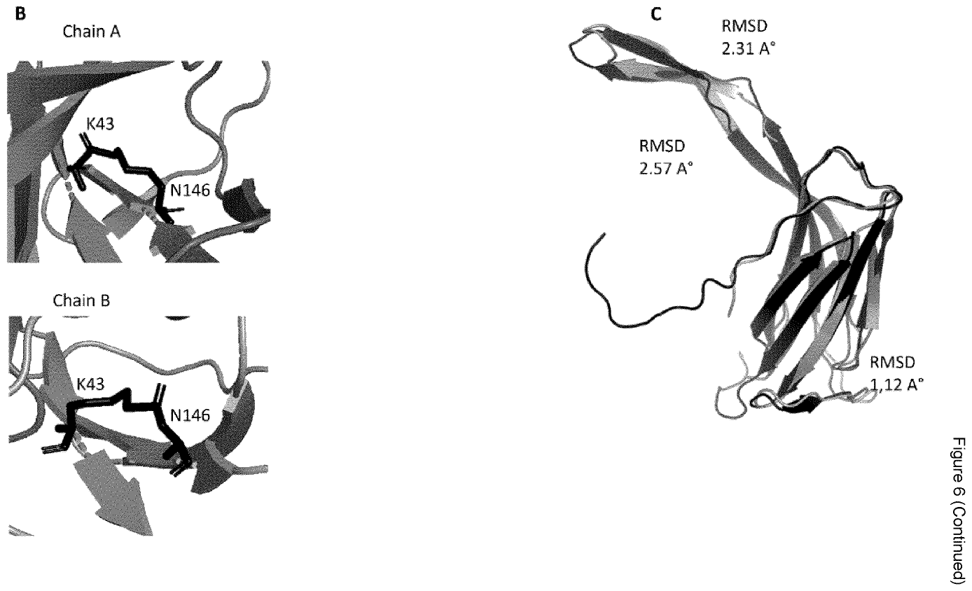

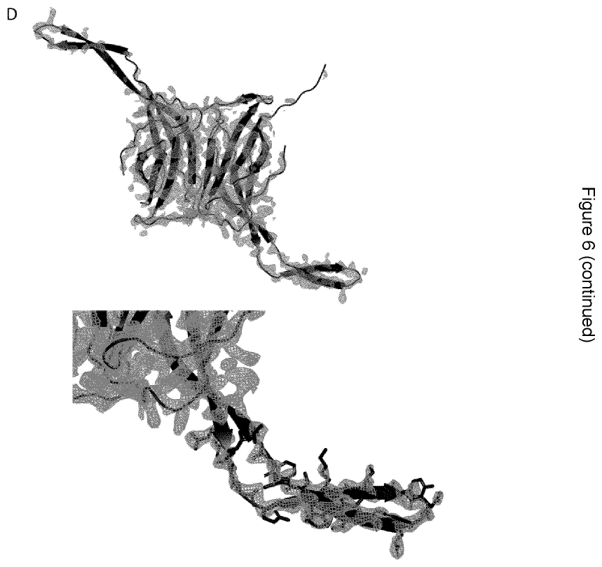

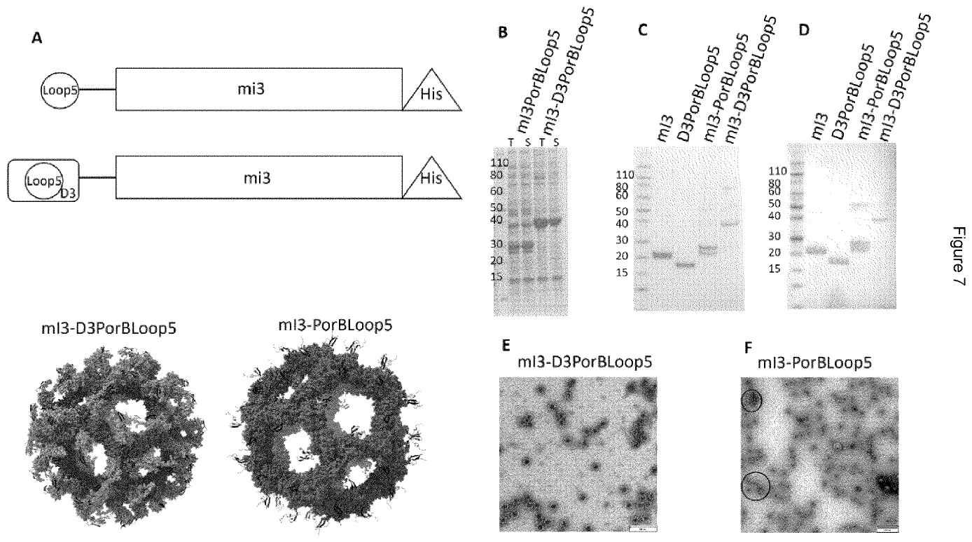

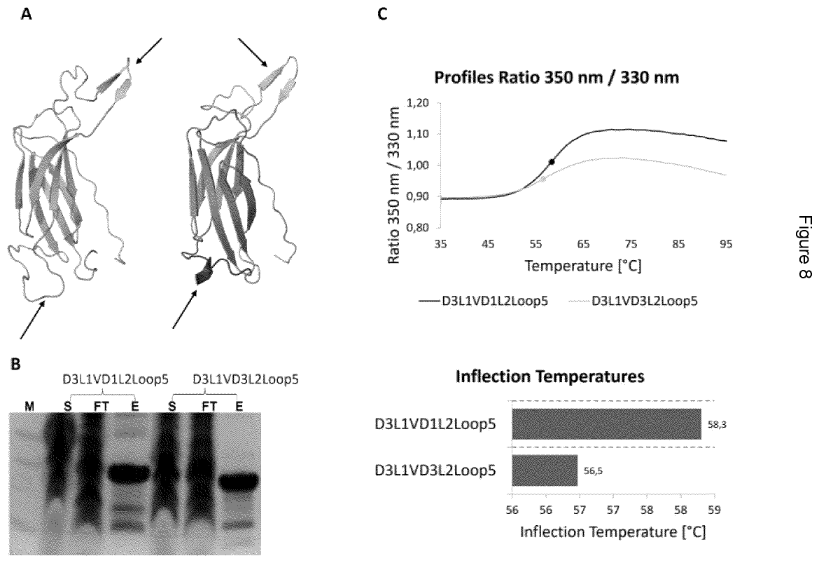

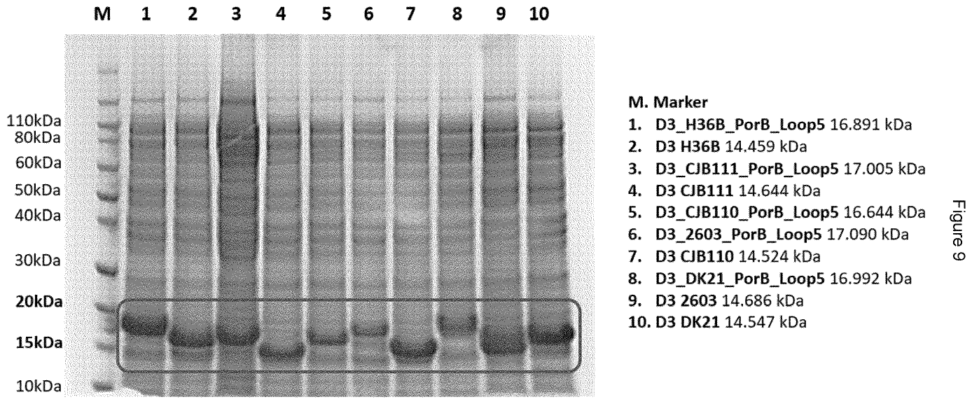

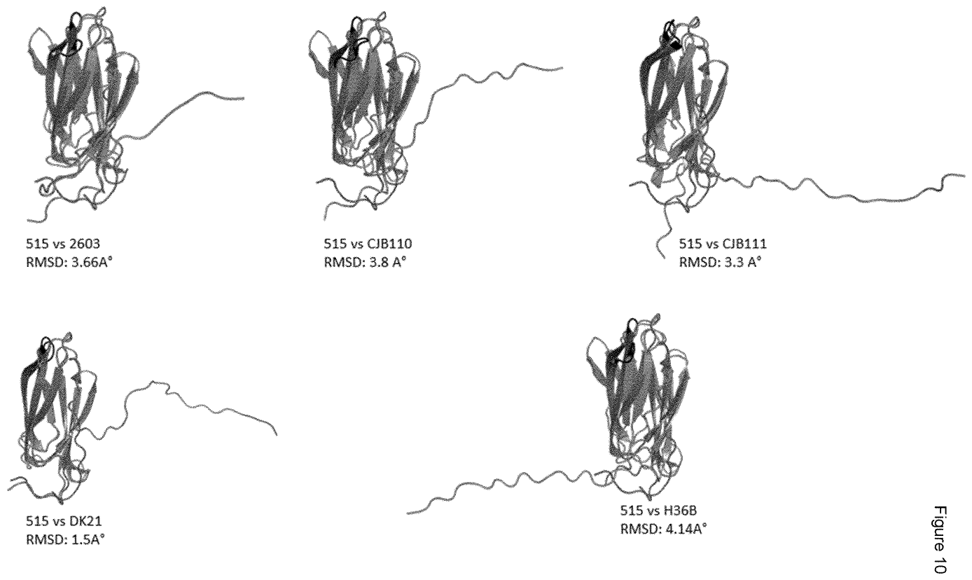

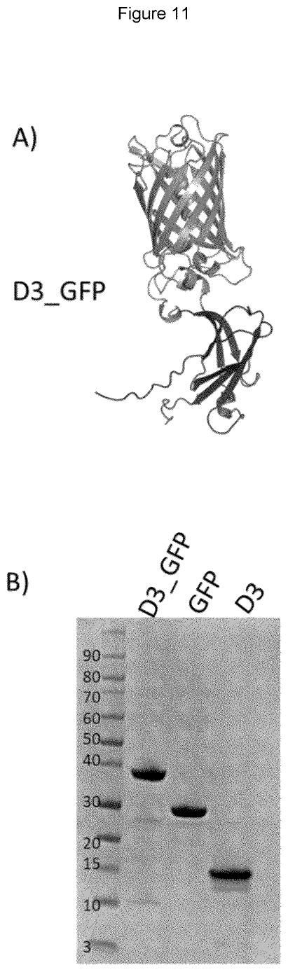

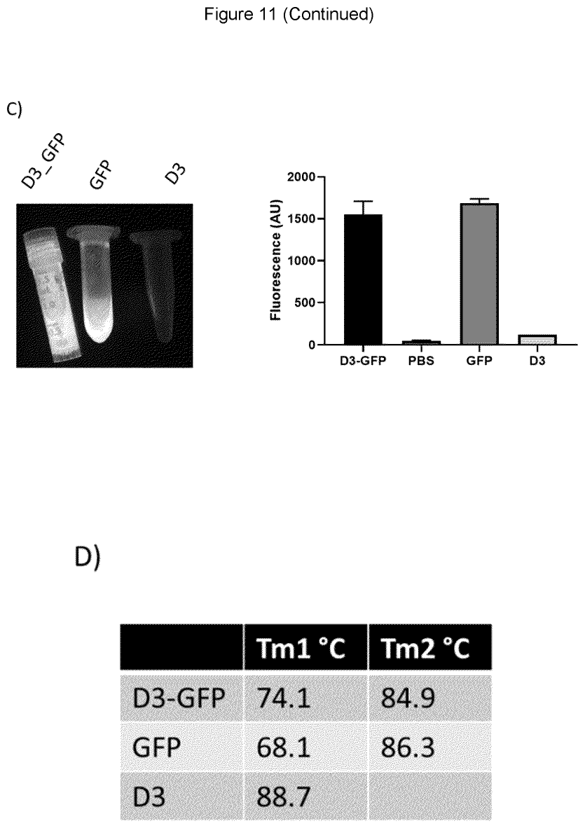

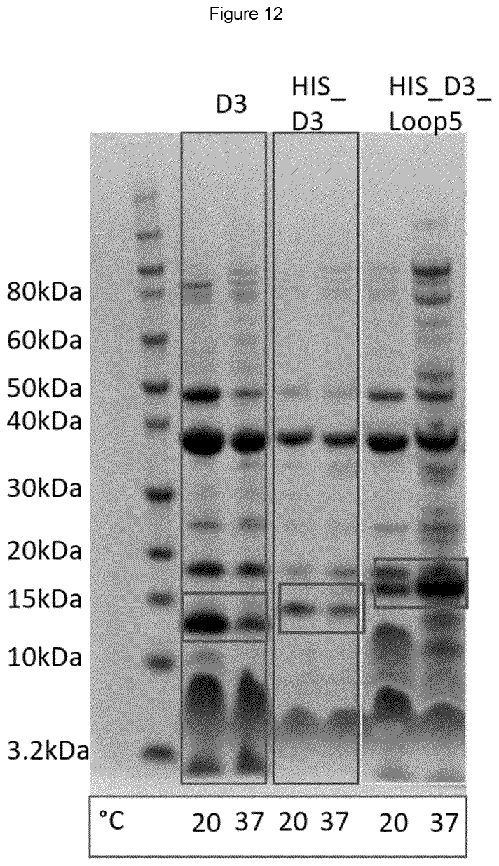

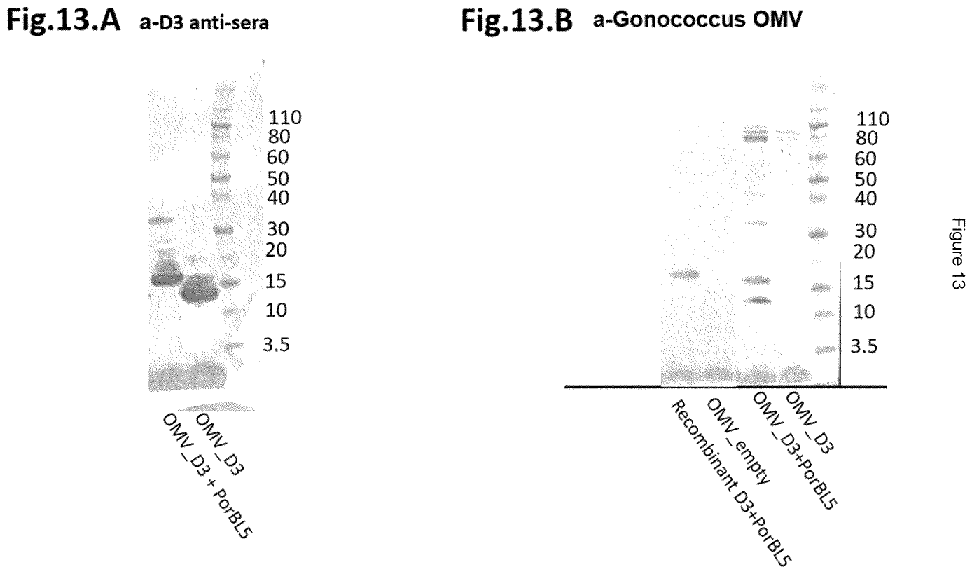

10 μg of each purified recombinant chimera mixed with AS01 adjuvant was used to intraperitoneally immunize 10 female CD1 mice at 7 weeks old. Three different immunizations were performed at days 0, 21 and 37 collecting sera samples at each point (FIGS. 1A, 1B, 1C).

### NanoDSF

The analysis was performed with Tycho NT 6 (Nanotemper). A capillary was filled with the sample [0.5 mg/mL] and a linear temperature ramp from 25° C. to 91° C. was applied to unfold proteins. During the scanning the increase of intrinsic tryptophan or tyrosine fluorescence was recorded and the ‘melting temperature’ or ‘Tm’, corresponding to the midpoint of the transition from folded to unfolded was evaluated. Data were analyzed in triplicate and analysed with Excel.

### Differential Scanning Calorimetry (DSC)

Using a MicroCal VP-Capillary DSC instrument (GE Healthcare), 10-μM concentration of each protein was analysed with a temperature scan range from 10-110° C., a thermal ramp rate of 180° C./h, and a 4-s filter period. Data were analysed with Origin 7 software.

### Western Blot

Purified recombinant proteins were transferred to a nitrocellulose membrane after an SDS-PAGE run using i-transfer and i-Blot Mini/regular kit (ThermoFisher scientific). The membrane was then blocked with 3% milk in PBS+0.1% TWEEN for 1 h at room temperature (RT). After incubations with primary and secondary antibodies (Ab), the membrane was washed three times with PBS+0.1% TWEEN for 5 min under shaking in order to remove unbound Ab. The result was detected via the colorimetric reaction between horseradish peroxidase (HRP) conjugated to the secondary antibody and its substrate 4-chloro-1-naphthol.

### Luminex Assay

Luminex Magplex beads were equilibrated at RT. 100 μL of resuspended beads (1.25×10⁶) were transferred to a LoBind Eppendorf tube and placed into a magnetic separator for 2 min. The supernatant was removed, and the beads were washed with water and activated for 20 min with NHS EDC in 100 mM of monobasic sodium phosphate pH 6.2 buffer and washed twice with 50 mM MES. The activated beads were incubated for 2 h with 200 μg of the molecule of interest. Coupled beads were finally washed twice with 1×PBS+0.05% Tween and stored in 500 μL of assay buffer (1×PBS, 0.5% TWEEN and 0.05% BSA) at 4°.

Standard sera and sera were pre-diluted in assay buffer 3-fold dilution were performed in 50 μL final volume for each well of Grainer microtiter plate. 50 μL of coupled beads were added to sera and the plate was incubated for 1 h at RT in the dark on a plate shaker at 700 rpm. Unbound Ab were removed by washing plates three times with 200 μL of PBS using an automatic plate washer with magnetic plate holder. Each well was then loaded with 50 μL of secondary antibody conjugated with R-phycoerythrin-AffiniPure and incubated for 1 h at RT in the dark on a plate shaker at 700 rpm. After washing, beads were suspended in 100 μL of PBS and analysed with Bioplex 200. Data were acquired in real time by Bioplex Manager Software 6.1 (BioRad).

### Negative Staining Electron Microscopy

The electron microscopy analysis was performed loading 5 μl of sample concentrated 20 ng/μL onto a glow discharged copper 300-square mesh grid for 30 s. Blotted the excess, the grid was negatively stained using NanoW for 30 seconds. The samples were analysed using a Tecnai G2 spirit and the images were acquired using a Tvips TemCam-F216 (EM-Menu software).

### Protein Crystallization and Structure Determination

Purified recombinant protein D3PorBLoop5 was concentrated to a concentration of 25 mg/mL in 20 mM Tris-HCl, 150 mM NaCl at pH 8.0. Using a Crystal Gryphon robot (Art Robbins Instruments), 800 different crystallization experiments were setup by using 200 nL reservoir and 200 nL protein sample. The best crystal was grown in buffer containing 0.1M HEPES with 20% w/v jeff ED-2001 as precipitant at pH 6.5. Crystals were soaked in the original mother liquor supplemented with 15% ethylene glycol prior to cryo-cooling in liquid nitrogen. Diffraction of the crystals was performed at beamline ID30 Å-1 of the European Synchrotron Radiation Facility (ESRF) and several data were collected at 100K, at wavelength λ=0.96546 Å. 0.96546 frames. Data were processed using autoProc and they were reduced using Scala within the CCP4 program suite. Crystals of the D3-Loop 5 chimera belong to space group F222, with the asymmetric unit containing five copies and a solvent content of 49.2% (Matthew's coefficient of 2.42 Å3/Da). The structure of the D3PorBLoop5 was determined at 2.6 Å resolution by molecular replacement with Phaser using two separate search models obtained from homology modelling simulation and from 2XTL data collected in PDB database. Rigid body and restrained refinement were carried out with Refmac5 (from CCP4i suite). Rigid body and restrained refinement were carried out with Refmac5 (from CCP4i suite). Structure quality was assessed using Molprobit, while protein-protein interface areas were analysed and calculated using the Protein Interfaces, Surfaces and Assemblies service (PISA) by PDBePISA. Figures were generated using PyMOL.

### GFP Fluorescence Assay

The fluorescence of recombinant purified D3-GFP protein was measured via fluorescence spectroscopy in black flat bottom 96-well plates using a Tecan Infinite M200 reader (Tecan, Mannedorf, Switzerland). A final volume of 100 μL of 0.85 mg\ml of each protein sample was transferred into the 96-black flat bottom well plate (Greiner Bio-One, Frickenhausen, Germany) and the fluorescence emission was recorded. The excitation wavelength was 395 nm (bandwidth 10 nm) and the emission spectrum was recorder with gain 80 at 448 nm. The intensity recorded for the D3 alone in PBS buffer at 448 nm was subtracted from that recorded for GFP and D3-GFP to remove background.

### OMVs Preparation

Recombinant E. coli strains expressing D3 empty and D3-PorBloop5 were pre-cultured for 6 to 7 hours in 5 ml of LB supplemented with kanamicin and grown at 37° C., at 180 rpm. Pre-cultures were then diluted 1:100 in 75 ml High Throughput Medium Complex (HTMC) (3% yeast extract, 1.5% glycerol, 40 mM KH2PO4, 90 mM K2HPO4, 2 mM MgSO4·7H2O, pH 7.4) supplemented with kanamicin and grown overnight at 20° C., 160 rpm. The cultures were then centrifuged for 10 min at 1800×g at 20° C. Supernatants were discarded to remove Empty OMVs and the bacterial pellet was resuspended in 75 ml of fresh HTMC supplemented with the antibiotic. Expression was induced with iso-propyl β-d-1-thiogalactopyranoside (IPTG) (Sigma-Aldrich) at a final concentration of 1 mM for 6 h at 37° C., 160 rpm. The cultures were clarified by centrifugation for 20 min at 3000×g and the culture media were filtered through a 0.22-μm pore size filter (Millipore). Supernatants were subjected to high-speed centrifugation at 119,000×g for 2 h at 4° C. (Beckman Coulter Optima Ultracentrifuge) and the pellets containing the OMVs were washed with phosphate buffer saline (PBS) two times and then filtered with 0.22-μm filter. OMVs total protein content was quantified through the Lowry assay (DC Protein Assay, Bio-Rad).

### Results

### Example 2: Structural Analysis of GBS D3 Allowed the Identification of Potential Engineerable Sites

By using a structure-based design approach six different D3 sites corresponding to its loops were identified to be potentially engineerable. In fact, D3 folded into a 3-barrel structure independently protruding from BP-2a. The 3-barrel was composed of 6 spanning segments connected by 6 flexible loops (FIG. 2).

### Example 3: Structural Prediction and Identification of Extracellular Loops of PorB1b and OpaB

A structural analysis was also performed for the identification of extracellular PorB and OpaB loops. As the best of our knowledge the 3D structures of both antigens, PorB1b and OpaB, were not publicly available and only computational in silico predictions are reported based on the sequence homology with structurally known proteins. From the reported predictions, PorB1b should form a homotrimer in which each monomer should be formed by 16 transmembrane-spanning segments and 8 highly flexible extracellular loops (FIG. 3A). While OpaB, one out of eight Opa variants encoded by the genome of N. gonorrhoeae FA1090, was predicted to be structured as eight spanning stranded forming a β-barrel, connected by four extracellular loops. The specificity for host receptors is conferred by the semi- and hyper-variable regions found in these flexible loops.

However, thanks to the advances in the structural prediction field it is now possible to obtain models with a level of accuracy comparable to the experimental structures exploiting deep learning techniques. Following this direction, Artificial Intelligence (AI) approach named AlphaFold2 was applied to predict PorB1b and OpaB 3D structure. The analysis revealed the canonical 3-fold PorB symmetry with 16-stranded β-barrel, short turns connecting the strands on the periplasm and long interstrand loops on the extracellular part of the pore for each of the monomer (FIG. 3a). Interestingly, it was observed that two of the eight loops present a secondary structure. In particular, Loop5 is structured as 3-hairpin which is solvent exposed and oriented towards the central channel pore of the monomer. While Loop3 present two short-helical turns and it is predicted to be directed inside the pore (FIG. 3B). The per-residue measurement of model local confidence (pLDDT) revealed high confidence for the transmembrane β-barrel predicted region (pLDDT≥50) but low confidence (pLDDT≤30) in the loop's region. This is probably due to the intrinsic flexibility of the loops. However, this data was further confirmed crystallographically (see FIG. 6).

Alphafold prediction of OpaB models was in agreement with the secondary structure prediction of 8 antiparallel β-strands, forming a barrel structure in the bacterial outer membrane, linked by 4 extracellular loops (FIG. 3C). Two out of four loops, namely Loop2 and Loop3, showed the same beta-hairpin structure previously observed in PorB1b/L5 (FIG. 3D). The pLDDT score revealed high confidence for the β-strands region (pLDDT≥50) but low confidence in the OpaB/L2-L3 region (pLDDT≤30). Despite the lack of OpaB X-ray structure to further validate the model, the results obtained for loop5 PorB1b were encouraging to validate in silico OpaB models too.

Finally, the 3D structure prediction of both PorB1b and OpaB antigens allowed the identification of 8 (PorB1b) and 4 (opaB) extracellular loops (FIG. 3B-D). In order to identify most immunogenic/immunodominant epitopes, they were extrapolated and inserted into a D3 site2.

### Example 4: D3 Site2 is the Optimal Site for the Insertion of OpaB and PorB1b Loops

The best engineerable D3 site was chosen taking in consideration the expression level (FIG. 4A), the thermal stability (FIG. 4B) and the ability to preserve the epitope conformation of 6 different chimeras. The analysis was performed by inserting the longest epitope identified in the model antigens (PorB loop3) into each of the six D3 sites. All chimeras were expressed in soluble form in E. coli at levels comparable to the empty D3, only the engineering of D3 site4 seems to generate a less soluble chimera not correctly folded. In fact, from Nano DSF analysis it was not possible to appreciate for this chimera a transition between the folded and unfolded states suggesting that the protein is not properly structured (FIG. 4B). Whereas the engineering of all the other D3 sites generated chimeras that presented a shift in the measured fluorescence at temperatures in the range of 45-76° C. (FIG. 4C) suggesting that they are folded. Each chimeras presented a Tm lower than the Tm detected for the scaffold alone (88° C.), but this was reasonably due to the insertion of a long and flexible foreign portion. Among all tested positions, the engineering of site1 and site2 led to the formation of most stable chimeras. The only note is that the higher initial ratio 350/330 nm detected for site1 chimera suggested the presence of partially unfolded regions. While the D3-Site2 showed a profile similar to the empty D3 scaffold with the lowest initial 350/330 nm ratio. Another key factor for the choice of best insertion position was the ability to preserve the native epitope conformation. For this reason, the 3D structure of each chimeric D3 displaying PorB1B loop3 was computationally predicted with Alphafold (FIG. 4D) and the epitope conformation was compared with the one predicted in the native protein (FIG. 4D). From this analysis, loop3 PorB1b resulted to maintain its α-helices conformation when inserted into D3 site2 and site6. In all the other sites the epitope was partially restructured with respect to its predicted native conformation in the whole protein. Combining all these data, the engineering of D3 site2 resulted to be the most promising strategy to produce soluble and stable chimeras able to correctly display the epitope of interest. For these reasons, all PorB1b and OpaB loops were extrapolated and inserted into D3 site2.

### Example 5: D3 Correctly Displays Gonococcal Epitopes

Once the best D3 site was identified, all the PorB1b and OpaB loops were extrapolated and inserted into D3 site2. All chimeras were obtained as His-tagged soluble proteins in the cytoplasm of E. coli.

Structural characterization of purified protein revealed that the insertion of large epitopes (tested up to 34 amino acids) did not destroy scaffold structure. In fact, thermostability analysis revealed that each molecule was properly folded with a Tm ranging from 70 to 82° C. (FIGS. 5A,B). Furthermore, foreign epitopes were correctly displayed on the scaffold and accessible to the antibodies. As summarised by the below data:
PorB1b loops were well recognized in Western Blot by two different specific sera. In particular, PorB1b loops 1-3-5 and 6 were recognized by α-rPorB1b serum (FIG. 5C-i). It is needed to precise that to avoid a specific recognition due to the presence of α-His in the α-rPorB serum, the His-tag of each construct was removed by TEV cleavage. While only the loop5 was recognized by α-OMV-FA1090 serum (FIG. 5C-ii). This result was also confirmed by Luminex assay (FIG. 5D-i).
On the other hand, α-OMV-FA1090 serum was able to recognize only loop2 and loop3 (FIG. 5C-iii). In fact, despite OpaB loop1 being poorly recognized in Western Blot by α-OMV-FA1090, it was not detected by the same serum with Luminex assay and OpaB loop4 was not been detected with these assays. This is in accordance with the information reported by Cole at al. (Cole, J. G. and A. E. Jerse, Functional characterization of antibodies against Neisseria gonorrhoeae opacity protein loops. PLoS One, 2009. 4(12): p. e8108), which defined Loop2 and Loop3 as the hypervariable loops of Opa proteins containing also the most immunogenic and functional epitope. Cole also reported that, although loop1 and loop4 were highly conserved among strains and variants, they were not able to induce the production of specific antibodies.
Moreover, the in vivo study conducted in mice revealed that the designed chimeras, when administered to mice, were able to elicit immune response against the target epitopes. In this sense, sera α-D3PorBLoop5 and α-D30paBLoop2 are able to recognize in Western Blot native proteins present in the total cell-extracts and exposed on the surface of OMV of N. gonorrhoeae FA1090 (FIG. 5C-iv-v). Accordingly, due to the high sequence diversity of PorB Loop5 and OpaB loop2 between strains FA1090 and F62, both tested sera do not recognize the total extract of F62 strain (FIG. 5E). From the analysis conducted, PorB1b loop5 and OpaB loop2 and loop3 resulted to be the most immunodominant epitopes of the two tested antigens.

### Example 6: Crystal Structure Resolution of D3PorBloop5 Confirmed the Correct Epitope Display

From the computational structural prediction PorB loop5 presented a β-hairpin structure and it was also predicted that the engineering of D3 site 2 preserves the epitope conformation. In order to confirm the results of the in-silico analysis, the 3D structure of D3PorBLoop5 (without a fusion tag) was solved by X-ray crystallography. Crystals were obtained after 6 days in buffer containing 0.1M HEPES with 20% w/v jeff ED-2001 precipitant at pH 6.5. The X-ray diffraction data were processed and the crystal structure of D3PorBloop5 was determined by molecular replacement using Phaser (Suite Phenix). Electron density maps were of high quality and allowed the model building and structure refinement to a final resolution of 2.7 Å. Although crystallization was carried out using entire D3loop5 chimera (139 amino acids), 8 N-terminal and 7 C-terminal residues were absent in the crystal (FIG. 6A). Based on the computational prediction, this was expected to be due to the high flexibility of these regions. The crystal asymmetric unit contained a dimer of two independent chains arranged in a mirror image. The second chain was rotated by 1800 about the y-axis compared to the first chain (FIG. 6A). The interface analysis performed with PISA revealed that the interface area represents about the 9% of the entire surface and the two monomers are taken together by multiple hydrogen bonds occurring between residues located in a β-strand (from residue 98 to 114) (FIG. 6A). In addition, for each chain the presence of the internal isopeptide bond between residues K43 and N146 (numbering of isopeptide bond location is in relation to D3PorBLoop5 i.e. SEQ ID NO:58) was detected (FIG. 6B). This result suggests that the D3 engineering with a foreign epitope did not alter its structure. Moreover, the foreign epitope displayed maintained its native conformation. Density map of PorB loop5 region, confirmed the 3-hairpin organization of this epitope (FIG. 6D). This result validated the computational structural prediction reported above of PorB1b. In addition, the model of chimeric D3PorBloop5 was computationally predicted and compared with the crystal structure obtained (FIG. 6C). The structures were aligned with an overall calculated RMSD of 2.31 Å. In particular, the structure alignment presented the highest similarity between the structures of the scaffold with an RMSD of 1.12. While the structure of PorBloop5 between crystallographic and predicted model presented a higher structural diversity with an RMSD of 2.53 (FIG. 6C). It is expected that the structural diversity detected was due to the flexibility of this region, nevertheless its 3-hairpin secondary structure was maintained. These results suggest that the insertion of exogenous polypeptides comprising epitopes into D3 site 2 (partially or wholly replacing endogenous loop 2) preserves native epitope conformation without destroying the scaffold structure.

### Example 7: D3 Scaffold Prevented Epitope Degradation when Displayed on mI3 Surface

After the identification of most immunodominant epitopes, and the confirmation that the native structure of the epitope was maintained when inserted into D3 scaffold, PorB1b loop5 was displayed on mI3 surface. The nanoparticle was decorated with both linear peptide (mI3-PorBloop5) and with chimeric D3 displaying PorB loop5 (mI3-D3PorBloop5). In both cases the antigens were genetically fused to the N-terminus of the mI3 nanoparticle (“NP”). The molecules were produced in E. coli and purified from the soluble fraction by affinity chromatography exploiting the 6×-His tag fused at the C-terminus of the scaffold (FIG. 7A). Then assembled particles were separated from monomers by SEC. Expression analysis as well as SDS-PAGE analysis of purified protein of mI3-PorBloop5 revealed the presence of double bands at the expected molecular weights (FIG. 7B-C). This could be due to a mixed population of intact mI3-PorBloop5 and partially degraded molecules. In order to understand which region, the N- or the C-terminus, of the chimera was degraded, a Western Blot was performed using an α-His antibody (FIG. 7D). In this analysis both bands were detected, indicating that the shift in the molecular weight was due to a degradation of the N-terminus corresponding to the linear peptide of PorB1b loop5. On the other hand, mI3-D3PorBloop5 was produced as a soluble chimera with the expected molecular weight (38.7 kDa) and no degradation was observed in any phase of the study. Negative stain electron microscopy analysis was conducted that confirmed the formation of properly assembled mI3-D3PorBloop5 NPs (FIG. 7E) and as regarding mI3-PorBloop5, a mixed population of nanoparticles was observed (FIG. 7F). Properly folded molecules were detected among partially structured particles as well as aggregated proteins. All these data demonstrate the importance of using a chimeric polypeptide in accordance with the invention for the display of epitopes on NP surface. This strategy facilitates the prevention of possible epitope degradation as well as the maintenance of native epitope conformation.

### Example 8: Simultaneous Engineering of Multiple D3 Sites

The presence of multiple sites (endogenous loops) in the BP-2a D3 polypeptide that can accommodate exogenous polypeptide make it suitable for use as a scaffold for the simultaneous display of two different epitopes. Although D3 site 2 resulted to be the best position for the insertion of foreign epitopes, additional sites (endogenous loops) were engineered by the insertion of unstructured epitopes. Two multivalent D3 based chimeric polypeptides were generated that simultaneously displayed both PorB loop5 and MOMP variable domain 1, or PorB loop5 and MOMP variable domain 3. MOMP porin is the major outer membrane protein of Chlamydia trachomatis and it is predicted to be structured as 3-barrel with 6 flexible loops. Four different highly immunogenic variable domains (VD) are located within the loops. The VD1 and VD3 were alternately extrapolated from loops 2 and loop5 respectively and inserted into D3 site1. In the same molecule, D3 site 2 was engineered by the insertion of PorB loop5. Two different multimeric D3 constructs (D3L1V D1L2Loop5, D3L1VD3L2Loop5) were designed, produced and characterized (FIG. 8). The 3D structure prediction of designed chimeras suggests that the two D3 sites chosen for the display of the two target epitopes prevented clashes between them. Moreover, each epitope maintained its native conformation even after the insertion into the chimeric polypeptide scaffold. The double engineering of D3 did not alter its solubility or overall structure. In fact, both D3L1VD1 L2Loop5 and D3L1VD3L2Loop5 expressed well and in soluble form in E. coli and were stable with Tms of 58 and 56° C., respectively (FIG. 8 B-C).

### Example 9: The D3 Scaffold from Five Additional GBS Strains that Express Different BP2a Variants were Capable of Displaying Exogenous Epitopes

Aim: To determine whether BP-2a D3 sequences from other GBS strains (i.e. not from the 515 strain) were capable of displaying exogenous epitopes. Note that previous Examples (see above) were performed using the BP-2a D3 sequence of strain 515.

Using the methods described in Example 1, chimeric polypeptides were produced displaying the PorB loop 5 epitope (SEQ ID NO: 21) as an exemplar epitope inserted in place of (either by partial or entire replacement) an endogenous loop of the BP-2a D3 polypeptide using the BP-2a D3 sequence from five additional GBS strains as follows (see table 5 below)

TABLE 5

| | | | Endogenous Loop | |
|---|---|---|---|---|
| | Sequence | PorB | of Bp2a Domain 3 | D3-PorBL5 |
| | of BP-2a | epitope | selected for | chimera |
| Strain | Domain 3 | inserted | engineering | sequence |
| H36B | SEQ ID | SEQ ID | 1 (SEQ ID | SEQ ID |
| | NO: 101 | NO: 21 | NO: 102) | NO: 190 |
| CJB111 | SEQ ID | (PorB | 2 (SEQ ID | SEQ ID |
| | NO: 116 | Loop 5) | NO: 118) | NO: 191 |
| CJB110 | SEQ ID | | 1 (SEQ ID | SEQ ID |
| | NO: 146 | | NO: 147) | NO: 192 |
| 2603 | SEQ ID | | 2 (SEQ ID | SEQ ID |
| | NO: 131 | | NO: 133) | NO: 193 |
| DK21 | SEQ ID | | 2 (SEQ ID | SEQ ID |
| | NO: 161 | | NO: 163) | NO: 194 |

As can be observed from the data in FIG. 9, chimeras that were engineered using D3 sequences from all GBS strains tested were successfully able to support the display of the PorB loop 5 foreign epitope. All chimeras were successfully expressed, soluble and at the expected molecular weight.

In addition, structural alignments were performed to examine the structural difference between the BP2a D3 from strain 515 with the Bp2a D3 from the other five GBS strains (i.e. H36_{B}, CJB111, CJB110, 2603 and DK21). Structural alignments and RMSD were calculated using pymol with the default setting of cealign function. As can be observed from FIG. 10, the root mean square deviation (RMSD) values indicate that the D3 scaffold from all GBS strains have very similar 3-D structures.

### Example 10: D3 Scaffold Capable of Displaying Entire Proteins

The green fluorescent protein (GFP) from the jellyfish Aequorea victoria is a soluble protein displaying visible fluorescent light at a wavelength of 508 nm after excitation with ultraviolet light (Ward et al., 1980). To produce a fluorescent signal, GFP must form and maintain its tight β-barrel structure. For this reason, GFP is usually used as protein reporter, for example to distinguish proteins that are correctly folded and soluble when expressed in E. coli from those which misfold and aggregate. In this study, GFP was used as a model protein to investigate if D3 could accommodate and correctly display an entire protein, instead of a loop only.

The AlphaFold2 3D structural model of D3-GFP was generated which demonstrated that flexible residues at the N-term and C-term of GFP could allow the correct β-barrel structure of GFP when inserted into the D3 scaffold without requiring linker residues (FIG. 11A). GFP (SEQ ID NO: 195) was thus engineered into D3Loop2 to produce the sequence of SEQ ID NO: 196. The plasmid pET29b+ encoding D3-GFP (inserted in D3 Loop2) was purchased from Twist Bioscience.

After cell lysis, the protein samples containing D3-GFP and GFP alone immediately appeared highly yellow suggesting the presence of folded GFP in the cell lysate. The two proteins were successfully purified from E. Coli cytoplasm by IMAC chromatography and were visualized by SDS-PAGE (FIG. 11B). Bands were observed at the anticipated MW (41 kDa for D3-GFP, 14.7 kDa for D3 alone and 26.7 kDa for GFP alone). UV-light exposure (FIG. 11C) also confirmed the presence of a folded GFP. Fluorescence of D3-GFP and GFP was measured at 0.85 mg\ml protein concentration in triplicate, confirming a similar fluorescence intensity for D3-GFP and GFP alone. No fluorescence was detected for D3 alone or PBS buffer, as expected.

Finally, thermostability analysis (using NanoDSF) confirmed the presence of two thermal transitions (FIG. 11D) for the D3-GFP construct suggesting that both D3 and GFP are properly folded.

All data collected show that D3 is able to correctly display an entire structured protein (such as GFP which is fluorescent only when correctly folded), confirming that the conformation of the inserted protein is maintained following engineering. These data show that D3 is an ideal protein scaffold not only for foreign protein loops of variable length but also with entire proteins.

### Example 11: D3 Scaffold Displays Epitopes when Accumulated in Outer Membrane Vesicles

Outer membrane vesicles (OMVs) are an important vaccine platform (see for example Irene et al 2019, PNAS, 116 (43) 21780-21788). Heterologous antigens can be expressed in the OMVs using different synthetic biology approaches. For example, adding a signal sequence at the N-terminus of the mature sequence of the target antigen, OMVs can be successfully decorated at high levels with the target antigens by directing them to the lipoprotein transport machinery.

In this study we investigated if D3 empty (i.e. not displaying any foreign peptides) and D3 displaying PorB Loop5 (as an exemplar epitope) could be successfully expressed in E. coli BL21 (DE3)ΔompAΔmsbBapagP OMVs, a strain with LPS detoxified and over-blebbing. A leader sequence was added to the N-terminus of the mature amino acid sequence of the D3 and D3-Loop5. A His-tag sequence was also added between the Leader sequence and the D3 starting first residue. OMV's were prepared according to the methods outlined above.

SDS-PAGE analysis of collected OMVs (see FIG. 12) confirmed the presence of a band at the correct MW of the target proteins which present only in the induced samples. Furthermore, Western blot data demonstrated that sera containing anti-D3 antibodies (see FIG. 13A) and anti-PorB antibodies (see FIG. 13B) successfully recognised D3 (FIG. 13A) and the PorB loop 5 epitope (FIG. 13.B) when expressed in engineered OMVs.

### DISCUSSION AND CONCLUSIONS

These results demonstrate the use of BP2a D3 as a scaffold for exogenous polypeptides. Several different chimeric polypeptides were produced recombinantly and in soluble form in E. coli. Biochemical characterization of the chimeric polypeptides revealed that they can accommodate large exogenous polypeptides while maintaining structure and correct folding of both the overall scaffold itself and the exogenous polypeptides inserted therein. Moreover, mouse immunization with a chimeric polypeptide comprising an exogenous polypeptide from N. gonorrhoeae yielded functional antibodies that were able to specifically recognize native proteins in N. gonorrhoeae total extract as well as N. gonorrhoeae OMVs. Furthermore, the D3 scaffold (both on its own and containing exogenous polypeptides) can be displayed using nanoparticles and OMV's as vaccine platforms.

1. A chimeric polypeptide comprising:
(i) a scaffold polypeptide; and
(ii) one or more exogenous polypeptide(s),
wherein the scaffold polypeptide comprises a backbone protein 2a (BP-2a) Domain 3 (D3) polypeptide, wherein in the chimeric polypeptide at least one endogenous loop of the BP-2a D3 polypeptide is partially or wholly replaced by an exogenous polypeptide.

2. The chimeric polypeptide of claim 1, wherein the at least one endogenous loop of the BP-2a D3 polypeptide is selected from the first, second, third, fourth, fifth and sixth endogenous loops of the BP-2a D3 polypeptide, wherein:
(i) the first endogenous loop of the BP-2a D3 polypeptide spans the amino acids at positions corresponding to positions 360-372 of SEQ ID NO:1;
(ii) the second endogenous loop of the BP-2a D3 polypeptide spans the amino acids at positions corresponding to positions 380-384 of SEQ ID NO:1;
(iii) the third endogenous loop of the BP-2a D3 polypeptide spans the amino acids at positions corresponding to positions 393-399 of SEQ ID NO:1;
(iv) the fourth endogenous loop of the BP-2a D3 polypeptide spans the amino acids at positions corresponding to positions 404-411 of SEQ ID NO:1;
(v) the fifth endogenous loop of the BP-2a D3 polypeptide spans the amino acids at positions corresponding to positions 418-422 of SEQ ID NO:1; and
(vi) the sixth endogenous loop of the BP-2a D3 polypeptide spans the amino acids at positions corresponding to positions 429-432 of SEQ ID NO:1,
wherein the amino acid positions are numbered according to the sequence shown in SEQ ID NO:1, wherein amino acids 332-447 are the native BP-2a D3 polypeptide.

3. The chimeric polypeptide of claim 1, wherein the BP-2a D3 polypeptide comprises seven scaffold regions, wherein:
(i) the first scaffold region of the BP-2a D3 polypeptide spans the amino acids at positions corresponding to positions 332-359 of SEQ ID NO:1;
(ii) the second scaffold region of the BP-2a D3 polypeptide spans the amino acids at positions corresponding to positions 373-379 of SEQ ID NO:1;
(iii) the third scaffold region of the BP-2a D3 polypeptide spans the amino acids at positions corresponding to positions 385-392 of SEQ ID NO:1;
(iv) the fourth scaffold region of the BP-2a D3 polypeptide spans the amino acids at positions corresponding to positions 400-403 of SEQ ID NO:1;
(v) the fifth scaffold region of the BP-2a D3 polypeptide spans the amino acids at positions corresponding to positions 412-417 of SEQ ID NO:1;
(vi) the sixth scaffold region spans the amino acids at positions corresponding to positions 423-428 of SEQ ID NO:1; and
(vii) the seventh scaffold region of the BP-2a D3 polypeptide spans the amino acids at positions corresponding to positions 433-447 of SEQ ID NO:1,
wherein the amino acids are numbered according to the sequence shown in SEQ ID NO:1, wherein amino acids 332-447 are the native BP-2a D3 polypeptide.

4. The chimeric polypeptide of claim 1, wherein the BP-2a D3 polypeptide is at least 40%, at least 50%, at least 60%, at least 70%, at least 80%, at least 90%, at least 95%, at least 97%, at least 98%, at least 99% or 100% identical to the amino acid sequence shown in SEQ ID NO:2; SEQ ID NO:101; SEQ ID NO:116; SEQ ID NO:131; SEQ ID NO:146; or SEQ ID NO:161, but for the one or more exogenous polypeptides replacing in whole or in part at least one of the endogenous loops.

5. (canceled)

6. (canceled)

7. The chimeric polypeptide of claim 1, wherein the BP-2a D3 polypeptide comprises seven scaffold regions, wherein:
(i) the first scaffold region is at least 40%, at least 50%, at least 60%, at least 70%, at least 80%, at least 90%, at least 95%, at least 97%, at least 98%, or at least 99% identical to the amino acid sequence shown in SEQ ID NO:10;
(ii) the second scaffold region is at least 40%, at least 50%, at least 60%, at least 70%, at least 80%, at least 90%, at least 95%, at least 97%, at least 98%, or at least 99% identical to the amino acid sequence shown in SEQ ID NO:11;
(iii) the third scaffold region is at least 40%, at least 50%, at least 60%, at least 70%, at least 80%, at least 90%, at least 95%, at least 97%, at least 98%, or at least 99% identical to the amino acid sequence shown in SEQ ID NO:12;
(iv) the fourth scaffold region is at least 40%, at least 50%, at least 60%, at least 70%, at least 80%, at least 90%, at least 95%, at least 97%, at least 98%, or at least 99% identical to the amino acid sequence shown in SEQ ID NO:13;
(v) the fifth scaffold region is at least 40%, at least 50%, at least 60%, at least 70%, at least 80%, at least 90%, at least 95%, at least 97%, at least 98%, or at least 99% identical to the amino acid sequence shown in SEQ ID NO:14;
(vi) the sixth scaffold region is at least 40%, at least 50%, at least 60%, at least 70%, at least 80%, at least 90%, at least 95%, at least 97%, at least 98%, or at least 99% identical to the amino acid sequence shown in SEQ ID NO:15; and
(vii) the seventh scaffold region is at least 40%, at least 50%, at least 60%, at least 70%, at least 80%, at least 90%, at least 95%, at least 97%, at least 98%, or at least 99% identical to the amino acid sequence shown in SEQ ID NO:16,
wherein:
(viii) the first scaffold region comprises the amino acid sequence shown in SEQ ID NO:10;
(ix) the second scaffold region comprises the amino acid sequence shown in SEQ ID NO:11;
(x) the third scaffold region comprises the amino acid sequence shown in SEQ ID NO:12;
(xi) the fourth scaffold region comprises the amino acid sequence shown in SEQ ID NO:13;
(xii) the fifth scaffold region comprises the amino acid sequence shown in SEQ ID NO:14;
(xiii) the sixth scaffold region comprises the amino acid sequence shown in SEQ ID NO:15; and
(xiv) the seventh scaffold region comprises the amino acid sequence shown in SEQ ID NO:16.

8. The chimeric polypeptide of claim 3, wherein the at least one endogenous loop of the BP-2a D3 polypeptide partially or wholly replaced with an exogenous polypeptide is selected from the first, second, third, fifth and sixth endogenous loops, preferably wherein the second endogenous loop of the BP-2a D3 polypeptide is partially or wholly replaced by an exogenous polypeptide.

9. The chimeric polypeptide of claim 3, wherein, when present:
(i) the first endogenous loop of the BP-2a D3 polypeptide is at least 40%, at least 50%, at least 60%, at least 70%, at least 80%, at least 90%, at least 95%, at least 97%, at least 98%, or at least 99% to the amino acid sequence shown in SEQ ID NO:4, SEQ ID NO:102, SEQ ID NO:117, SEQ ID NO:132, SEQ ID NO:147, or SEQ ID NO:162,
(ii) the second endogenous loop of the BP-2a D3 polypeptide is at least 40%, at least 50%, at least 60%, at least 70%, at least 80%, at least 90%, at least 95%, at least 97%, at least 98%, or at least 99% identical to the amino acid sequence shown in SEQ ID NO:5, SEQ ID NO:103, SEQ ID NO:118, SEQ ID NO:133, SEQ ID NO:148, or SEQ ID NO:163,
(iii) the third endogenous loop of the BP-2a D3 polypeptide is at least 40%, at least 50%, at least 60%, at least 70%, at least 80%, at least 90%, at least 95%, at least 97%, at least 98%, or at least 99% identical to the amino acid sequence shown in SEQ ID NO:6, SEQ ID NO:104, SEQ ID NO:119, SEQ ID NO:134, SEQ ID NO:149, or SEQ ID NO:164,
(iv) the fourth endogenous loop of the BP-2a D3 polypeptide is at least 40%, at least 50%, at least 60%, at least 70%, at least 80%, at least 90%, at least 95%, at least 97%, at least 98%, or at least 99% identical to the amino acid sequence shown in SEQ ID NO:7, SEQ ID NO:105, SEQ ID NO:120, SEQ ID NO:135, SEQ ID NO:150, or SEQ ID NO:165,
(v) the fifth endogenous loop of the BP-2a D3 polypeptide is at least 40%, at least 50%, at least 60%, at least 70%, at least 80%, at least 90%, at least 95%, at least 97%, at least 98%, or at least 99% identical to the amino acid sequence shown in SEQ ID NO:8, SEQ ID NO:106, SEQ ID NO:121, SEQ ID NO:136, SEQ ID NO:151, or SEQ ID NO:166 and
(vi) the sixth endogenous loop of the BP-2a D3 polypeptide is at least 40%, at least 50%, at least 60%, at least 70%, at least 80%, at least 90%, at least 95%, at least 97%, at least 98%, or at least 99% identical to the amino acid sequence shown in SEQ ID NO:9, SEQ ID NO:107, SEQ ID NO:122, SEQ ID NO:137, SEQ ID NO:152, or SEQ ID NO:167,
wherein, when present:
(vii) the first endogenous loop of the BP-2a D3 polypeptide comprises the amino acid sequence shown in SEQ ID NO:4; SEQ ID NO:102, SEQ ID NO:117, SEQ ID NO:132, SEQ ID NO:147, or SEQ ID NO:162,
(viii) the second endogenous loop of the BP-2a D3 polypeptide comprises the amino acid sequence shown in SEQ ID NO:5; SEQ ID NO:103, SEQ ID NO:118, SEQ ID NO:133, SEQ ID NO:148, or SEQ ID NO:163,
(ix) the third endogenous loop of the BP-2a D3 polypeptide comprises the amino acid sequence shown in SEQ ID NO:6; SEQ ID NO:104, SEQ ID NO:119, SEQ ID NO:134, SEQ ID NO:149, or SEQ ID NO:164,
(x) the fourth endogenous loop of the BP-2a D3 polypeptide comprises the amino acid sequence shown in SEQ ID NO:7; SEQ ID NO:105, SEQ ID NO:120, SEQ ID NO:135, SEQ ID NO:150, or SEQ ID NO:165,
(xi) the fifth endogenous loop of the BP-2a D3 polypeptide comprises the amino acid sequence shown in SEQ ID NO:8; SEQ ID NO:106, SEQ ID NO:121, SEQ ID NO:136, SEQ ID NO:151, or SEQ ID NO:166 and
(xii) the sixth endogenous loop of the BP-2a D3 polypeptide comprises the amino acid sequence shown in SEQ ID NO:9, SEQ ID NO:107, SEQ ID NO:122, SEQ ID NO:137, SEQ ID NO:152, or SEQ ID NO:167.

10. The chimeric polypeptide of claim 3, wherein the fourth endogenous loop of the BP-2a D3 polypeptide is present and is at least 40%, at least 50%, at least 60%, at least 70%, at least 80%, at least 90%, at least 95%, at least 97%, at least 98%, or at least 99% identical to the amino acid sequence shown in SEQ ID NO:7, SEQ ID NO:105, SEQ ID NO:120, SEQ ID NO:135, SEQ ID NO:150, or SEQ ID NO:165, wherein the fourth endogenous loop of the BP-2a D3 polypeptide is present and comprises or consists of the amino acid sequence shown in SEQ ID NO:7, SEQ ID NO:105, SEQ ID NO:120, SEQ ID NO:135, SEQ ID NO:150, or SEQ ID NO:165.

11. The chimeric polypeptide of claim 1, wherein the exogenous polypeptide comprises an antigenic fragment of a target protein, wherein the target protein is a membrane protein from a microorganism or virus.

12. (canceled)

13. The chimeric polypeptide of claim 1, comprising one or more exogenous polypeptide(s) each independently comprising an amino acid sequence selected from: SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:51, SEQ ID NO:52, SEQ ID NO:53, SEQ ID NO:182, SEQ ID NO:183, SEQ ID NO: 195, SEQ ID NO: 197, SEQ ID NO: 198, SEQ ID NO: 199 and SEQ ID NO: 200.

14. The chimeric polypeptide of claim 1 comprising an amino acid sequence selected from: SEQ ID NO:54, SEQ ID NO:55, SEQ ID NO:56, SEQ ID NO:57, SEQ ID NO:58, SEQ ID NO:59, SEQ ID NO:60, SEQ ID NO:61, SEQ ID NO:62, SEQ ID NO:63, SEQ ID NO:64, SEQ ID NO:65, SEQ ID NO:66, SEQ ID NO:67, SEQ ID NO:68, SEQ ID NO:69, SEQ ID NO:70, SEQ ID NO:71, SEQ ID NO:72, SEQ ID NO:73, SEQ ID NO:74, SEQ ID NO:75, SEQ ID NO:76, SEQ ID NO:77, SEQ ID NO:78, SEQ ID NO:79, SEQ ID NO:80, SEQ ID NO:81, SEQ ID NO:82, SEQ ID NO:83, SEQ ID NO:84, SEQ ID NO:85, SEQ ID NO:86, SEQ ID NO:87, SEQ ID NO:88, SEQ ID NO:89, SEQ ID NO:90, SEQ ID NO:91, SEQ ID NO:92, SEQ ID NO:93, SEQ ID NO:94, SEQ ID NO:95, SEQ ID NO:96, SEQ ID NO:97, SEQ ID NO:184, SEQ ID NO:185, SEQ ID NO:186, SEQ ID NO: 190, SEQ ID NO: 191, SEQ ID NO: 192, SEQ ID NO: 193, SEQ ID NO: 194 and SEQ ID NO: 196.

15. The chimeric polypeptide of claim 1, wherein the BP-2a D3 polypeptide is from Streptococcus agalactiae.

16. A nanoparticle comprising a chimeric polypeptide according to claim 1.

17. An outer membrane vesicle comprising a chimeric polypeptide according to claim 1, wherein the chimeric polypeptide is expressed on the surface of the outer membrane vesicle.

18. An isolated polynucleotide encoding the chimeric polypeptide of claim 1.

19. An expression vector comprising the polynucleotide of claim 18 operably linked to regulatory sequences which permit expression of the chimeric polypeptide or nanoparticle.

20. A host cell or cell-free expression system containing the expression vector of claim 19.

21. A method of producing a chimeric polypeptide or nanoparticle comprising culturing the host cell or cell-free expression system of claim 20 under conditions which permit expression of chimeric polypeptide or nanoparticle and recovering the expressed chimeric polypeptide or nanoparticle.

22. A pharmaceutical composition comprising a chimeric polypeptide according to claim 1, and at least one pharmaceutically acceptable adjuvant, carrier or excipient.

23. (canceled)

24. A method for raising an immune response in a mammal, comprising administering a chimeric polypeptide of claim 1.

25. (canceled)

26. (canceled)
